(19) United States
(12) Patent Application Publication
(10) Pub. No.: US 2005/0191654 A1
(43) Pub. Date: Sep. 1, 2005

(54) Compositions and methods for diagnosing and treating chronic lymphocytic leukemia

(76) Inventors: Daniela Drandi, Torino (IT); John Gerard Gribben, London (GB); Charles Lee, Marlborough, MA (US); Paola Dal Cin, Brookline, MA (US)

Correspondence Address: FISH & RICHARDSON PC, 225 FRANKLIN ST, BOSTON, MA, 02110, US

(21) Appl. No.: 10/984,723

(22) Filed: Nov. 8, 2004

Related U.S. Application Data
(60) Provisional application No. 60/518,118, filed on Nov. 6, 2003.

(51) Int. Cl. C12Q 1/68; C07H 21/04

(52) U.S. Cl. 435/6; 536/24.3

(57) ABSTRACT

The invention features compositions and methods for diagnosing and treating chronic lymphocytic leukemia (CLL) based upon the absence or presence of a lesion, e.g., a deletion, at chromosome 14q32.33.

This application claims the benefit of the filing date of U.S. Ser. No. 60/518,118, filed Nov. 6, 2003. For the purpose of any United States patent that may issue from the present application, the entire content of the prior provisional application is hereby incorporated by reference herein.

### BACKGROUND OF THE INVENTION

Each year, nearly 7,300 people in the United States learn that they have chronic lymphocytic leukemia (CLL).

CLL results from an acquired injury to the DNA of a single cell in the bone marrow. This change in the cell's DNA confers a growth and survival advantage on the cell, which becomes abnormal and malignant (leukemic). The result of this injury is the uncontrolled growth of lymphocytic cells in the marrow leading invariably to an increase in the concentration of lymphocytes in the blood. The leukemic cells that accumulate in the marrow in CLL do not impede normal blood cell production as profoundly as in the case of acute lymphocytic leukemia (ALL). This important distinction from acute leukemia accounts for the less severe early course of the disease.

### SUMMARY OF THE INVENTION

The invention is based, in part, on the discovery of a deletion at 14q32.33 of chromosome 14 that correlates with poor prognosis in subjects having CLL. The outcome of being diagnosed with CLL can vary drastically among patients. Some patients can live for many years without therapy whereas other patients can rapidly progress and require treatment. The present invention allows for subjects having risk factors associated with CLL or having CLL, e.g., early or intermediate CLL, to be diagnosed with a risk of developing a more advanced stage of CLL. This can allow for treatment to be administered to these subjects prior to, or early on in, the development of the disease. The invention includes compositions and methods for diagnosing and treating CLL.

Accordingly, in one aspect, the invention features a nucleic acid probe, e.g., a DNA probe, that hybridizes with a nucleotide sequence within human chromosome 14, e.g., 14q32.33, e.g., within the V_{H} locus at 14q32.33, and detects a lesion, e.g., a deletion, e.g., a deletion of nucleotides, within 14q32.33, e.g., within the V_{H} locus.

In one embodiment, the nucleic acid probe, e.g., the DNA probe, hybridizes with a nucleotide sequence within 14q32.33 of chromosome 14, e.g., within the V_{H} locus of 14q32.33. In one embodiment, the lesion, e.g., the deletion, is within the V_{H} locus of 14q32.33.

In one embodiment, the nucleic acid probe, e.g., the DNA probe, hybridizes with a nucleotide sequence that is involved in the lesion, e.g., the deletion, e.g., the probe hybridizes with a nucleotide sequence that is deleted. In another embodiment, the probe hybridizes with a nucleotide sequence that is adjacent to the lesion (e.g., deletion), e.g., a nucleotide sequence that is 5′ or 3′ to the lesion, e.g., deletion. Alternatively, the nucleic acid probe, e.g., the DNA probe, can hybridize with a nucleotide sequence that includes nucleotides 5′ to the lesion and nucleotides 3′ to the lesion, wherein the probe does not hybridize to the nucleotide sequence in the absence of a lesion. For example, the nucleotides 5′ to the lesion and 3′ to the lesion are each insufficient for hybridization of the nucleic acid probe, and only in the presence of a lesion are the nucleotides 5′ to the lesion and 3′ to the lesion in proximity to provide a nucleotide sequence to which the nucleic acid probe hybridizes.

In one embodiment, the nucleic acid probe hybridizes with a fragment of DNA that differs in size or sequence from a fragment of DNA that does not include the lesion.

In one embodiment, the nucleic acid probe, e.g., DNA probe, is labeled, e.g., with a fluorescent label, e.g., 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, Cascade™ blue acetylazide (Molecular Probes, Inc., Eugene, Oreg.), cy3, cy3.5, cy7 (Amersham), DEAC, or Spectrum Aqua (Vysis).

Preferably, the nucleic acid probe is, e.g., at least 50 kb, e.g., at least 100 kb, e.g., at least 200 kb, e.g., at least 300 kb, e.g., at least 400 kb, e.g., at least 500 kb, at least 600 kb, e.g., at least 700 kb, e.g., at least 800 kb, e.g., at least 900 kb.

Preferably, the nucleic acid probe includes a nucleotide sequence having at least 70%, e.g., at least 80%, e.g., at least 85%, e.g., at least 90%, e.g., at least 95%, e.g., at least 98%, or more homology with SEQ ID NO:1.

In another embodiment, the nucleic acid probe includes the DNA sequence of SEQ ID NO:1. Preferably, the nucleic acid probe is labeled as described herein. Preferably, the nucleic acid probe is about 300 kb, e.g., about 350 kb, e.g., about 400 kb, e.g., about 500 kb, e.g., about 600 kb, e.g., about 700 kb, e.g., about 800 kb, e.g., about 900 kb, e.g., about 1,000 kb. Preferably, the nucleic acid probe has a nucleotide sequence that differs from SEQ ID NO:1 by at least, e.g., 1, 2, 3, 5, 10, 20, 30, but not more than, e.g., 40, 50, or 100, nucleotides. In another embodiment, the nucleic acid probe has the nucleic acid sequence of SEQ ID NO:1. Preferably, the nucleic acid probe has at least 75, e.g., at least 100, e.g., at least 150, e.g., at least 200, e.g., at least 250 contiguous nucleotides of SEQ ID NO:1. Preferably, the nucleic acid probe is labeled as described herein.

Another aspect of the invention features a kit including a nucleic acid probe that hybridizes with a nucleotide sequence within human chromosome 14, e.g., within 14q32.33, e.g., within the V_{H} locus at 14q32.33, and detects a lesion, e.g., a deletion, within 14q32.33, e.g., within the V_{H} locus, e.g., a nucleic acid probe described herein; and instructions for evaluating a subject's risk of CLL, or lymphoma, by determining the absence or presence of a chromosomal lesion occurring in the IgH locus, e.g., in the V_{H} locus. The evaluating can be, e.g., for prognosis, diagnosis, or assessment of risk. Preferably, the nucleic acid probe is labeled as described herein.

In one embodiment, the nucleic acid probe, e.g., the DNA probe, hybridizes with a nucleotide sequence that is involved in the lesion, e.g., the deletion, e.g., the probe hybridizes with a nucleotide sequence that is deleted. In another embodiment, the probe hybridizes with a nucleotide sequence that is adjacent to the lesion (e.g., deletion), e.g., a nucleotide sequence that is 5′ or 3′ to the lesion, e.g., deletion. Alternatively, the nucleic acid probe, e.g., the DNA probe, can hybridize with a nucleotide sequence that includes nucleotides 5′ to the lesion and nucleotides 3′ to the lesion, wherein the probe does not hybridize to the nucleotide sequence in the absence of a lesion. For example, the nucleotides 5′ to the lesion and 3′ to the lesion are each insufficient for hybridization of the nucleic acid probe, and only in the presence of a lesion are the nucleotides 5′ to the lesion and 3′ to the lesion in proximity to provide a nucleotide sequence to which the nucleic acid probe hybridizes.

In one embodiment, the probe is not labeled, and a label is included in the kit. Preferably, the kit contains instructions and/or reagents for labeling the probe.

In another embodiment, the kit contains at least one additional probe that hybridizes with 13q14 or a portion thereof, e.g., 13q14.3, of human chromosome 13. Preferably, the additional probe detects a deletion in this region of chromosome 13. Preferably, the additional probe is labeled, e.g., both the nucleic acid probe that hybridizes within human chromosome 14, e.g., within 14q32.33, e.g., within the V_{H} locus at 14q32.33,and the additional probe that hybridizes with 13q14 are labeled, e.g., are labeled with the same or different labels.

In another embodiment, the kit contains at least one additional probe that hybridizes with 11q22-23, or a portion thereof, e.g., 11q22.3, of human chromosome 11. Preferably, the additional probe detects a deletion in this region of chromosome 11. Preferably, the additional probe is labeled, e.g., both the nucleic acid probe that hybridizes within human chromosome 14, e.g., within 14q32.33, e.g., within the V_{H} locus at 14q32.33, and the additional probe that hybridizes with 11q22-23 are labeled, e.g., are labeled with the same or different labels.

In another embodiment, the kit contains at least one additional probe that hybridizes with 17p13, or a portion thereof, e.g., 17p13.1, of human chromosome 17. Preferably, the additional probe detects a deletion in this region of chromosome 17. Preferably, the additional probe is labeled, e.g., both the nucleic acid probe that hybridizes within human chromosome 14, e.g., within 14q32.33, e.g., within the V_{H} locus at 14q32.33, and the additional probe that hybridizes with 17p13 are labeled, e.g., are labeled with the same or different labels.

In another embodiment, the kit contains an additional probe that hybridizes with human chromosome 12, or a portion thereof. Preferably, the kit contains additional instructions that provide that the presence of more than two copies of chromosome 12, or a portion thereof, is indicative of a risk for CLL. Preferably, the additional probe is labeled, e.g., both the nucleic acid probe that hybridizes within human chromosome 14, e.g., within 14q32.33, e.g., within the V_{H} locus at 14q32.33, and the additional probe that hybridizes with chromosome 12 are labeled, e.g., are labeled with the same or different labels.

In another embodiment, the kit contains two or more additional probes that hybridize with a human chromosome or a portion thereof, such as 13q14, or a portion thereof, e.g., 13q14.3, of human chromosome 13; 11q22-23, or a portion thereof, e.g., 11q22.3, of human chromosome 11; 17p13, or a portion thereof, e.g., 17p13.1, of human chromosome 17; and human chromosome 12, or a portion thereof. Preferably, the additional probes are labeled, e.g., are labeled with the same or different labels.

Another aspect of the invention features a primer capable of amplifying at least a portion of human chromosome 14, e.g., at least a portion of 14q32.33, e.g., at least a portion of the V_{H} locus at 14q32.33.

In one embodiment, the primer amplifies at least a portion of 14q32.33, e.g., a portion of the V_{H} locus at 14q32.33, that can include a lesion, e.g., a deletion associated with a risk of leukemia, e.g., CLL, or lymphoma, e.g., a risk of intermediate or aggressive CLL, or lymphoma.

In one embodiment, the primer hybridizes with a nucleotide sequence that is adjacent to the lesion (e.g., deletion), e.g., a nucleotide sequence that is 5′ or 3′ to the lesion, e.g., deletion. Alternatively, the primer can hybridize with a nucleotide sequence that includes nucleotides 5′ to the lesion and nucleotides 3′ to the lesion, wherein the primer does not hybridize to the nucleotide sequence in the absence of a lesion. For example, the nucleotides 5′ to the lesion and 3′ to the lesion are each insufficient for hybridization of the primer, and only in the presence of a lesion are the nucleotides 5′ to the lesion and 3′ to the lesion in proximity to provide a nucleotide sequence to which the primer hybridizes.

In one embodiment, the primer amplifies a fragment of DNA that differs in size or sequence from a fragment of DNA that does not include the lesion.

In one embodiment, the primer is at least 10, e.g., at least 12, e.g., at least 15, e.g., at least 20, e.g., at least 30, e.g., at least 50, e.g., at least 100 nucleotides in length.

In another embodiment, the primer includes at least 10, e.g., at least 20, e.g., at least 50, e.g., at least 100 consecutive nucleotides of SEQ ID NO:1.

In another embodiment, the primer differs from SEQ ID NO:1 by at least 1, 2, 3, or 5, but not more than, e.g., 10, 20, or 50 nucleotides.

Another aspect of the invention features a method of evaluating a subject, e.g., a subject at risk for or having leukemia, e.g., chronic lymphocytic leukemia, e.g., intermediate or aggressive CLL, or lymphoma, including providing a primer or set of primers described herein; amplifying all or a portion of human chromosome 14, e.g., 14q32.33, e.g., the V_{H} locus at 14q32.33, that can include a lesion, e.g., a deletion; and detecting the absence or presence of the lesion, e.g., the deletion, to thereby determine if the subject is at risk for CLL or lymphoma, e.g., at risk for intermediate or aggressive CLL, or lymphoma. The evaluating can be, e.g., for prognosis, diagnosis, or assessment of risk.

Another aspect of the invention features an array of a plurality of capture probes, wherein each of the capture probes is positionally distinguishable from other capture probes of the plurality on the array, wherein at least one capture probe hybridizes specifically with human chromosome 14, e.g., with 14q32.33, e.g., with the V_{H} locus at 14q32.33. Preferably, the capture probes are nucleic acid probes described herein.

In one embodiment, the capture probe that hybridizes with 14q32.33, e.g., with the V_{H} locus at 14q32.33, detects a lesion, e.g., a deletion, within 14q32.33, e.g., within the V_{H} locus at 14q32.33.

In another embodiment, the array further includes at least one additional capture probe that hybridizes to 13q14, or a portion thereof, e.g., 13q14.3, of human chromosome 13. Preferably, the additional capture probe detects a deletion within this portion of chromosome 13. Preferably, the additional capture probe is a probe described herein.

In another embodiment, the array further includes at least one additional capture probe that hybridizes to 11q22-23, or a portion thereof, e.g., 11q22.3, of human chromosome 11. Preferably, the additional capture probe detects a deletion within this portion of chromosome 11. Preferably, the additional capture probe is a probe described herein.

In another embodiment, the array further includes at least one additional capture probe that hybridizes to 17p13, or a portion thereof, e.g., 17p13.1, of human chromosome 17. Preferably, the additional capture probe detects a deletion within this portion of chromosome 17. Preferably, the additional capture probe is a probe described herein.

In another embodiment, the array further includes at least one additional capture probe that hybridizes to human chromosome 12, or a portion thereof. Preferably, the additional capture probe detects the presence of more than two copies of chromosome 12, or portion thereof. Preferably, the additional capture probe is a probe described herein.

In another embodiment, the array further includes at least two additional capture probes that specifically hybridize with a human chromosome, or portion thereof, such as 13q14, or a portion thereof, e.g., 13q14.3, of human chromosome 13; 11q22-23, or a portion thereof, e.g., 11q22.3, of human chromosome 11; chromosome 17p13, or a portion thereof, e.g., 17p13.1, of human chromosome 17; and human chromosome 12, or a portion thereof. Preferably, the additional capture probes are probes described herein.

In another embodiment, the capture probe is a probe that hybridizes with human chromosome 14, e.g., with 14q32.33, e.g., with the V_{H} locus at 14q32.33 of human chromosome 14, described herein.

Another aspect of the invention features a method of evaluating a subject, e.g., a subject at risk for or having leukemia, e.g., chronic lymphocytic leukemia, e.g., intermediate or aggressive CLL, or lymphoma, including providing an array described herein; detecting the absence or presence of a lesion, e.g., a deletion, within 14q32.33, e.g., within the V_{H} locus at 14q32.33; and thereby determining if the subject is at risk for CLL or lymphoma, e.g., at risk for intermediate or aggressive CLL, or lymphoma. The evaluating can be, e.g., for prognosis, diagnosis, or assessment of risk.

Another aspect of the invention features a method of evaluating a subject, e.g., a subject at risk for or having leukemia, e.g., CLL, or lymphoma. The method includes providing a sample of genomic DNA from the subject and determining the absence or presence of a lesion, e.g., a deletion, within human chromosome 14, e.g., within 14q32.33, e.g., within the V_{H} locus of 14q32.33. The evaluating can be, e.g., for prognosis, diagnosis, or assessment of risk.

In one embodiment, the absence or presence of a lesion, e.g., a deletion, within 14q32.33, e.g., within the V_{H} locus at 14q32.33, is determined using a nucleic acid probe, e.g., a DNA probe, that hybridizes with 14q32.33, e.g., with the V_{H} locus at 14q32.33, e.g., a nucleic acid probe described herein.

In one embodiment, the nucleic acid probe, e.g., the DNA probe, hybridizes with a nucleotide sequence within 14q32.33 of chromosome 14, e.g., within the V_{H} locus of 14q32.33. In one embodiment, the lesion, e.g., the deletion, is within the V_{H} locus of 14q32.33.

In one embodiment, the nucleic acid probe, e.g., the DNA probe, hybridizes with a nucleotide sequence that is involved in the lesion, e.g., the deletion, e.g., the probe hybridizes with a nucleotide sequence that is deleted. In another embodiment, the probe hybridizes with a nucleotide sequence that is adjacent to the lesion (e.g., deletion), e.g., a nucleotide sequence that is 5′ or 3′ to the lesion, e.g., deletion. Alternatively, the nucleic acid probe, e.g., the DNA probe, can hybridize with a nucleotide sequence that includes nucleotides 5′ to the lesion and nucleotides 3′ to the lesion, wherein the probe does not hybridize to the nucleotide sequence in the absence of a lesion. For example, the nucleotides 5′ to the lesion and 3′ to the lesion are each insufficient for hybridization of the nucleic acid probe, and only in the presence of a lesion are the nucleotides 5′ to the lesion and 3′ to the lesion in proximity to provide a nucleotide sequence to which the nucleic acid probe hybridizes.

In one embodiment, the nucleic acid probe hybridizes with a fragment of DNA that differs in size or sequence from a fragment of DNA that does not include the lesion.

Preferably, the probe is labeled, e.g., with a fluorescent label, e.g., 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, or Cascade™ blue acetylazide (Molecular Probes, Inc., Eugene, Oreg.), cy3, cy3.5, cy7 (Amersham), DEAC, or Spectrum Aqua (Vysis).

In another embodiment, the presence of a deletion within 14q32.33, e.g., within the V_{H} locus at 14q32.33, indicates a risk of CLL or lymphoma, and the absence of a deletion within 14q32.33, e.g., within the V_{H} locus at 14q32.33, is indicative that the subject is not at risk of developing CLL or lymphoma.

In another embodiment, the method further includes determining the absence or presence of a lesion, e.g., a deletion, within 13q14, or a portion thereof, e.g., 13q14.3, of human chromosome 13. Preferably, the absence or presence of a lesion, e.g., a deletion, at 13q14, or a portion thereof, e.g., 13q14.3, is determined using at least one additional probe that hybridizes with 13q14, or a portion thereof, e.g., 13q14.3. Preferably, the additional probe detects a lesion, e.g., a deletion, in this region of chromosome 13. The probe can be, e.g., a nucleic acid probe that hybridizes with 13q14, or a portion thereof, e.g., 13q14.3, described herein. Preferably, the additional probe is labeled, e.g., both the DNA probe that hybridizes with 14q32.33, e.g., with the V_{H} locus at 14q32.33, and the additional probe that hybridizes with 13q14 are labeled, e.g., are labeled with the same or different labels.

In another embodiment, the method further includes determining the absence or presence of a lesion, e.g., a deletion, within 11q22-23, or a portion thereof, e.g., 11q22.3, of human chromosome 11. Preferably, the absence or presence of a lesion, e.g., a deletion, at 11q22-23, or a portion thereof, e.g., 11q22.3, is determined using at least one additional probe that hybridizes with 11q22-23, or a portion thereof, e.g., 11q22.3. Preferably, the additional probe detects a lesion, e.g., a deletion, in this region of chromosome 11. The probe can be, e.g., a nucleic acid probe that hybridizes with 11q22-23, or a portion thereof, e.g., 11q22.3, described herein. Preferably, the additional probe is labeled, e.g., both the DNA probe that hybridizes with 14q32.33, e.g., with the V_{H} locus at 14q32.33, and the additional probe that hybridizes with 11q22-23 are labeled, e.g., are labeled with the same or different labels.

In another embodiment, the method further includes determining the absence or presence of a lesion, e.g., a deletion, within 17p13, or a portion thereof, e.g., 17p13.1, of human chromosome 17. Preferably, the absence or presence of a lesion, e.g., a deletion, at 17p13, or a portion thereof, e.g., 17p13.1, is determined using at least one additional probe that hybridizes with 17p13, or a portion thereof, e.g., 17p13.1. Preferably, the additional probe detects a lesion, e.g., a deletion, in this region of chromosome 17. The probe can be, e.g., a nucleic acid probe that hybridizes with 17p13, or a portion thereof, e.g., 17p13.1, described herein. Preferably, the additional probe is labeled, e.g., both the DNA probe that hybridizes with 14q32.33, e.g., with the V_{H} locus at 14q32.33, and the additional probe that hybridizes with 17p13 are labeled, e.g., are labeled with the same or different labels.

In another embodiment, the method further includes determining the absence or presence of more than two copies of human chromosome 12, or portion thereof. Preferably, the absence or presence of more than two copies of chromosome 12, or portion thereof, is determined using at least one additional probe that hybridizes with human chromosome 12, or a portion thereof. Preferably, the additional probe detects more than two copies of chromosome 12, or a portion thereof. The probe can be, e.g., a nucleic acid probe that hybridizes with human chromosome 12, or a portion thereof, described herein. Preferably, the additional probe is labeled, e.g., both the DNA probe that hybridizes with 14q32.33, e.g., with the V_{H} locus at 14q32.33, and the additional probe that hybridizes with chromosome 12 are labeled, e.g., are labeled with the same or different labels.

In another embodiment, the method further includes amplifying a portion of human chromosome 14, e.g., a portion of 14q32.33, e.g., a portion of the V_{H} locus at 14q32.33, that can include a lesion, e.g., a deletion, in this region of human chromosome 14, e.g., within 14q32.33, e.g., within the V_{H} locus at 14q32.33, from the genomic DNA. Preferably, the portion of 14q32.33, e.g., the portion of the V_{H} locus at 14q32.33, that includes the lesion, e.g., the deletion, is amplified using a primer, e.g., a primer described herein.

In one embodiment, the primer hybridizes with a nucleotide sequence that is adjacent to the lesion (e.g., deletion), e.g., a nucleotide sequence that is 5′ or 3′ to the lesion, e.g., deletion. Alternatively, the primer can hybridize with a nucleotide sequence that includes nucleotides 5′ to the lesion and nucleotides 3′ to the lesion, wherein the primer does not hybridize to the nucleotide sequence in the absence of a lesion. For example, the nucleotides 5′ to the lesion and 3′ to the lesion are each insufficient for hybridization of the primer, and only in the presence of a lesion are the nucleotides 5′ to the lesion and 3′ to the lesion in proximity to provide a nucleotide sequence to which the primer hybridizes.

In one embodiment, the primer amplifies a fragment of DNA that differs in size or sequence from a fragment of DNA that does not include the lesion.

In one embodiment, the primer is at least 10, e.g., at least 12, e.g., at least 15, e.g., at least 20, e.g., at least 30, e.g., at least 50, e.g., at least 100 nucleotides in length.

In another embodiment, the primer includes at least 10, e.g., at least 20, e.g., at least 50, e.g., at least 100 consecutive nucleotides of SEQ ID NO:1.

In another embodiment, the primer differs from SEQ ID NO:1 by at least 1, 2, 3, or 5, but not more than, e.g., 10, 20, or 50 nucleotides.

In another embodiment, the subject has a lesion, e.g., a deletion, within one or more of 13q14, or portion thereof, e.g., 13q14.3, of human chromosome 13; 11q22-23, or portion thereof, of human chromosome 11; 17p13, or portion thereof, e.g., 17p13.1, of human chromosome 17; and/or has more than two copies of human chromosome 12, or portion thereof.

In another embodiment, an array is used to determine the absence or presence of a lesion, e.g., a deletion, within human chromosome 14, e.g., within 14q32.33, e.g., within the V_{H} locus of 14q32.33. Preferably, the array is an array described herein that includes at least one capture probe that hybridizes specifically with 14q32.33, e.g., with the V_{H} locus at 14q32.33, or the array is a bacterial artificial chromosome (BAC) array, e.g., a BAC array described herein.

In another embodiment, the method further includes providing a result of the determination to a third party, e.g., a hospital, clinic, government entity, reimbursing party or insurance company. In one embodiment, payment for a therapeutic treatment or modality, e.g., a therapeutic treatment or modality described herein, or payment by a reimbursing party, e.g., a government entity or insurance company, is conditional on the outcome of the determination.

Another aspect of the invention features a method of evaluating a subject, e.g., a subject at risk for or having intermediate or aggressive CLL, or lymphoma. The evaluating can be, e.g., for prognosis, diagnosis, or assessment of risk. The method includes providing a sample of genomic DNA from the subject and determining the absence or presence of a lesion, e.g., a deletion, within human chromosome 14, e.g., within 14q32.33, e.g., within the V_{H} locus of 14q32.33.

In one embodiment, the presence of a lesion, e.g., a deletion, within 14q32.33, e.g., within the V_{H} locus at 14q32.33, of the subject is indicative of a risk for developing intermediate or aggressive CLL or lymphoma.

In another embodiment, the absence or presence of a lesion, e.g., a deletion, within 14q32.33, e.g., within the V_{H} locus at 14q32.33, is determined using a probe that hybridizes with 14q32.33, e.g., with the V_{H} locus at 14q32.33, e.g., a nucleic acid probe described herein. In one embodiment, the nucleic acid probe, e.g., the DNA probe, hybridizes with a nucleotide sequence within 14q32.33 of chromosome 14, e.g., within the V_{H} locus of 14q32.33. In one embodiment, the lesion, e.g., the deletion, is within the V_{H} locus of 14q32.33.

In one embodiment, the nucleic acid probe, e.g., the DNA probe, hybridizes with a nucleotide sequence that is involved in the lesion, e.g., the deletion, e.g., the probe hybridizes with a nucleotide sequence that is deleted. In another embodiment, the probe hybridizes with a nucleotide sequence that is adjacent to the lesion (e.g., deletion), e.g., a nucleotide sequence that is 5′ or 3′ to the lesion, e.g., deletion. Alternatively, the nucleic acid probe, e.g., the DNA probe, can hybridize with a nucleotide sequence that includes nucleotides 5′ to the lesion and nucleotides 3′ to the lesion, wherein the probe does not hybridize to the nucleotide sequence in the absence of a lesion. For example, the nucleotides 5′ to the lesion and 3′ to the lesion are each insufficient for hybridization of the nucleic acid probe, and only in the presence of a lesion are the nucleotides 5′ to the lesion and 3′ to the lesion in proximity to provide a nucleotide sequence to which the nucleic acid probe hybridizes.

In one embodiment, the nucleic acid probe hybridizes with a fragment of DNA that differs in size or sequence from a fragment of DNA that does not include the lesion.

Preferably, the probe is labeled, e.g., with a fluorescent label, e.g., 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyan ate, erythrosin-5-isothiocyanate, or Cascade™ blue acetylazide (Molecular Probes, Inc., Eugene, Oreg.), cy3, cy3.5, cy7 (Amersham), DEAC, or Spectrum Aqua (Vysis).

In another embodiment, the method further includes determining the absence or presence of a lesion, e.g., a deletion, within 13q14, or a portion thereof, e.g., 13q14.3, of human chromosome 13. Preferably, the absence or presence of a lesion, e.g., a deletion, at 13q14, or a portion thereof, e.g., 13q14.3, is determined using at least one additional probe that hybridizes with 13q14, or a portion thereof, e.g., 13q14.3. Preferably, the additional probe detects a lesion, e.g., a deletion, in this region of chromosome 13. The probe can be, e.g., a nucleic acid probe that hybridizes with 13q14, or a portion thereof, e.g., 13q14.3, described herein. Preferably, the additional probe is labeled, e.g., both the nucleic acid probe that hybridizes with 14q32.33, e.g., with the V_{H} locus at 14q32.33, and the additional probe that hybridizes with 13q14 are labeled, e.g., are labeled with the same or different labels.

In another embodiment, the method further includes determining the absence or presence of a lesion, e.g., a deletion, within 11q22-23, or a portion thereof, e.g., 11q22.3, of human chromosome 11. Preferably, the absence or presence of a lesion, e.g., a deletion, at 11q22-23, or a portion thereof, e.g., 11q22.3, is determined using at least one additional probe that hybridizes with 11q22-23, or a portion thereof, e.g., 11q22.3. Preferably, the additional probe detects a lesion, e.g., a deletion, in this region of chromosome 11. The probe can be, e.g., a nucleic acid probe that hybridizes with 11q22-23, or a portion thereof, e.g., 11q22.3, described herein. Preferably, the additional probe is labeled, e.g., both the nucleic acid probe that hybridizes with 14q32.33, e.g., with the V_{H} locus at 14q32.33, and the additional probe that hybridizes with 11q22-23 are labeled, e.g., are labeled with the same or different labels.

In another embodiment, the method further includes determining the absence or presence of a lesion, e.g., a deletion, within 17p13, or a portion thereof, e.g., 17p13.1, of human chromosome 17. Preferably, the absence or presence of a lesion, e.g., a deletion, at 17p13, or a portion thereof, e.g., 17p13.1, is determined using at least one additional probe that hybridizes with 17p13, or a portion thereof, e.g., 17p13.1. Preferably, the additional probe detects a lesion, e.g., a deletion, in this region of chromosome 17. The probe can be, e.g., a probe that hybridizes with 17p13, or a portion thereof, e.g., 17p13.1, described herein. Preferably, the additional probe is labeled, e.g., both the nucleic acid probe that hybridizes with 14q32.33, e.g., with the V_{H} locus at 14q32.33, and the additional probe that hybridizes with 17p13 are labeled, e.g., are labeled with the same or different labels.

In another embodiment, the method further includes determining the absence or presence of more than two copies of human chromosome 12, or portion thereof. Preferably, the absence or presence of more than two copies of chromosome 12, or portion thereof, is determined using at least one additional probe that hybridizes with human chromosome 12, or a portion thereof. Preferably, the additional probe detects more than two copies of chromosome 12, or a portion thereof. The probe can be, e.g., a nucleic acid probe that hybridizes with human chromosome 12, or a portion thereof, described herein. Preferably, the additional probe is labeled, e.g., both the nucleic acid probe that hybridizes with 14q32.33, e.g., with the V_{H} locus at 14q32.33, and the additional probe that hybridizes with chromosome 12 are labeled, e.g., are labeled with the same or different labels.

In another embodiment, the method further includes amplifying a portion of human chromosome 14, e.g., a portion of 14q32.33, e.g., a portion of the V_{H} locus at 14q32.33, that can include a lesion, e.g., a deletion, in this region of human chromosome 14, from the genomic DNA. Preferably, the portion of 14q32.33, e.g., the portion of V_{H} locus at 14q32.33, that includes the lesion, e.g., the deletion, is amplified using a primer, e.g., a primer described herein.

In one embodiment, the primer hybridizes with a nucleotide sequence that is adjacent to the lesion (e.g., deletion), e.g., a nucleotide sequence that is 5′ or 3′ to the lesion, e.g., deletion. Alternatively, the primer can hybridize with a nucleotide sequence that includes nucleotides 5′ to the lesion and nucleotides 3′ to the lesion, wherein the primer does not hybridize to the nucleotide sequence in the absence of a lesion. For example, the nucleotides 5′ to the lesion and 3′ to the lesion are each insufficient for hybridization of the primer, and only in the presence of a lesion are the nucleotides 5′ to the lesion and 3′ to the lesion in proximity to provide a nucleotide sequence to which the primer hybridizes.

In one embodiment, the primer amplifies a fragment of DNA that differs in size or sequence from a fragment of DNA that does not include the lesion.

In one embodiment, the primer is at least 10, e.g., at least 12, e.g., at least 15, e.g., at least 20, e.g., at least 30, e.g., at least 50, e.g., at least 100 nucleotides in length.

In another embodiment, the primer includes at least 10, e.g., at least 20, e.g., at least 50, e.g., at least 100 consecutive nucleotides of SEQ ID NO:1.

In another embodiment, the primer differs from SEQ ID NO:1 by at least 1, 2, 3, or 5, but not more than, e.g., 10, 20, or 50 nucleotides.

In another embodiment, the subject has a lesion, e.g., a deletion, within one or more of 13q14, or portion thereof, e.g., 13q14.3, of human chromosome 13; 11q22-23, or portion thereof, e.g., 11q22.3, of human chromosome 11; 17p13, or portion thereof, e.g., 17p13.1, of human chromosome 17; and/or has more than two copies of human chromosome 12, or portion thereof.

In another embodiment, an array is used to determine the absence or presence of a lesion, e.g., a deletion, within 14q32.33, e.g., within the V_{H} locus at 14q32.33. Preferably, the array is an array described herein that includes at least one capture probe that hybridizes specifically with 14q32.33, e.g., with the V_{H} locus at 14q32.33, or the array is a bacterial artificial chromosome (BAC) array, e.g., a BAC array described herein.

In another embodiment, the subject has CLL, e.g., the subject has early CLL, e.g., the subject has CLL and is being diagnosed for risk of intermediate or aggressive CLL.

In another embodiment, the subject has CLL, e.g., the subject has intermediate CLL, e.g., the subject has intermediate CLL and is being diagnosed for risk of aggressive CLL.

In another embodiment, the subject has one or more risk factors associated with CLL.

In another embodiment, the subject has early lymphoma.

In another embodiment, the subject has lymphoma.

In another embodiment, the subject has one or more risk factors associated with lymphoma.

In another embodiment, the subject has a lesion, e.g., a deletion, within 13q14, or a portion thereof, e.g., 13q14.3, of human chromosome 13.

In another embodiment, the subject has a lesion, e.g., a deletion, within 11q22-23, or a portion thereof, e.g., 11q22.3, of human chromosome 11.

In another embodiment, the subject has a lesion, e.g., a deletion, within 17p13, or a portion thereof, e.g., 17p13.1, of human chromosome 11.

In another embodiment, the subject has one or more copies of human chromosome 12, or a portion thereof.

In another embodiment, the subject has two or more chromosomal abnormalities, e.g., a lesion, e.g., a deletion, within 13q14, or a portion thereof, e.g., 13q14.3, of human chromosome 13; a lesion, e.g., a deletion, within 11q22-23, or a portion thereof, e.g., 11q22.3, of human chromosome 11; a lesion, e.g., a deletion, within 17p13, or a portion thereof, e.g., 17p13.1, of human chromosome 17; and/or two or more copies of human chromosome 12, or a portion thereof.

In another embodiment, the subject has a lesion, e.g., a deletion, within 14q32.33, e.g., within the V_{H} locus at 14q32.33, and the method further includes administering a treatment, or a combination of treatments, to the subject prior to the onset of one or more symptoms of intermediate or aggressive CLL, or lymphoma. Preferably, the treatment, or combination of treatments, is the treatment described herein. Preferably, the subject further has one or more chromosomal abnormalities, e.g., a lesion, e.g., a deletion, within 13q14, or a portion thereof, e.g., 13q14.3, of human chromosome 13; a lesion, e.g., a deletion, within 11q22-23, or a portion thereof, e.g., 11q22.3, of human chromosome 11; a lesion, e.g., a deletion, within 17p13, or a portion thereof, e.g., 17p13.1, of human chromosome 17; and/or two or more copies of human chromosome 12, or a portion thereof.

In another embodiment, the absence of a lesion, e.g., a deletion, within 14q32.33, e.g., within the V_{H} locus at 14q32.33, is indicative that the subject is not at risk for developing intermediate or aggressive CLL, or lymphoma. Preferably, the subject does not have a lesion, e.g., a deletion, within 14q32.33, e.g., within the V_{H} locus at 14q32.33, and a decision is made not to administer a treatment, or combination of treatments, for CLL or lymphoma, e.g., a treatment described herein, to the subject.

In another embodiment, the method further includes providing a result of the determination to a third party, e.g., a hospital, clinic, government entity, reimbursing party or insurance company. In one embodiment, payment for a therapeutic treatment or modality, e.g., a therapeutic treatment or modality described herein, or payment by a reimbursing party, e.g., a government entity or insurance company, is conditional on the outcome of the determination.

Another aspect of the invention features a method for selecting a treatment regimen for a subject with CLL or lymphoma, where the method includes providing a subject having a lesion, e.g., a deletion, within 14q32.33, e.g., within the V_{H} locus at 14q32.33; and providing the subject with a treatment regimen for CLL or lymphoma.

In one embodiment, the treatment for CLL or lymphoma is one or more therapeutic agents or therapeutic modalities. Preferably, the therapeutic agent is a cytotoxic agent. Examples of cytotoxic agents that can be selected include antimicrotubule agents, topoisomerase inhibitors, antimetabolites, mitotic inhibitors, alkylating agents, intercalating agents, agents capable of interfering with a signal transduction pathway, and agents that promote apoptosis. In one embodiment, the cytotoxic agent is one or more of: chlorambucil, cyclosphamide, prednisone, fludarabine, pentostatin, clarribine, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and maytansinoid. In a preferred embodiment, the treatment regimen includes administering an effective amount of one or more of: chlorambucil, cyclosphamide, prednisone, fludarabine, pentostatin, clarribine, vincristine and doxorubicin. The treatment regimen can also be surgical and/or radiation procedures. Such procedures can be used alone or in combination with a therapeutic agent, e.g., a cytotoxic agent. Other treatment regimens can include administering one or more antibodies, or antigen binding portion thereof, that bind an antigen associated with CLL or lymphoma. Examples of such antibodies include anti-CD52 antibodies, anti-common CLL antigen (cCLL) antibodies, and anti-CD3 antibodies. The antibody can be conjugated or unconjugated. The antibody or antibodies can be administered with one or more additional treatment modalities, e.g., a treatment modality described herein.

In another embodiment, the method further includes monitoring the effects of the treatment. Preferably, the method can further include altering the course of treatment if one or more symptoms of CLL or lymphoma is not treated or prevented.

In another embodiment, the method further includes determining the absence or presence of a lesion, e.g., a deletion, within 14q32.33, e.g., within the V_{H} locus at 14q32.33. Preferably, nucleic acid a probe, e.g., a nucleic acid probe described herein, is used to determine the absence or presence of a lesion, e.g., deletion, within 14q32.33, e.g., within the V_{H} locus at 14q32.33. Preferably, the nucleic acid probe is labeled as described herein. Preferably, an array, e.g., an array described herein, is used to determine the absence or presence of a lesion, e.g., a deletion, within 14q32.33, e.g., within the V_{H} locus at 14q32.33.

In another embodiment, the method further includes determining the absence or presence of a lesion, e.g., a deletion, within 13q14, or a portion thereof, e.g., 13q14.3, of human chromosome 13. Preferably, the determination can be made using, e.g., a probe that hybridizes with 13q14, or a portion thereof, e.g., 13q14.3, e.g., a nucleic acid probe described herein, or an array, e.g., an array described herein. Preferably, the subject further has a lesion, e.g., a deletion, within 13q14, or a portion thereof, e.g., 13q14.3.

In another embodiment, the method further includes determining the absence or presence of a lesion, e.g., a deletion, within 11q22-23, or a portion thereof, e.g., 11q22.3, of human chromosome 11. Preferably, the determination can be made using, e.g., a nucleic acid probe that hybridizes with 11q22-23, or a portion thereof, e.g., 11q22.3, e.g., a probe described herein, or an array, e.g., an array described herein. Preferably, the subject further has a lesion, e.g., a deletion, within 11q22-23, or a portion thereof, e.g., 11q22.3.

In another embodiment, the method further includes determining the absence or presence of a lesion, e.g., a deletion, within 17p13, or a portion thereof, e.g., 17p13.1, of human chromosome 17. Preferably, the determination can be made using, e.g., a nucleic acid probe that hybridizes with 17p13, or a portion thereof, e.g., 17p13.1, e.g., a probe described herein, or an array, e.g., an array described herein. Preferably, the subject further has a lesion, e.g., a deletion, within 17p13, or a portion thereof, e.g., 17p13.1.

In another embodiment, the method further includes determining the absence or presence of more than two copies of human chromosome 12, or a portion thereof. Preferably, the determination can be made using, e.g., a nucleic acid probe that hybridizes with human chromosome 12, or a portion thereof, e.g., a probe described herein, or an array, e.g., an array described herein. Preferably, the subject further has more than two copies of chromosome 12, or a portion thereof.

In another embodiment, the subject has early CLL. In another embodiment, the subject has intermediate CLL. In another embodiment, the subject has lymphoma.

Another aspect of the invention features a method for evaluating a treatment, or combination of treatments, for CLL or lymphoma. The method includes providing a first subject having a lesion, e.g., a deletion, within 14q32.33, e.g., within the V_{H} locus at 14q32.33, and a second subject not having a lesion, e.g., a deletion, within 14q32.33, e.g., within the V_{H} locus at 14q32.33; providing the first and second subjects with a treatment, or combination of treatments, for CLL or lymphoma, wherein both the first and second subjects receive the same treatment, or combination of treatments; and comparing the effects of the treatment, or combination of treatments, on the first and second subjects, to thereby evaluate if the treatment, or combination of treatments, is effective to treat or to prevent CLL or lymphoma in subjects having the lesion, e.g., the deletion, subjects not having the lesion, e.g., the deletion, or both.

In one embodiment, the second subject has a lesion, e.g., a deletion, within, e.g., 13q14, or a portion thereof, e.g., 13q14.3, of human chromosome 13; 11q22-23, or a portion thereof, e.g., 11q22.3, of human chromosome 11; or 17p13, or a portion thereof, e.g., 17p13.1, of human chromosome 17; or has two or more copies of human chromosome 12, or a portion thereof.

In another embodiment, the method further includes changing the selected treatment, or combination of treatments, in one or both of the first or second subjects based upon the evaluation.

In another embodiment, both the first and second subjects have one or more chromosomal abnormalities, e.g., a lesion, e.g., a deletion, within 13q14, or a portion thereof, e.g., 13q14.3, of human chromosome 13; a lesion, e.g., a deletion, within 11q22-23, or a portion thereof, e.g., 11q22.3, of human chromosome 11; a lesion, e.g., a deletion, within 17p13, or a portion thereof, e.g., 17p13.1, of human chromosome 17; and/or two or more copies of human chromosome 12, or a portion thereof.

In another embodiment, the method further includes determining the absence or presence of a lesion, e.g., a deletion, within 14q32.33, e.g., within the V_{H} locus at 14q32.33, in the first and/or second subject. Preferably, a nucleic acid probe, e.g., a probe described herein, is used to determine the absence or presence of a lesion, e.g., a deletion, within 14q32.33, e.g., within the V_{H} locus at 14q32.33. Preferably, the probe is labeled as described herein. Preferably, an array, e.g., an array described herein, is used to determine the absence or presence of a lesion, e.g., a deletion, within 14q32.33, e.g., within the V_{H} locus at 14q32.33.

In another embodiment, the method further includes determining the absence or presence of one or more chromosomal abnormalities, e.g., a lesion, e.g., a deletion, within 13q14, or a portion thereof, e.g., 13q14.3, of human chromosome 13; a lesion, e.g., a deletion, within 11q22-23, or a portion thereof, e.g., 11q22.3, of human chromosome 11; a lesion, e.g., a deletion, within 17p13, or a portion thereof, e.g., 17p13.1, of human chromosome 17; and/or two or more copies of human chromosome 12, or a portion thereof.

Preferably, a nucleic acid probe, e.g., a probe described herein, is used to determine the absence or presence of a lesion, e.g., a deletion, within 13q14, or a portion thereof, e.g., 13q14.3, of human chromosome 13. Preferably, the probe is labeled as described herein. Preferably, an array, e.g., an array described herein, is used to determine the absence or presence of a lesion, e.g., a deletion, within 13q14, or a portion thereof, e.g., 13q14.3.

Preferably, a nucleic acid probe, e.g., a probe described herein, is used to determine the absence or presence of a lesion, e.g., a deletion, within 11q22-23, or a portion thereof, e.g., 11q22.3, of human chromosome 11. Preferably, the probe is labeled as described herein. Preferably, an array, e.g., an array described herein, is used to determine the absence or presence of a lesion, e.g., a deletion, within 11q22-23, or a portion thereof, e.g., 11q22.3.

Preferably, a nucleic acid probe, e.g., a probe described herein, is used to determine the absence or presence of a lesion, e.g., a deletion, within 17p13, or a portion thereof, e.g., 17p13.1, of human chromosome 17. Preferably, the probe is labeled as described herein. Preferably, an array, e.g., an array described herein, is used to determine the absence or presence of a lesion, e.g., a deletion, within 17p13, or a portion thereof, e.g., 17p13.1

Preferably, a probe, e.g., a probe described herein, is used to determine the absence or presence of more than two copies of human chromosome 12, or a portion thereof. Preferably, the probe is labeled as described herein. Preferably, an array, e.g., an array described herein, is used to determine the absence or presence of more than two copies of human chromosome 12, or a portion thereof.

In another embodiment, both the first and second subjects have aggressive CLL. In another embodiment, both the first and second subjects have intermediate CLL. In another embodiment, both the first and second subjects have lymphoma.

Another aspect of the invention features a method for selecting a treatment regimen for a subject. The method includes determining the absence or presence of a lesion, e.g., a deletion, within 14q32.33, within the V_{H} locus of 14q32.33, of the subject, and if the lesion, e.g., the deletion, is present, providing a treatment regimen for CLL or lymphoma to the subject.

In one embodiment, the subject has CLL, e.g., early or intermediate CLL.

In one embodiment, the subject has lymphoma.

In another embodiment, a nucleic acid probe, e.g., a probe described herein, is used to determine the absence or presence of a lesion, e.g., a deletion, within 14q32.33, within the V_{H} locus at 14q32.33. Preferably, the probe is labeled as described herein.

In another embodiment, an array, e.g., an array described herein, is used to determine the absence or presence of a lesion, e.g., a deletion, within 14q32.33, e.g., within the V_{H} locus at 14q32.33.

In another embodiment, a primer, or set of primers, e.g., a primer or set of primers as described herein, is used to determine the absence or presence of a lesion, e.g., a deletion, within 14q32.33, e.g., within the V_{H} locus at 14q32.33.

In another embodiment, the presence of a lesion, e.g., a deletion, within 14q32.33, e.g., within the V_{H} locus at 14q32.33, is indicative of a risk of CLL or lymphoma. Preferably, the presence of the lesion, e.g., the deletion, is indicative of a risk of developing intermediate or aggressive CLL or lymphoma.

In another embodiment, the treatment is a treatment, or a combination of treatments, described herein.

In another embodiment, the subject has a lesion, e.g., a deletion, within, e.g., 13q14, or a portion thereof, e.g., 13q14.3, of human chromosome 13; 11q22-23, or a portion thereof, e.g., 11q22.3, of human chromosome 11; or 17p13, or a portion thereof, e.g., 17p13.1, of human chromosome 17; or has two or more copies of human chromosome 12, or a portion thereof.

Another aspect of the invention features a method for evaluating a treatment, or combination of treatments, for CLL or lymphoma. The method includes providing at least two subjects having a lesion, e.g., a deletion, within 14q32.33, e.g., within the V_{H} locus at 14q32.33; administering a treatment, or combination of treatments, to at least one of the subjects and administering a different treatment, or combination of treatments, to the other subject; and comparing the effects of different treatments, or combination of treatments, in the subjects.

In one embodiment, the subjects have early CLL. In another embodiment, the subjects have intermediate CLL. In another embodiment, the subjects have lymphoma.

In another embodiment, a nucleic acid probe, e.g., a probe described herein, is used to determine the absence or presence of a lesion, e.g., a deletion, within 14q32.33, e.g., within the V_{H} locus at 14q32.33. Preferably, the nucleic acid probe is labeled as described herein.

In another embodiment, an array, e.g., an array described herein, is used to determine the absence or presence of a lesion, e.g., a deletion, within 14q32.33, e.g., within the V_{H} locus at 14q32.33.

In another embodiment, a primer, or set of primers, e.g., a primer or set of primers as described herein, is used to determine the absence or presence of a lesion, e.g., a deletion, within 14q32.33, e.g., within the V_{H} locus at 14q32.33.

In another embodiment, the presence of a lesion, e.g., a deletion, within 14q32.33, e.g., within the V_{H} locus at 14q32.33, is indicative of a risk of CLL or lymphoma. Preferably, the presence of the lesion, e.g., the deletion, is indicative of a risk of developing intermediate or aggressive CLL or lymphoma.

In another embodiment, the treatment is a treatment, or a combination of treatments, described herein.

In another embodiment, the subject has a lesion, e.g., a deletion, within, e.g., 13q14, or a portion thereof, e.g., 13q14.3, of human chromosome 13; 11q22-23, or a portion thereof, e.g., 11q22.3, of human chromosome 11; or 17p13, or a portion thereof, e.g., 17p13.1, of human chromosome 17; or has two or more copies of human chromosome 12, or a portion thereof.

In another aspect, the invention features, a method of making a decision, e.g., a medical or financial decision. The method includes:
generating or receiving data on the absence or presence of a lesion, e.g., a deletion, within human chromosome 14, e.g., within 14q32.33, e.g., within the V_{H} locus of 14q32.33, e.g., receiving the data generated by a method described herein; and
using the data to make the decision, e.g., selecting between a first course of action and a second course of action.

In a preferred embodiment, the decision includes comparing the data to a standard and making the decision based on the relationship of the data to the standard. For example, the data can be a value or other term for the likelihood of CLL or lymphoma and if the value or other term has a preselected relationship to the standard, e.g., if the value or term in the data is greater than a reference standard, selecting a first course of action and if the data is less than a reference standard selecting a second course of action. A course of action can be, e.g., providing or not providing service or treatment, or paying for or not paying for all or part of a service or treatment.

In a preferred embodiment, the first course of action is suggesting or providing a first course of medical treatment, e.g., any treatment described herein, and the second course of action is suggesting or deciding that the treatment not be given or not providing the treatment. In a preferred embodiment the first course of action includes or results in the authorization or transfer of funds to pay for a service or treatment provided to a subject and the second course of action includes or results in the refusal to pay for a service or treatment provided to a subject. For example, an entity, e.g., a hospital, caregiver, government entity, or an insurance company or other entity, that pays for, or reimburses, medical expenses, can use the outcome of a method described herein to determine whether a party, e.g., a party other than the subject patient, will pay for services or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient.

### BRIEF DESCRIPTION OF THE DRAWINGS

FIG. 1 is a summary of array-CGH and conventional cytogenetic findings of 21 CLL patients.

FIG. 2 is chromosome 14 array-CGH ratio plot profiles for three CLL patients.

FIG. 3 is a micrograph of dual-color interphase FISH.

FIG. 4 depicts prognostic significance of the V_{H} deletion in 22 previously untreated patients selected for 13q14.3 deletions as their sole genetic abnormality. FIG. 4A is the number of patients treated as a function of V_{H} deletion status, based on the sample median V_{H} deletion of 40%. FIG. 4B is a graph of Kaplan Maier analysis.

FIG. 5 is a schematic representation of the V_{H} region within the IgH locus at 14q32.33.

FIG. 6 depicts the nucleotide sequence of probe CTC-200D12 (SEQ ID NO:1) that hybridizes within 14q32.33 of human chromosome 14.

### DETAILED DESCRIPTION OF THE INVENTION

### DEFINITIONS

The term “chromosome” refers to a gene carrier of a cell which is derived from chromatin and which comprises DNA and protein components (especially histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome 1, contains about 2.4×10⁸ bp while the smallest chromosome, chromosome 22, contains about 5.3×10⁷ bp (Yunis, J. J. Science 191:1268-1270 (1976), and Kavenoff, R., et al. Cold Spring Harbor Symposia on Quantitative Biology 38:1-8 (1973)).

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The terms “probe” and “primer” refer to a polynucleotide such as DNA sequence. Each such polynucleotide of a probe can be single stranded at the time of hybridization to a target. A primer can be used to prime a reaction, e.g., a PCR reaction.

The term “label” or “label containing moiety” refers in a moiety capable of detection, such as a radioactive isotope or group containing same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The term “target”, “DNA target” or “DNA target region” refers to a nucleotide sequence that occurs at a specific chromosomal location. Each such sequence or portion is preferably, at least partially, single stranded (i.e. denatured) at the time of hybridization. When the target nucleotide sequences are located only in a single region or fraction of a given chromosome, the term “target region” is sometimes used. Targets for hybridization can be derived from specimens which include but are not limited to chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or zygotes, embryos, chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “fluorescent compound” or “fluorophore group” generally refers to an organic moiety. A fluorescent compound is capable of reacting, and a fluorophore group may have already reacted, with a linking group. A fluorescent compound can include an organic chelator that binds a luminescent inorganic ion such as a rare earth like terbium, europium, ruthenium, or the like.

The term “linking compound” or “linking group” as used herein generally refers to a hydrocarbonaceous moiety. A linking compound is capable of reacting, and a linking group may have already reacted, with a nucleotide sequence (or nucleotide segment). A linking compound is also capable of reacting, and a linking group may have already reacted, with a label, e.g., fluorophore compound.

The term “in situ” means that the chromosomes are exposed from the cell nucleus without substantial disruption or relocation of the chromosomes with respect to each other and with the chromosomes being accessible to fluorescently labeled DNA probes.

The term “hybridizing conditions” has general reference to the combinations of conditions that are employable in a given hybridization procedure to produce hybrids, such conditions typically involving controlled temperature, liquid phase, and contacting between a probe (or probe composition) and a target. Conveniently and preferably, at least one denaturation step precedes a step wherein a probe or probe composition is contacted with a target. Alternatively, a probe can be contacted with a specimen comprising a DNA target region and both subjected to denaturing conditions together as described by Bhatt et al. in Nucleic Acids Research 16: 3951-3961. Using, for example, about a 50:50 volume ratio mixture of water and formamide, an illustrative temperature for contacting and hybridization between probe (or probe composition) and target is in the range of about 35 to about 55 ° C. applied for a time that is illustratively in the range of about 1 to about 18 hours. Other hybridizing conditions can be employed. The ratio of numbers of probes to number of target sequences or segments can vary widely, but generally, the higher this ratio, the higher the probability of hybrid formation under hybridizing conditions within limits. As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

As used herein, the term “substantially identical” (or “substantially homologous”) is used to refer to a first nucleotide sequence that contains a sufficient number of identical or equivalent nucleotides to a second nucleotide sequence such that the first and second nucleotide sequences have similar activities.

Calculations of “homology” between two sequences can be performed as follows. The sequences are aligned for optimal comparison purposes (e.g., gaps can be introduced in one or both of a first and a second nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The nucleotides at corresponding nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent homology between two sequences can be accomplished using a mathematical algorithm. In a preferred embodiment, the percent homology between two nucleotide sequences is determined using the GAP program in the GCG software package, using an NWSgapdna.CNW matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a homology limitation of the invention) are a Blossum 62 scoring matrix with a gap penalty of 12, a gap extend penalty of 4, and a frameshift gap penalty of 5.

The term “nonspecific binding DNA” refers to DNA which is complementary to DNA segments of a probe and which DNA occurs in at least one other position in a genome, which other position is outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

Probes

Hybridization probes are widely used to detect and/or quantify the presence of a particular nucleic acid sequence within a sample of nucleic acid sequences. Hybridization probes detect the presence of a particular nucleic acid sequence, referred to herein as a target sequence, through the use of a complimentary nucleic acid sequence that selectively hybridizes to the target nucleic acid sequence. In order for a hybridization probe to hybridize to a target sequence, the hybridization probe must have sufficient identity with the target sequence. Preferably, the probe has at least 70%, 80%, 90%, 95%, 98% or more homology to the target sequence. The probe sequence must also be sufficiently long so that the probe exhibits selectivity for the target sequence over non-target sequences. For example, the probe can be at least 50 kb, 100 kb, 200 kb, 300 kb, 400 kb, 500 kb, 600 kb, 700 kb, 800 kb, 900 kb or more, in length. Probes are typically about 50 to about 1×10⁶ nucleotides in length. Probes used in accordance with the invention are about 1×10⁵ to about 1×10⁶ nucleotides in length.

Depending on the chromosomal abnormality to be detected, the probe can be a probe that hybridizes to repetitive DNA associated with the centromere of a chromosome or the probe can hybridize to a critical chromosomal region, such as all or a portion of 14q32.33 of chromosome 14, 13q14.3 of chromosome 13, 11q22-23 of chromosome 11, 17p13.1 of chromosome 17, and chromosome 12. Control probes can also be used, e.g., in the FISH analysis. For example, a probe that binds a centromere can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Alternatively, probes can be made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, Biotechnic Histochem., 1998, 73(1):6-22, Wheeless et al., Cytometry 1994, 17:319-326, and U.S. Pat. No. 5,491,224.

For FISH analysis, the chromosomal probes are preferably labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, U.S. Pat. No. 5,491,224.

Fluorophores of different colors can be chosen such that each chromosomal probe in the set can be distinctly visualized. For example, a combination of the following fluorophores may be used: 7-amino-4-methylcoumarin-3-acetic acid (AMCA), Texas Red™. (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the chromosomal probes.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Arrays and Uses Thereof

In another aspect, the invention features an array that includes a substrate having a plurality of addresses. At least one address of the plurality includes a nucleic acid capture probe that binds specifically to all or a portion of chromosome 14q32.33 and detects the absence or presence of a lesion, e.g., a deletion, in this region. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array. In some embodiments, the array further includes at least one address that includes a nucleic acid capture probe that binds specifically to all or a portion of chromosome having a structural abnormality associated with CLL, e.g., a structural abnormality described herein. For example, the array can further include a nucleic acid probe that binds all or a portion of: 13q14 (e.g., 13q14.3), chromosome 17p13 (e.g., 17q13.1), chromosome 11q22-23, or chromosome 12.

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another aspect, the invention features a method of analyzing the absence or presence of a lesion, e.g., a deletion, at chromosome 14q32.33. The method includes providing an array as described above; contacting the array with a sample, e.g., a portion of genomic DNA that includes at least a portion of human chromosome 14q32.33, and detecting binding of a nucleic acid from the sample to the array. Optionally, the method further includes amplifying nucleic acid from the sample, e.g., genomic DNA that includes a portion of human chromosome 14q32.33 (e.g., a portion that can include a deleted region of human chromosome 14q32.33), prior or during contact with the array.

In some aspects, the methods of the invention can include using an array that can ascertain differential expression patterns or copy numbers of one or more genes in normal and abnormal cells. For example, arrays of genomic bacterial artificial chromosomes (BAC), P1 or cosmid clones can be used to measure differences in chromosomal structure between DNA from a subject having or at risk for CLL and control DNA, e.g., DNA obtained from an individual that does not have CLL and is not as risk for CLL. Since the clones on the array contain sequence tags, their positions on the array are accurately known relative to the genomic sequence. Different hybridization patterns between the CLL DNA and the normal DNA at positions in the array corresponding to human chromosome 14q32.33, are indicative of a risk of CLL. Methods for array production, hybridization, and analysis are described, e.g., in Snijders et al. (2001) Nat. Genetics 29:263-264; Klein et al. (1999) Proc. Natl Acad. Sci. USA 96:4494-4499; Albertson et al. (2003) Breast Cancer Research and Treatment 78:289-298; and Snijders et al. “BAC microarray based comparative genomic hybridization.” In: Zhao et al. (eds), Bacterial Artificial Chromosomes: Methods and Protocols, Methods in Molecular Biology, Humana Press, 2002.

In another aspect, the invention features a method of determining the absence or presence of a lesion, e.g., a deletion, in human chromosome 14q32.33, using an array described above. The method is useful, e.g., for analyzing differences in chromosome number or structure, e.g., within chromosome 14q32.33 and/or other chromosomes or portions thereof associated with CLL. The method includes: providing a two dimensional array having a plurality of addresses, each address of the plurality being positionally distinguishable from each other address of the plurality having a unique nucleic acid capture probe, contacting the array with a first sample from a cell or subject having or at risk for CLL; contacting the array with a second sample from a cell or subject that does not have CLL and is not at risk for CLL; and comparing the binding of the first sample with the binding of the second sample. Binding, e.g., in the case of a nucleic acid hybridization, with a capture probe at an address of the plurality, is detected, e.g., by signal generated from a label attached to the nucleic acid.

Kits

Also within the scope of the invention are kits comprising a probe that hybridizes with human chromosome 14q32.33 described herein. The kit can include one or more other elements including: instructions for use; and other reagents, e.g., a label, or an agent useful for attaching a label to the probe. Instructions for use can include instructions for diagnostic applications of the probe for assessing risk of CLL. Other instructions can include instructions for attaching a label to the probe, instructions for performing in situ analysis with the probe, and/or instructions for obtaining a sample to be analyzed from a subject. As discussed above, the kit can include a label, e.g., any of the labels described herein. In some embodiments, the kit includes a labeled probe that hybridizes to human chromosome 14q32.33, e.g., a labeled probe as described herein.

The kit can also include one or more additional probes that hybridize to a chromosome or portion of a chromosome that can have an abnormality associated with risk for CLL. For example, the additional probe can be: a probe that hybridizes to human chromosome 13q14 or a portion thereof, e.g., 13q14.3, (e.g., a probe that detects a deletion in this region of chromosome 13); a probe that hybridizes to human chromosome 11q22-23 or a portion thereof (e.g., a probe that detects a deletion in this region of chromosome 11); a probe that hybridizes to human chromosome 17p13 or a portion thereof, e.g., 17p13.1, (e.g., a probe that detects a deletion in this region of chromosome 17); a probe that hybridizes to human chromosome 12 or a portion thereof. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

The kit can further contain at least one treatment (e.g., a cytotoxic agent described herein) or instructions for treatment if one or more of the chromosomal abnormalities described above is detected.

Methods of Diagnosing CLL

The invention includes method of diagnosing if a subject is at risk for CLL. The method includes providing genomic DNA from the subject, and determining the absence or presence of a lesion, e.g., a deletion, at human chromosome 14q32.33.

The subject can be a mammal, e.g., a primate, preferably a higher primate, e.g., a human (e.g., a patient having, or at risk of, CLL). In one embodiment, the subject is a patient having CLL (e.g., a patient suffering from early, intermediate or aggressive CLL).

In one embodiment, a subject to be evaluated by the methods is a subject having one or more risk factors associated with CLL. For example, the subject may have a relative afflicted with CLL, e.g., a subject with one or more of a grandparent, parent, uncle or aunt, sibling, or child who has or had CLL; the subject may have a genetic trait associated with risk for CLL (e.g., the subject is Jewish); or the subject may have had contact, e.g., prolonged contact, with one or more chemicals (e.g., benzene, and various herbicides, solvents and pesticides) associated with increase risk of CLL. In other embodiments, the risk factor can be one or more chromosomal abnormalities known to be associated with CLL. Known chromosomal abnormalities associated with CLL include: a lesion, e.g., a deletion, at chromosome 13q14 (e.g., 13q14.3); a lesion, e.g., a deletion, at chromosome 17p13 (e.g., 17p13.1), a lesion, e.g., a deletion, at chromosome 11q22-23, or more than two copies of chromosome 12. In some embodiments, the methods further include determining if a subject has one or more of chromosomal abnormality associated with CLL.

The invention also features methods of determining if a subject having CLL, e.g., early or intermediate CLL, is at risk for developing intermediate or aggressive CLL. The method includes evaluating a subject having CLL, e.g., early or intermediate CLL, for the absence or presence of a lesion, e.g., a deletion, at chromosome 14q32.33. The presence of a lesion, e.g., a deletion, at chromosome 14q32.33 is indicative of a risk of developing intermediate or aggressive CLL. Subjects can be evaluated, e.g., using the compositions, kits and methods described herein.

Subjects having CLL can be categorized into low- (or “early”), intermediate- and high-risk (or “aggressive”) CLL, e.g., by determining the stage of the cancer. The “stage” of a cancer as used herein refers to how far the cancer has progressed. Various methods of determining the stage of CLL such as Binet and Rai classification are known and described, e.g., in Binet J. L., et al. (1981) Cancer 48:198-206 and Rai K. R., et al. (1975) Blood 46:219-234, respectively. Binet staging classifies CLL according to the number of lymphoid tissues that are involved. These tissues include the spleen and lymph nodes of the neck, groin and underarms. The presence of low red blood cells (anemia) and platelet counts (thrombocytopenia) are also considered in classifying CLL according to Binet staging. Binet classification assigns stages A, B and C to CLL. “Stage A” refers to subjects having fewer than three areas of enlarged lymphoid tissue selected from the spleen, lymph nodes of the neck, lymph nodes of the groin and lymph nodes of the underarm. “Stage B” refers to subjects having more than three areas of enlarged lymphoid tissue. “Stage C” subjects have more than three areas of enlarged lymphoid tissue as well as anemia and/or thrombocytopenia. Anemia can be classified as subjects having red blood cells counts of less than 10 g/dL. Thrombocytopenia can be classified as subjects having platelet counts of less than 100×10³/μL. Rai staging classifies CLL into low-, intermediate and high-risk categories by evaluating a subject for lymphocytosis (i.e., high numbers of lymphocytes), lymphoadenopathy (i.e., lymphatic disease), heptomegaly (i.e., enlarged liver) and splenomegaly(i.e., enlarged spleen). Rai classification assigns stages 0, I, II, III and IV to subjects having CLL. “Stage 0” refers to subjects having lymphocytosis. Lymphocytosis is a lymphocyte count of more than 15,000 lymphocytes per cubic millimeter. “Stage I” refers to subjects having lymphocytosis and enlarged lymph nodes (lymphoadenopathy). “Stage II” refers to subjects having lymphocytosis plus an enlarged liver (hepatomegaly) or enlarge spleen (splenomegaly), with or without lymphoadenopathy. “Stage III” refers to subjects having lymphocytosis and anemia, with or without lymphoadenopathy, hepatomegaly or splenomegaly. Lastly, “Stage IV” refers to subjects having lymphocytosis plus thrombocytopenia.

A subject is classified as having “early” or “low-risk” CLL if the subject is considered to have Stage A CLL by Binet staging, or Stage 0 CLL by Rai staging. “Intermediate-risk” refers to subjects classified as having Stage B CLL by Binet staging or Stage I or Stage II by Rai staging. “Aggressive” or “high-risk” CLL refers to subjects classified as having Stage C CLL by Binet staging or Stage III or Stage IV CLL by Rai staging.

The absence or presence of a chromosomal abnormality, e.g., a chromosomal abnormality described herein, can be determined using methods known in the art such as fluorescent in situ hybridization (FISH), spectral karotyping (MFISH) and array CGH. FISH analysis and assay-CHG can be performed, e.g., as described herein.

The methods of the invention can include determining the presence or absence of aneusomic cells by, e.g., in situ hybridization. “Aneusomic cells” are cells having an abnormal number of chromosomes and/or having chromosomal structural alterations such as hemizygous or homozygous loss of a specific chromosomal region. In general, in situ hybridization includes the steps of fixing a biological sample, hybridizing a probe, e.g., a probe described herein, to target DNA contained within the fixed biological sample, removing unbound probe, e.g., by washing, and detecting the absence or presence of hybridized probe.

With regard to in situ hybridization, a “biological sample” is a sample that contains cells or cellular material. The biological sample can be concentrated prior to hybridization to increase cell density. Non-limiting examples of biological samples include urine, blood, and tissue samples. For tissue samples, the tissue can be fixed and placed in paraffin for sectioning, or frozen and cut into thin sections.

Cells can be harvested from a biological sample using standard techniques known in the art. For example, cells can be harvested by centrifuging a biological sample and resuspending the pelleted cells. The cells can be resuspended in a buffered solution such as phosphate-buffered saline (PBS). After centrifuging the cell suspension to obtain a cell pellet, the cells can be fixed, for example, in acid alcohol solutions, acid acetone solutions, or aldehydes such as formaldehyde, paraformaldehyde, and glutaraldehyde. For example, a fixative comprising methanol and glacial acetic acid in a 3:1 ratio, respectively, can be used as a fixative. A neutral buffered formalin solution also can be used. Slides containing the cells can be prepared by removing a majority of the fixative, leaving the concentrated cells suspended in only a portion of the solution.

The cell suspension is applied to slides such that the cells do not overlap on the slide. Cell density can be measured by a light or phase contrast microscope.

Prior to in situ hybridization, chromosomal probes and chromosomal DNA contained within the cell each can be denatured. Denaturation, typically, is performed by incubating in the presence of high pH, heat (e.g., temperatures from about 70° C. to about 95° C.), and/or organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Denaturation conditions typically are established such that cell morphology is preserved. Chromosomal probes can be denatured by heat. For example, probes can be heated to about 73° C. for about five minutes.

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations are typically performed in hybridization buffer containing 1-2×SSC, 50% formamide and blocking DNA to suppress non-specific hybridization. In general, hybridization conditions, as described above, include temperatures of about 25° C. to about 55° C., and incubation lengths of about 0.5 hours to about 96 hours. More particularly, hybridization can be performed at about 32° C. to about 40° C. for about 2 to about 16 hours.

Non-specific binding of chromosomal probes to DNA outside of the target region can be removed, e.g., by one or more washes. Temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

Methods of Determining Treatment Regimens and Methods of Treating CLL

Methods of the invention also include selecting a treatment regimen for a patient with CLL (e.g., early, intermediate or aggressive CLL) based upon the absence or presence of a deletion at 14q32.33. The determination of a treatment regimen can also be based upon the absence or presence of other chromosomal abnormalities associated with CLL, e.g., one or more chromosomal abnormalities described herein. Currently, subjects having early and sometimes even intermediate CLL, are not treated. As described herein, the presence of a lesion, e.g., a deletion, at chromosome 14q32.33 has been correlated with poor patient prognosis. Therefore, the methods of the invention can include selecting a treatment regimen for a subject having early or intermediate CLL, and having a deletion at chromosome 14q32.33. The methods can also include administering a treatment regimen to a subject having CLL, e.g., early or intermediate CLL, to thereby treat, prevent or delay further progression of the disease.

As used herein, the term “treat” or “treatment” is defined as the application or administration of a treatment regimen, e.g., a therapeutic agent or modality, to a subject, e.g., a patient. The subject can be a patient having CLL (e.g., early, intermediate or aggressive CLL), a symptom of CLL or a predisposition toward CLL. The treatment can be to cure, heal, alleviate, relieve, alter, remedy, ameliorate, palliate, improve or affect CLL, the symptoms of CLL or the predisposition toward CLL.

The methods of the invention, e.g., methods of determining a treatment regimen and methods of treatment or prevention of CLL, can further include the step of monitoring the subject, e.g., for a change (e.g., an increase or decrease) in one or more of: levels of a cancer marker, e.g., uric acid; blood count (e.g., lymphocyte, red blood cell and/or platelet levels); the number and types of cells present in bone marrow; the appearance of new disease-related symptoms; the size of various organs such as lymph nodes, spleen and liver; quality of life, e.g., amount of disease associated pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same or a different therapeutic agent or modality. Generally, a decrease in one or more of the parameters described above is indicative of the improved condition of the subject, although with red blood cell and platelet levels, an increase can be associated with the improved condition of the subject.

The methods can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response genotype. In a preferred embodiment, a treatment for CLL can be evaluated by administering the same treatment or combinations or treatments to a subject having CLL and a deletion at human chromosome 14q32.33 and to a subject that has CLL but does not have a deletion at human chromosome 14q32.33. The effects of the treatment or combination of treatments on each of these subjects can be used to determine if a treatment or combination of treatments is particularly effective on a sub-group of subjects having CLL. In other embodiments, various treatments or combinations of treatments can be evaluated by administering two different treatments or combinations of treatments to at least two different subjects having CLL and a deletion in human chromosome 14q32.33. Such methods can be used to determine if a particular treatment or combination of treatments is more effective than others in treating this subset of CLL patients.

Various treatment regimens are known for treating cancer. These include various cytotoxic agents or combinations of cytotoxic agents. Examples of cytotoxic agents that can be selected include antimicrotubule agents, topoisomerase inhibitors, antimetabolites, mitotic inhibitors, alkylating agents, intercalating agents, agents capable of interfering with a signal transduction pathway, and agents that promote apoptosis. In one embodiment, the cytotoxic agent is one or more of: chlorambucil, cyclosphamide, prednisone, fludarabine, pentostatin, clarribine, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and maytansinoid. In a preferred embodiment, the treatment regimen includes administering an effective amount of one or more of: chlorambucil, cyclosphamide, prednisone, fludarabine, pentostatin, clarribine, vincristine and doxorubicin. The treatment regimen can also be surgical and/or radiation procedures. Such procedures can be used alone or in combination with a therapeutic agent, e.g., a cytotoxic agent. Other treatment regimens can include administering one or more antibodies, or antigen binding portion thereof, that bind an antigen associated with CLL. Examples of such antibodies include anti-CD52 antibodies, anti-common CLL antigen (cCLL) antibodies, and anti-CD3 antibodies. The antibody can be conjugated or unconjugated. The antibody or antibodies can be administered with one or more additional treatment modalities, e.g., a treatment modality described herein.

In yet other embodiments, treatment regimen can include administering the cytotoxic agent in combination with a therapeutic modality that enhances blood counts. For example, a therapeutic modality that enhances blood counts can be administered prior to, in conjunction with, or after a subsequent dose (or doses) of a cytotoxic agent is administered to the subject. Examples of therapeutic modalities that enhance blood counts include: platelet transfusion, administration of a growth factor and/or immunomodulatory agents and bone marrow transplantation. Examples of growth factors and immunomodulatory agents include interleukins (e.g., IL-1, 2, 4, 6, 11 or 12), interferon alpha, interferon gamma, thrombopoietin, epoietin □, erythropoietin, G-CSF, CSF and GM-CSF.

Pharmacogenomics

With regards to both prophylactic and therapeutic methods of treatment of CLL, such treatments may be specifically tailored or modified, based on knowledge obtained from the field of pharmacogenomics. “Pharmacogenomics”, as used herein, refers to the application of genomics technologies such as structural chromosomal analysis, to drugs in clinical development and on the market. See, for example, Eichelbaum, M. et al. (1996) Clin. Exp. Pharmacol. Physiol. 23:983-985 and Linder, M. W. et al. (1997) Clin. Chem. 43:254-266. Specifically, as used herein, the term refers the study of how a patient's genes determine his or her response to a drug (e.g., a patient's “drug response phenotype,” or “drug response genotype.”) Thus, another aspect of the invention provides methods for tailoring an individual's prophylactic or therapeutic treatment according to that individual's drug response genotype.

Information generated from pharmacogenomic research can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when administering a therapeutic composition, e.g., a cytotoxic agent or combination of cytotoxic agents, to a patient, as a means of treating or preventing CLL.

In one embodiment, a physician or clinician may consider applying knowledge obtained in relevant pharmacogenomics studies when determining whether to administer a pharmaceutical composition, e.g., a cytotoxic agent or a combination of cytotoxic agents, to a subject. In another embodiment, a physician or clinician may consider applying such knowledge when determining the dosage, e.g., amount per treatment or frequency of treatments, of a treatment, e.g., a cytotoxic agent or combination of cytotoxic agents, administered to a patient.

A physician or clinician may determine the genotypes, at one or more genetic loci, e.g., one or more of chromosome 14q32.33, chromosome 13q14.3, chromosome 11q22-23, chromosome 17p13.1, and chromosome 12, of a group of subjects participating in a clinical trial, wherein the subjects have CLL, and the clinical trial is designed to test the efficacy of a pharmaceutical composition, e.g., a cytotoxic or combination of cytotoxic agents, and wherein the physician or clinician attempts to correlate the genotypes of the subjects with their response to the pharmaceutical composition.

The following invention is further illustrated by the following examples, which should not be construed as further limiting. The contents of all references, pending patent applications and published patents, cited throughout this application, are hereby expressly incorporated by reference.

### EXAMPLES

### Example 1

### Array-CGH in Comparison to FISH in CLL

Array-CGH analysis was performed using BAC (bacteria artificial chromosomes) clones spotted onto glass slide (Spectral Genomics) on 21 patients with CLL all of whom had their disease characterized by FISH. Array-CGH analysis confirmed the cytogenetic abnormalities previously characterized by FISH, but also revealed a number of cryptic abnormalities, many of which are shared among patients. Correlating the array-CGH finding with patient outcome data, a cryptic deletion at 14q23 was identified that appears to be associated with patient prognosis. The presence of this deletion was verified by FISH and extended this study to a larger cohort of patients.

The initial aim was to evaluate results obtained by array-CGH in comparison to those obtained by cytogenetics and FISH. The BAC arrays used consisted of 1003 clones, each clone containing 200-300 kb of genomic DNA fragments spotted in 12 duplicate sub grids onto glass slides. These arrays provide an approximate 3 Mb resolution scan of the whole human genome in a single hybridization. The relative fluorescence signal intensities of each clone reflect variations in DNA sequence copy number for those particular BAC clones.

For array-CGH analysis, genomic DNA was extracted according to the manufacturers instructions (Purgene DNA Purification Kit, Gentra Systems, Inc.) from peripheral blood mononuclear cells (PBMC) of 21 patients with CLL. The cytogenetic profile for each of these patients was previously characterized by cytogenetic analysis and by interphase FISH analyses using the standard CLL panel of probes; ATM at 11q22.3; D12Z3 at 12 centromere; D13S319 at 13q14.3; LSI13q34 at 13q34; P53 at 17p13.1 (Vysis, Downers Grove, Ill.). For each patient, the array-CGH procedure consisted of hybridization of two BAC arrays (Spectral Genomic Inc., Houston, Tex.) with differentially labeled patient (test) and healthy (reference) samples combined together in order to evaluate differences in fluorescence intensities resulting from the co-hybridization reaction onto BAC arrays. According to the gender of each patient analyzed, karyotypically normal human genomic DNA from male or female (Promega Corporation) was used as the reference sample for each reaction. Two μg of genomic DNA from test and reference were prepared in a final volume of 50 μl and sonicated (1 minute and 20 second at 100 amplitude at 10 second impulses) to generate a relatively homogenous smear of fragments of molecular weights greater than or equal to 20 Kbp to approximately 600 bp. DNA was quantified the by 1% agarose gel electrophoresis to ensure equal amounts of fragmentation test and reference samples. After sonication, DNA was further purified (DNA Clean & Concentrator™-5, Zymo Research) according to the manufacturers instructions.

In each experiment test and reference DNA samples were differentially labeled with Cy3-dCTP and Cy5-dCTP dyes (Amersham Biosciences), using the BioPrime DNA Labeling System (Invitrogen, Carlsbad, Calif.). After 5-6 hours of incubation at 37° C., the appropriate probe size distribution of 100 to 500 bp was verified by agarose gel electrophoresis and the reaction stopped by 0.5 M EDTA pH 8.0. Test and reference samples were combined and co-precipitated by addition of NaCl and Isopropanol. This DNA was washed and resuspended in a hybridization solution and denatured (72° C. for 10 minutes) and prehybridized (37° C. for 30 minutes). The denatured DNA was immediately placed onto the arrays, under a sealed cover slip and hybridized at 42° C. for 36-48 hours followed by four post-hybridization washes at 55° C. The arrays were finally soaked in distilled water and dried by brief centrifugation. For each patient, both arrays were scanned using a dual laser wavelengths scanner (GenePix 4000B, Axon instruments, Foster City, Calif.). Simultaneous acquisition of fluorescence at 532 nm and 635 nm provided 4 images that were processed by SpectralWare software (Spectral Genomics Inc., Houston, Tex.) in order to obtain the fluorescence intensities ratio plot for each chromosome.

Two of the CLL cases had trisomy 12 and in each case array CGH detected this amplification. In one of these cases, trisomy 12 was detected by FISH in only 12% of the cells, but could still be detected by array-CGH. One CLL case had deletion 11q22 and again this was detected by array CGH. Eight CLL patients had deletion 13q14.3 detected by FISH and in each case this deletion was also detected by array-CGH. Two of the cases with deletion 13q14.3 also had a small population of cells with deletion 17q13.1 detected by FISH. Although the array-CGH was able to detect the major population of cells with the deletion 13q14.3, array-CGH did not detect the deletion 17p13.1. Two additional cases had deletion 17p13.1 as a single abnormality identified by FISH in 67% and 11% of cells. Array-CGH was able to detect the first case only.

Array-CGH was able to detect 83% of the abnormalities detected by FISH. However, unlike FISH, which detects only the previously characterized abnormality, array-CGH also allows for whole genome screening and was able to detect a number of other previously unidentified deletions and amplification. A number of these abnormalities occurred within the same BAC clones in more then one patient, suggesting that these are recurrent events that may be important in CLL pathogenesis. Recurrent deletions were identified in chromosome 1 (three patients), chromosome 3 (three patients), chromosome 5 (two patients), chromosome 8 (two patients) and chromosome 14 (seven patients) as well as an amplification in the long arm of chromosome 8 in two patients. The identification of new abnormalities by array-CGH and the increased sensitivity of deletion of small proportions of cells with known deletion identified by FISH demonstrate the complementary nature of these two techniques.

The most common of the new deletions identified involved the distal region of the long arm of the chromosome 14. Using array-CGH assay, a deletion at locus 14q32.33 was detected involving the two most telomeric BAC clones. Of note, all of these patients also had other cytogenetic abnormalities. Structural abnormalities involving the locus 14q32 have been frequently reported to be balanced translocations commonly associate with complex karyotype changing in B-cell lymphoid disease. Two of the cases studied here had such balanced translocations, t(1;14) and t(7;14). Although previous studies have described rare cases in CLL involving deletions of chromosome 14, these did not involve the deletion 14q32.33 that was frequently detected in this study. One case studied here had the previously described deletion 14q24 as well as the deletion at 14q32.33

### Example 2

### Deletion 14q32.33 Confirmed by Interphase FISH

Three of the cases with deletion 14q32 were patients with deletions at 13q14.3 detected by FISH as a sole abnormality. Such patients generally have good prognosis. However, it was noted that all three of these patients had rapidly progressive disease, and hypothesized that the deletion 14q32.33 might be associated with poor prognosis. In order to confirm the presence of the deletion detected by array-CGH at 14q32.33 was real, and to validate that this might be associated with poor outcome, the BAC clone involved in 14q32.33 deletion was identified and prepared a cy3-dUTP labeled FISH probe from DNA isolated from the BAC clone (CTC-200D12). The BAC clones involved in all recurrent DNA copy number changes were identified by software analysis. BAC RP11-80H2 (chromosome 13q14.3-q21) was obtained from BACPAC Resources (Children's Hospital Oakland, Calif.) and GS-200D12 (chromosome 14q32.33) from Spectral Genomics. BAC DNA was isolated using the PhasePrep BAC DNA kit (Sigma-Aldrich Inc. St. Louis, Mo.) and sequenced to confirm the identity of the clone. Subsequently, the clones were labeled by nick translation with fluorescein-11-dUTP. Single and dual-color FISH analysis were performed on interphase nuclei preparations from patient and healthy donor samples using standard protocols. FISH results were visualized by an Olympus AX70 fluorescent microscope equipped with DAPI, FITC, dual and triple band pass filters and images acquired with a CCD camera and Genus software (Applied Imaging, Santa Clara, Calif.).

In all seven patients who had the deletion 14q32.33 detected by array-CGH, this deletion was confirmed by interphase FISH.

To evaluate the cut-off level for sensitivity and specificity of the FISH analysis samples from 10 healthy volunteer were used as controls. In addition, samples were obtained from patients, previously characterized as having 13q14.3 deletion by standard FISH probes and performed dual color FISH experiment using as probes DNA from the identified BAC clones for 13q14.3 (RP11-80H2) and 14q32.33. Among these patients with deletion 13q14.3, several patients also had deletion 14q32.33. Only a proportion of the cells with deletion 13q14.3 had deletion 14q32.33, suggesting that this abnormality is probably a secondary event occurring after development of CLL. There was an association between the detection of deletion 14q32.33 and requirement for treatment, in support of the hypothesis that the presence of this deletion has poor prognostic significance.

### Example 3

### Identification of V_{H} Deletions by Array-CGH

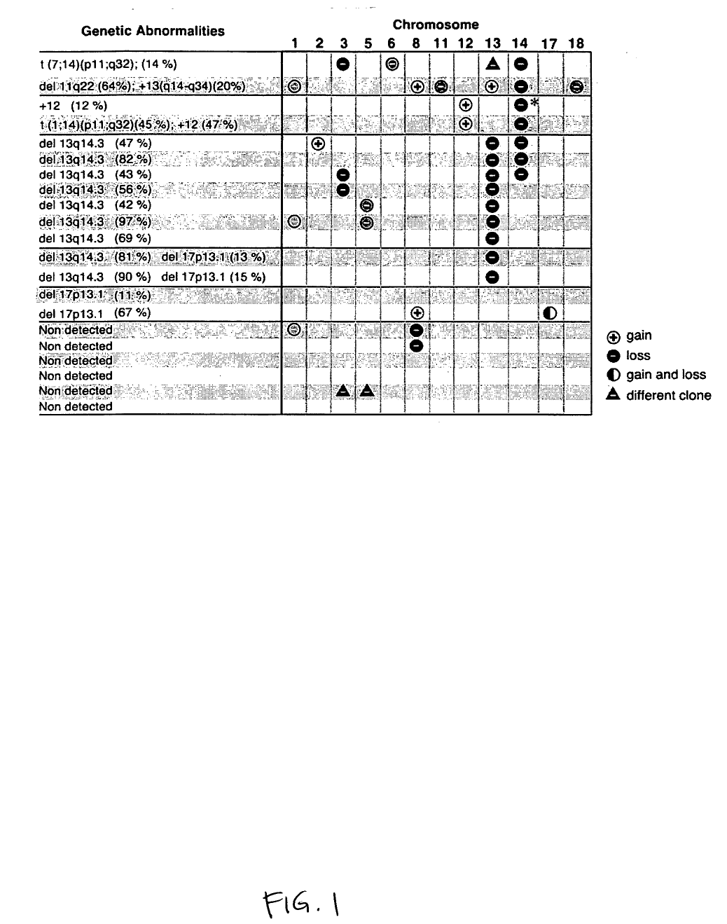

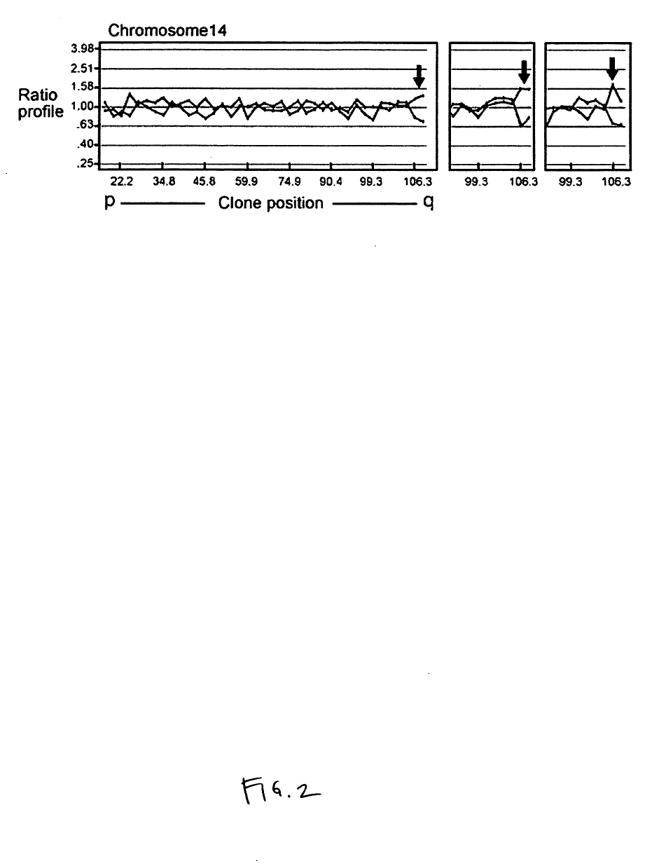

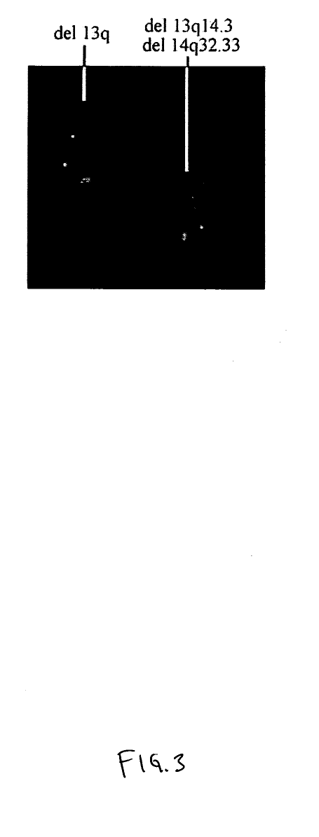

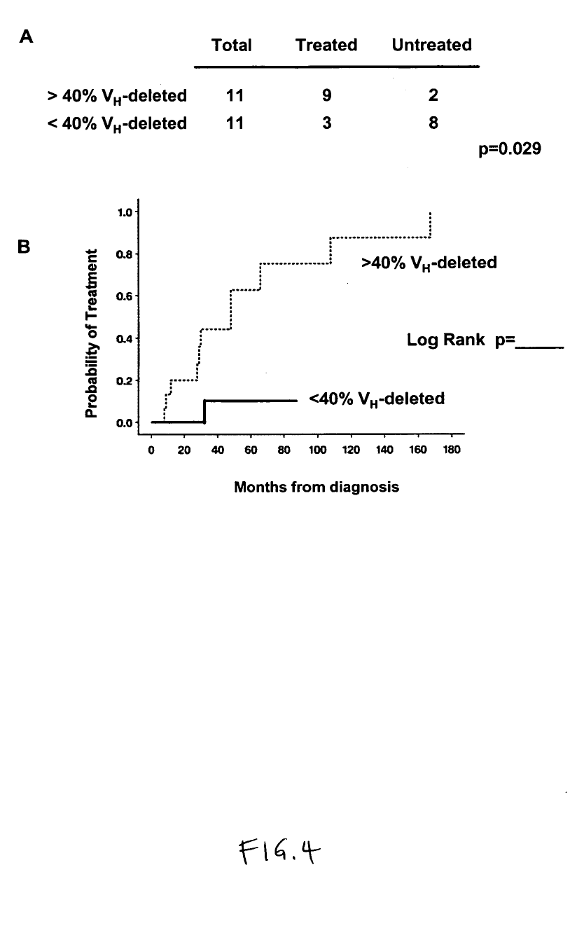

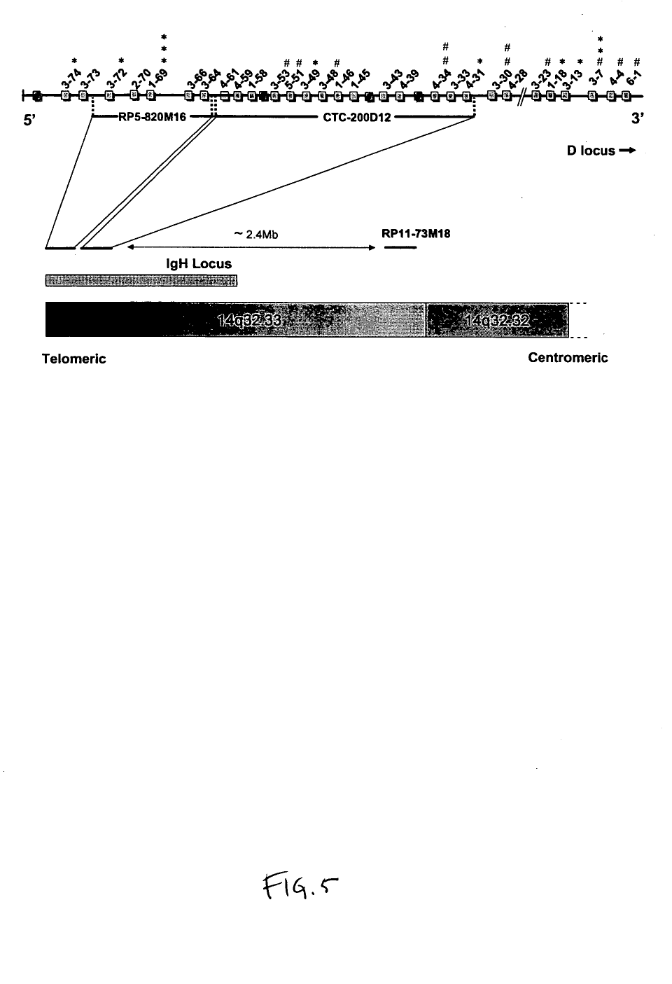

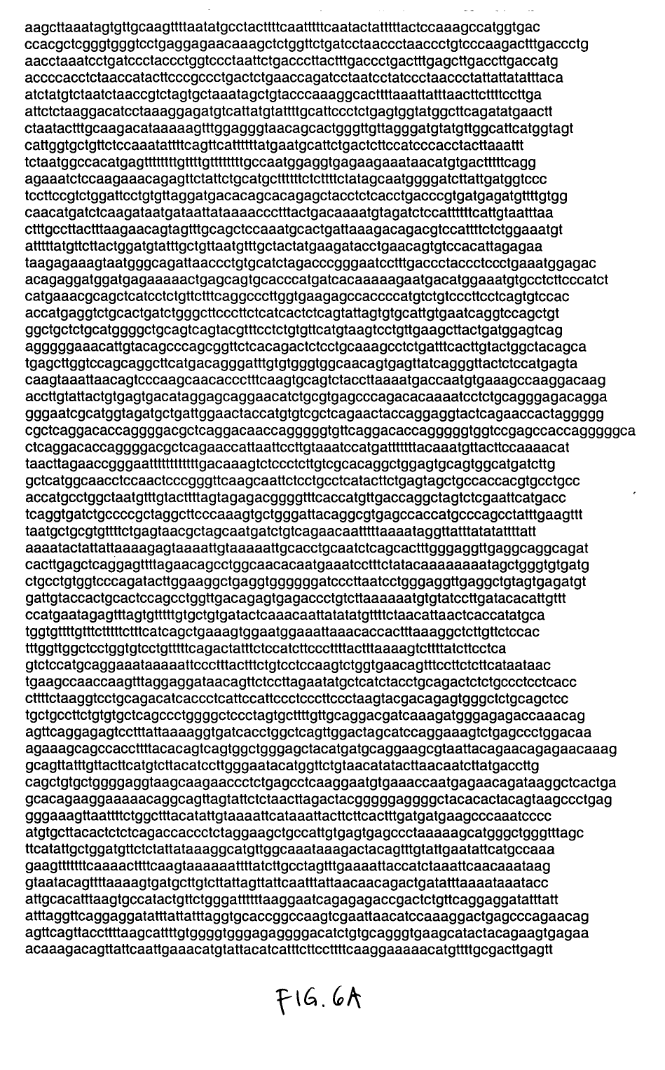

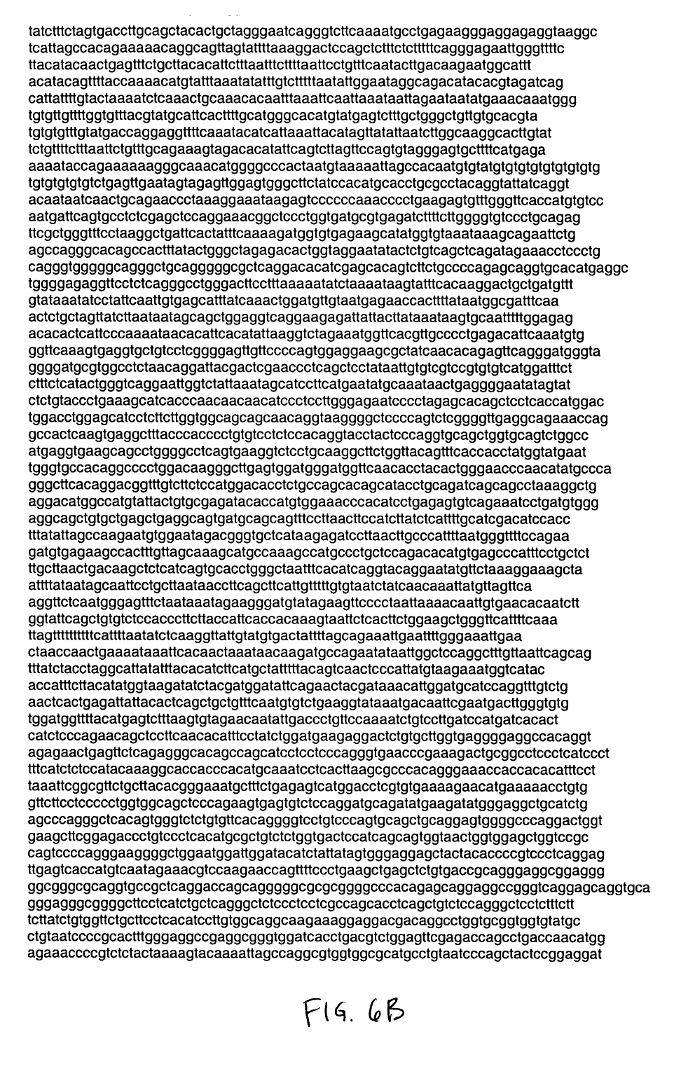

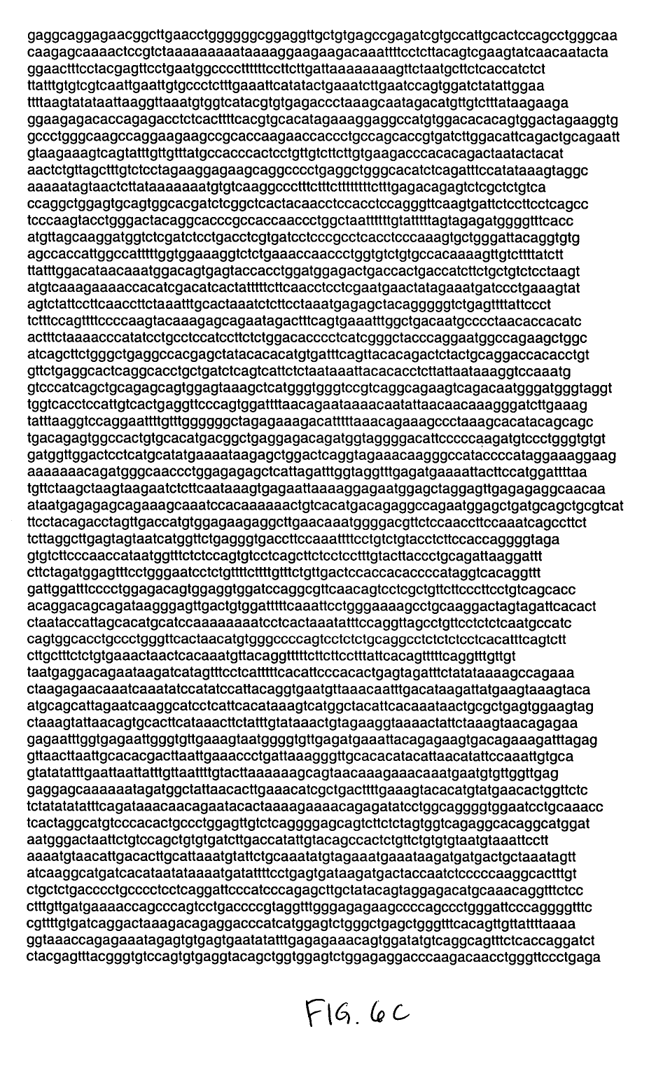

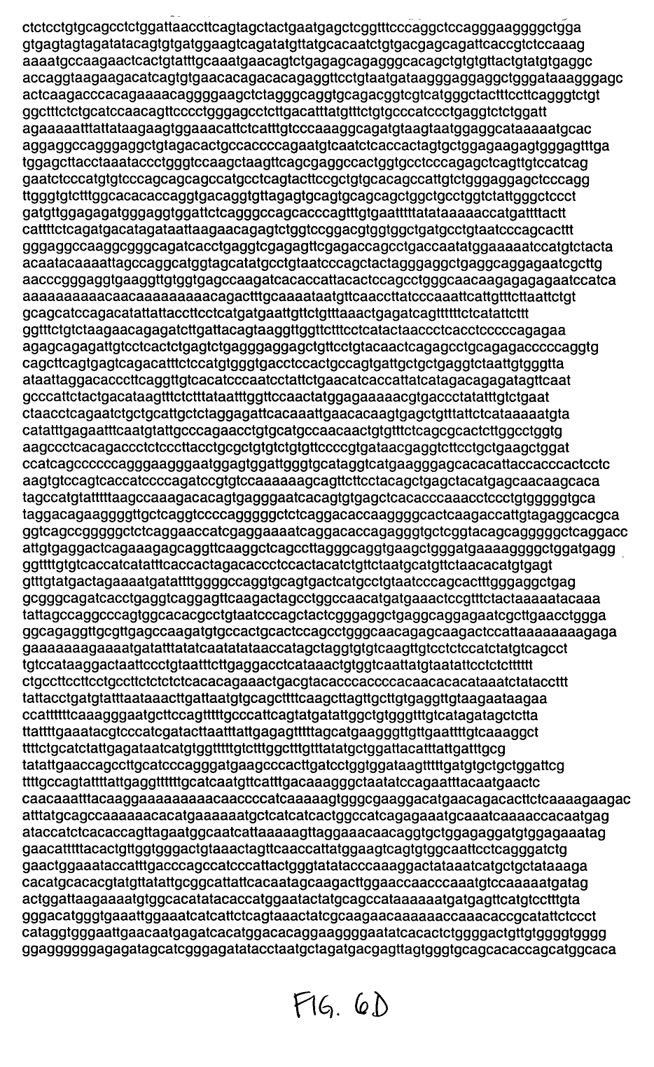

Array-CGH was performed on 21 previously untreated CLL patients with a variety of cytogenic abnormalities, including six patients with no detectable abnormalities. Array-CGH detected the majority of abnormalities detected by conventional cytogenetics and also revealed novel recurrent genomic imbalances (FIG. 1; the percentage of cells with each abnormality detected by conventional cytogenetics and/or FISH is indicted in parentheses). A total of 40 genomic imbalances (seven gains and 33 losses) on 12 chromosomes were identified by array-CGH, including 14 imbalances detected by the previous FISH analysis (FIG. 1; for each chromosome, aberrations involving the same BAC clone are indicated in the same color).

As expected, balanced translocations (2 cases) and 17p13.1 deletions occurring in 15% or less of the cells (3 cases) were not detected by array-CGH. All other abnormalities were confirmed by array-CGH, including 13q14, 11q22.23, and 17p13.1 deletions. In two cases with trisomy 12, array-CGH confirmed gain of the whole chromosome despite the detection of this abnormality in only 12% of cells in one case by FISH. The remaining 26 imbalances were not previously identified and 19 (73%) of these represented recurrent imbalances involving the same region in more than one patient. Recurrent balances were identified at loci on 1p13, 3q26, 5p14, 8q24, and 14q32.33, and involving an extended region on chromosome 8q (FIG. 1; the asterisk denotes detection by array-CGH of a large 14q24-32 deletion in addition to the smaller IgH locus deletion at 14q32.33. Abbreviations: del (deletion), +(trisomy)).

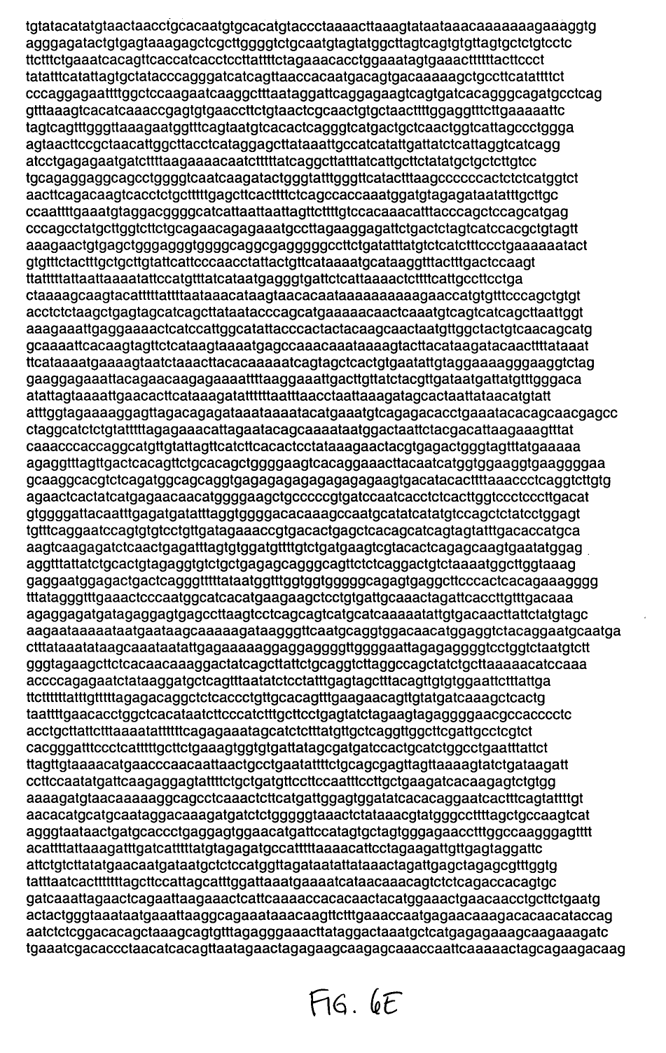

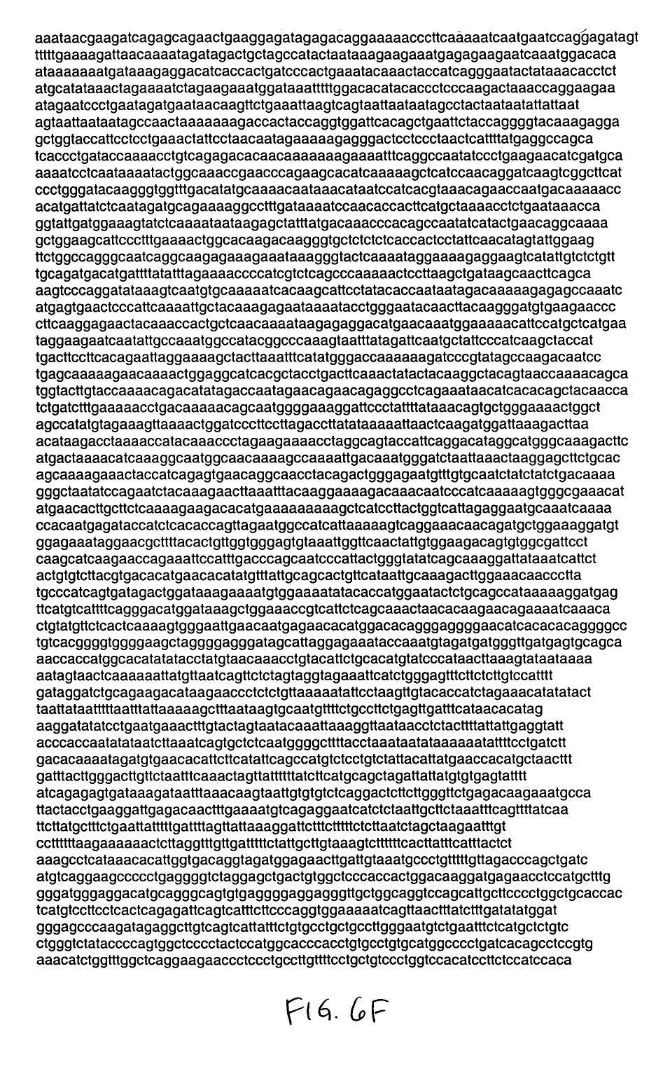

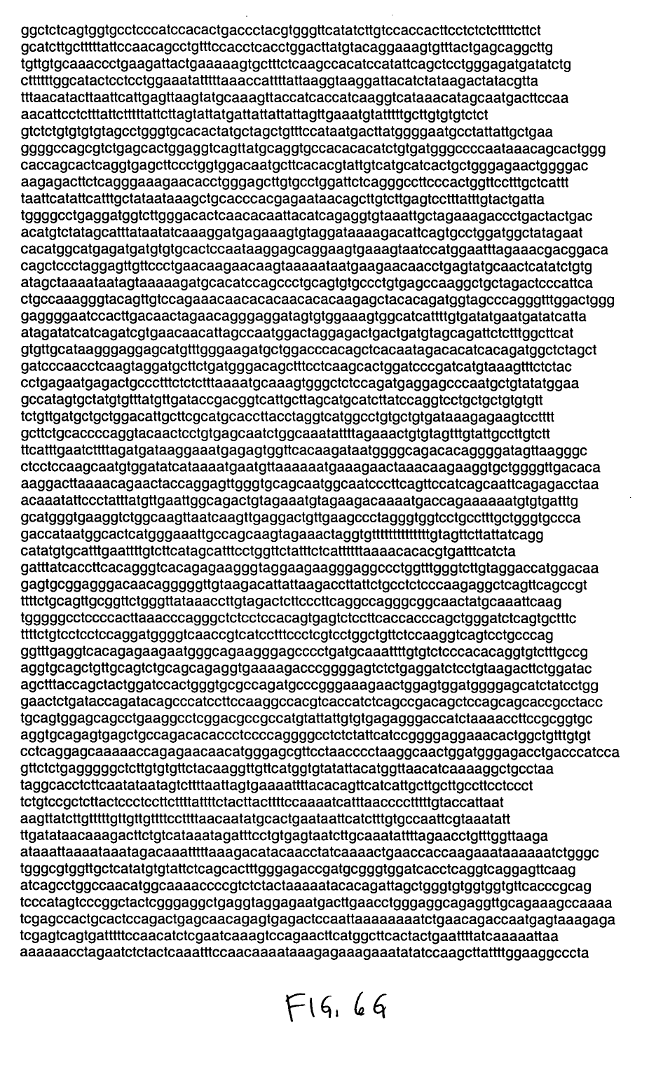

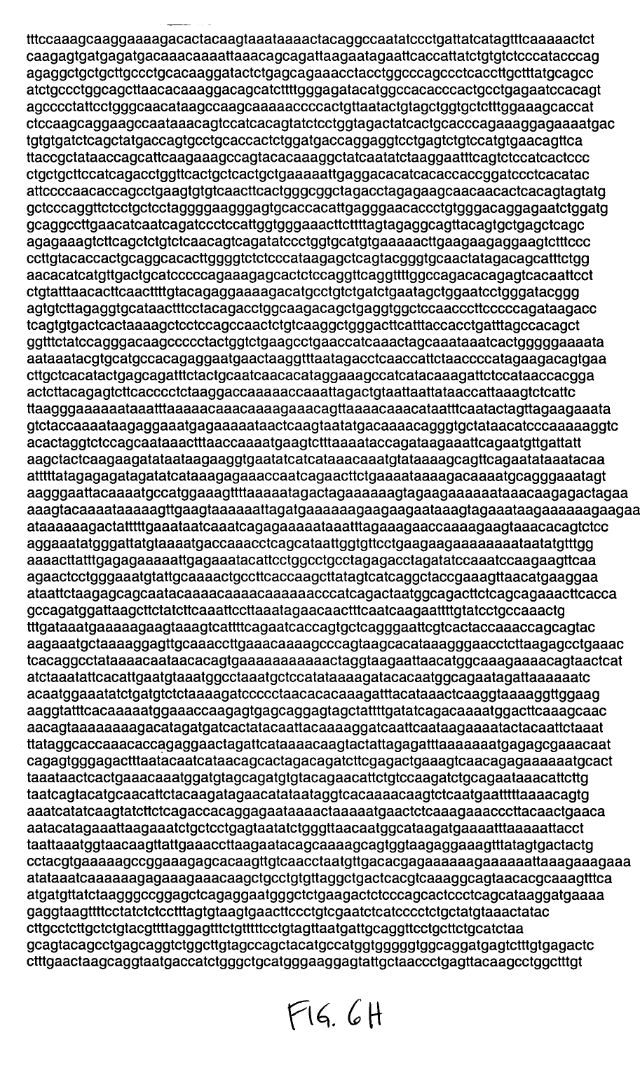

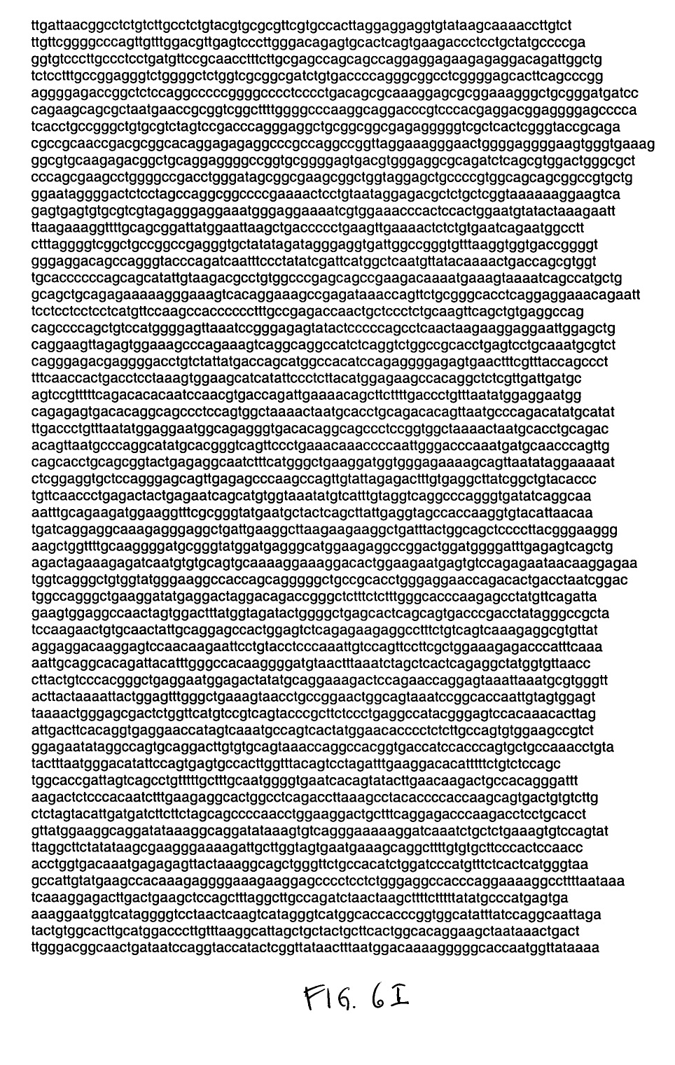

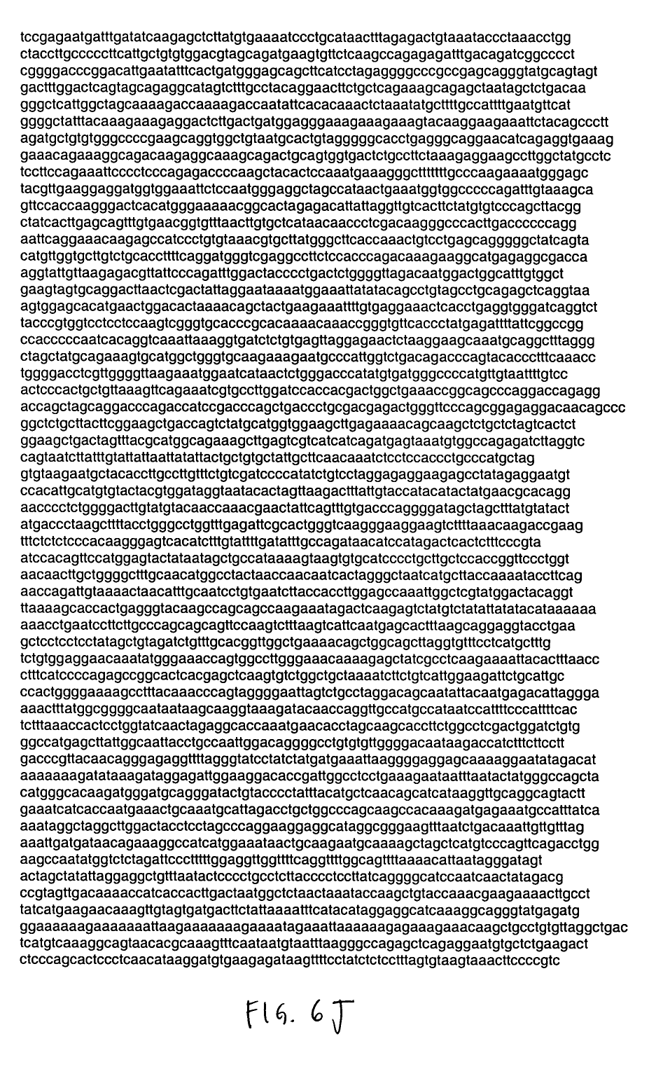

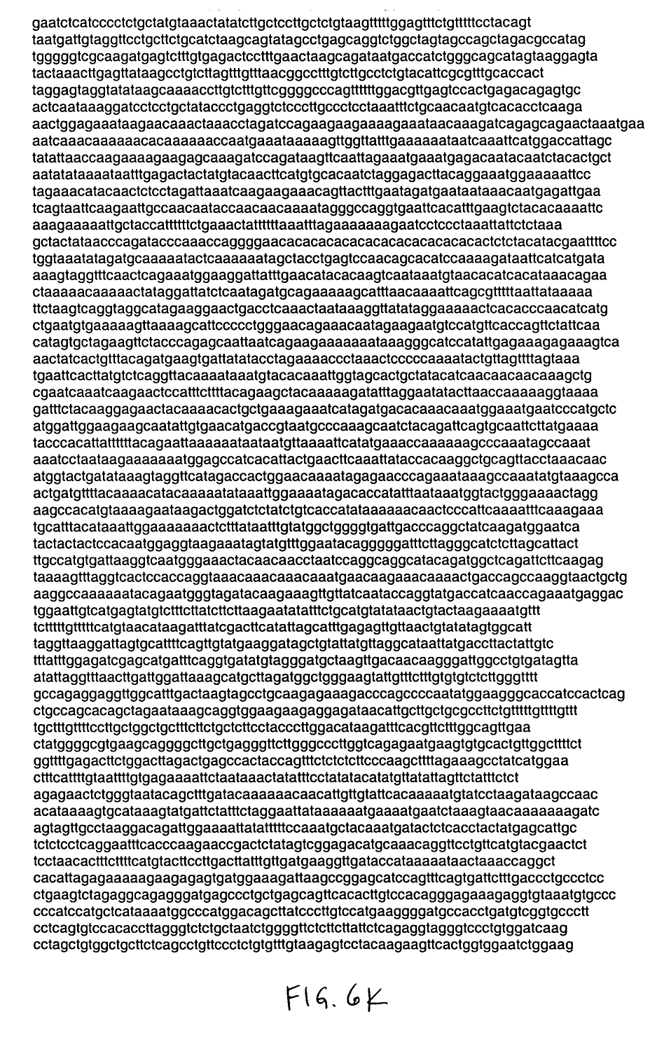

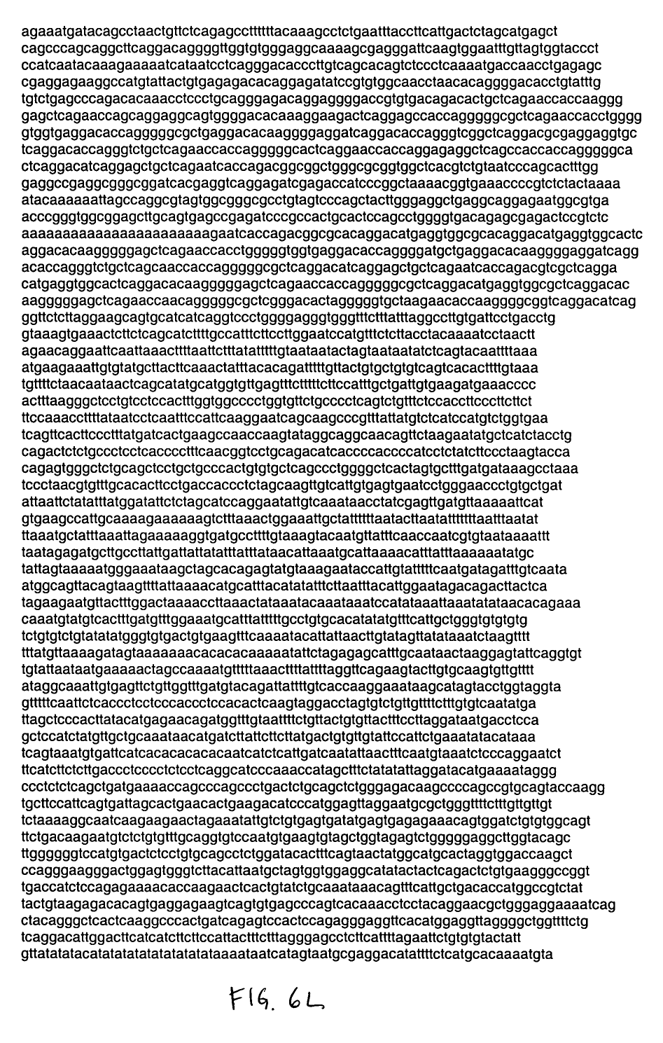

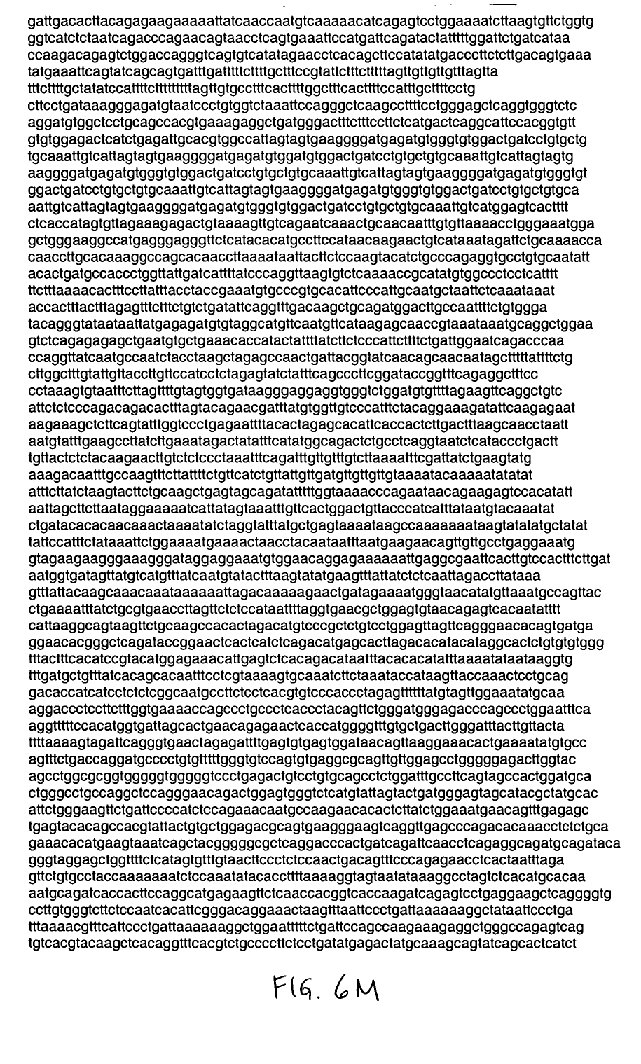

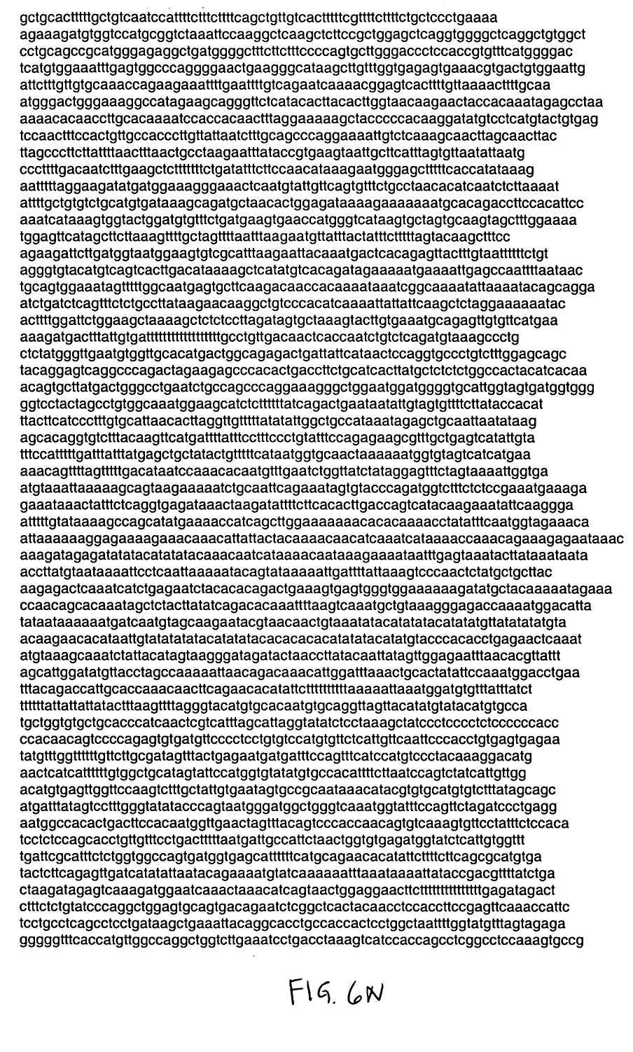

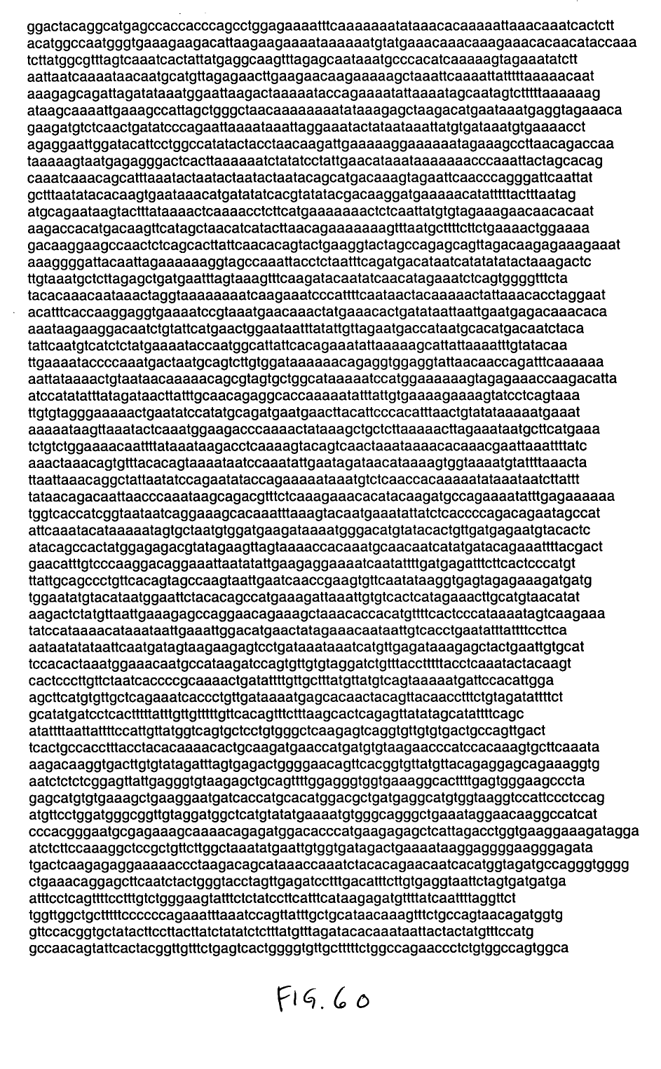

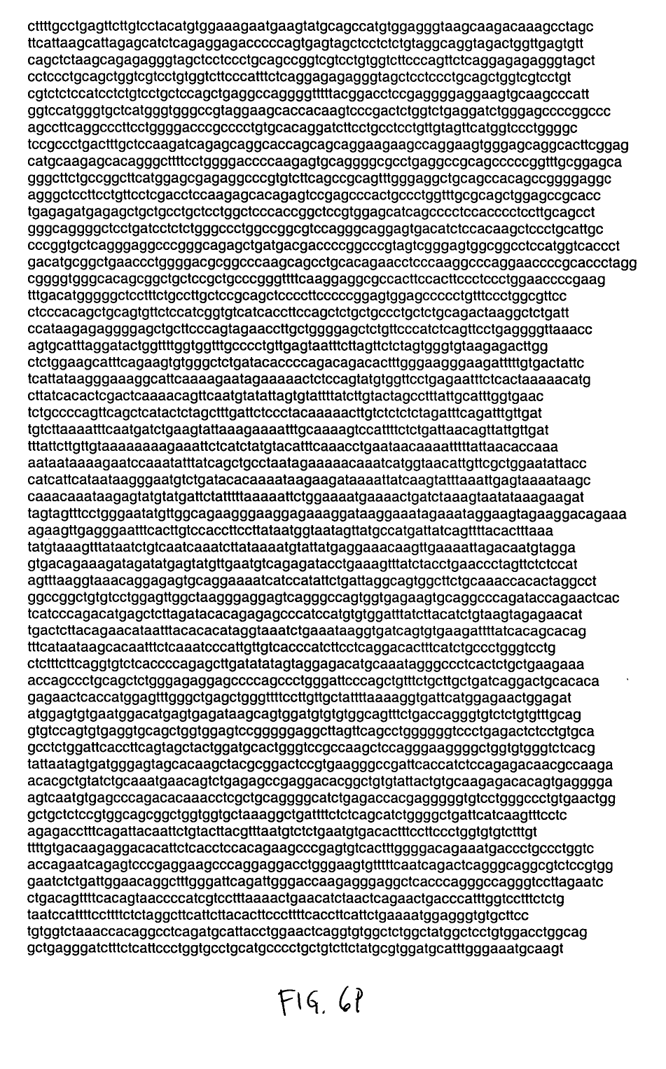

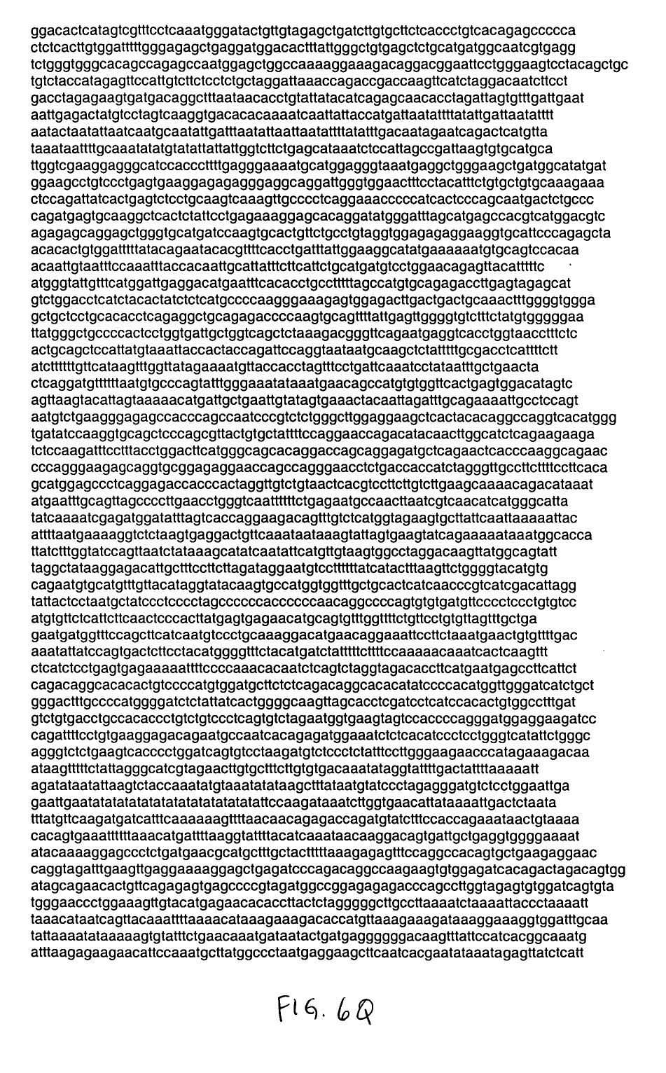

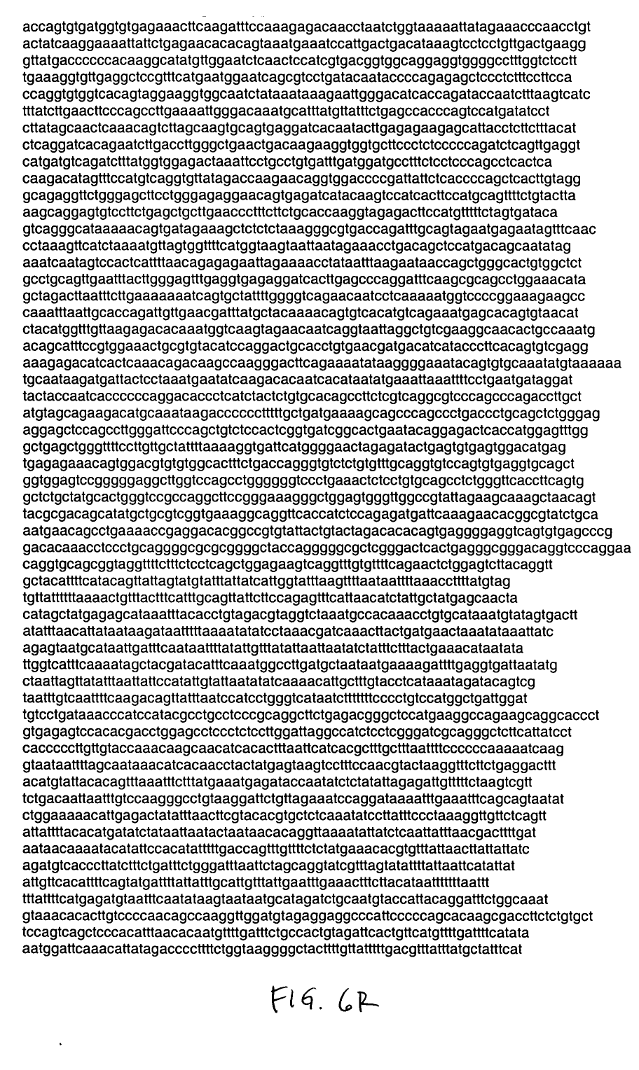

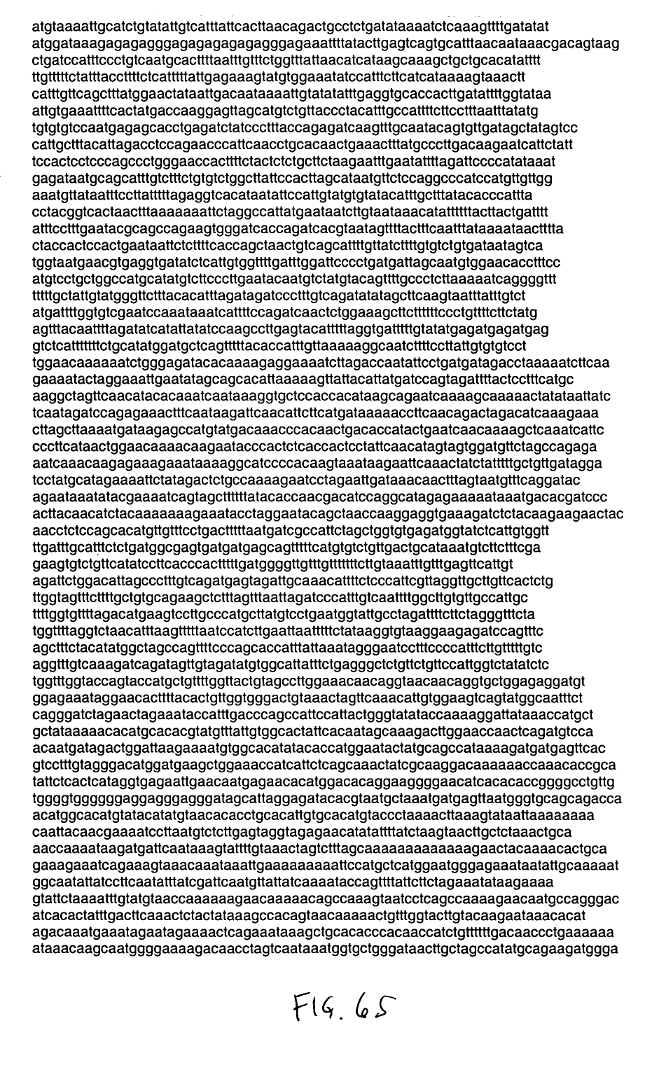

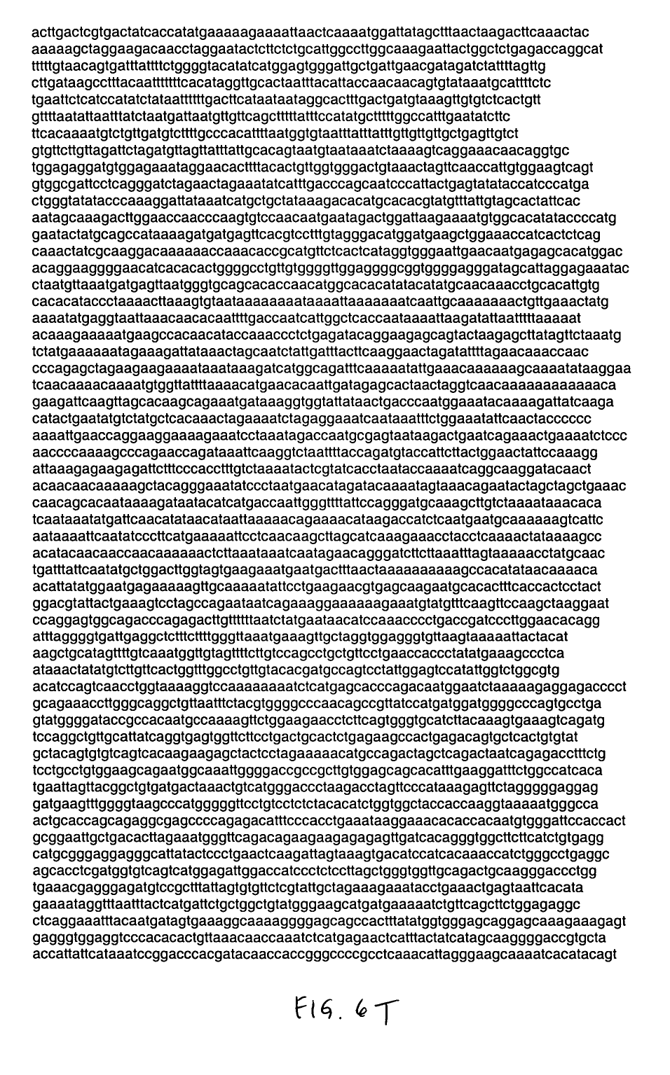

The most common imbalance, detected in seven of 21 patients (33%), was a deletion involving the two most telomeric 14q clones on the CGH array (CTC-200D12 and RP5-820M16), which map to the subtelomeric V_{H} region of the IgH locus at 14q32.33 (FIG. 2; CLL specimens were analyzed in duplicate on two arrays using a dye-swap experimental design: red lines represent hybridization of Cy3-labeled tumor DNA with Cy5-labeled reference DNA, and blue lines represent Cy5-labeled tumor DNA hybridized with Cy3-labeled reference DNA. Genomic losses are represented as red lines above and blue lines below the abscissa). These deletions were subsequently confirmed by interphase FISH (see below and FIG. 3, in which dual-color interphase FISH shows mosaic V_{H} deletion. One cell shows isolated deletion at 13q14.3 (RP11-80H2) and an adjacent cell shows both 13q14.3 and V_{H} (CTC-200D12) deletions. Hybridized probes were visualized using an Olympus AX70 fluorescent microscope and images were acquired with a Photometrics KAF1400 CCD camera using Genus software (Applied Imaging)).

CLL Patients and Controls

Peripheral blood mononuclear (PBMC) were obtained after IRB approval and signed informed consent from 37 patients with previously untreated CLL, including 6 patients with no detectable cytogenetic abnormalities, 23 patients with isolated 13q14.3 deletions, and 8 patients with other cytogenetic abnormalities. Ficoll-purified PBMC from CLL patients contained an average of 97% lymphocytes (range 82-100%). Each patient met standard diagnostic criteria for CLL (Cheson et al., Blood 87:4990-4997, 1996) and was previously characterized by conventional karyotyping and FISH analysis using ATM (11q22.3), D12Z3 (12cen), D13S319 (13q14.3), and TP53 (17p13.1) probes (Vysis, Downers Grove, Ill.).

Array-CGH

Two micrograms of purified genomic DNA from CLL PBMC were labeled with Cy3-(or Cy5-) dCTP, and hybridized along with Cy5-(or Cy3-) labeled normal reference DNA (Promega) to DNA microarrays containing 1003 independent bacterial artificial chromosome (BAC) and P1-derived artificial chromosome (PAC) clones chosen to represent the entire human genome at 2-4 megabase resolution (Spectral Genomics, Houston, Tex.). Hybridized arrays were washed, scanned on a GenePix 4000B laser scanner (Axon Instruments, Union City, Calif.) and analyzed using SpectralWare software (Spectral Genomics) (lafrate et al., Nat. Genet. 36:949-51, 2004).

### Example 4

### V_{H} Deletions in CLL Patients with 13q14.3 Deletions

V_{H} region deletions were detected by array-CGH in 3 of 7 patients whose sole previously detected molecular cytogenetic abnormality was a deletion at 13q14.3. To further characterize the newly identified V_{H} deletions in a cytogenetically well-defined cohort of CLL patients, dual-color interphase FISH was performed using 13q14.3 and V_{H} region (CTC-200D12) probes on PBMC from 22 previously untreated CLL patients with otherwise isolated 13q14.3 deletions.

Dual-color interphase FISH using 13q14.3 (RP11-80H2) and V_{H} region (CTC-200D12) probes was performed using standard protocols (Dohner et al., Blood 85:1580-1589, 1995; Dohner et al., Blood 89:2516-2522, 1997). In control experiments using PBMC isolated from 10 control patients, <=1% of cells showed deletion with the RP11-80H2 probe and <=6% showed deletion with the CTC-200D12 probe.

As shown in Table 1, V_{H} region deletions were detected in the majority of CLL patients in a mosaic pattern, the proportion of V_{H}-deleted cells in a given patient ranging from 0% to 94% (median 40%). Table 1 shows clinical characteristics on 22 CLL patients with otherwise isolated 13q14.3 deletions. International CLL working group staging (Ref.16) at time of PBMC sampling and current statuses are shown. Abbreviations: M (male), F (female), CR (complete remission), PR (partial remission), Mut (mutated; <=98% homology to IgBlast germline sequence), U (unmutated; >98% homology to germline sequence), del (deletion), T (treated), NT (remains untreated).

Similar mosaic deletion patterns were seen in FISH experiments using other V_{H} region probes that mapped near CTC-200D12 but not with IgH constant region probes (data not shown), confirming that the mosaicism cytogenetic abnormalities in CLL—including trisomy 12 (Raghoebier et al., Leukemia 6:1220-1226, 1992) and deletions at 11q22 (Fegan et al., Leukemia 9:2003-2008, 1995; Dohner et al., Blood 89:2516-2522, 1997), 17p13 (Chevallier et al., Br. J. Haematol. 116:142-150, 2002), and 13q14 (Dewald et al., Br. J. Haematol. 121:287-295, 2003; Navarro et al., Br. J. Haematol. 102:1330-1334, 1998) in addition to the V_{H} region deletions described here—suggests that such secondary aberrations are a common feature of CLL and may result in emergence of CLL subclones with more aggressive growth properties and/or resistance to chemotherapeutic agents (Rosenwald et al., Blood 104:1428-1434, 2004).

TABLE 1
Clinical features and incidence of V_{H} deletions in CLL patients with 13q14 deletions

| | | | | | | | V_{H} | del(13q) | del(13q) | del(V_{H}) | | Time to |
|---|---|---|---|---|---|---|---|---|---|---|---|---|
| | | | White | Stage | | Mutation | gene | only | del(V_{H}) | Total | Out- | Treatment |
| Patient | Age | Sex | Count | at sample | current | status | used | % | % | % | come | (months) |
| 1 | 69 | M | 105 | IIB | PR | U | 1-69 | 4 | 86 | 94 | T | 12 |
| 2 | 43 | F | 145 | 0A | Treating | Mut | 3-7 | 1 | 52 | 90 | T | 66 |
| 3 | 49 | M | 181.5 | IIB | Relapsed | U | 1-69 | 7 | 78 | 86 | T | 48 |
| 4 | 65 | F | 18.1 | IIIC | Relapsed | Mut | 3-49 | 5 | 63 | 83 | T | 168 |
| 5 | 53 | F | 29.7 | 0A | PR | Mut | 4-31 | 14 | 82 | 82 | T | 108 |
| 6 | 52 | M | 11.1 | 0A | 0A | Mut | 3-72 | 9 | 52 | 74 | NT | 32+ |
| 7 | 57 | F | 96.1 | IB | IB | U | 3-13 | 25 | 54 | 59 | NT | 55+ |
| 8 | 70 | M | 28 | IIIC | PR | U | 1-69 | 23 | 49 | 53 | T | 48 |
| 9 | 51 | M | 91.9 | IIB | CR | U | 1-18 | 37 | 50 | 53 | T | 9 |
| 10 | 53 | M | 64.2 | 0A | IB | Mut | 3-74 | 33 | 31 | 42 | T | 30 |
| 11 | 65 | M | 41.3 | 0A | Treating | Mut | 3-7 | 55 | 38 | 42 | T | 28 |
| 12 | 61 | F | 24.2 | 0A | 0A | U | 6-1 | 49 | 36 | 38 | NT | 30+ |
| 13 | 56 | M | 19.7 | IA | PR | Mut | 3-23 | 38 | 23 | 32 | T | 8 |
| 14 | 65 | F | 16.0 | 0A | CR | U | 3-30 | 39 | 11 | 17 | T | 29 |
| 15 | 42 | M | 13.6 | 0A | 0A | Mut | 3-7 | 55 | 16 | 16 | NT | 24+ |
| 16 | 50 | F | 20.9 | 0A | 0A | Mut | 4-34 | 55 | 9 | 13 | NT | 34+ |
| 17 | 49 | M | 31.1 | IA | IA | Mut | 3-48 | 59 | 8 | 12 | NT | 36+ |
| 18 | 56 | F | 21.9 | 0A | 0A | Mut | 5-51 | 68 | 9 | 9 | NT | 68+ |
| 19 | 45 | M | 27.5 | 0A | 0A | Mut | 3-30 | 41 | 7 | 8 | NT | 42+ |
| 20 | 52 | M | 51.6 | IIB | IIB | Mut | 3-53 | 61 | 4 | 4 | NT | 81+ |
| 21 | 58 | M | 65.1 | IIB | CR | Mut | 4-34 | 92 | 3 | 3 | T | 84 |
| 22 | 35 | F | 20.3 | IB | IB | Mut | 4-4 | 78 | 0 | 0 | NT | 77+ |

### Example 5

### V_{H} Deletions and Prognosis

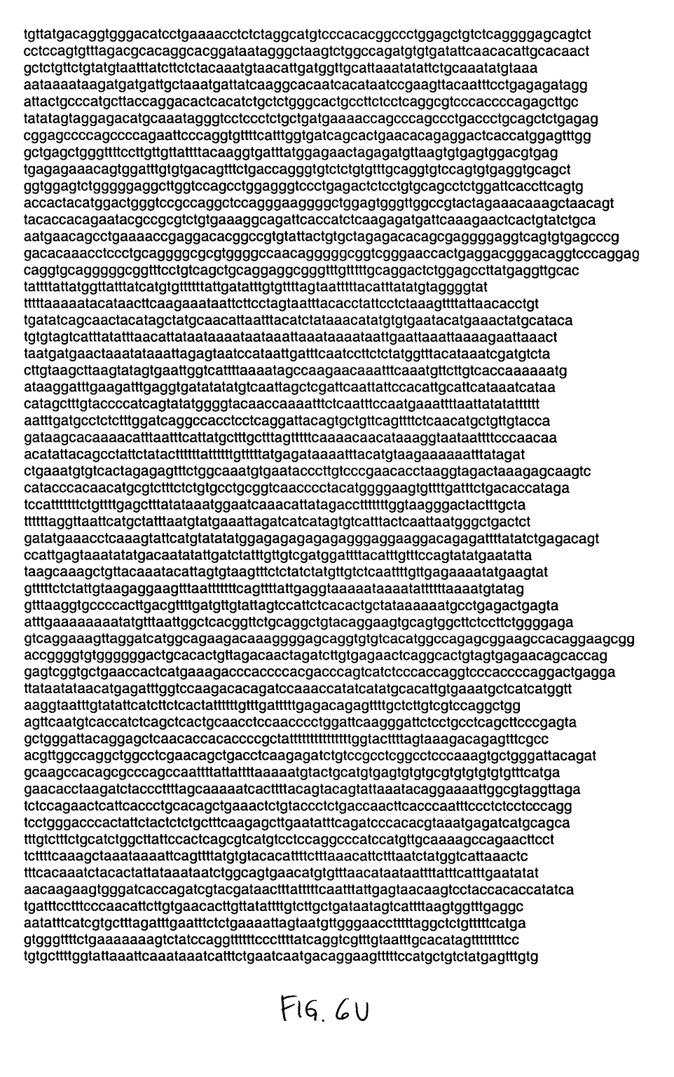

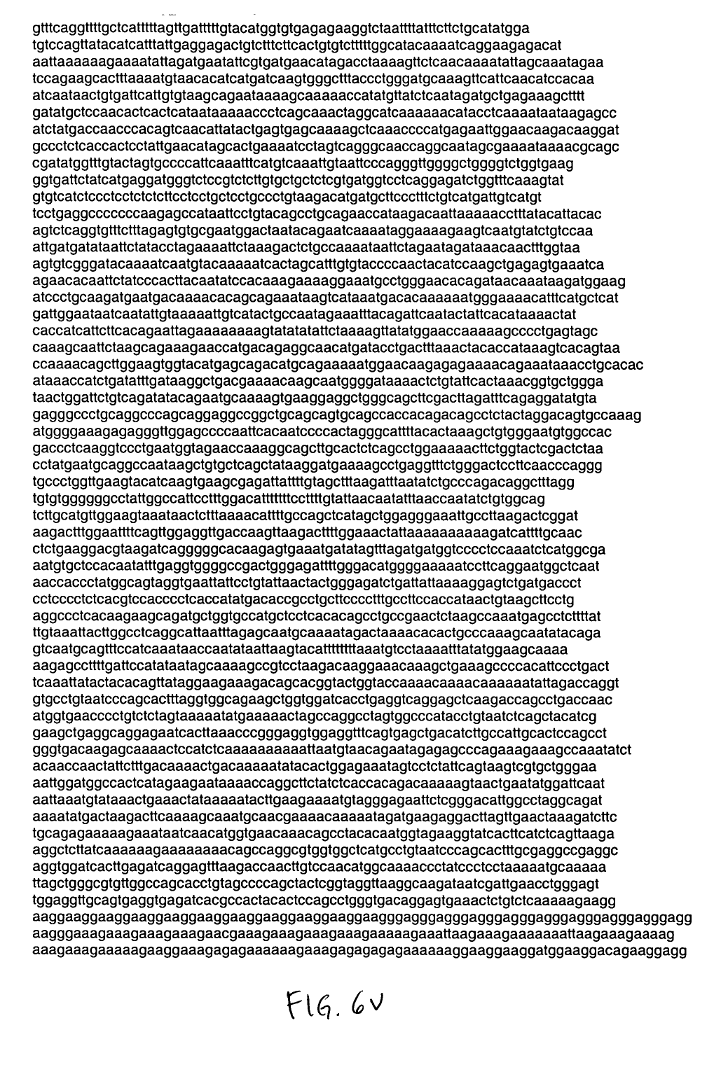

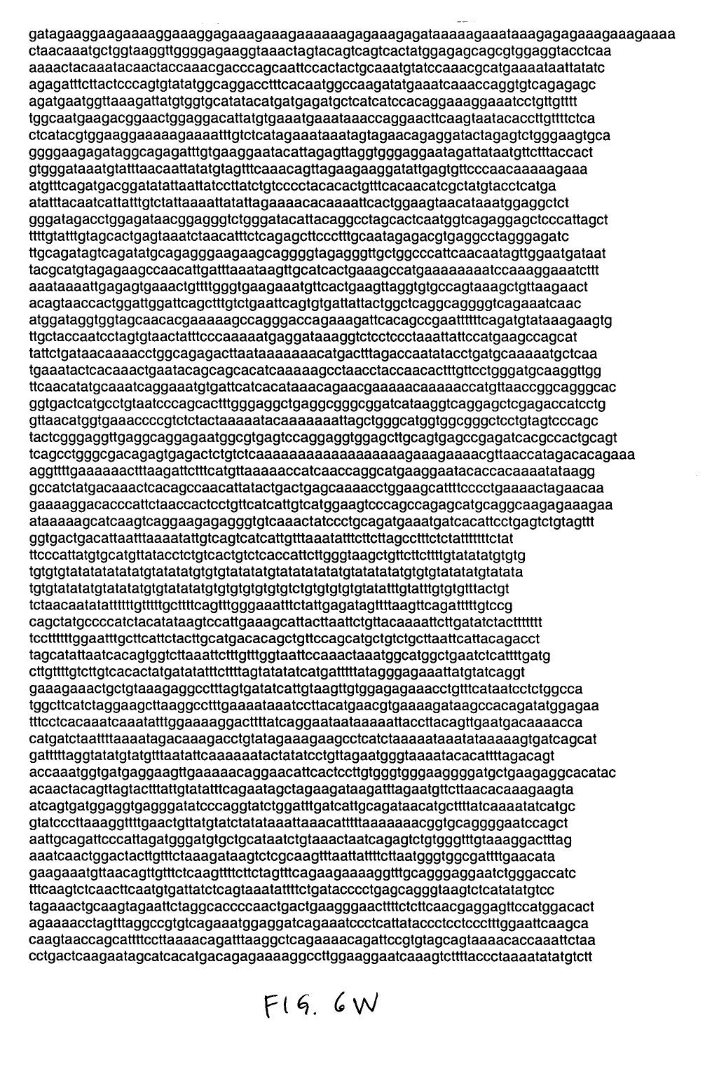

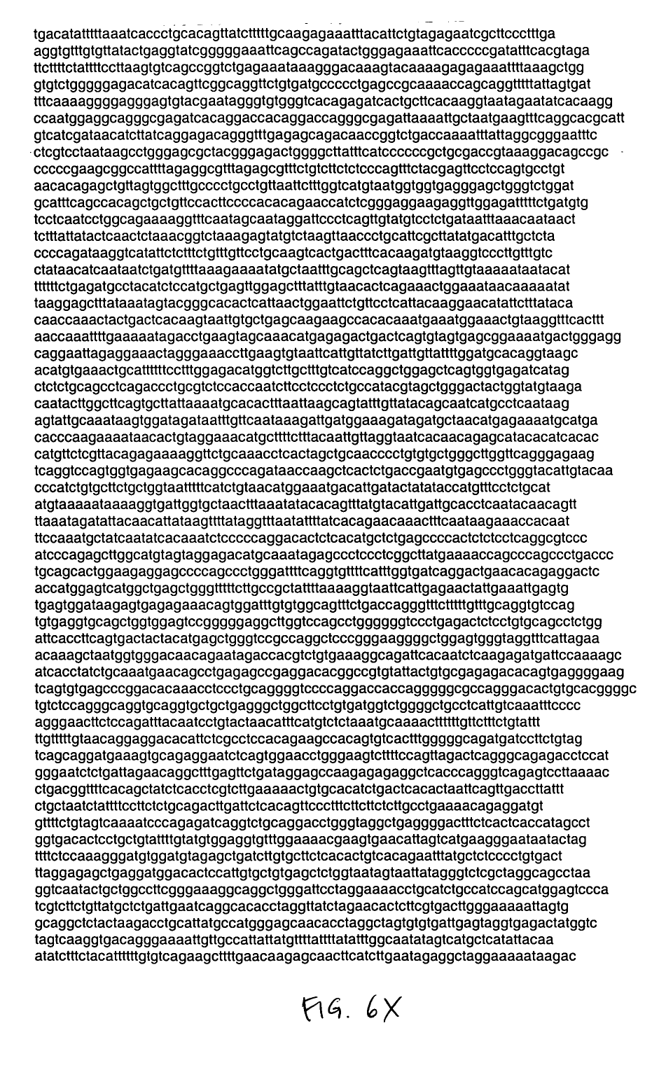

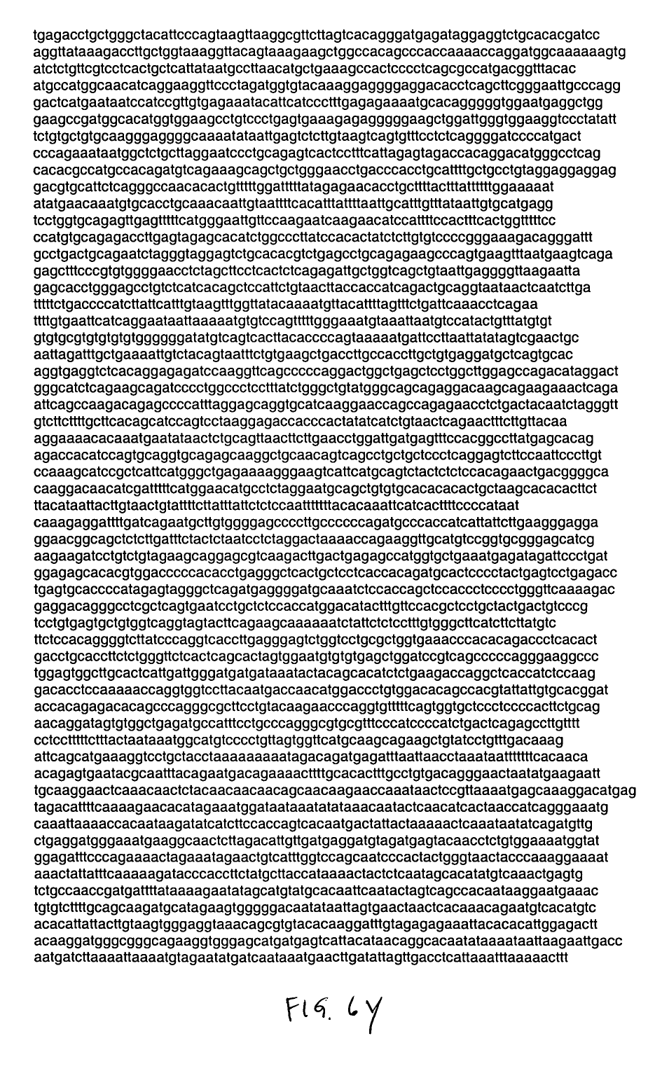

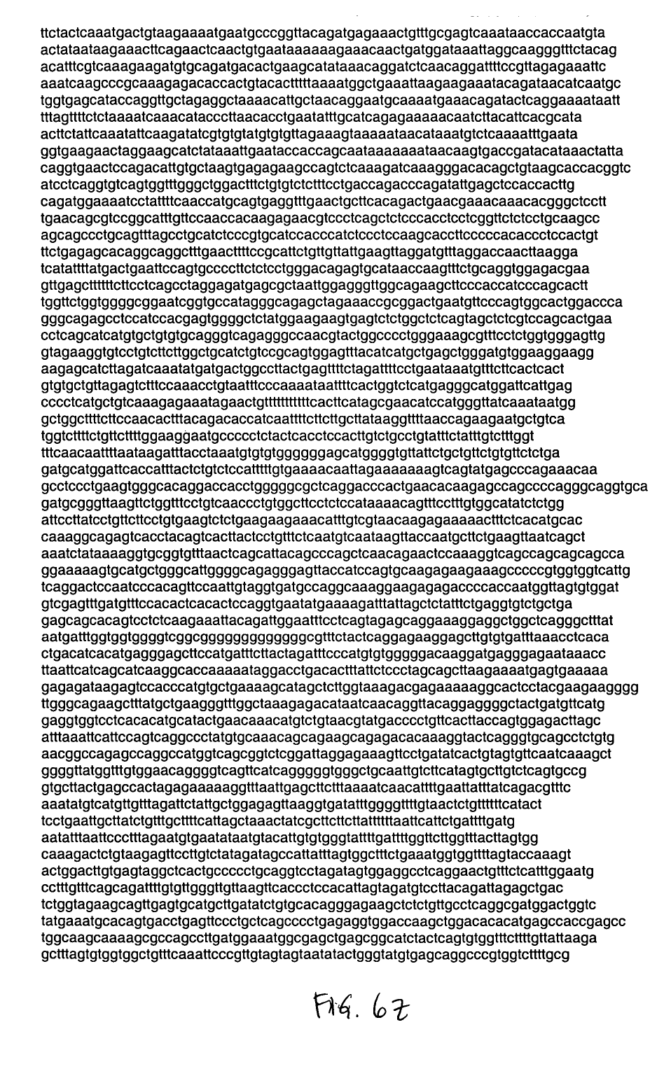

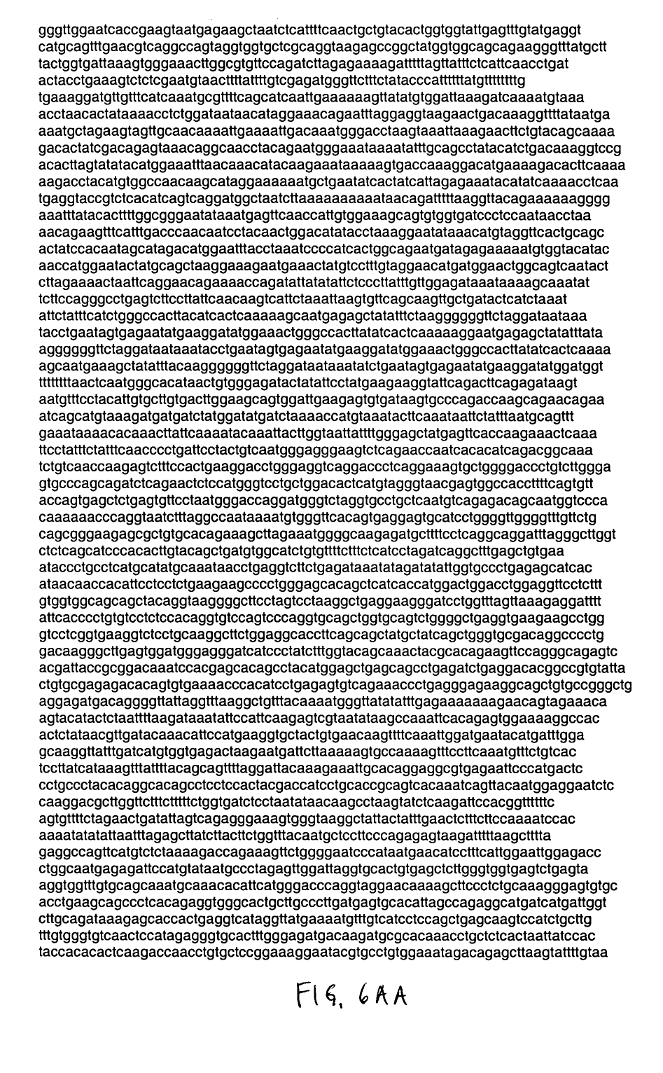

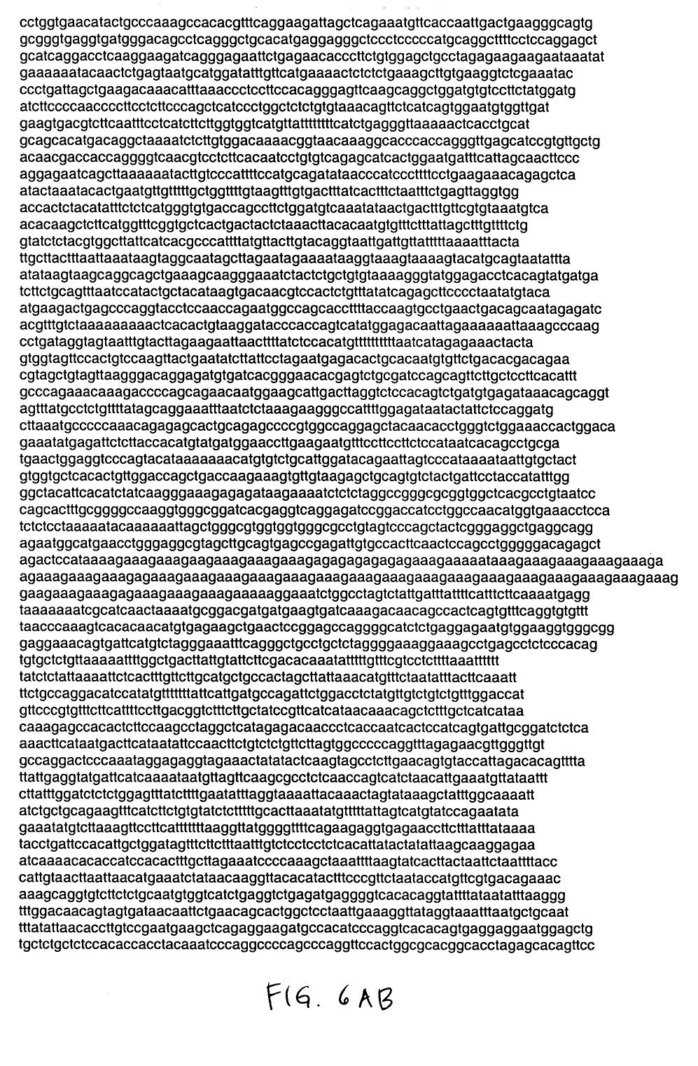

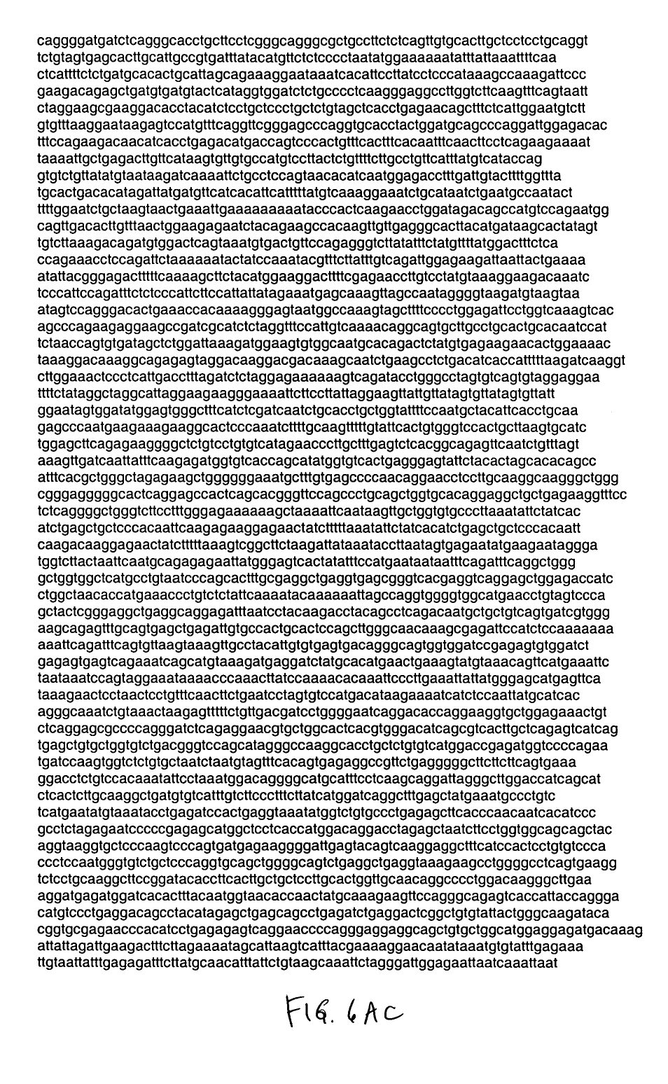

CLL patients with isolated deletions at 13q14.3 generally have favorable prognosis, while patients having 13q14.3 deletions together with additional cytogenetic aberration(s) tend to have more aggressive disease (Dohner et al., N. Engl. J. Med. 343:1910-1916, 2000). To determine the prognostic impact of V_{H} deletion in patients with otherwise isolated 13q14.3 deletions, the clinical outcomes of patients in the cohort that had greater than or less than the median of 40% V_{H}-deleted cells were determined. Patients having >40% V_{H}-deleted cells (n=11) were more likely to have been treated (p=0.029 by Fisher's exact test) than those having <40% V_{H}-deleted cells (n=11) and had a shorter time from diagnosis until treatment (p=_ by log-rank test) (FIG. 4). Patients that had >40% V_{H}-deleted cells were more likely to have unmutated IgH sequences (5/11 patients) than those having <40% V_{H}-deleted cells (2/11 patients). However, the presence of >40% V_{H}-deleted cells was still associated with treated status (p=0.04) in those patients with mutated IgH sequences (n=15).

### Example 6

### V_{H} Deletions and V_{H} Gene Utilization

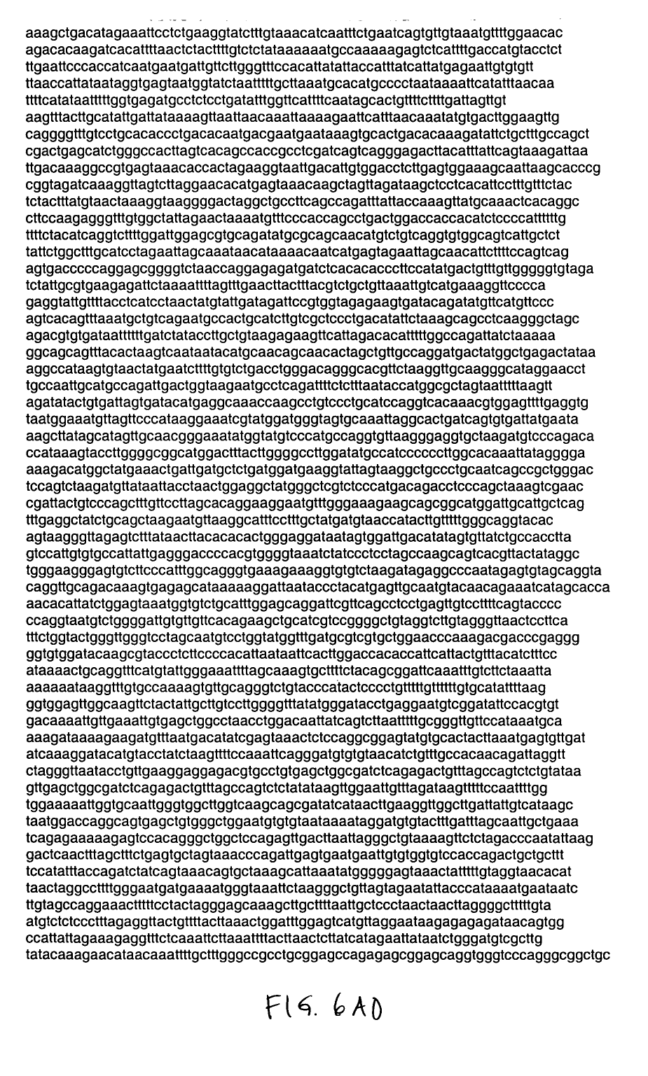

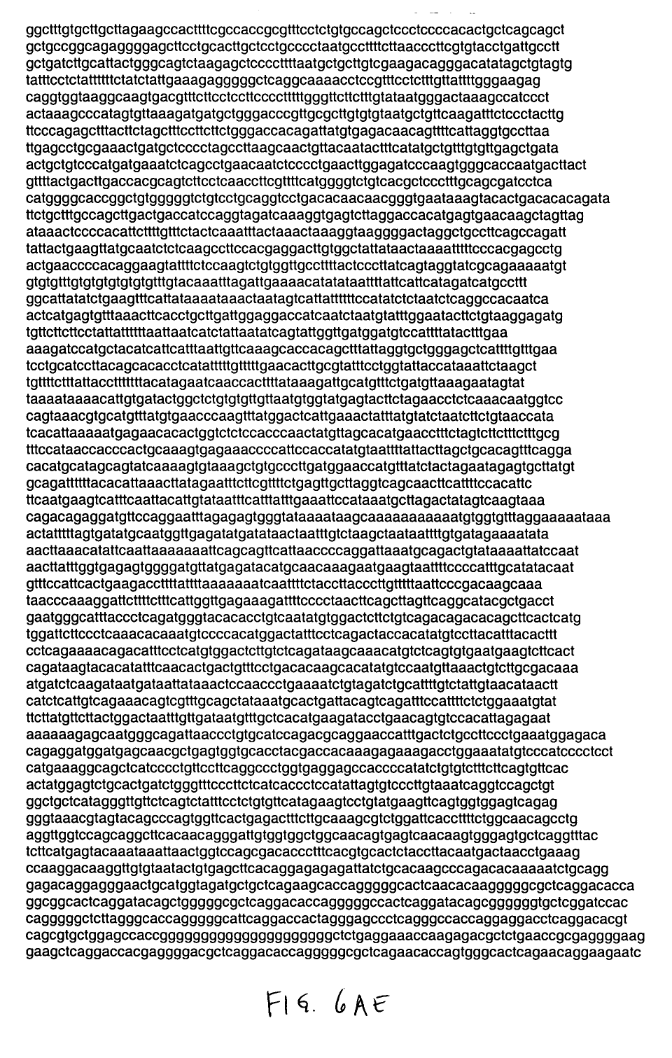

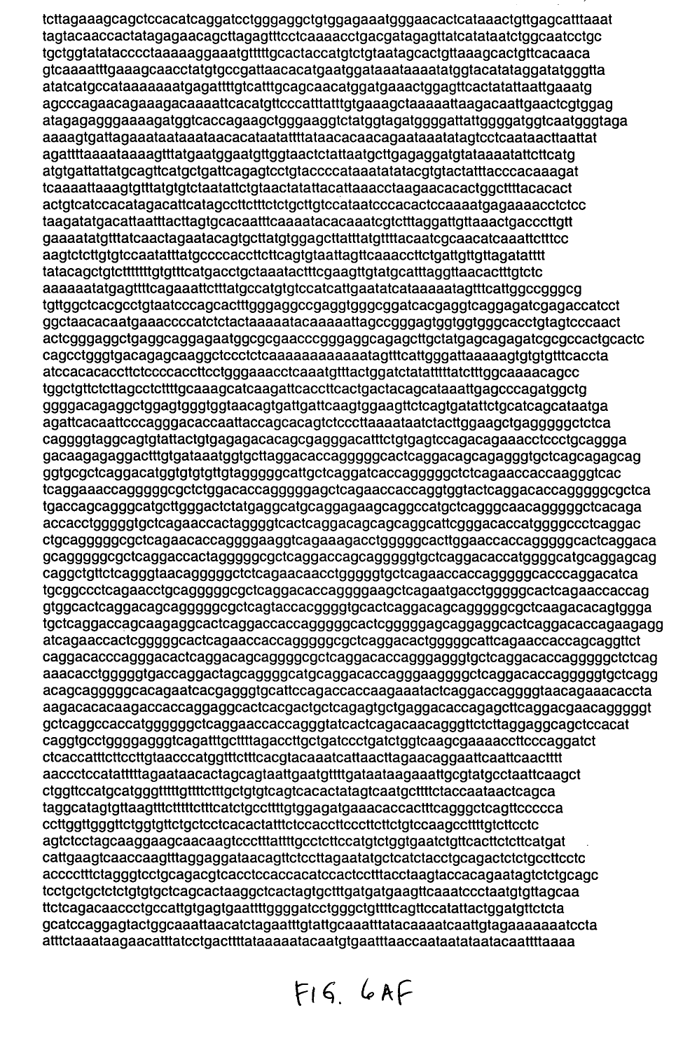

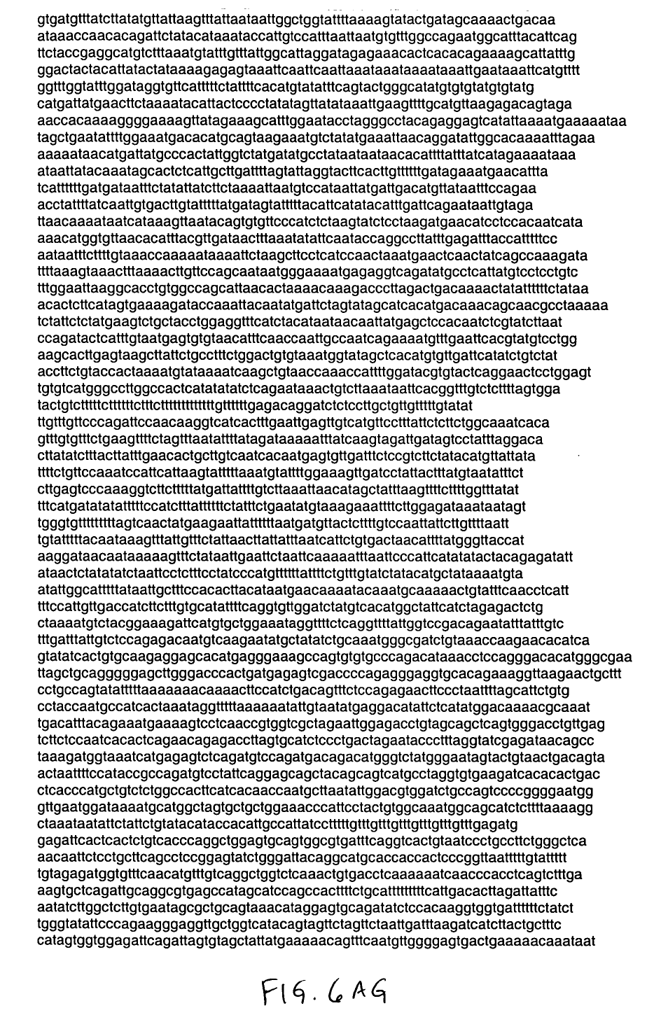

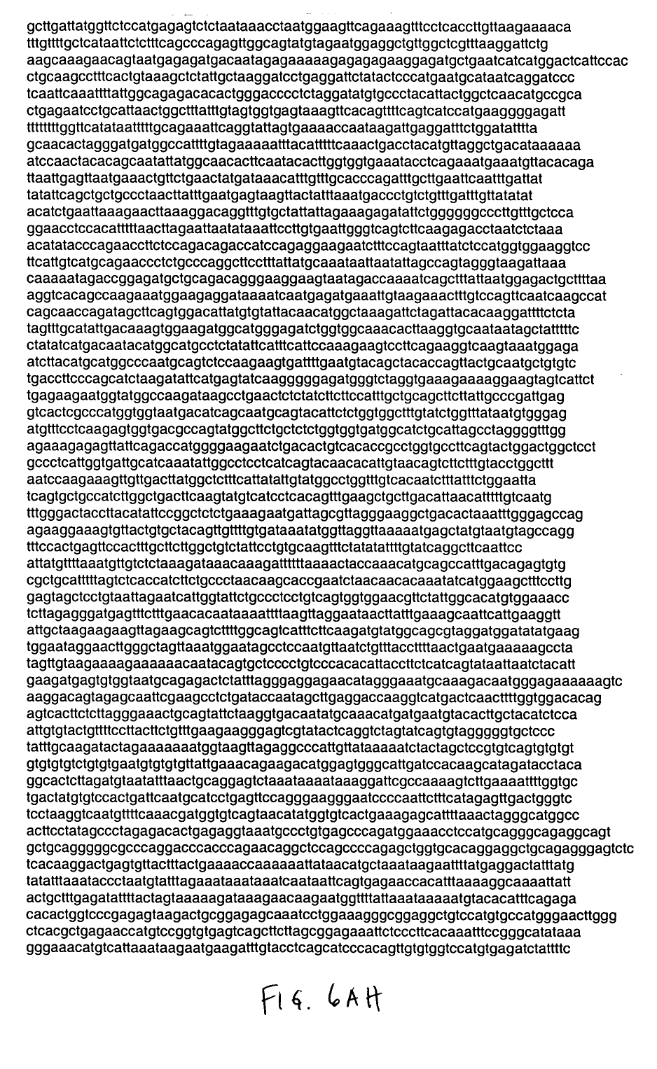

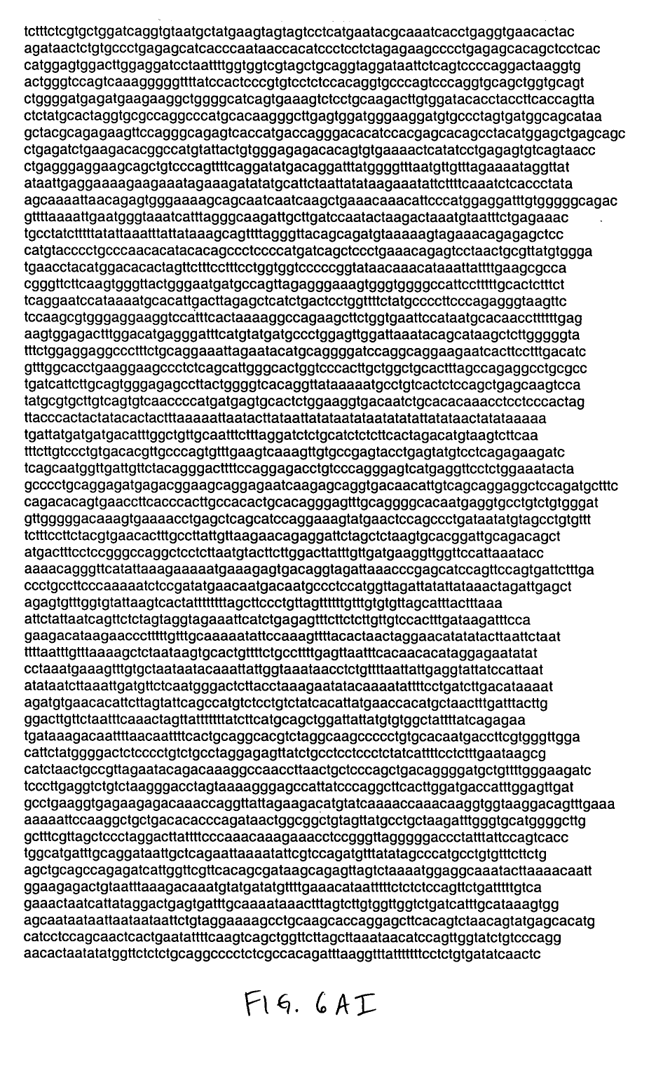

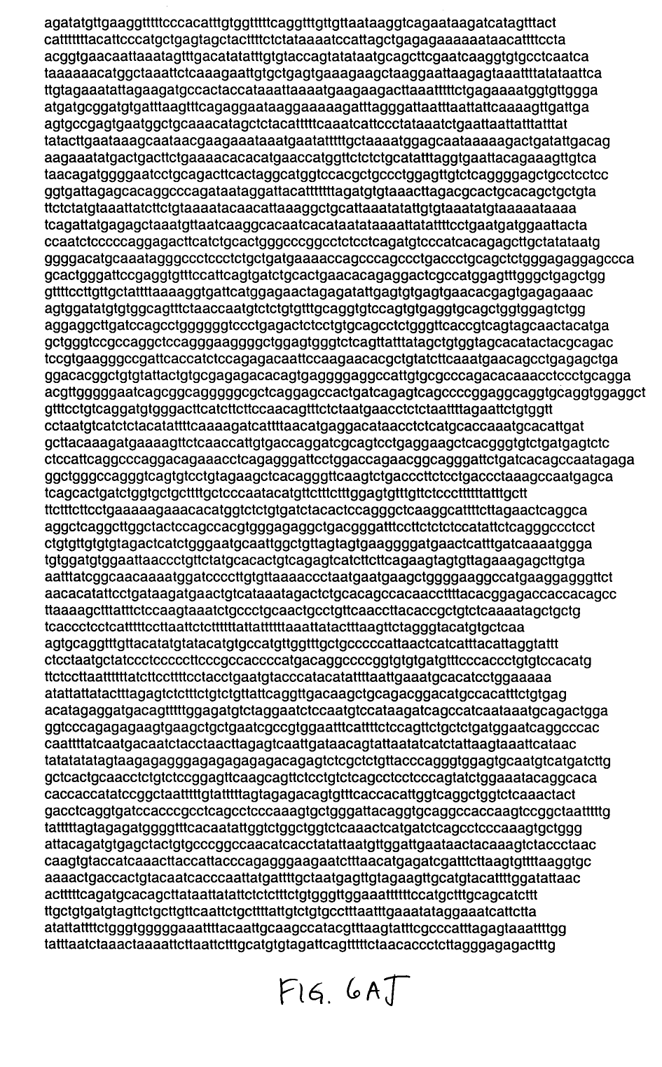

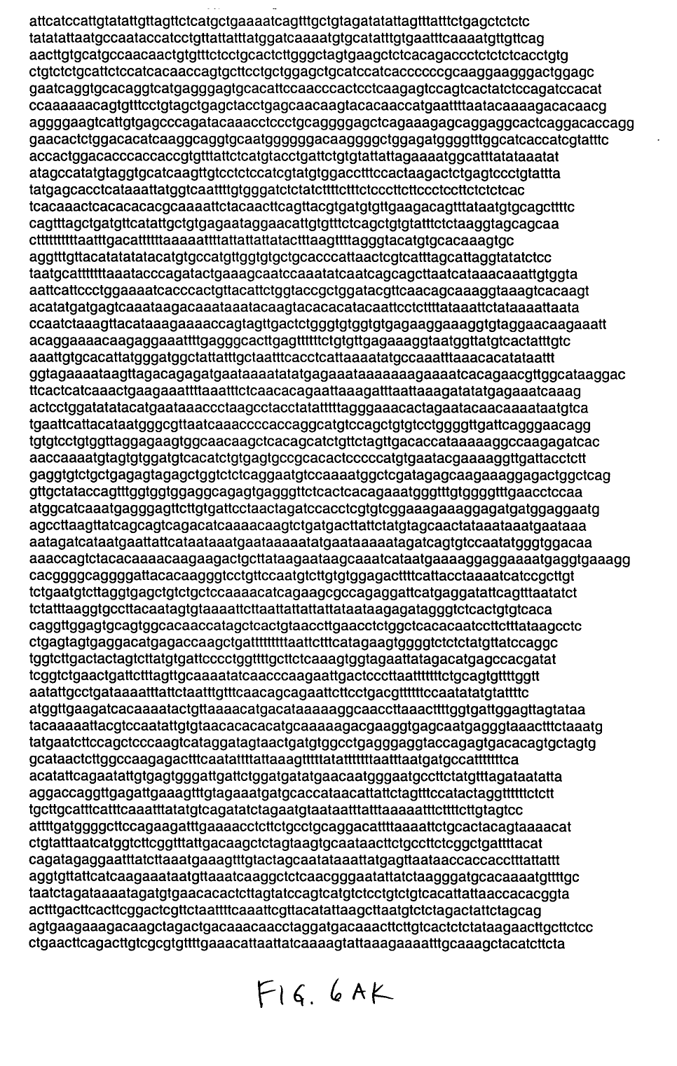

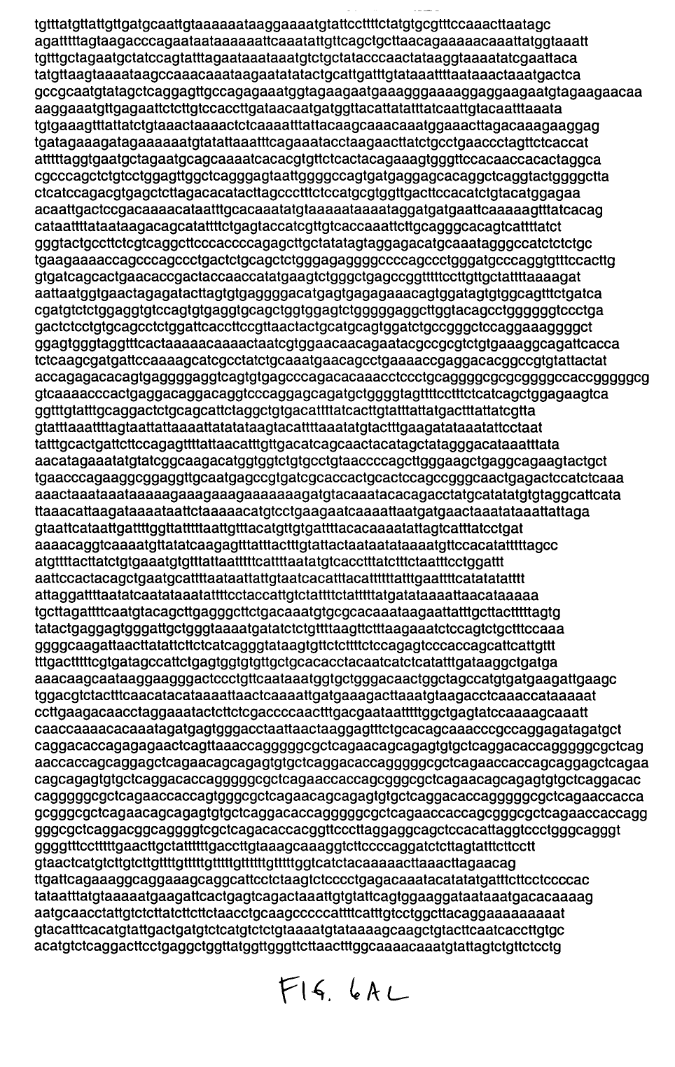

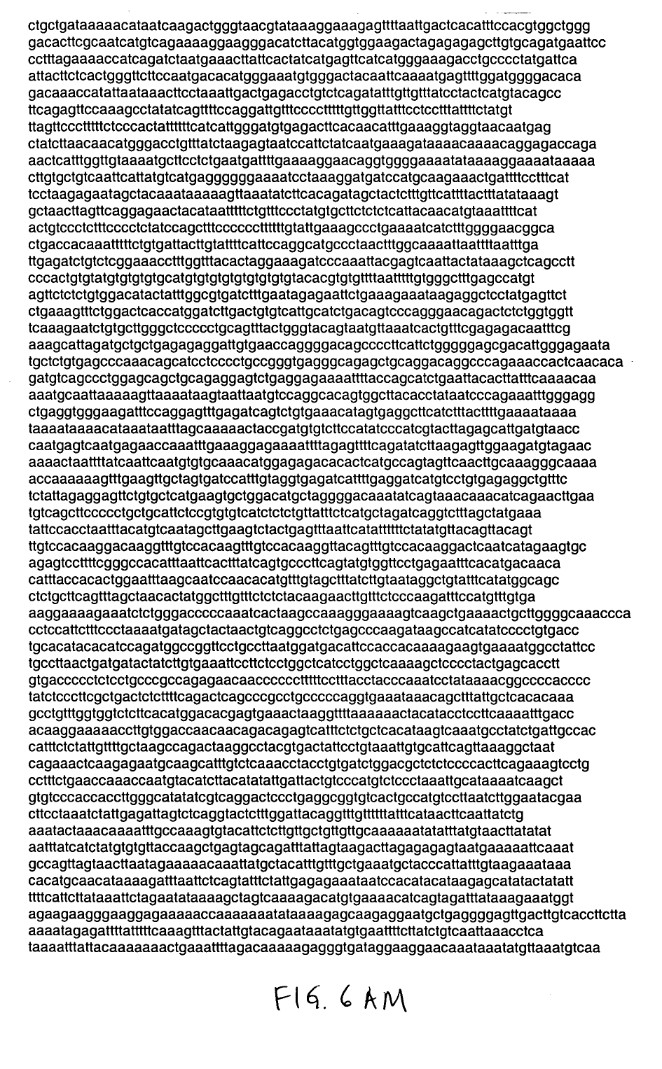

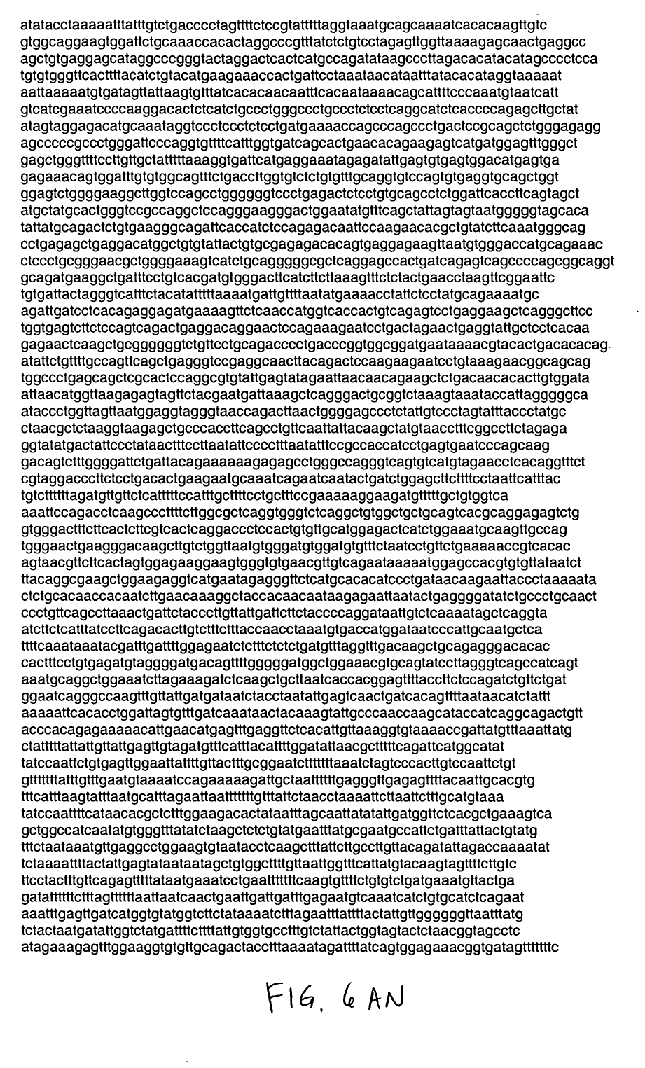

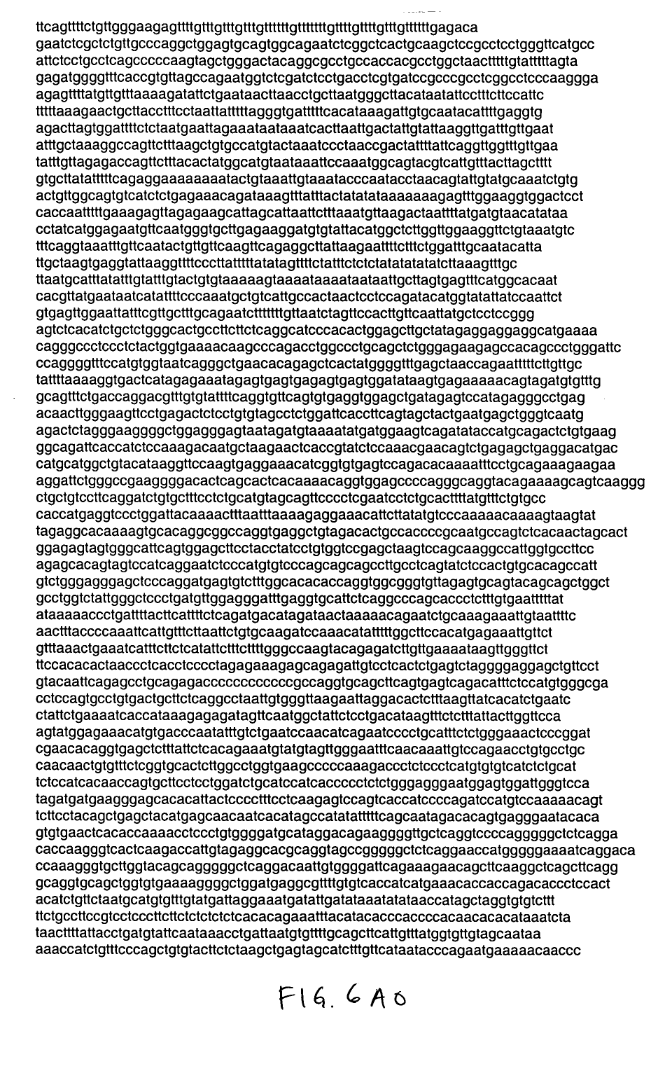

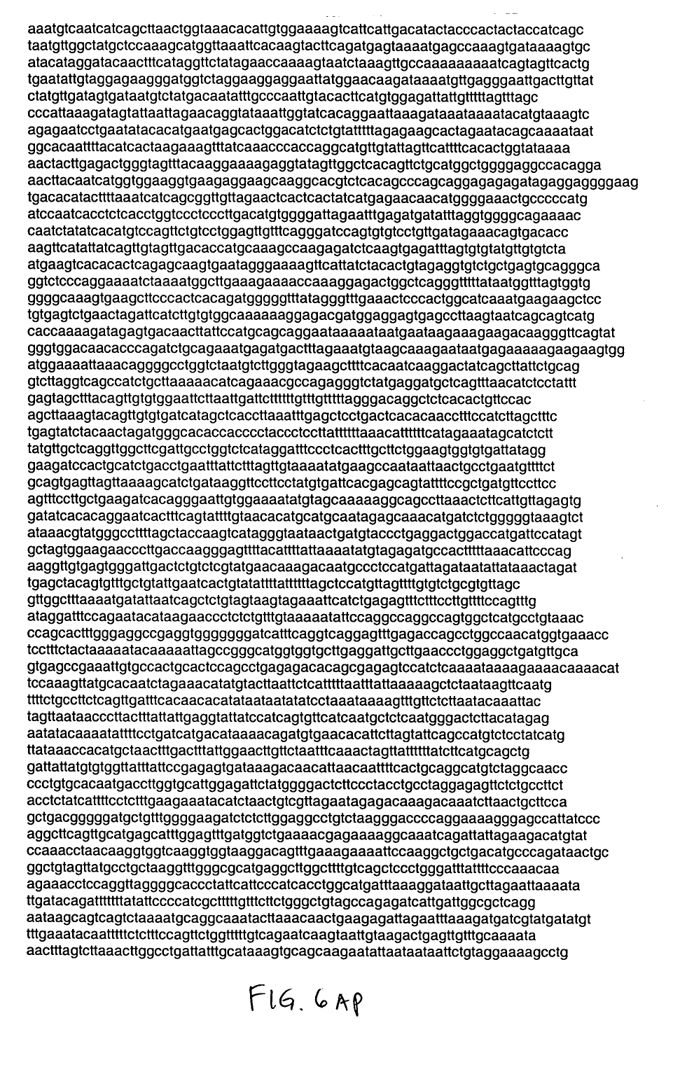

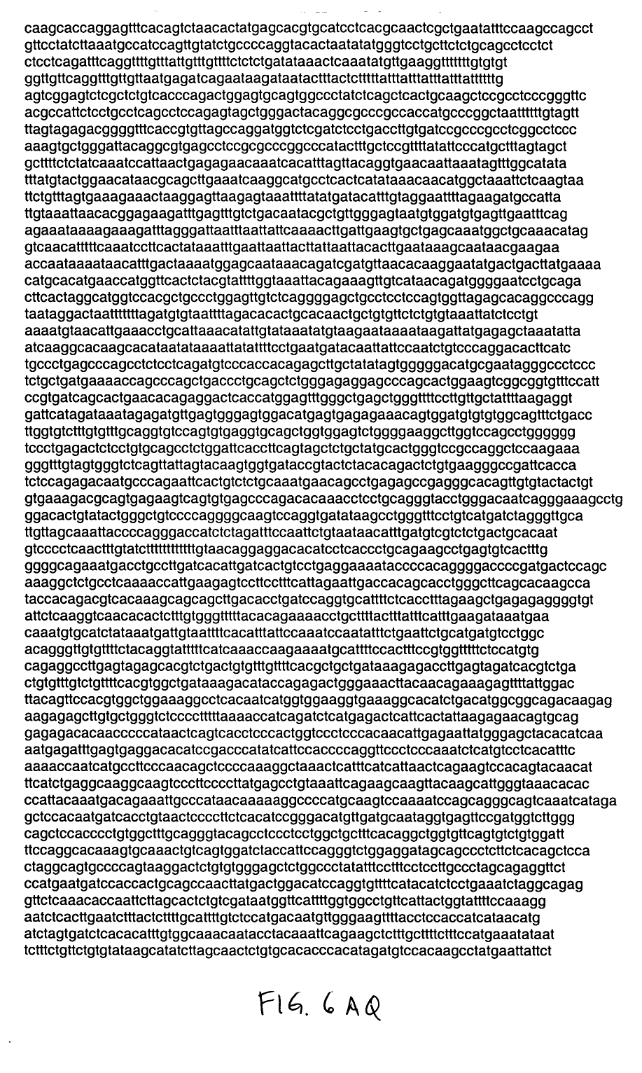

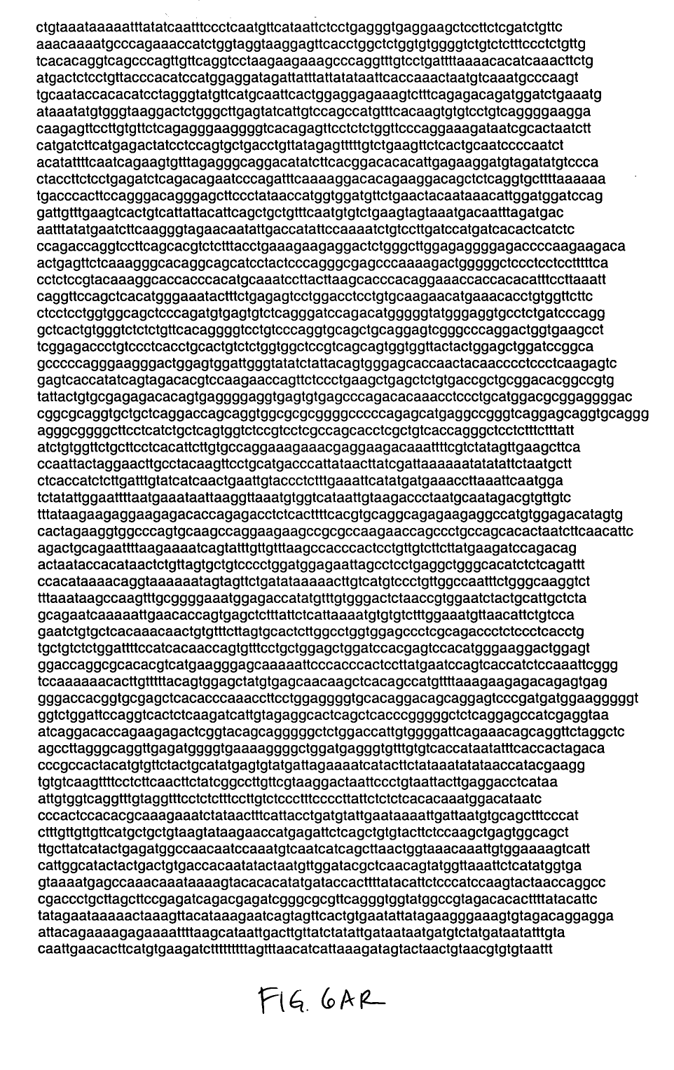

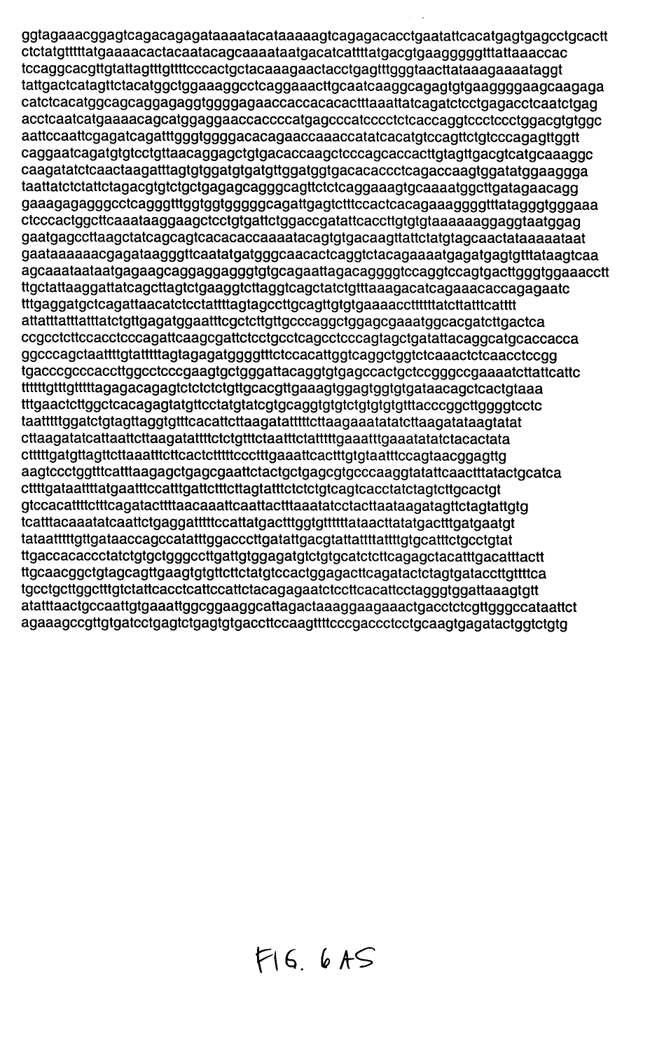

VDJ rearrangements normally produce IgH deletions extending from the D_{H}-J_{H} locus to the recombined V_{H} gene segment and generally preserve genomic regions telomeric to the utilized segment. Thus, functional VDJ rearrangements causing V_{H} deletions that span the region of the CTC-200D12 probe would be expected to utilize V_{H} segments telomeric to this region (FIG. 5). The V_{H} gene usage in the 22 CLL patients analyzed by interphase FISH (Table 1) was determined. Rearranged IgH sequences were amplified from genomic DNA (Bahler et al., Blood 78:1561-1568, 1991), sequenced directly and analyzed (Damle et al., Blood 94:1840-1847, 1999; Hamblin et al., Blood 94:1848-1854, 1999) using IgBlast (http://www.ncbi.nlm.nih.,gov/igblast).

As expected, all 5 CLL cases utilizing V_{H} genes telomeric to the CTC-200D12 region exhibited deletions with this probe, include 3 cases with V_{H}1-69 rearrangements previously associated with unmutated IgH status and unfavorable prognosis (Hamblin et al., Blood 94:1848-1854, 1999; Fais et al., J. Clin. Invest. 102:1515-1525, 1998). However, most cases exhibiting CTC-200D12 deletions unexpectedly utilized V_{H} genes that mapped centromeric to this probe (FIG. 5; the two DNA clones RP5-820M16 and CTC-200D12 involved in the deletion are located at the most distal (telomeric) region of the long arm of the chromosome 14 within the V_{H} region of the IgH locus. The next most proximal clone on these arrays is RP11-73M18, which maps outside the IgH locus and is ˜2.4 megabases centromeric to the CTC-200D12 clone. Asterisks (*) denote V_{H} gene segments used in patients carrying V_{H} deletions in >40% of cells and (#) denotes V_{H} gene segments used in patients with V_{H} deletions in <40% of cells). The V_{H} region deletions in these latter cases cannot be explained as byproducts of conventional VDJ rearrangements involving the utilized V_{H} segments. The array-CGH data (FIG. 2) and subsequent FISH experiments employing more telomeric V_{H} region probes (data not shown) also indicate that at least some of these deletions may extend very close to the 14q telomere.

A number of embodiments of the invention have been described. Nevertheless, it will be understood that various modifications may be made without departing from the spirit and scope of the invention. Accordingly, other embodiments are within the scope of the following claims.

1. A nucleic acid probe comprising a nucleotide sequence that hybridizes with a nucleotide sequence within human chromosome 14 and that allows detection of a lesion at 14q32.33.

2. A kit comprising a nucleic acid probe comprising a nucleotide sequence that hybridizes with a nucleotide sequence within human chromosome 14 and that allows detection of a lesion, e.g., a deletion, e.g., a deletion of nucleotides, at 14q32.33, and instructions for use of said probe for the diagnosis of a chromosomal lesion occurring within the V_{H} locus of 14q32.33.

3. A method of evaluating a subject at risk for chronic lymphocytic leukemia, the method comprising:
a) providing a sample of genomic DNA from the subject, and
b) determining the absence or presence of a lesion at 14q32.33 of human chromosome 14.

4. The method of claim 3, wherein the determining step comprises determining the absence or presence of a lesion at the V_{H} locus of 14q32.33.

5. The method of claim 3, wherein determining the absence or presence of a lesion comprises contacting the sample with a nucleic acid probe comprising a nucleotide sequence that hybridizes with a nucleotide sequence within human chromosome 14 and that allows detection of a lesion at 14q32.33.

6. The method of claim 5, wherein the probe hybridizes with a nucleotide sequence within 14q32.33 of chromosome 14.

7. The method of claim 5, wherein the probe hybridizes with a nucleotide sequence within the V_{H} locus of 14q32.33.

8. The method of claim 5, wherein the lesion is within the V_{H} locus of 14q32.33.

9. The method of claim 5, wherein the lesion comprises the nucleotide sequence with which the probe hybridizes.

10. The method of claim 5, wherein the probe hybridizes with a nucleotide sequence that is adjacent to the lesion.

11. The method of claim 10, wherein the probe hybridizes with a nucleotide sequence that is 5′ to the lesion.

12. The method of claim 10, wherein the probe hybridizes with a nucleotide sequence that is 3′ to the lesion.

13. The method of claim 10, wherein the probe hybridizes with a nucleotide sequence that comprises nucleotides 5′ to the lesion and nucleotides 3′ to the lesion, wherein the probe does not hybridize to the nucleotide sequence in the absence of a lesion.

14. The method of claim 10, wherein determining the absence or presence of a lesion comprises contacting the sample with a nucleic acid probe that hybridizes with a fragment of DNA that differs in size from a fragment of DNA that does not include the lesion.

15. The method of claim 5, wherein the probe further comprising a label.

16. The method of claim 15, wherein the label is a fluorescent label.

17. The method of claim 16, wherein the label is chosen from 7-amino-4-methylcoumarin-3-acetic acid (AMCA), Texas Red, 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade blue acetylazide.

18. The method of claim 3, wherein determining the absence or presence of a lesion comprises contacting the sample with a primer capable of amplifying a portion of 14q32.33 and that allows detection of a lesion at 14q32.33.

19. The method of claim 18, wherein the primer hybridizes with a nucleotide sequence that is adjacent to the lesion.

20. The method of claim 19, wherein the primer amplifies a fragment of DNA that differs in size from a fragment of DNA that does not include the lesion.

21. The method of claim 3, wherein the presence of a lesion at 14q32.33 of human chromosome 14 indicates a predisposition to CLL.

22. A method for selecting a treatment for a subject with chronic lymphocytic leukemia, comprising determining the absence or presence of a lesion at 14q32.33 of human chromosome 14, and providing the subject with treatment for CLL.


SEQUENCE LISTING (1 sequence)

SEQ ID NO: 1 - DNA, 200000 bp, Artificial Sequence
  Probe sequence from human
aagcttaaatagtgttgcaagttttaatatgcctacttttcaatttttcaatactatttttactccaaagccatggtgacccacgctcgggtgggtcctgaggagaacaaagctctggttctgatcctaaccctaaccctgtcccaagactttgaccctgaacctaaatcctgatccctaccctggtccctaattctgacccttactttgaccctgactttgagcttgaccttgaccatgaccccacctctaaccatacttcccgccctgactctgaaccagatcctaatcctatccctaaccctattattatatttacaatctatgtctaatctaaccgtctagtgctaaatagctgtacccaaaggcacttttaaattatttaacttcttttccttgaattctctaaggacatcctaaaggagatgtcattatgtattttgcattccctctgagtggtatggcttcagatatgaacttctaatactttgcaagacataaaaagtttggagggtaacagcactgggttgttagggatgtatgttggcattcatggtagtcattggtgctgttctccaaatattttcagttcattttttatgaatgcattctgactcttccatcccacctacttaaattttctaatggccacatgagttttttttgttttgttttttttgccaatggaggtgagaagaaataacatgtgactttttcaggagaaatctccaagaaacagagttctattctgcatgcttttttctcttttctatagcaatggggatcttattgatggtccctccttccgtctggattcctgtgttaggatgacacagcacagagctacctctcacctgacccgtgatgagatgttttgtggcaacatgatctcaagataatgataattataaaaccctttactgacaaaatgtagatctccattttttcattgtaatttaactttgccttactttaagaacagtagtttgcagctccaaatgcactgattaaagacagacgtccattttctctggaaatgtatttttatgttcttactggatgtatttgctgttaatgtttgctactatgaagatacctgaacagtgtccacattagagaataagagaaagtaatgggcagattaaccctgtgcatctagacccgggaatcctttgaccctaccctccctgaaatggagacacagaggatggatgagaaaaactgagcagtgcacccatgatcacaaaaagaatgacatggaaatgtgcctcttcccatctcatgaaacgcagctcatcctctgttctttcaggcccttggtgaagagccaccccatgtctgtcccttcctcagtgtccacaccatgaggtctgcactgatctgggcttcccttctcatcactctcagtattagtgtgcattgtgaatcaggtccagctgtggctgctctgcatggggctgcagtcagtacgtttcctctgtgttcatgtaagtcctgttgaagcttactgatggagtcagagggggaaacattgtacagcccagcggttctcacagactctcctgcaaagcctctgatttcacttgtactggctacagcatgagcttggtccagcaggcttcatgacagggatttgtgtgggtggcaacagtgagttatcagggttactctccatgagtacaagtaaattaacagtcccaagcaacaccctttcaagtgcagtctaccttaaaatgaccaatgtgaaagccaaggacaagaccttgtattactgtgagtgacataggagcaggaacatctgcgtgagcccagacacaaaatcctctgcagggagacaggagggaatcgcatggtagatgctgattggaactaccatgtgtcgctcagaactaccaggaggtactcagaaccactagggggcgctcaggacaccaggggacgctcaggacaaccagggggtgttcaggacaccagggggtggtccgagccaccagggggcactcaggacaccaggggacgctcagaaccattaattccttgtaaatccatgatttttttacaaatgttacttccaaaacattaacttagaaccgggaattttttttttttgacaaagtctccctcttgtcgcacaggctggagtgcagtggcatgatcttggctcatggcaacctccaactcccgggttcaagcaattctcctgcctcatacttctgagtagctgccaccacgtgcctgccaccatgcctggctaatgtttgtacttttagtagagacggggtttcaccatgttgaccaggctagtctcgaattcatgacctcaggtgatctgccccgctaggcttcccaaagtgctgggattacaggcgtgagccaccatgcccagcctatttgaagttttaatgctgcgtgttttctgagtaacgctagcaatgatctgtcagaacaatttttaaaataggttatttatatattttattaaaatactattattaaaagagtaaaattgtaaaaattgcacctgcaatctcagcactttgggaggttgaggcaggcagatcacttgagctcaggagttttagaacagcctggcaacacaatgaaatcctttctatacaaaaaaaatagctgggtgtgatgctgcctgtggtcccagatacttggaaggctgaggtggggggatcccttaatcctgggaggttgaggctgtagtgagatgtgattgtaccactgcactccagcctggttgacagagtgagaccctgtcttaaaaaatgtgtatccttgatacacattgtttccatgaatagagtttagtgtttttgtgctgtgatactcaaacaattatatatgttttctaacattaactcaccatatgcatggtgttttgtttctttttctttcatcagctgaaagtggaatggaaattaaacaccactttaaaggctcttgttctccactttggttggctcctggtgtcctgtttttcagactatttctccatcttcccttttactttaaaagtcttttatcttcctcagtctccatgcaggaaataaaaattccctttactttctgtcctccaagtctggtgaacagtttccttctcttcataataactgaagccaaccaagtttaggaggataacagttctccttagaatatgctcatctacctgcagactctctgccctcctcacccttttctaaggtcctgcagacatcaccctcattccattccctcccttccctaagtacgacagagtgggctctgcagctcctgctgccttctgtgtgctcagccctggggctccctagtgcttttgttgcaggacgatcaaagatgggagagaccaaacagagttcaggagagtcctttattaaaaggtgatcacctggctcagttggactagcatccaggaaagtctgagccctggacaaagaaagcagccaccttttacacagtcagtggctgggagctacatgatgcaggaagcgtaattacagaacagagaacaaaggcagttatttgttacttcatgtcttacatccttgggaatacatggttctgtaacatatacttaacaatcttatgaccttgcagctgtgctggggaggtaagcaagaaccctctgagcctcaaggaatgtgaaaccaatgagaacagataaggctcactgagcacagaaggaaaaacaggcagttagtattctctaacttagactacgggggaggggctacacactacagtaagccctgaggggaaagttaattttctggctttacatattgtaaaattcataaattacttcttcactttgatgatgaagcccaaatccccatgtgcttacactctctcagaccaccctctaggaagctgccattgtgagtgagccctaaaaagcatgggctgggtttagcttcatattgctggatgttctctattataaaggcatgttggcaaataaagactacagtttgtattgaatattcatgccaaagaagtttttttcaaaacttttcaagtaaaaaattttatcttgcctagtttgaaaattaccatctaaattcaacaaataaggtaatacagttttaaaagtgatgcttgtcttattagttattcaatttattaacaacagactgatatttaaaataaataccattgcacatttaagtgccatactgttctgggattttttaaggaatcagagagaccgactctgttcaggaggatatttattatttaggttcaggaggatatttattatttaggtgcaccggccaagtcgaattaacatccaaaggactgagcccagaacagagttcagttaccttttaagcattttgtggggtgggagaggggacatctgtgcagggtgaagcatactacagaagtgagaaacaaagacagttattcaattgaaacatgtattacatcatttcttccttttcaaggaaaaacatgttttgcgacttgagtttatctttctagtgaccttgcagctacactgctagggaatcagggtcttcaaaatgcctgagaagggaggagaggtaaggctcattagccacagaaaaacaggcagttagtattttaaaggactccagctctttctctttttcagggagaattgggttttcttacatacaactgagtttctgcttacacattctttaatttcttttaattcctgtttcaatacttgacaagaatggcatttacatacagttttaccaaaacatgtatttaaatatatttgtctttttaatattggaataggcagacatacacgtagatcagcattattttgtactaaaatctcaaactgcaaacacaatttaaattcaattaaataattagaataatatgaaacaaatgggtgtgttgttttggtgtttacgtatgcattcacttttgcatgggcacatgtatgagtctttgctgggctgttgtgcacgtatgtgtgtttgtatgaccaggaggttttcaaatacatcattaaattacatagttatattaatcttggcaaggcacttgtattctgttttctttaattctgtttgcagaaagtagacacatattcagtcttagttccagtgtagggagtgcttttcatgagaaaaataccagaaaaaagggcaaacatggggcccactaatgtaaaaattagccacaatgtgtatgtgtgtgtgtgtgtgtgtgtgtgtgtgtctgagttgaatagtagagttggagtgggcttctatccacatgcacctgcgcctacaggtattatcaggtacaataatcaactgcagaaccctaaaggaaataagagtccccccaaacccctgaagagtgtttgggttcaccatgtgtccaatgattcagtgcctctcgagctccaggaaacggctccctggtgatgcgtgagatcttttcttggggtgtccctgcagagttcgctgggtttcctaaggctgattcactatttcaaaagatggtgtgagaagcatatggtgtaaataaagcagaattctgagccagggcacagccactttatactgggctagagacactggtaggaatatactctgtcagctcagatagaaacctccctgcagggtgggggcagggctgcagggggcgctcaggacacatcgagcacagtcttctgccccagagcaggtgcacatgaggctggggagaggttcctctcagggcctgggacttcctttaaaaatatctaaaataagtatttcacaaggactgctgatgtttgtataaatatcctattcaattgtgagcatttatcaaactggatgttgtaatgagaaccacttttataatggcgatttcaaactctgctagttatcttaataatagcagctggaggtcaggaagagattattacttataaataagtgcaatttttggagagacacactcattcccaaaataacacattcacatattaaggtctagaaatggttcacgttgcccctgagacattcaaatgtgggttcaaagtgaggtgctgtcctcggggagttgttccccagtggaggaagcgctatcaacacagagttcagggatgggtaggggatgcgtggcctctaacaggattacgactcgaaccctcagctcctataattgtgtcgtccgtgtgtcatggatttctctttctcatactgggtcaggaattggtctattaaatagcatccttcatgaatatgcaaataactgaggggaatatagtatctctgtaccctgaaagcatcacccaacaacaacatccctccttgggagaatcccctagagcacagctcctcaccatggactggacctggagcatcctcttcttggtggcagcagcaacaggtaaggggctccccagtctcggggttgaggcagaaaccaggccactcaagtgaggctttacccacccctgtgtcctctccacaggtacctactcccaggtgcagctggtgcagtctggccatgaggtgaagcagcctggggcctcagtgaaggtctcctgcaaggcttctggttacagtttcaccacctatggtatgaattgggtgccacaggcccctggacaagggcttgagtggatgggatggttcaacacctacactgggaacccaacatatgcccagggcttcacaggacggtttgtcttctccatggacacctctgccagcacagcatacctgcagatcagcagcctaaaggctgaggacatggccatgtattactgtgcgagatacaccatgtggaaacccacatcctgagagtgtcagaaatcctgatgtgggaggcagctgtgctgagctgaggcagtgatgcagcagtttccttaacttccatcttatctcattttgcatcgacatccacctttatattagccaagaatgtggaatagacgggtgctcataagagatccttaacttgcccattttaatgggttttccagaagatgtgagaagccactttgttagcaaagcatgccaaagccatgccctgctccagacacatgtgagcccatttcctgctctttgcttaactgacaagctctcatcagtgcacctgggctaatttcacatcaggtacaggaatatgttctaaaggaaagctaattttataatagcaattcctgcttaataaccttcagcttcattgtttttgtgtaatctatcaacaaattatgttagttcaaggttctcaatgggagtttctaataaatagaagggatgtatagaagttcccctaattaaaacaattgtgaacacaatcttggtattcagctgtgtctccacccttcttaccattcaccacaaagtaattctcacttctggaagctgggttcattttcaaattagttttttttttcattttaatatctcaaggttattgtatgtgactattttagcagaaattgaattttgggaaattgaactaaccaactgaaaataaattcacaactaaataacaagatgccagaatataattggctccaggctttgttaattcagcagtttatctacctaggcattatatttacacatcttcatgctatttttacagtcaactcccattatgtaagaaatggtcatacaccatttcttacatatggtaagatatctacgatggatattcagaactacgataaacattggatgcatccaggtttgtctgaactcactgagattattacactcagctgctgtttcaatgtgtctgaaggtataaatgacaattcgaatgacttgggtgtgtggatggttttacatgagtctttaagtgtagaacaatattgaccctgttccaaaatctgtccttgatccatgatcacactcatctcccagaacagctccttcaacacatttcctatctggatgaagaggactctgtgcttggtgaggggaggccacaggtagagaactgagttctcagagggcacagccagcatcctcctcccagggtgaacccgaaagactgcggcctccctcatcccttttcatctctccatacaaaggcaccacccacatgcaaatcctcacttaagcgcccacagggaaaccaccacacatttccttaaattcggcgttctgcttacacgggaaatgctttctgagagtcatggacctcgtgtgaaaagaacatgaaaaacctgtggttcttcctccccctggtggcagctcccagaagtgagtgtctccaggatgcagatatgaagatatgggaggctgcatctgagcccagggctcacagtgggtctctgtgttcacaggggtcctgtcccagtgcagctgcaggagtggggcccaggactggtgaagcttcggagaccctgtccctcacatgcgctgtctctggtgactccatcagcagtggtaactggtggagctggtccgccagtccccagggaaggggctggaatggattggatacatctattatagtgggaggagctactacaccccgtccctcaggagttgagtcaccatgtcaatagaaacgtccaagaaccagttttccctgaagctgagctctgtgaccgcagggaggcggagggggcgggcgcaggtgccgctcaggaccagcagggggcgcgcggggcccacagagcaggaggccgggtcaggagcaggtgcagggagggcggggcttcctcatctgctcagggctctccctcctcgccagcacctcagctgtctccagggctcctctttctttcttatctgtggttctgcttcctcacatccttgtggcaggcaagaaaggaggacgacaggcctggtgcggtggtgtatgcctgtaatccccgcactttgggaggccgaggcgggtggatcacctgacgtctggagttcgagaccagcctgaccaacatggagaaaccccgtctctactaaaagtacaaaattagccaggcgtggtggcgcatgcctgtaatcccagctactccggaggatgaggcaggagaacggcttgaacctggggggcggaggttgctgtgagccgagatcgtgccattgcactccagcctgggcaacaagagcaaaactccgtctaaaaaaaaataaaaggaagaagacaaattttcctcttacagtcgaagtatcaacaatactaggaactttcctacgagttcctgaatggccccttttttccttcttgattaaaaaaaagttctaatgcttctcaccatctctttatttgtgtcgtcaattgaattgtgccctctttgaaattcatatactgaaatcttgaatccagtggatctatattggaattttaagtatataattaaggttaaatgtggtcatacgtgtgagaccctaaagcaatagacatgttgtctttataagaagaggaagagacaccagagacctctcacttttcacgtgcacatagaaaggaggccatgtggacacacagtggactagaaggtggccctgggcaagccaggaagaagccgcaccaagaaccaccctgccagcaccgtgatcttggacattcagactgcagaattgtaagaaagtcagtatttgttgtttatgccacccactcctgttgtcttcttgtgaagacccacacagactaatactacataactctgttagctttgtctcctagaaggagaagcaggcccctgaggctgggcacatctcagatttccatataaagtaggcaaaaatagtaactcttataaaaaaatgtgtcaaggccctttctttcttttttttctttgagacagagtctcgctctgtcaccaggctggagtgcagtggcacgatctcggctcactacaacctccacctccagggttcaagtgattctccttcctcagcctcccaagtacctgggactacaggcacccgccaccaaccctggctaattttttgtatttttagtagagatggggtttcaccatgttagcaaggatggtctcgatctcctgacctcgtgatcctcccgcctcacctcccaaagtgctgggattacaggtgtgagccaccattggccatttttggtggaaaggtctctgaaaccaaccctggtgtctgtgccacaaaagttgtcttttatcttttatttggacataacaaatggacagtgagtaccacctggatggagactgaccactgaccatcttctgctgtctcctaagtatgtcaaagaaaaccacatcgacatcactatttttcttcaacctcctcgaatgaactatagaaatgatccctgaaagtatagtctattccttcaaccttctaaatttgcactaaatctcttcctaaatgagagctacagggggtctgagttttattcccttctttccagttttccccaagtacaaagagcagaatagactttcagtgaaatttggctgacaatgcccctaacaccacatcactttctaaaacccatatcctgcctccatccttctctggacacccctcatcgggctacccaggaatggccagaagctggcatcagcttctgggctgaggccacgagctatacacacatgtgatttcagttacacagactctactgcaggaccacacctgtgttctgaggcactcaggcacctgctgatctcagtcattctctaataaattacacacctcttattaataaaggtccaaatggtcccatcagctgcagagcagtggagtaaagctcatgggtgggtccgtcaggcagaagtcagacaatgggatgggtaggttggtcacctccattgtcactgaggttcccagtggattttaacagaataaaacaatattaacaacaaagggatcttgaaagtatttaaggtccaggaattttgtttggggggctagagaaagacatttttaaacagaaagccctaaagcacatacagcagctgacagagtggccactgtgcacatgacggctgaggagacagatggtaggggacattcccccaagatgtccctgggtgtgtgatggttggactcctcatgcatatgaaaataagagctggactcaggtagaaacaagggccataccccataggaaaggaagaaaaaaacagatgggcaaccctggagagagctcattagatttggtaggtttgagatgaaaattacttccatggattttaatgttctaagctaagtaagaatctcttcaataaagtgagaattaaaaggagaatggagctaggagttgagagaggcaacaaataatgagagagcagaaagcaaatccacaaaaaactgtcacatgacagaggccagaatggagctgatgcagctgcgtcatttcctacagacctagttgaccatgtggagaagaggcttgaacaaatggggacgttctccaaccttccaaatcagccttcttcttaggcttgagtagtaatcatggttctgagggtgaccttccaaattttcctgtctgtacctcttccaccaggggtagagtgtcttcccaaccataatggtttctctccagtgtcctcagcttctcctcctttgtacttaccctgcagattaaggatttcttctagatggagtttcctgggaatcctctgttttcttttgtttctgttgactccaccacaccccataggtcacaggtttgattggatttcccctggagacagtggaggtggatccaggcgttcaacagtcctcgctgttcttcccttcctgtcagcaccacaggacagcagataagggagttgactgtggatttttcaaattcctgggaaaagcctgcaaggactagtagattcacactctaataccattagcacatgcatccaaaaaaaatcctcactaaatatttccaggttagcctgttcctctctcaatgccatccagtggcacctgccctgggttcactaacatgtgggccccagtcctctctgcaggcctctctctcctcacatttcagtcttcttgctttctctgtgaaactaactcacaaatgttacaggtttttcttcttcctttattcacagtttttcaggtttgttgttaatgaggacagaataagatcatagtttcctcatttttcacattcccacactgagtagatttctatataaaagccagaaactaagagaacaaatcaaatatccatatccattacaggtgaatgttaaacaatttgacataagattatgaagtaaagtacaatgcagcattagaatcaaggcatcctcattcacataaagtcatggctacattcacaaataactgcgctgagtggaagtagctaaagtattaacagtgcacttcataaacttctatttgtataaactgtagaaggtaaaactattctaaagtaacagagaagagaatttggtgagaattgggtgttgaaagtaatggggtgttgagatgaaattacagagaagtgacagaaagatttagaggttaacttaattgcacacgacttaattgaaaccctgattaaagggttgcacacatacattaacatattccaaattgtgcagtatatatttgaattaattatttgttaattttgtacttaaaaaagcagtaacaaagaaacaaatgaatgtgttggttgaggaggagcaaaaaatagatggctattaacacttgaaacatcgctgacttttgaaagtacacatgtatgaacactggttctctctatatatatttcagataaacaacagaatacactaaaagaaaacagagatatcctggcaggggtggaatcctgcaaacctcactaggcatgtcccacactgccctggagttgtctcaggggagcagtcttctctagtggtcagaggcacaggcatggataatgggactaattctgtccagctgtgtgatcttgaccatattgtacagccactctgttctgtgtgtaatgtaaattccttaaaatgtaacattgacacttgcattaaatgtattctgcaaatatgtagaaatgaaataagatgatgactgctaaatagttatcaaggcatgatcacataatataaaatgatattttcctgagtgataagatgactaccaatctcccccaaggcactttgtctgctctgacccctgcccctcctcaggattcccatcccagagcttgctatacagtaggagacatgcaaacaggtttctccctttgttgatgaaaaccagcccagtcctgaccccgtaggtttgggagagaagccccagccctgggattcccaggggtttccgttttgtgatcaggactaaagacagaggacccatcatggagtctgggctgagctgggtttcacagttgttattttaaaaggtaaaccagagaaatagagtgtgagtgaatatatttgagagaaacagtggatatgtcaggcagtttctcaccaggatctctacgagtttacgggtgtccagtgtgaggtacagctggtggagtctggagaggacccaagacaacctgggttccctgagactctcctgtgcagcctctggattaaccttcagtagctactgaatgagctcggtttcccaggctccagggaaggggctggagtgagtagtagatatacagtgtgatggaagtcagatatgttatgcacaatctgtgacgagcagattcaccgtctccaaagaaaatgccaagaactcactgtatttgcaaatgaacagtctgagagcagagggcacagctgtgtgttactgtatgtgaggcaccaggtaagaagacatcagtgtgaacacagacacagaggttcctgtaatgataagggaggaggctgggataaagggagcactcaagacccacagaaaacaggggaagctctagggcaggtgcagacggtcgtcatgggctactttccttcagggtctgtggctttctctgcatccaacagttcccctgggagcctcttgacatttatgtttctgtgcccatccctgaggtctctggattagaaaaatttattataagaagtggaaacattctcatttgtcccaaaggcagatgtaagtaatggaggcataaaaatgcacaggaggccagggaggctgtagacactgccaccccagaatgtcaatctcaccactagtgctggagaagagtgggagtttgatggagcttacctaaataccctgggtccaagctaagttcagcgaggccactggtgcctcccagagctcagttgtccatcaggaatctcccatgtgtcccagcagcagccatgcctcagtacttccgctgtgcacagccattgtctgggaggagctcccaggttgggtgtctttggcacacaccaggtgacaggtgttagagtgcagtgcagcagctggctgcctggtctattgggctccctgatgttggagagatgggaggtggattctcagggccagcacccagtttgtgaatttttatataaaaaccatgattttacttcattttctcagatgacatagataattaagaacagagtctggtccggacgtggtggctgatgcctgtaatcccagcactttgggaggccaaggcgggcagatcacctgaggtcgagagttcgagaccagcctgaccaatatggaaaaatccatgtctactaacaatacaaaattagccaggcatggtagcatatgcctgtaatcccagctactagggaggctgaggcaggagaatcgcttgaacccgggaggtgaaggttgtggtgagccaagatcacaccattacactccagcctgggcaacaagagagagaatccatcaaaaaaaaaaacaacaaaaaaaaacagactttgcaaaataatgttcaaccttatcccaaattcattgtttcttaattctgtgcagcatccagacatattattaccttcctcatgatgaattgttctgtttaaactgagatcagttttttctcatattctttggtttctgtctaagaacagagatcttgattacagtaaggttggttctttcctcatactaaccctcacctcccccagagaaagagcagagattgtcctcactctgagtctgagggaggagctgttcctgtacaactcagagcctgcagagacccccaggtgcagcttcagtgagtcagacatttctccatgtgggtgacctccactgccagtgattgctgctgaggtctaattgtgggttaataattaggacacccttcaggttgtcacatcccaatcctattctgaacatcaccattatcatagacagagatagttcaatgcccattctactgacataagtttctctttataatttggttccaactatggagaaaaacgtgaccctatatttgtctgaatctaacctcagaatctgctgcattgctctaggagattcacaaattgaacacaagtgagctgtttattctcataaaaatgtacatatttgagaatttcaatgtattgcccagaacctgtgcatgccaacaactgtgtttctcagcgcactcttggcctggtgaagccctcacagaccctctcccttacctgcgctgtgtctgtgttccccgtgataacgaggtcttcctgctgaagctggatccatcagccccccagggaagggaatggagtggattgggtgcataggtcatgaagggagcacacattaccacccactcctcaagtgtccagtcaccatccccagatccgtgtccaaaaaagcagttcttcctacagctgagctacatgagcaacaagcacatagccatgtatttttaagccaaagacacagtgagggaatcacagtgtgagctcacacccaaacctccctgtgggggtgcataggacagaaggggttgctcaggtccccagggggctctcaggacaccaaggggcactcaagaccattgtagaggcacgcaggtcagccgggggctctcaggaaccatcgaggaaaatcaggacaccagagggtgctcggtacagcagggggctcaggaccattgtgaggactcagaaagagcaggttcaaggctcagccttagggcaggtgaagctgggatgaaaaggggctggatgaggggttttgtgtcaccatcatatttcaccactagacaccctccactacatctgttctaatgcatgttctaacacatgtgagtgtttgtatgactagaaaatgatattttggggccaggtgcagtgactcatgcctgtaatcccagcactttgggaggctgaggcgggcagatcacctgaggtcaggagttcaagactagcctggccaacatgatgaaactccgtttctactaaaaatacaaatattagccaggcccagtggcacacgcctgtaatcccagctactcgggaggctgaggcaggagaatcgcttgaacctgggaggcagaggttgcgttgagccaagatgtgccactgcactccagcctgggcaacagagcaagactccattaaaaaaaagagagaaaaaaagaaaatgatatttatatcaatatataaccatagctaggtgtgtcaagttgtcctctccatctatgtcagccttgtccataaggactaattccctgtaatttcttgaggacctcataaactgtggtcaattatgtaatattcctctcttttttctgccttccttcctgccttctctctctcacacagaaactgacgtacacccaccccacaacacacataaatctataccttttattacctgatgtatttaataaacttgattaatgtgcagcttttcaagcttagttgcttgtgaggttgtaagaataagaaccattttttcaaagggaatgcttccagtttttgcccattcagtatgatattggctgtgggtttgtcatagatagctcttattattttgaaatacgtcccatcgatacttaatttattgagagtttttagcatgaagggttgttgaattttgtcaaaggctttttctgcatctattgagataatcatgtggtttttgtctttggctttgtttatatgctggattacatttattgatttgcgtatattgaaccagccttgcatcccagggatgaagcccacttgatcctggtggataagtttttgatgtgctgctggattcgttttgccagtattttattgaggttttttgcatcaatgttcatttgacaaagggctaatatccagaatttacaatgaactccaacaaatttacaaggaaaaaaaaacaaccccatcaaaaagtgggcgaaggacatgaacagacacttctcaaaagaagacatttatgcagccaaaaaacacatgaaaaaatgctcatcatcactggccatcagagaaatgcaaatcaaaaccacaatgagataccatctcacaccagttagaatggcaatcattaaaaagttaggaaacaacaggtgctggagaggatgtggagaaataggaacatttttacactgttggtgggactgtaaactagttcaaccattatggaagtcagtgtggcaattcctcagggatctggaactggaaataccatttgacccagccatcccattactgggtatatacccaaaggactataaatcatgctgctataaagacacatgcacacgtatgttatattgcggcattattcacaatagcaagacttggaaccaacccaaatgtccaaaaatgatagactggattaagaaaatgtggcacatatacaccatggaatactatgcagccataaaaaatgatgagttcatgtcctttgtagggacatgggtgaaattggaaatcatcattctcagtaaactatcgcaagaacaaaaaaccaaacaccgcatattctccctcataggtgggaattgaacaatgagatcacatggacacaggaaggggaatatcacactctggggactgttgtggggtggggggaggggggagagatagcatcgggagatatacctaatgctagatgacgagttagtgggtgcagcacaccagcatggcacatgtatacatatgtaactaacctgcacaatgtgcacatgtaccctaaaacttaaagtataataaacaaaaaaagaaaggtgagggagatactgtgagtaaagagctcgcttggggtctgcaatgtagtatggcttagtcagtgtgttagtgctctgtcctcttctttctgaaatcacagttcaccatcacctccttattttctagaaacacctggaaatagtgaaacttttttacttcccttatatttcatattagtgctatacccagggatcatcagttaaccacaatgacagtgacaaaaagctgccttcatattttctcccaggagaattttggctccaagaatcaaggctttaataggattcaggagaagtcagtgatcacagggcagatgcctcaggtttaaagtcacatcaaaccgagtgtgaaccttctgtaactcgcaactgtgctaacttttggaggtttcttgaaaaattctagtcagtttgggttaaagaatggtttcagtaatgtcacactcagggtcatgactgctcaactggtcattagccctgggaagtaacttccgctaacattggcttacctcataggagcttataaattgccatcatattgattatctcattaggtcatcaggatcctgagagaatgatcttttaagaaaacaatctttttatcaggcttatttatcattgcttctatatgctgctcttgtcctgcagaggaggcagcctggggtcaatcaagatactgggtatttgggttcatactttaagccccccactctctcatggtctaacttcagacaagtcacctctgctttttgagcttcacttttctcagccaccaaatggatgtagagataatatttgcttgcccaattttgaaatgtaggacggggcatcattaattaattagttcttttgtccacaaacatttacccagctccagcatgagcccagcctatgcttggtcttctgcagaacagagaaatgccttagaaggagattctgactctagtcatccacgctgtagttaaagaactgtgagctgggagggtggggcaggcgagggggccttctgatatttatgtctcatctttccctgaaaaaatactgtgtttctactttgctgcttgtattcattcccaacctattactgttcataaaatgcataaggtttactttgactccaagtttatttttattaattaaaatattccatgtttatcataatgagggtgattctcattaaaactcttttcattgccttcctgactaaaagcaagtacatttttattttaataaacataagtaacacaataaaaaaaaaagaaccatgtgtttcccagctgtgtacctctctaagctgagtagcatcagcttataatacccagcatgaaaaacaactcaaatgtcagtcatcagcttaattggtaaagaaattgaggaaaactcatccattggcatattacccactactacaagcaactaatgttggctactgtcaacagcatggcaaaattcacaagtagttctcataagtaaaatgagccaaacaaataaaagtacttacataagatacaacttttataaatttcataaaatgaaaagtaatctaaacttacacaaaaatcagtagctcactgtgaatattgtaggaaaagggaaggtctaggaaggagaaattacagaacaagagaaaattttaaggaaattgacttgttatctacgttgataatgattatgtttgggacaatattagtaaaattgaacacttcataaagatattttttaatttaacctaattaaagatagcactaattataacatgtattatttggtagaaaaggagttagacagagataaataaaatacatgaaatgtcagagacacctgaaatacacagcaacgagccctaggcatctctgtatttttagagaaacattagaatacagcaaaataatggactaattctacgacattaagaaagtttatcaaacccaccaggcatgttgtattagttcatcttcacactcctataaagaactacgtgagactgggtagtttatgaaaaaagaggtttagttgactcacagttctgcacagctggggaagtcacaggaaacttacaatcatggtggaaggtgaaggggaagcaaggcacgtctcagatggcagcaggtgagagagagagagagagaagtgacatacacttttaaaccctcaggtcttgtgagaactcactatcatgagaacaacatggggaagctgcccccgtgatccaatcacctctcacttggtccctcccttgacatgtggggattacaatttgagatgatatttaggtggggacacaaagccaatgcatatcatatgtccagctctatcctggagttgtttcaggaatccagtgtgtcctgttgatagaaaccgtgacactgagctcacagcatcagtagtatttgacaccatgcaaagtcaagagatctcaactgagatttagtgtggatgttttgtctgatgaagtcgtacactcagagcaagtgaatatggagaggtttattatctgcactgtagaggtgtctgctgagagcagggcagttctctcaggactgtctaaaatggcttggtaaaggaggaatggagactgactcagggtttttataatggtttggtggtgggggcagagtgaggcttcccactcacagaaaggggtttatagggtttgaaactcccaatggcatcacatgaagaagctcctgtgattgcaaactagattcaccttgtttgacaaaagaggagatgatagaggagtgagccttaagtcctcagcagtcatgcatcaaaaatattgtgacaacttattctatgtagcaagaataaaaataatgaataagcaaaaagataagggttcaatgcaggtggacaacatggaggtctacaggaatgcaatgactttataaatataagcaaataatattgagaaaaaggaggaggggttggggaattagagaggggtcctggtctaatgtcttgggtagaagcttctcacaacaaaggactatcagcttattctgcaggtcttaggccagctatctgcttaaaaacatccaaaaccccagagaatctataaggatgctcagtttaatatctcctatttgagtagctttacagttgtgtggaattctttattgattcttttttatttgtttttagagacaggctctcaccctgttgcacagtttgaagaacagttgtatgatcaaagctcactgtaattttgaacacctggctcacataatcttcccatctttgcttcctgagtatctagaagtagaggggaacgccacccctcacctgcttattctttaaaatattttttcagagaaatagcatctctttatgttgctcaggttggcttcgattgcctcgtctcacgggatttccctcatttttgcttctgaaagtggtgtgattatagcgatgatccactgcatctggcctgaatttattctttagttgtaaaacatgaacccaacaattaactgcctgaatattttctgcagcgagttagttaaaagtatctgataagattccttccaatatgattcaagaggagtattttctgctgatgttccttccaatttccttgctgaagatcacaagagtctgtggaaaagatgtaacaaaaaggcagcctcaaactcttcatgattggagtggatatcacacaggaatcactttcagtattttgtaacacatgcatgcaataggacaaagatgatctctgggggtaaactctataaacgtatgggccttttagctgccaagtcatagggtaataactgatgcaccctgaggagtggaacatgattccatagtgctagtgggagaacctttggccaagggagttttacattttattaaagatttgatcatttttatgtagagatgccatttttaaaacattcctagaagattgttgagtaggattcattctgtcttatatgaacaatgataatgctctccatggttagataatattataaactagattgagctagagcgtttggtgtatttaatcactttttttagcttccattagcatttggattaaatgaaaatcataacaaacagtctctcagaccacagtgcgatcaaattagaactcagaattaagaaactcattcaaaaccacacaactacatggaaactgaacaacctgcttctgaatgactactgggtaaataatgaaattaaggcagaaataaacaagttctttgaaaccaatgagaacaaagacacaacataccagaatctctcggacacagctaaagcagtgtttagagggaaacttataggactaaatgctcatgagagaaagcaagaaagatctgaaatcgacaccctaacatcacagttaatagaactagagaagcaagagcaaaccaattcaaaaactagcagaagacaagaaataacgaagatcagagcagaactgaaggagatagagacaggaaaaacccttcaaaaatcaatgaatccaggagatagttttttgaaaagattaacaaaatagatagactgctagccatactaataaagaagaaatgagagaagaatcaaatggacacaataaaaaaatgataaagaggacatcaccactgatcccactgaaatacaaactaccatcagggaatactataaacacctctatgcatataaactagaaaatctagaagaaatggataaatttttggacacatacaccctcccaagactaaaccaggaagaaatagaatccctgaatagatgaataacaagttctgaaattaagtcagtaattaataatagcctactaataatattattaatagtaattaataatagccaactaaaaaaagaccactaccaggtggattcacagctgaattctaccaggggtacaaagaggagctggtaccattcctcctgaaactattcctaacaatagaaaaagagggactcctccctaactcattttatgaggccagcatcaccctgataccaaaacctgtcagagacacaacaaaaaaagaaaatttcaggccaatatccctgaagaacatcgatgcaaaaatcctcaataaaatactggcaaaccgaacccagaagcacatcaaaaagctcatccaacaggatcaagtcggcttcatccctgggatacaagggtggtttgacatatgcaaaacaataaacataatccatcacgtaaacagaaccaatgacaaaaaccacatgattatctcaatagatgcagaaaaggcctttgataaaatccaacaccacttcatgctaaaacctctgaataaaccaggtattgatggaaagtatctcaaaataataagagctatttatgacaaacccacagccaatatcatactgaacaggcaaaagctggaagcattccctttgaaaactggcacaagacaagggtgctctctctcaccactcctattcaacatagtattggaagttctggccagggcaatcaggcaagagaaagaaataaagggtactcaaaataggaaaagaggaagtcatattgtctctgtttgcagatgacatgattttatatttagaaaaccccatcgtctcagcccaaaaactccttaagctgataagcaacttcagcaaagtcccaggatataaagtcaatgtgcaaaaatcacaagcattcctatacaccaataatagacaaaaagagagccaaatcatgagtgaactcccattcaaaattgctacaaagagaataaaatacctgggaatacaacttacaagggatgtgaagaaccccttcaaggagaactacaaaccactgctcaacaaaataagagaggacatgaacaaatggaaaaacattccatgctcatgaataggaagaatcaatattgccaaatggccatacggcccaaagtaatttatagattcaatgctattcccatcaagctaccattgacttccttcacagaattaggaaaagctacttaaatttcatatgggaccaaaaaagatcccgtatagccaagacaatcctgagcaaaaagaacaaaactggaggcatcacgctacctgacttcaaactatactacaaggctacagtaaccaaaacagcatggtacttgtaccaaaacagacatatagaccaatagaacagaacagaggcctcagaaataacatcacacagctacaaccatctgatctttgaaaaacctgacaaaaacagcaatggggaaaggattccctattttataaacagtgctgggaaaactggctagccatatgtagaaagttaaaactggatcccttccttagaccttatataaaaattaactcaagatggattaaagacttaaacataagacctaaaaccatacaaaccctagaagaaaacctaggcagtaccattcaggacataggcatgggcaaagacttcatgactaaaacatcaaaggcaatggcaacaaaagccaaaattgacaaatgggatctaattaaactaaggagcttctgcacagcaaaagaaactaccatcagagtgaacaggcaacctacagactgggagaatgtttgtgcaatctatctatctgacaaaagggctaatatccagaatctacaaagaacttaaatttacaaggaaaagacaaacaatcccatcaaaaagtgggcgaaacatatgaacacttgcttctcaaaagaagacacatgaaaaaaaaagctcatccttactggtcattagaggaatgcaaatcaaaaccacaatgagataccatctcacaccagttagaatggccatcattaaaaagtcaggaaacaacagatgctggaaaggatgtggagaaataggaacgcttttacactgttggtgggagtgtaaattggttcaactattgtggaagacagtgtggcgattcctcaagcatcaagaaccagaaattccatttgacccagcaatcccattactgggtatatcagcaaaggattataaatcattctactgtgtcttacgtgacacatgaacacatatgtttattgcagcactgttcataattgcaaagacttggaaacaacccttatgcccatcagtgatagactggataaagaaaatgtggaaaatatacaccatggaatactctgcagccataaaaaggatgagttcatgtcattttcagggacatggataaagctggaaaccgtcattctcagcaaactaacacaagaacagaaaatcaaacactgtatgttctcactcaaaagtgggaattgaacaatgagaacacatggacacagggaggggaacatcacacacaggggcctgtcacggggtggggaagctaggggagggatagcattaggagaaataccaaatgtagatgatgggttgatgagtgcagcaaaccaccatggcacatatatacctatgtaacaaacctgtacattctgcacatgtatcccataacttaaagtataataaaaaatagtaactcaaaaaattatgttaatcagttctctagtaggtagaaattcatctgggagtttcttctcttgtccattttgataggatctgcagaagacataagaaccctctctgttaaaaatattcctaagttgtacaccatctagaaacatatatacttaattataatttttaatttattaaaaagctttaataagtgcaatgttttctgccttctgagttgatttcataacacatagaaggatatatcctgaatgaaactttgtactagtaatacaaattaaaggttaataacctctacttttattattgaggtattacccaccaatatataatcttaaatcagtgctctcaatggggcttttacctaaataatataaaaaatattttcctgatcttgacacaaaatagatgtgaacacattcttcatattcagccatgtctcctgtctattacattatgaaccacatgctaactttgatttacttgggacttgttctaatttcaaactagttattttttatcttcatgcagctagattattatgtgtgagtattttatcagagagtgataaagataatttaaacaagtaattgtgtgtctcaggactcttcttgggttctgagacaagaaatgccattactacctgaaggattgagacaactttgaaaatgtcagaggaatcatctctaattgcttctaaatttcagttttatcaattcttatgctttctgaattatttttgattttagttattaaaggattctttctttttctcttaatctagctaagaatttgtccttttttaagaaaaaactcttaggtttgttgatttttctattgcttgtaaagtcttttttcacttatttcatttactctaaagcctcataaacacattggtgacaggtagatggagaacttgattgtaaatgccctgtttttgttagacccagctgatcatgtcaggaagccccctgaggggtctaggagctgactgtggctcccaccactggacaaggatgagaacctccatgctttggggatgggaggacatgcagggcagtgtgaggggaggagggttgctggcaggtccagcattgcttcccctggctgcaccactcatgtccttcctcactcagagattcagtcatttcttcccaggtggaaaaatcagttaactttatctttgatatatggatgggagcccaagatagaggcttgtcagtcattatttctgtgcctgctgccttgggaatgtctgaatttctcatgctctgtcctgggtctataccccagtggctcccctactccatggcacccacctgtgcctgtgcatggcccctgatcacagcctccgtgaaacatctggtttggctcaggaagaaccctccctgccttgttttcctgctgtccctggtccacatccttctccatccacaggctctcagtggtgcctcccatccacactgaccctacgtgggttcatatcttgtccaccacttcctctctcttttcttctgcatcttgctttttattccaacagcctgtttccacctcacctggacttatgtacaggaaagtgtttactgagcaggcttgtgttgtgcaaaccctgaagattactgaaaaagtgctttctcaagccacatccatattcagctcctgggagatgatatctgcttttttggcatactcctcctggaaatatttttaaaccattttattaaggtaaggattacatctataagactatacgttatttaacatacttaattcattgagttaagtatgcaaagttaccatcaccatcaaggtcataaacatagcaatgacttccaaaacattcctctttattctttttattcttagtattatgattattattattagttgaaatgtatttttgcttgtgtgtctctgtctctgtgtgtgtagcctgggtgcacactatgctagctgtttccataatgacttatggggaatgcctattattgctgaaggggccagcgtctgagcactggaggtcagttatgcaggtgccacacacatctgtgatgggccccaataaacagcactgggcaccagcactcaggtgagcttccctggtggacaatgcttcacacgtattgtcatgcatcactgctgggagaactggggacaagagacttctcagggaaagaacacctgggagcttgtgcctggattctcagggccttcccactggttcctttgctcattttaattcatattcatttgctataataaagctgcacccacgagaataacagcttgtcttgagtcctttatttgtactgattatggggcctgaggatggtcttgggacactcaacacaattacatcagaggtgtaaattgctagaaagaccctgactactgacacatgtctatagcatttataatatcaaaggatgagaaagtgtaggataaaagacattcagtgcctggatggctatagaatcacatggcatgagatgatgtgtgcactccaataaggagcaggaagtgaaagtaatccatggaatttagaaacgacggacacagctccctaggagttgttccctgaacaagaacaagtaaaaataatgaagaacaacctgagtatgcaactcatatctgtgatagctaaaataatagtaaaaagatgcacatccagccctgcagtgtgccctgtgagccaaggctgctagactcccattcactgccaaagggtacagttgtccagaaacaacacacaacacacaagagctacacagatggtagcccagggtttggactggggaggggaatccacttgacaactagaacagggaggatagtgtggaaagtggcatcattttgtgatatgaatgatatcattaatagatatcatcagatcgtgaacaacattagccaatggactaggagactgactgatgtagcagattctctttggcttcatgtgttgcataagggaggagcatgtttgggaagatgctggacccacagctcacaatagacacatcacagatggctctagctgatcccaacctcaagtaggatgcttctgatgggacagctttcctcaagcactggatcccgatcatgtaaagtttctctaccctgagaatgagactgccctttctctctttaaaatgcaaagtgggctctccagatgaggagcccaatgctgtatatggaagccatagtgctatgtgtttatgttgataccgacggtcattgcttagcatgcatcttatccaggtcctgctgctgtgtgtttctgttgatgctgctggacattgcttcgcatgcaccttacctaggtcatggcctgtgctgtgataaagagaagtccttttgcttctgcaccccaggtacaactcctgtgagcaatctggcaaatattttagaaactgtgtagtttgtattgccttgtcttttcatttgaatcttttagatgataaggaaatgagagtggttcacaagataatggggcagacacaggggatagttaagggcctcctccaagcaatgtggatatcataaaatgaatgttaaaaaatgaaagaactaaacaagaaggtgctggggttgacacaaaggacttaaaacagaactaccaggagttgggtgcagcaatggcaatcccttcagttccatcagcaattcagagacctaaacaaatattccctatttatgttgaattggcagactgtagaaatgtagaagacaaaatgaccagaaaaaatgtgtgatttggcatgggtgaaggtctggcaagttaatcaagttgaggactgttgaagccctagggtggtcctgcctttgctgggtgcccagaccataatggcactcatgggaaattgccagcaagtagaaactaggtgttttttttttttttgtagttcttattatcaggcatatgtgcatttgaattttgtcttcatagcatttcctggttctatttctcattttttaaaacacacgtgatttcatctagatttatcaccttcacagggtcacagagaagggtaggaagaagggaggccctggtttgggtcttgtaggaccatggacaagagtgcggagggacaacagggggttgtaagacattattaagaccttattctgcctctcccaagaggctcagttcagccgtttttctgcagttgcggttctgggttataaaccttgtagactcttcccttcaggccagggcggcaactatgcaaattcaagtgggggcctccccacttaaacccagggctctcctccacagtgagtctccttcaccacccagctgggatctcagtgctttcttttctgtcctcctccaggatggggtcaaccgtcatcctttccctcgtcctggctgttctccaaggtcagtcctgcccagggtttgaggtcacagagaagaatgggcagaagggagcccctgatgcaaattttgtgtctcccacacaggtgtctttgccgaggtgcagctgttgcagtctgcagcagaggtgaaaagacccggggagtctctgaggatctcctgtaagacttctggatacagctttaccagctactggatccactgggtgcgccagatgcccgggaaagaactggagtggatggggagcatctatcctgggaactctgataccagatacagcccatccttccaaggccacgtcaccatctcagccgacagctccagcagcaccgcctacctgcagtggagcagcctgaaggcctcggacgccgccatgtattattgtgtgagagggaccatctaaaaccttccgcggtgcaggtgcagagtgagctgccagacacaccctccccaggggcctctctattcatccggggaggaaacactggctgtttgtgtcctcaggagcaaaaaccagagaacaacatgggagcgttcctaacccctaaggcaactggatgggagacctgacccatccagttctctgagggggctcttgtgtgttctacaaggttgttcatggtgtatattacatggttaacatcaaaaggctgcctaataggcacctcttcaatataatagtcttttaattagtgaaaattttacacagttcatcattgcttgcttgccttcctcccttctgtccgctcttactccctccttcttttattttctacttacttttccaaaatcatttaacccctttttgtaccattaataagttatcttgtttttgttgttgttttccttttaacaatatgcactgaataattcatctttgtgccaattcgtaaatattttgatataacaaagacttctgtcataaatagatttcctgtgagtaatcttgcaaatattttagaacctgtttggttaagaataaattaaaataaatagacaaatttttaaagacatacaacctatcaaaactgaaccaccaagaaataaaaaatctgggctgggcgtggttgctcatatgtgtattctcagcactttgggagaccgatgcgggtggatcacctcaggtcaggagttcaagatcagcctggccaacatggcaaaaccccgtctctactaaaaatacacagattagctgggtgtggtggtgttcacccgcagtcccatagtcccggctactcgggaggctgaggtaggagaatgacttgaacctgggaggcagaggttgcagaaagccaaaatcgagccactgcactccagactgagcaacagagtgagactccaattaaaaaaaatctgaacagaccaatgagtaaagagatcgagtcagtgatttttccaacatctcgaatcaaagtccagaacttcatggcttcactactgaattttatcaaaaattaaaaaaaacctagaatctctactcaaatttccaacaaaataaagagaaagaaatatatccaagcttattttggaaggccctatttccaaagcaaggaaaagacactacaagtaaataaaactacaggccaatatccctgattatcatagtttcaaaaactctcaagagtgatgagatgacaaacaaaattaaacagcagattaagaatagaattcaccattatctgtgtctcccatacccagagaggctgctgcttgccctgcacaaggatactctgagcagaaacctacctggcccagccctcaccttgctttatgcagccatctgccctggcagcttaacacaaaggacagcatcttttgggagatacatggccacacccactgcctgagaatccacagtagcccctattcctgggcaacataagccaagcaaaaaccccactgttaatactgtagctggtgctctttggaaagcaccatctccaagcaggaagccaataaacagtccatcacagtatctcctggtagactatcactgcacccagaaaggagaaaatgactgtgtgatctcagctatgaccagtgcctgcaccactctggatgaccaggaggtcctgagtctgtccatgtgaacagttcattaccgctataaccagcattcaagaaagccagtacacaaaggctatcaatatctaaggaatttcagtctccatcactcccctgctgcttccatcagacctggttcactgctcactgctgaaaaattgaggacacatcacaccaccggatccctcacatacattccccaacaccagcctgaagtgtgtcaacttcactgggcggctagacctagagaagcaacaacactcacagtagtatggctcccaggttctcctgctcctaggggaagggagtgcaccacattgagggaacaccctgtgggacaggagaatctggatggcaggccttgaacatcaatcagatccctccattggtgggaaacttcttttagtagaggcagttacagtgctgagctcagcagagaaagtcttcagctctgtctcaacagtcagatatccctggtgcatgtgaaaaacttgaagaagaggaagtctttcccccttgtacaccactgcaggcacacttggggtctctcccataagagctcagtacgggtgcaactatagacagcatttctggaacacatcatgttgactgcatcccccagaaagagcactctccaggttcaggttttggccagacacagagtcacaattcctctgtatttaacacttcaacttttgtacagaggaaaagacatgcctgtctgatctgaatagctggaatcctgggatacgggagtgtcttagaggtgcataactttcctacagacctggcaagacagctgaggtggctccaacccttcccccagataagacctcagtgtgactcactaaaagctcctccagccaactctgtcaaggctgggacttcatttaccacctgatttagccacagctggtttctatccagggacaagccccctactggtctgaagcctgaaccatcaaactagcaaataaatcactgggggaaaataaataaatacgtgcatgccacagaggaatgaactaaggtttaatagacctcaaccattctaaccccatagaagacagtgaacttgctcacatactgagcagatttctactgcaatcaacacataggaaagccatcatacaaagattctccataaccacggaactcttacagagtcttcacccctctaaggaccaaaaaccaaattagactgtaattaattataaccattaaagtctcattcttaagggaaaaaataaatttaaaaacaaacaaaagaaacagttaaaacaaacataatttcaatactagttagaagaaatagtctaccaaaataagaggaaatgagaaaaataactcaagtaatatgacaaaacagggtgctataacatcccaaaaaggtcacactaggtctccagcaataaactttaaccaaaatgaagtctttaaaataccagataagaaattcagaatgttgattattaagctactcaagaagatataataagaaggtgaatatcatcataaacaaatgtataaaagcagttcagaatataaatacaaatttttatagagagatagatatcataaagagaaaccaatcagaacttctgaaaataaaagacaaaatgcagggaaatagtaagggaattacaaaatgccatggaaagttttaaaaatagactagaaaaaagtagaagaaaaaataaacaagagactagaaaaagtacaaaataaaaagttgaagtaaaaaattagatgaaaaaagaagaagaataaagtagaaataagaaaaaagaagaaataaaaaagactatttttgaaataatcaaatcagagaaaaataaatttagaaagaaccaaaagaagtaaacacagtctccaggaaatatgggattatgtaaaatgaccaaacctcagcataattggtgttcctgaagaagaaaaaaaataatatgtttggaaaacttatttgagagaaaaattgagaaatacattcctggcctgcctagagacctagatatccaaatccaagaagttcaaagaactcctgggaaatgtattgcaaaactgccttcaccaagcttatagtcatcaggctaccgaaagttaacatgaaggaaataattctaagagcagcaatacaaaacaaaacaaaaaacccatcagactaatggcagacttctcagcagaaacttcaccagccagatggattaagcttctatcttcaaattccttaaatagaacaactttcaatcaagaattttgtatcctgccaaactgtttgataaatgaaaaagaagtaaagtcattttcagaatcaccagtgctcagggaattcgtcactaccaaaccagcagtacaagaaatgctaaaaggagttgcaaaccttgaaacaaaagcccagtaagcacataaagggaacctcttaagagcctgaaactcacaggcctataaaacaataacacagtgaaaaaaaaaaactaggtaagaattaacatggcaaagaaaacagtaactcatatctaaatattcacattgaatgtaaatggcctaaatgctccatataaaagatacacaatggcagaatagattaaaaaatcacaatggaaatatctgatgtctctaaaagatccccctaacacacaaagatttacataaactcaaggtaaaaggttggaagaaggtatttcacaaaaatggaaaccaagagtgagcaggagtagctattttgatatcagacaaaatggacttcaaagcaacaacagtaaaaaaaagacatagatgatcactatacaattacaaaaggatcaattcaataagaaaatactacaattctaaatttataggcaccaaacaccagaggaactagattcataaaacaagtactattagagatttaaaaaaatgagagcgaaacaatcagagtgggagactttaatacaatcataacagcactagacagatcttcgagactgaaagtcaacagagaaaaaatgcacttaaataactcactgaaacaaatggatgtagcagatgtgtacagaacattctgtccaagatctgcagaataaacattcttgtaatcagtacatgcaacattctacaagatagaacatataataggtcacaaaacaagtctcaatgaatttttaaaacagtgaaatcatatcaagtatcttctcagaccacaggagaataaaactaaaaatgaactctcaaagaaacccttacaactgaacaaatacatagaaattaagaaatctgctcctgagtaatatctgggttaacaatggcataagatgaaaatttaaaaattaccttaattaaatggtaacaagttattgaaaccttaagaatacagcaaaagcagtggtaagaggaaagtttatagtgactactgcctacgtgaaaaagccggaaagagcacaagttgtcaacctaatgttgacacgagaaaaaagaaaaaattaaagaaagaaaatataaatcaaaaaagagaaagaaacaagctgcctgtgttaggctgactcacgtcaaaggcagtaacacgcaaagtttcaatgatgttatctaagggccggagctcagaggaatgggctctgaagactctcccagcactccctcagcataaggatgaaaagaggtaagttttcctatctctcctttagtgtaagtgaacttccctgtcgaatctcatcccctctgctatgtaaactataccttgcctcttgctctgtacgttttaggagtttctgtttttcctgtagttaatgattgcaggttcctgcttctgcatctaagcagtacagcctgagcaggtctggcttgtagccagctacatgccatggtgggggtggcaggatgagtctttgtgagactcctttgaactaagcaggtaatgaccatctgggctgcatgggaaggagtattgctaaccctgagttacaagcctggctttgtttgattaacggcctctgtcttgcctctgtacgtgcgcgttcgtgccacttaggaggaggtgtataagcaaaaccttgtctttgttcggggcccagttgtttggacgttgagtcccttgggacagagtgcactcagtgaagaccctcctgctatgccccgaggtgtcccttgccctcctgatgttccgcaacctttcttgcgagccagcagccaggaggagaagagaggacagattggctgtctcctttgccggagggtctggggctctggtcgcggcgatctgtgaccccagggcggcctcggggagcacttcagcccggaggggagaccggctctccaggcccccggggcccctcccctgacagcgcaaaggagcgcggaaagggctgcgggatgatcccagaagcagcgctaatgaaccgcggtcggcttttggggcccaaggcaggacccgtcccacgaggacggaggggagccccatcacctgccgggctgtgcgtctagtccgacccagggaggctgcggcggcgagagggggtcgctcactcgggtaccgcagacgccgcaaccgacgcggcacaggagagaggcccgccaggccggttaggaaagggaactggggaggggaagtgggtgaaagggcgtgcaagagacggctgcaggaggggccggtgcggggagtgacgtgggaggcgcagatctcagcgtggactgggcgctcccagcgaagcctggggccgacctgggatagcggcgaagcggctggtaggagctgccccgtggcagcagcggccgtgctgggaataggggactctcctagccaggcggccccgaaaactcctgtaataggagacgctctgctcggtaaaaaaggaagtcagagtgagtgtgcgtcgtagagggaggaaatgggaggaaaatcgtggaaacccactccactggaatgtatactaaagaattttaagaaaggttttgcagcggattatggaattaagctgaccccctgaagttgaaaactctctgtgaatcagaatggccttctttaggggtcggctgccggccgagggtgctatatagatagggaggtgattggccgggtgtttaaggtggtgaccggggtgggaggacagccagggtacccagatcaatttccctatatcgattcatggctcaatgttatacaaaactgaccagcgtggttgcaccccccagcagcatattgtaagacgcctgtggcccgagcagccgaagacaaaatgaaagtaaaatcagccatgctggcagctgcagagaaaaagggaaagtcacaggaaagccgagataaaccagttctgcgggcacctcaggaggaaacagaatttcctcctcctcctcatgttccaagccacccccctttgccgagaccaactgctccctctgcaagttcagctgtgaggccagcagccccagctgtccatggggagttaaatccgggagagtatactcccccagcctcaactaagaaggaggaattggagctgcaggaagttagagtggaaagcccagaaagtcaggcaggccatctcaggtctggccgcacctgagtcctgcaaatgcgtctcagggagacgaggggacctgtctattatgaccagcatggccacatccagaggggagagtgaactttcgtttaccagcccttttcaaccactgacctcctaaagtggaagcatcatattccctcttacatggagaagccacaggctctcgttgattgatgcagtccgtttttcagacacacaatccaacgtgaccagattgaaaacagcttcttttgaccctgtttaatatggaggaatggcagagagtgacacaggcagccctccagtggctaaaactaatgcacctgcagacacagttaatgcccagacatatgcatatttgaccctgtttaatatggaggaatggcagagggtgacacaggcagccctccggtggctaaaactaatgcacctgcagacacagttaatgcccaggcatatgcacgggtcagttccctgaaacaaaccccaattgggacccaaatgatgcaacccagttgcagcacctgcagcggtactgagaggcaatctttcatgggctgaaggatggtgggagaaaagcagttaatataggaaaaatctcggaggtgctccagggagcagttgagagcccaagccagttgtattagagactttgtgaggcttatcggctgtacaccctgttcaaccctgagactactgagaatcagcatgtggtaaatatgtcatttgtaggtcaggcccagggtgatatcaggcaaaatttgcagaagatggaaggtttcgcgggtatgaatgctactcagcttattgaggtagccaccaaggtgtacattaacaatgatcaggaggcaaagagggaggctgattgaaggcttaagaagaaggctgatttactggcagctccccttacgggaagggaagctggttttgcaaggggatgcgggtatggatgagggcatggaagaggccggactggatggggatttgagagtcagctgagactagaaagagatcaatgtgtgcagtgcaaaaggaaaggacactggaagaatgagtgtccagagaataacaaggagaatggtcagggctgtggtatgggaaggccaccagcagggggctgccgcacctgggaggaaccagacactgacctaatcggactggccagggctgaaggatatgaggactaggacagaccgggctctttctctttgggcacccaagagcctatgttcagattagaagtggaggccaactagtggactttatggtagatactggggctgagcactcagcagtgacccgacctatagggccgctatccaagaactgtgcaactattgcaggagccactggagtctcagagaagaggcctttctgtcagtcaaagaggcgtgttataggaggacaaggagtccaacaagaattcctgtacctcccaaattgtccagttccttcgctggaaagagacccatttcaaaaattgcaggcacagattacatttgggccacaaggggatgtaactttaaatctagctcactcagaggctatggtgttaacccttactgtcccacgggctgaggaatggagactatatgcaggaaagactccagaaccaggagtaaattaaatgcgtgggttacttactaaaattactggagtttgggctgaaagtaacctgccggaactggcagtaaatccggcaccaattgtagtggagttaaaactgggagcgactctggttcatgtccgtcagtacccgcttctccctgaggccatacgggagtccacaaacacttagattgacttcacaggtgaggaaccatagtcaaatgccagtcactatggaacacccctctcttgccagtgtggaagccgtctggagaatataggccagtgcaggacttgtgtgcagtaaaccaggccacggtgaccatccacccagtgctgccaaacctgtatactttaatgggacatattccagtgagtgccacttggtttacagtcctagatttgaaggacacatttttctgtctccagctggcaccgattagtcagcctgtttttgctttgcaatggggtgaatcacagtatacttgaacaagactgccacagggatttaagactctcccacaatctttgaagaggcactggcctcagaccttaaagcctacaccccaccaagcagtgactgtgtcttgctctagtacattgatgatcttcttctagcagccccaacctggaaggactgctttcaggagacccaagacctcctgcacctgttatggaaggcaggatataaaggcaggatataaagtgtcagggaaaaaggatcaaatctgctctgaaagtgtccagtatttaggcttctatataagcgaagggaaaagattgcttggtagtgaatgaaagcaggcttttgtgtgcttcccactccaaccacctggtgacaaatgagagagttactaaaggcagctgggttctgccacatctggatcccatgtttctcactcatgggtaagccattgtatgaagccacaaagaggggaaagaaggagcccctcctctgggaggccacccaggaaaaggccttttaataaatcaaaggagacttgactgaagctccagctttaggcttgccagatctaactaagcttttctttttatatgcccatgagtgaaaaggaatggtcataggggtcctaactcaagtcatagggtcatggcaccacccggtggcatatttatccaggcaattagatactgtggcacttgcatggacccttgtttaaggcattagctgctactgcttcactggcacaggaagctaataaactgactttgggacggcaactgataatccaggtaccatactcggttataactttaatggacaaaagggggcaccaatggttataaaatccgagaatgatttgatatcaagagctcttatgtgaaaatccctgcataactttagagactgtaaataccctaaacctggctaccttgcccccttcattgctgtgtggacgtagcagatgaagtgttctcaagccagagagatttgacagatcggcccctcggggacccggacattgaatatttcactgatgggagcagcttcatcctagaggggcccgccgagcagggtatgcagtagtgactttggactcagtagcagaggcatagtctttgcctacaggaacttctgctcagaaagcagagctaatagctctgacaagggctcattggctagcaaaagaccaaaagaccaatattcacacaaactctaaatatgcttttgccattttgaatgttcatggggctatttacaaagaaagaggactcttgactgatggagggaaagaaagaaagtacaaggaagaaattctacagcccttagatgctgtgtgggccccgaagcaggtggctgtaatgcactgtagggggcacctgagggcaggaacatcagaggtgaaaggaaacagaaaggcagacaagaggcaaagcagactgcagtggtgactctgccttctaaagaggaagccttggctatgcctctccttccagaaattcccctcccagagaccccaagctacactccaaatgaaagggctttttttgcccaagaaaatgggagctacgttgaaggaggatggtggaaattctccaatgggaggctagccataactgaaatggtggcccccagatttgtaaagcagttccaccaagggactcacatgggaaaaacggcactagagacattattaggttgtcacttctatgtgtcccagcttacggctatcacttgagcagtttgtgaacggtgtttaacttgtgctcataacaaccctcgacaagggcccacttgaccccccaggaattcaggaaacaagagccatccctgtgtaaacgtgcttatgggcttcaccaaactgtcctgagcagggggctatcagtacatgttggtgcttgtctgcaccttttcaggatgggtcgaggccttctccacccagacaaagaaggcatgagaggcgaccaaggtattgttaagagacgttattcccagatttggactacccctgactctggggttagacaatggactggcatttgtggctgaagtagtgcaggacttaactcgactattaggaataaaatggaaattatatacagcctgtagcctgcagagctcaggtaaagtggagcacatgaactggacactaaaacagctactgaagaaattttgtgaggaaactcacctgaggtgggatcaggtcttacccgtggtcctcctccaagtcgggtgcacccgcacaaaacaaaccgggtgttcaccctatgagattttattcggccggccacccccaatcacaggtcaaattaaaggtgatctctgtgagttaggagaactctaaggaagcaaatgcaggctttagggctagctatgcagaaagtgcatggctgggtgcaagaaagaatgcccattggtctgacagacccagtacaccctttcaaacctggggacctcgttggggttaagaaatggaatcataactctgggacccatatgtgatgggccccatgttgtaattttgtccactcccactgctgttaaagttcagaaatcgtgccttggatccaccacgactggctgaaaccggcagcccaggaccagaggaccagctagcaggacccagaccatccgacccagctgaccctgcgacgagactgggttcccagcggagaggacaacagcccggctctgcttacttcggaagctgaccagtctatgcatggtggaagcttgagaaaacagcaagctctgctctagtcactctggaagctgactagtttacgcatggcagaaagcttgagtcgtcatcatcagatgagtaaatgtggccagagatcttaggtccagtaatcttatttgtattattaattatattactgctgtgctattgcttcaacaaatctcctccaccctgcccatgctaggtgtaagaatgctacaccttgccttgtttctgtcgatccccatatctgtcctaggagaggaagagcctatagaggaatgtccacattgcatgtgtactacgtggataggtaatacactagttaagactttattgtaccatacatactatgaacgcacaggaacccctctggggacttgtatgtacaaccaaacgaactattcagtttgtgacccaggggatagctagctttatgtatactatgaccctaagcttttacctgggcctggtttgagattcgcactgggtcaagggaaggaagtcttttaaacaagaccgaagtttctctctcccacaagggagtcacatctttgtattttgatatttgccagataacatccatagactcactctttcccgtaatccacagttccatggagtactataatagctgccataaaagtaagtgtgcatcccctgcttgctccaccggttccctggtaacaacttgctggggctttgcaacatggcctactaaccaacaatcactagggctaatcatgcttaccaaaataccttcagaaccagattgtaaaactaacatttgcaatcctgtgaatcttaccaccttggagccaaattggctcgtatggactacaggtttaaaagcaccactgagggtacaagccagcagccaagaaatagactcaagagtctatgtctatattatatacataaaaaaaaacctgaatccttcttgcccagcagcagttccaagtctttaagtcattcaatgagcactttaagcaggaggtacctgaagctcctcctcctatagctgtagatctgtttgcacggttggctgaaaacagctggcagcttaggtgtttcctcatgctttgtctgtggaggaacaaatatgggaaaccagtggccttgggaaacaaaagagctatcgcctcaagaaaattacactttaaccctttcatccccagagccggcactcacgagctcaagtgtctggctgctaaaatcttctgtcattggaagattctgcattgcccactggggaaaagcctttacaaacccagtaggggaattagtctgcctaggacagcaatattacaatgagacattagggaaaactttatggcggggcaataataagcaaggtaaagatacaaccaggttgccatgccataatccattttcccattttcactctttaaaccactcctggtatcaactagaggcaccaaatgaacacctagcaagcaccttctggcctcgactggatctgtgggccatgagcttattggcaattacctgccaattggacaggggcctgtgtgttggggacaataagaccatctttcttccttgacccgttacaacagggagaggttttagggtatcctatctatgatgaaattaaggggaggagcaaaaggaatatagacataaaaaaagatataaagataggagattggaaggacaccgattggcctcctgaaagaataatttaatactatgggccagctacatgggcacaagatgggatgcagggatactgtacccctatttacatgctcaacagcatcataaggttgcaggcagtacttgaaatcatcaccaatgaaactgcaaatgcattagacctgctggcccagcaagccacaaagatgagaaatgccatttatcaaaataggctaggcttggactacctcctagcccaggaaggaggcataggcgggaagtttaatctgacaaattgttgtttagaaattgatgataacagaaaggccatcatggaaataactgcaagaatgcaaaagctagctcatgtcccagttcagacctggaagccaatatggtctctagattccctttttggaggttggttttcaggttttggcagttttaaaacattaatagggatagtactagctatattaggaggctgtttaatactcccctgcctcttacccctccttatcaggggcatccaatcaactatagacgccgtagttgacaaaaccatcaccacttgactaatggctctaactaaataccaagctgtaccaaacgaagaaaacttgccttatcatgaagaacaaagttgtagtgatgacttctattaaaatttcatacataggaggcatcaaaggcagggtatgagatgggaaaaaagaaaaaaattaagaaaaaaagaaaatagaaattaaaaaagagaaagaaacaagctgcctgtgttaggctgactcatgtcaaaggcagtaacacgcaaagtttcaataatgtaatttaagggccagagctcagaggaatgtgctctgaagactctcccagcactccctcaacataaggatgtgaagagataagttttcctatctctcctttagtgtaagtaaacttccccgtcgaatctcatcccctctgctatgtaaactatatcttgctccttgctctgtaagtttttggagtttctgtttttcctacagttaatgattgtaggttcctgcttctgcatctaagcagtatagcctgagcaggtctggctagtagccagctagacgccatagtgggggtcgcaagatgagtctttgtgagactcctttgaactaagcagataatgaccatctgggcagcatagtaaggagtatactaaacttgagttataagcctgtcttagtttgtttaacggcctttgtcttgcctctgtacattcgcgtttgcaccacttaggagtaggtatataagcaaaaccttgtctttgttcggggcccagttttttggacgttgagtccactgagacagagtgcactcaataaaggatcctcctgctataccctgaggtctcccttgccctcctaaatttctgcaacaatgtcacacctcaagaaactggagaaataagaacaaactaaacctagatccagaagaagaaaagaaataacaaagatcagagcagaactaaatgaaaatcaaacaaaaaacacaaaaaaccaatgaaataaaaagttggttatttgaaaaaataatcaaattcatggaccattagctatattaaccaagaaaagaagagcaaagatccagataagttcaattagaaatgaaatgagacaatacaatctacactgctaatatataaaataatttgagactactatgtacaacttcatgtgcacaatctaggagacttacaggaaatggaaaaattcctagaaacatacaactctcctagattaaatcaagaagaaacagttactttgaatagatgaataataaacaatgagattgaatcagtaattcaagaattgccaacaataccaacaacaaaatagggccaggtgaattcacatttgaagtctacacaaaattcaaagaaaaattgctaccattttttctgaaactattttttaaatttagaaaaaaagaatcctccctaaattattctctaaagctactataacccagatacccaaaccaggggaacacacacacacacacacacacacacactctctacatacgaattttcctggtaaatatagatgcaaaaatactcaaaaaatagctacctgagtccaacagcacatccaaaagataattcatcatgataaaagtaggtttcaactcagaaatggaaggattatttgaacatacacaagtcaataaatgtaacacatcacataaacagaactaaaaacaaaaactataggattatctcaatagatgcagaaaaagcatttaacaaaattcagcgtttttaattataaaaattctaagtcaggtaggcatagaaggaactgacctcaaactaataaaggttatataggaaaaactcacacccaacatcatgctgaatgtgaaaaagttaaaagcattccccctgggaacagaaacaatagaagaatgtccatgttcaccagttctattcaacatagtgctagaagttctacccagagcaattaatcagaagaaaaaaataaagggcatccatattgagaaagagaaagtcaaactatcactgtttacagatgaagtgattatatacctagaaaaccctaaactcccccaaaatactgttagttttagtaaatgaattcacttatgtctcaggttacaaaataaatgtacacaaattggtagcactgctatacatcaacaacaacaaagctgcgaatcaaatcaagaactccatttcttttacagaagctacaaaaagatatttaggaatatacttaaccaaaaaggtaaaagatttctacaaggagaactacaaaacactgctgaaagaaatcatagatgacacaaacaaatggaaatgaatcccatgctcatggattggaagaagcaatattgtgaacatgaccgtaatgcccaaagcaatctacagattcagtgcaattcttatgaaaatacccacattattttttacagaattaaaaaataataatgttaaaattcatatgaaaccaaaaaagcccaaatagccaaataaatcctaataagaaaaaaatggagccatcacattactgaacttcaaattataccacaaggctgcagttacctaaacaacatggtactgatataaagtaggttcatagaccactggaacaaaatagagaacccagaaataaagccaaatatgtaaagccaactgatgttttacaaaacatacaaaaatataaattggaaaatagacaccatatttaataaatggtactgggaaaactaggaagccacatgtaaaagaataagactggatctctatctgtcaccatataaaaaacaactcccattcaaaatttcaaagaaatgcatttacataaattggaaaaaaactctttataatttgtatggctggggtgattgacccaggctatcaagatggaatcatactactactccacaatggaggtaagaaatagtatgtttggaatacagggggatttcttagggcatctcttagcattactttgccatgtgattaaggtcaatgggaaactacaacaacctaatccaggcaggcatacagatggctcagattcttcaagagtaaaagtttaggtcactccaccaggtaaacaaacaaacaaatgaacaagaaacaaaactgaccagccaaggtaactgctgaaggccaaaaaatacagaatgggtagatacaagaaagttgttatcaataccaggtatgaccatcaaccagaaatgaggactggaattgtcatgagtatgtctttcttatcttcttaagaatatatttctgcatgtatataactgtactaagaaaatgttttctttttgtttttcatgtaacataagatttatcgacttcatattagcatttgagagttgttaactgtatatagtggcatttaggttaaggattagtgcattttcagttgtatgaaggatagctgtattatgttaggcataattatgaccttactattgtctttatttggagatcgagcatgatttcaggtgatatgtagggatgctaagttgacaacaagggattggcctgtgatagttaatattaggtttaacttgattggattaaagcatgcttagatggctgggaagtattgtttctttgtgtgtctcttgggttttgccagaggaggttggcatttgactaagtagcctgcaagagaaagacccagccccaatatggaagggcaccatccactcagctgccagcacagctagaataaagcaggtggaagaagaggagataacattgcttgctgcgccttctgtttttgttttgttttgctttgttttccttgctggctgctttcttctgctcttcctacccttggacataagatttcacgttctttggcagttgaactatggggcgtgaagcaggggcttgctgagggttcttgggcccttggtcagagaatgaagtgtgcactgttggcttttctggttttgagacttctggacttagactgagccactaccagtttctctctcttcccaagcttttagaaagcctatcatggaactttcattttgtaattttgtgagaaaattctaataaactatatttcctatatacatatgttatattagttctatttctctagagaactctgggtaatacagctttgatacaaaaaacaacattgttgtattcacaaaaatgtatcctaagataagccaacacataaaagtgcataaagtatgattctatttctaggaattataaaaaatgaaaatgaatctaaagtaacaaaaaaagatcagtagttgcctaaggacagattggaaaattatatttttccaaatgctacaaatgatactctcacctactatgagcattgctctctcctcaggaatttcacccaagaaccgactctatagtcggagacatgcaaacaggttcctgttcatgtacgaactcttcctaacactttcttttcatgtacttccttgacttatttgttgatgaaggttgataccataaaaataactaaaccaggctcacattagagaaaaagaagagagtgatggaaagattaagccggagcatccagtttcagtgattctttgaccctgccctccctgaagtctagaggcagagggatgagccctgctgagcagttcacacttgtccacagggagaaagaggtgtaaatgtgccccccatccatgctcataaaatggcccatggacagcttatcccttgtccatgaaggggatgccacctgatgtcggtgcccttcctcagtgtccacaccttagggtctctgctaatctggggttctcttcttattctcagaggtagggtccctgtggatcaagcctagctgtggctgcttctcagcctgttccctctgtgtttgtaagagtcctacaagaagttcactggtggaatctggaagagaaatgatacagcctaactgttctcagagccttttttacaaagcctctgaatttaccttcattgactctagcatgagctcagcccagcaggcttcaggacaggggttggtgtgggaggcaaaagcgagggattcaagtggaatttgttagtggtaccctccatcaatacaaagaaaaatcataatcctcagggacacccttgtcagcacagtctccctcaaaatgaccaacctgagagccgaggagaaggccatgtattactgtgagagacacaggagatatccgtgtggcaacctaacacaggggacacctgtatttgtgtctgagcccagacacaaacctccctgcagggagacaggaggggaccgtgtgacagacactgctcagaaccaccaaggggagctcagaaccagcaggaggcagtggggacacaaaggaagactcaggagccaccagggggcgctcagaaccacctgggggtggtgaggacaccagggggcgctgaggacacaaggggaggatcaggacaccagggtcggctcaggacgcgaggaggtgctcaggacaccagggtctgctcagaaccaccagggggcactcaggaaccaccaggagaggctcagccaccaccagggggcactcaggacatcaggagctgctcagaatcaccagacggcggctgggcgcggtggctcacgtctgtaatcccagcactttgggaggccgaggcgggcggatcacgaggtcaggagatcgagaccatcccggctaaaacggtgaaaccccgtctctactaaaaatacaaaaaattagccaggcgtagtggcgggcgcctgtagtcccagctacttgggaggctgaggcaggagaatggcgtgaacccgggtggcggagcttgcagtgagccgagatcccgccactgcactccagcctggggtgacagagcgagactccgtctcaaaaaaaaaaaaaaaaaaaaaaagaatcaccagacggcgcacaggacatgaggtggcgcacaggacatgaggtggcactcaggacacaagggggagctcagaaccacctgggggtggtgaggacaccaggggatgctgaggacacaaggggaggatcaggacaccagggtctgctcagcaaccaccagggggcgctcaggacatcaggagctgctcagaatcaccagacgtcgctcaggacatgaggtggcactcaggacacaagggggagctcagaaccaccagggggcgctcaggacatgaggtggcgctcaggacacaagggggagctcagaaccaacagggggcgctcgggacactagggggtgctaagaacaccaaggggcggtcaggacatcagggttctcttaggaagcagtgcatcatcaggtccctggggagggtgggtttctttatttaggccttgtgattcctgacctggtaaagtgaaactcttctcagcatcttttgccatttcttccttggaatccatgtttctcttacctacaaaatcctaacttagaacaggaattcaattaaacttttaattctttatatttttgtaataatactagtaataatatctcagtacaattttaaaatgaagaaattgtgtatgcttacttcaaactatttacacagatttttgttactgtgctgtgtcagtcacacttttgtaaatgttttctaacaataactcagcatatgcatggtgttgagtttctttttcttccatttgctgattgtgaagatgaaaccccactttaagggctcctgtcctccactttggtggcccctggtgttctgcccctcagtctgtttctccaccttcccttcttctttccaaaccttttataatcctcaatttccattcaaggaatcagcaagcccgtttattatgtctcatccatgtctggtgaatcagttcacttccctttatgatcactgaagccaaccaagtataggcaggcaacagttctaagaatatgctcatctacctgcagactctctgccctcctcacccctttcaacggtcctgcagacatcaccccaccccatcctctatcttccctaagtaccacagagtgggctctgcagctcctgctgcccactgtgtgctcagccctggggctcactagtgctttgatgataaagcctaaatccctaacgtgtttgcacacttcctgaccaccctctagcaagttgtcattgtgagtgaatcctgggaaccctgtgctgatattaattctatatttatggatattctctagcatccaggaatattgtcaaataacctatcgagttgatgttaaaaattcatgtgaagccattgcaaaagaaaaaagtctttaaactggaaattgctattttttaatacttaatatttttttaatttaatatttaaatgctatttaaattagaaaaaggtgatgccttttgtaaagtacaatgttatttcaaccaatcgtgtaataaaattttaatagagatgcttgccttattgattattatatttatttataacattaaatgcattaaaacatttatttaaaaaatatgctattagtaaaaatgggaaataagctagcacagagtatgtaaagaataccattgtatttttcaatgatagatttgtcaataatggcagttacagtaagttttattaaaacatgcatttacatatatttcttaatttacattggaatagacagacttactcatagaagaatgttactttggactaaaaccttaaactataaatacaaataaatccatataaattaaatatataacacagaaacaaatgtatgtcactttgatgtttggaaatgcatttatttttgcctgtgcacatatatgtttcattgctgggtgtgtgtgtctgtgtctgtatatatgggtgtgactgtgaagtttcaaaatacattattaacttgtatagttatataaatctaagtttttttatgttaaaagatagtaaaaaaacacacacaaaaatattctagagagcatttgcaataactaaggagtattcaggtgttgtattaataatgaaaaactagccaaaatgtttttaaacttttattttaggttcagaagtacttgtgcaagtgttgttttataggcaaattgtgagttctgttggtttgatgtacagattattttgtcaccaaggaaataagcatagtacctggtaggtagtttttcaattctcaccctcctcccaccctccacactcaagtaggacctagtgtctgttgttttctttgtgtcaatatgattagctcccacttatacatgagaacagatggtttgtaattttctgttactgtgttactttccttaggataatgacctccagctccatctatgttgctgcaaataacatgatcttattcttcttatgactgtgttgtattccattctgaaatatacataaatcagtaaatgtgattcatcacacacacacaatcatctcattgatcaatattaactttcaatgtaaatctcccaggaatctttcatcttctcttgaccctcccctctcctcaggcatcccaaaccatagctttctatatattaggatacatgaaaatagggccctctctcagctgatgaaaaccagcccagccctgactctgcagctctgggagacaagccccagccgtgcagtaccaaggtgcttccattcagtgattagcactgaacactgaagacatcccatggagttaggaatgcgctgggttttctttgttgttgttctaaaaggcaatcaagaagaactagaaatattgtctgtgagtgatatgagtgagagaaacagtggatctgtgtggcagtttctgacaagaatgtctctgtgtttgcaggtgtccaatgtgaagtgtagctggtagagtctgggggaggcttggtacagcttggggggtccatgtgactctcctgtgcagcctctggatacactttcagtaactatggcatgcactaggtggaccaagctccagggaagggactggagtgggtcttacattaatgctagtggtggaggcatatactactcagactctgtgaagggccggttgaccatctccagagaaaacaccaagaactcactgtatctgcaaataaacagtttcattgctgacaccatggccgtctattactgtaagagacacagtgaggagaagtcagtgtgagcccagtcacaaacctcctacaggaacgctgggaggaaaatcagctacagggctcactcaaggcccactgatcagagtccactccagagggaggttcacatggaggttaggggctggttttctgtcaggacattggacttcatcatcttcttccattactttctttagggagcctcttcattttagaattctgtgtgtactattgttatatatacatatatatatatatatatataaaataatcatagtaatgcgaggacatattttctcatgcacaaaatgtagattgacacttacagagaagaaaaattatcaaccaatgtcaaaaacatcagagtcctggaaaatcttaagtgttctggtgggtcatctctaatcagacccagaacagtaacctcagtgaaattccatgattcagatactatttttggattctgatcataaccaagacagagtctggaccagggtcagtgtcatatagaacctcacagcttccatatatgacccttctcttgacagtgaaatatgaaattcagtatcagcagtgatttgatttttcttttgctttccgtattctttctttttagttgttgttgtttagttatttcttttgctatatccattttctttttttttagttgtgcctttcacttttggctttcacttttccatttgcttttcctgcttcctgataaagggagatgtaatccctgtggtctaaattccagggctcaagccttttcctgggagctcaggtgggtctcaggatgtggctcctgcagccacgtgaaagaggctgatgggactttctttccttctcatgactcaggcattccacggtgttgtgtggagactcatctgagattgcacgtggccattagtagtgaaggggatgagatgtgggtgtggactgatcctgtgctgtgcaaattgtcattagtagtgaaggggatgagatgtggatgtggactgatcctgtgctgtgcaaattgtcattagtagtgaaggggatgagatgtgggtgtggactgatcctgtgctgtgcaaattgtcattagtagtgaaggggatgagatgtgggtgtggactgatcctgtgctgtgcaaattgtcattagtagtgaaggggatgagatgtgggtgtggactgatcctgtgctgtgcaaattgtcattagtagtgaaggggatgagatgtgggtgtggactgatcctgtgctgtgcaaattgtcatggagtcacttttctcaccatagtgttagaaagagactgtaaaagttgtcagaatcaaactgcaacaatttgtgttaaaacctgggaaatggagctgggaaggccatgagggagggttctcatacacatgccttccataacaagaactgtcataaatagattctgcaaaaccacaaccttgcacaaaggccagcacaaccttaaaataattacttctccaagtacatctgcccagaggtgcctgtgcaatattacactgatgccaccctggttattgatcattttatcccaggttaagtgtctcaaaaccgcatatgtggccctcctcattttttctttaaaacactttccttatttacctaccgaaatgtgcccgtgcacattcccattgcaatgctaattctcaaataaataccactttactttagagtttctttctgtctgatattcaggtttgacaagctgcagatggacttgccaattttctgtgggatacagggtataataattatgagagatgtgtaggcatgttcaatgttcataagagcaaccgtaaataaatgcaggctggaagtctcagagagagctgaatgtgctgaaacaccatactattttatcttctcccattcttttctgattggaatcagacccaaccaggttatcaatgccaatctacctaagctagagccaactgattacggtatcaacagcaacaatagctttttattttctgcttggctttgtattgttaccttgttccatcctctagagtatctatttcagcccttcggataccggtttcagaggctttcccctaaagtgtaatttcttagttttgtagtggtgataagggaggaggtgggtctggatgtgttttagaagttcaggctgtcattctctcccagacagacactttagtacagaacgatttatgtggttgtcccatttctacaggaaagatattcaagagaataagaaagctcttcagtatttggtccctgagaattttacactagagcacattcaccactcttgactttaagcaacctaattaatgtatttgaagccttatcttgaaatagactatatttcatatggcagactctgcctcaggtaatctcataccctgactttgttactctctacaagaacttgtctctccctaaatttcagatttgttgtttgtcttaaaatttcgattatctgaagtatgaaagacaatttgccaagtttcttattttctgttcatctgttattgttgatgttgttgttgtaaaatacaaaaatatatatatttcttatctaagtacttctgcaagctgagtagcagatatttttggtaaaacccagaataacagaagagtccacatattaattagcttcttaataggaaaaatcattatagtaaatttgttcactggactgttacccatcatttataatgtacaaatatctgatacacacaacaaactaaaatatctaggtatttatgctgagtaaaataagccaaaaaaataagtatatatgctatattattccatttctataaattctggaaaatgaaaactaacctacaataatttaatgaagaacagttgttgcctgaggaaatggtagaagaagggaaagggataggaggaaatgtggaacaggagaaaaaattgaggcgaattcacttgtccactttcttgataatggtgatagttatgtcatgtttatcaatgtatactttaagtatatgaagtttattatctctcaattagaccttataaagtttattacaagcaaacaaataaaaaattagacaaaaagaactgatagaaaatgggtaacatatgttaaatgccagttacctgaaaatttatctgcgtgaaccttagttctctccataattttaggtgaacgctggagtgtaacagagtcacaatattttcattaaggcagtaagttctgcaagccacactagacatgtcccgctctgtcctggagttagttcagggaacacagtgatgaggaacacgggctcagataccggaactcactcatctcagacatgagcacttagacacatacataggcactctgtgtgtgggtttactttcacatccgtacatggagaaacattgagtctcacagacataatttacacacatatttaaaatataataaggtgtttgatgctgtttatcacagcacaatttcctcgtaaaagtgcaaatcttctaaataccataagttaccaaactcctgcaggacaccatcatcctctctcggcaatgccttctcctcacgtgtcccaccctagagttttttatgtagttggaaatatgcaaaggaccctccttctttggtgaaaaccagccctgccctcaccctacagttctgggatgggagacccagccctggaatttcaaggtttttccacatggtgattagcactgaacagagaactcaccatggggtttgtgctgacttgggatttacttgttactattttaaaagtagattcagggtgaactagagattttgagtgtgagtggataacagttaaggaaacactgaaaatatgtgccagtttctgaccaggatgcccctgtgtttttgggtgtccagtgtgaggcgcagttgttggagcctgggggagacttggtacagcctggcgcggtgggggtgggggtccctgagactgtcctgtgcagcctctggatttgccttcagtagccactggatgcactgggcctgccaggctccagggaacagactggagtgggtctcatgtattagtactgatgggagtagcatacgctatgcacattctgggaagttctgattccccatctccagaaacaatgccaagaacacactcttatctggaaatgaacagtttgagagctgagtacacagccacgtattactgtgctggagacgcagtgaagggaagtcaggttgagcccagacacaaacctctctgcagaaacacatgaagtaaatcagctacgggggcgctcaggacccactgatcagattcaacctcagaggcagatgcagatacagggtaggagctggttttctcatagtgtttgtaacttccctctccaactgacagtttcccagagaacctcactaatttagagttctgtgcctaccaaaaaaatctccaaatatacaccttttaaaaggtagtaatataaaggcctagtctcacatgcacaaaatgcagatcaccacttccaggcatgagaagttctcaaccacggtcaccaagatcagagtcctgaggaagctcaggggtgccttgtgggtcttctccaatcacattcgggacaggaaactaagtttaattccctgattaaaaaaggctataattccctgatttaaaacgtttcattccctgattaaaaaaggctggaatttttctgattccagccaagaaagaggctgggccagagtcagtgtcacgtacaagctcacaggtttcacgtctgccccttctcctgatatgagactatgcaaagcagtatcagcactcatctgctgcactttttgctgtcaatccattttctttcttttcagctgttgtcactttttcgttttcttttctgctccctgaaaaagaaagatgtggtccatgcggtctaaattccaaggctcaagctcttccgctggagctcaggtggggctcaggctgtggctcctgcagccgcatgggagaggctgatggggctttcttctttccccagtgcttgggaccctccaccgtgtttcatggggactcatgtggaaatttgagtggcccaggggaactgaagggcataagcttgtttggtgagagtgaaacgtgactgtggaattgattctttgttgtgcaaaccagaagaaattttgaattttgtcagaatcaaaacggagtcacttttgttaaaacttttgcaaatgggactgggaaaggccatagaagcagggttctcatacacttacacttggtaacaagaactaccacaaatagagcctaaaaaacacaaccttgcacaaaatccaccacaactttaggaaaaagctacccccacaaggatatgtcctcatgtactgtgagtccaactttccactgttgccacccttgttattaatctttgcagcccaggaaaattgtctcaaagcaacttagcaacttacttagcccttcttattttaactttaactgcctaagaatttataccgtgaagtaattgcttcatttagtgttaatattaatgcccttttgacaatctttgaagctctttttttctgatatttcttccaacataaagaatgggagctttttcaccatataaagaatttttaggaagatatgatggaaagggaaactcaatgtattgttcagtgtttctgcctaacacatcaatctcttaaaatattttgctgtgtctgcatgtgataaagcagatgctaacactggagataaaagaaaaaaatgcacagaccttccacattccaaatcataaagtggtactggatgtgtttctgatgaagtgaaccatgggtcataagtgctagtgcaagtagctttggaaaatggagttcatagcttcttaaagttttgctagttttaatttaagaatgttatttactatttctttttagtacaagctttccagaagattcttgatggtaatggaagtgtcgcatttaagaattacaaatgactcacagagttactttgtaattttttctgtagggtgtacatgtcagtcacttgacataaaagctcatatgtcacagatagaaaaatgaaaattgagccaattttaataactgcagtggaaatagtttttggcaatgagtgcttcaagacaaccacaaaataaatcggcaaaatattaaaatacagcaggaatctgatctcagtttctctgccttataagaacaaggctgtcccacatcaaaattattattcaagctctaggaaaaaatacacttttggattctggaagctaaaagctctctccttagatagtgctaaagtacttgtgaaatgcagagttgtgttcatgaaaaagatgactttattgtgattttttttttttttttttgcctgttgacaactcaccaatctgtctcagatgtaaagccctgctctatgggttgaatgtggttgcacatgactggcagagactgattattcataactccaggtgccctgtctttggagcagctacaggagtcaggcccagactagaagagcccacactgaccttctgcatcacttatgctctctctggccactacatcacaaacagtgcttatgactgggcctgaatctgccagcccaggaaagggctggaatggatggggtgcattggtagtgatggtgggggtcctactagcctgtggcaaatggaagcatctcttttttatcagactgaataatattgtagtgttttcttataccacatttacttcatccctttgtgcattaacacttaggttgtttttatatattggctgccataaatagagctgcaattaatataagagcacaggtgtctttacaagttcatgattttatttcctttccctgtatttccagagaagcgtttgctgagtcatattgtatttccatttttgatttatttatgagctgctatactgtttttcataatggtgcaactaaaaaatggtgtagtcatcatgaaaaacagttttagtttttgacataatccaaacacaatgtttgaatctggttatctataggagtttctagtaaaattggtgaatgtaaattaaaaagcagtaagaaaaatctgcaattcagaaatagtgtacccagatggtctttctctccgaaatgaaagagaaataaactatttctcaggtgagataaactaagatattttcttcacacttgaccagtcatacaagaaatattcaagggaatttttgtataaaagccagcatatgaaaaccatcagcttggaaaaaaacacacaaaacctatatttcaatggtagaaacaattaaaaaaggagaaaagaaacaaacattattactacaaaacaacatcaaatcataaaaccaaacagaaagagaataaacaaagatagagatatatacatatatacaaacaatcataaaacaataaagaaaataatttgagtaaatacttataaataataaccttatgtaataaaattcctcaattaaaaatacagtataaaaattgattttattaaagtcccaactctatgctgcttacaagagactcaaatcatctgagaatctacacacagactgaaagtgagtgggtggaaaaaagatatgctacaaaaatagaaaccaacagcacaaatagctctacttatatcagacacaaattttaagtcaaatgctgtaaagggagaccaaaatggacattatataataaaaaatgatcaatgtagcaagaatacgtaacaactgtaaatatacatatatacatatatgttatatatatgtaacaagaacacataattgtatatatatacatatatacacacacacatatatacatatgtacccacacctgagaactcaaatatgtaaagcaaatctattacatagtaagggatagatactaaccttatacaattatagttggagaatttaacacgttatttagcattggatatgttacctagccaaaaattaacagacaaacattggatttaaactgcactatattccaaatggacctgaatttacagaccattgcaccaaacaacttcagaacacatattcttttttttttaaaaattaaatggatgtgtttatttatctttttttattattattatactttaagttttagggtacatgtgcacaatgtgcaggttagttacatatgtatacatgtgccatgctggtgtgctgcacccatcaactcgtcatttagcattaggtatatctcctaaagctatccctcccctctccccccaccccacaacagtccccagagtgtgatgttcccctcctgtgtccatgtgttctcattgttcaattcccacctgtgagtgagaatatgtttggttttttgttcttgcgatagtttactgagaatgatgatttccagtttcatccatgtccctacaaaggacatgaactcatcattttttgtggctgcatagtattccatggtgtatatgtgccacattttcttaatccagtctatcattgttggacatgtgagttggttccaagtctttgctattgtgaatagtgccgcaataaacatacgtgtgcatgtgtctttatagcagcatgatttatagtcctttgggtatatacccagtaatgggatggctgggtcaaatggtatttccagttctagatccctgaggaatggccacactgacttccacaatggttgaactagtttacagtcccaccaacagtgtcaaagtgttcctatttctccacatcctctccagcacctgttgtttcctgactttttaatgattgccattctaactggtgtgagatggtatctcattgtggttttgattcgcatttctctggtggccagtgatggtgagcattttttcatgcagaacacatattcttttcttcagcgcatgtgatactcttcagagttgatcatatattaatacagaaaatgtatcaaaaaatttaaataaaattataccgacgttttatctgactaagatagagtcaaagatggaatcaaactaaacatcagtaactggaggaacttctttttttttttttttgagatagactctttctctgtatcccaggctggagtgcagtgacagaatctcggctcactacaacctccaccttccgagttcaaaccattctcctgcctcagcctcctgataagctgaaattacaggcacctgccaccactcctggctaattttggtatgtttagtagagagggggtttcaccatgttggccaggctggtcttgaaatcctgacctaaagtcatccaccagcctcggcctccaaagtgccgggactacaggcatgagccaccacccagcctggagaaaatttcaaaaaaatataaacacaaaaattaaacaaatcactcttacatggccaatgggtgaaagaagacattaagaagaaaataaaaaatgtatgaaacaaacaaagaaacacaacataccaaatcttatggcgtttagtcaaatcactattatgaggcaagtttagagcaataaatgcccacatcaaaaagtagaaatatcttaattaatcaaaataacaatgcatgttagagaacttgaagaacaagaaaaagctaaattcaaaattatttttaaaaacaataaagagcagattagatataaatggaattaagactaaaaataccagaaaatattaaaatagcaatagtctttttaaaaaagataagcaaaattgaaagccattagctgggctaacaaaaaaaatataaagagctaagacatgaataaatgaggtagaaacagaagatgtctcaactgatatcccagaattaaaataaattaggaaatactataataaattatgtgataaatgtgaaaacctagaggaattggatacattcctggccatatactacctaacaagattgaaaaaggaaaaaatagaaagccttaacagaccaataaaaagtaatgagagggactcacttaaaaaatctatatcctattgaacataaataaaaaaacccaaattactagcacagcaaatcaaacagcatttaaatactaatactaatactaatacagcatgacaaagtagaattcaacccagggattcaattatgctttaatatacacaagtgaataaacatgatatatcacgtatatacgacaaggatgaaaaacatatttttactttaatagatgcagaataagtactttataaaactcaaaacctcttcatgaaaaaaactctcaattatgtgtagaaagaacaacacaataagaccacatgacaagttcatagctaacatcatacttaacagaaaaaaagtttaatgcttttcttctgaaaactggaaaagacaaggaagccaactctcagcacttattcaacacagtactgaaggtactagccagagcagttagacaagagaaagaaataaaggggattacaattagaaaaaaggtagccaaattacctctaatttcagatgacataatcatatatatactaaagactcttgtaaatgctcttagagctgatgaatttagtaaagtttcaagatacaatatcaacatagaaatctcagtggggtttctatacacaaacaataaactaggtaaaaaaaatcaagaaatcccattttcaataactacaaaaactattaaacacctaggaatacatttcaccaaggaggtgaaaatccgtaaatgaacaaactatgaaacactgatataattaattgaatgagacaaacacaaaataagaaggacaatctgtattcatgaactggaataatttatattgttagaatgaccataatgcacatgacaatctacatattcaatgtcatctctatgaaaataccaatggcattattcacagaaatattaaaaagcattattaaaatttgtatacaattgaaaataccccaaatgactaatgcagtcttgtggataaaaaacagaggtggaggtattaacaaccagatttcaaaaaaaattataaaactgtaataacaaaaacagcgtagtgctggcataaaaatccatggaaaaaagtagagaaaccaagacattaatccatatatttatagataacttatttgcaacagaggcaccaaaaatatttattgtgaaaagaaaagtatcctcagtaaattgtgtagggaaaaactgaatatccatatgcagatgaatgaacttacattcccacatttaactgtatataaaaatgaaataaaaataagttaaatactcaaatggaagacccaaaactataaagctgctcttaaaaacttagaaataatgcttcatgaaatctgtctggaaaacaattttataaataagacctcaaaagtacagtcaactaaataaaacacaaacgaattaaattttatcaaactaaacagtgtttacacagtaaaataatccaaatattgaatagataacataaaagtggtaaaatgtattttaaactattaattaaacaggctattaatatccagaatataccagaaaaataaatgtctcaaccacaaaaatataaataatcttattttataacagacaattaacccaaataagcagacgtttctcaaagaaacacatacaagatgccagaaaatatttgagaaaaaatggtcaccatcggtaataatcaggaaagcacaaatttaaagtacaatgaaatattatctcaccccagacagaatagccatattcaaatacataaaaatagtgctaatgtggatgaagataaaatgggacatgtatacactgttgatgagaatgtacactcatacagccactatggagagacgtatagaagttagtaaaaccacaaatgcaacaatcatatgatacagaaattttacgactgaacatttgtcccaaggacaggaaattaatatattgaagaggaaaatcaatattttgatgagatttcttcactcccatgtttattgcagccctgttcacagtagccaagtaattgaatcaaccgaagtgttcaatataaggtgagtagagaaagatgatgtggaatatgtacataatggaattctacacagccatgaaagattaaattgtgtcactcatagaaacttgcatgtaacatataagactctatgttaattgaaagagccaggaacagaaagctaaacaccacatgttttcactcccataaaatagtcaagaaatatccataaaacataaataattgaaattggacatgaactatagaaacaataattgtcacctgaatatttattttccttcaaataatatataattcaatgatagtaagaagagtcctgataaataaatcatgttgagataaagagctactgaattgtgcattccacactaaatggaaacaatgccataagatccagtgttgtgtaggatctgtttacctttttacctcaaatactacaagtcactcccttgttctaatcaccccgcaaaactgatattttgttgctttatgttatgtcagtaaaaatgattccacattggaagcttcatgtgttgctcagaaatcaccctgttgataaaatgagcacaactacagttacaacctttctgtagatattttctgcatatgatcctcactttttatttgttgtttttgttcacagtttctttaagcactcagagttatatagcatattttcagcatattttaattattttccattgttatggtcagtgctcctgtgggctcaagagtcaggtgttgtgtgactgccagttgacttcactgccacctttacctacacaaaacactgcaagatgaaccatgatgtgtaagaacccatccacaaagtgcttcaaataaagacaaggtgacttgtgtatagatttagtgagactggggaacagttcacggtgttatgttacagaggagcagaaaggtgaatctctctcggagttattgagggtgtaagagctgcagttttggagggtggtgaaaggcacttttgagtgggaagccctagagcatgtgtgaaagctgaaggaatgatcaccatgcacatggacgctgatgaggcatgtggtaaggtccattccctccagatgttcctggatgggcggttgtaggatggctcatgtatatgaaaatgtgggcagggctgaaataggaacaaggccatcatcccacgggaatgcgagaaagcaaaacagagatggacacccatgaagagagctcattagacctggtgaaggaaagataggaatctcttccaaaggctccgctgttcttggctaaatatgaattgtggtgatagactgaaaataaggaggggaagggagatatgactcaagagaggaaaaaccctaagacagcataaaccaaatctacacagaacaatcacatggtagatgccagggtggggctgaaacaggagcttcaatctactgggtacctagttgagatcctttgacatttcttgtgaggtaattctagtgatgatgaatttcctcagttttcctttgtctgggaagtatttctctatccttcatttcataagagatgttttatcaattttaggttcttggttggctgctttttccccccagaaatttaaatccagttatttgctgcataacaaagtttctgccagtaacagatggtggttccacggtgctatacttccttacttatctatatctctttatgtttagatacacaaataattactactatgtttccatggccaacagtattcactacggttgtttctgagtcactggggtgttgctttttctggccagaaccctctgtggccagtggcacttttgcctgagttcttgtcctacatgtggaaagaatgaagtatgcagccatgtggagggtaagcaagacaaagcctagcttcattaagcattagagcatctcagaggagacccccagtgagtagctcctctctgtaggcaggtagactggttgagtgttcagctctaagcagagagggtagctcctccctgcagccggtcgtcctgtggtcttcccagttctcaggagagagggtagctcctccctgcagctggtcgtcctgtggtcttcccatttctcaggagagagggtagctcctccctgcagctggtcgtcctgtcgtctctccatcctctgtcctgctccagctgaggccaggggtttttacggacctccgaggggaggaagtgcaagcccattggtccatgggtgctcatgggtgggccgtaggaagcaccacaagtcccgactctggtctgaggatctgggagccccggcccagccttcaggcccttcctggggacccgcccctgtgcacaggatcttcctgcctcctgttgtagttcatggtccctggggctccgccctgactttgctccaagatcagagcaggcaccagcagcaggaagaagccaggaagtgggagcaggcacttcggagcatgcaagagcacagggcttttcctggggaccccaagagtgcaggggcgcctgaggccgcagcccccggtttgcggagcagggcttctgccggcttcatggagcgagaggcccgtgtcttcagccgcagtttgggaggctgcagccacagccggggaggcagggctccttcctgttcctcgacctccaagagcacagagtccgagcccactgccctggtttgcgcagctggagccgcacctgagagatgagagctgctgcctgctcctggctcccaccggctccgtggagcatcagcccctccacccctccttgcagcctgggcaggggctcctgatcctctctgggccctggccggcgtccagggcaggagtgacatctccacaagctccctgcattgccccggtgctcagggaggcccgggcagagctgatgacgaccccggcccgtagtcgggagtggcggcctccatggtcaccctgacatgcggctgaaccctggggacgcggcccaagcagcctgcacagaacctcccaaggcccaggaaccccgcaccctaggcggggtgggcacagcggctgctccgctgcccgggttttcaaggaggcgccacttccacttccctccctggaaccccgaagtttgacatgggggctcctttctgccttgctccgcagctccccttcccccggagtggagccccctgtttccctggcgttccctcccacagctgcagtgttctccatcggtgtcatcaccttccagctctgctgccctgctctgcagactaaggctctgattccataagagaggggagctgcttcccagtagaaccttgctggggagctctgttcccatctcagttcctgaggggttaaaccagtgcatttaggatactggttttggtggtttgcccctgttgagtaatttcttagttctctagtgggtgtaagagacttggctctggaagcatttcagaagtgtgggctctgatacaccccagacagacactttgggaagggaagatttttgtgactattctcattataagggaaaggcattcaaaagaatagaaaaactctccagtatgtggttcctgagaatttctcactaaaaacatgcttatcacactcgactcaaaacagttcaatgtatattagtgtattttatcttgtactagcctttattgcatttggtgaactctgccccagttcagctcatactctagctttgattctccctacaaaaacttgtctctctctagatttcagatttgttgattgtcttaaaatttcaatgatctgaagtattaaagaaaatttgcaaaagtccattttctctgattaacagttattgttgattttattcttgttgtaaaaaaaagaaattctcatctatgtacatttcaaacctgaataacaaaatttttattaacaccaaaaataataaaagaatccaaatatttatcagctgcctaatagaaaaacaaatcatggtaacattgttcgctggaatattacccatcattcataataagggaatgtctgatacacaaaataagaagataaaattatcaagtatttaaattgagtaaaataagccaaacaaataagagtatgtatgattctatttttaaaaattctggaaaatgaaaactgatctaaagtaatataaagaagattagtagtttcctgggaatatgttggcagaagggaaggagaaaggataaggaaatagaaataggaagtagaaggacagaaaagaagttgagggaatttcacttgtccaccttccttataatggtaatagttatgccatgattatcagttttacactttaaatatgtaaagtttataatctgtcaatcaaatcttataaaatgtattatgaggaaacaagttgaaaattagacaatgtaggagtgacagaaagatagatatgagtatgttgaatgtcagagatacctgaaagtttatctacctgaaccctagttctctccatagtttaaggtaaacaggagagtgcaggaaaatcatccatattctgattaggcagtggcttctgcaaaccacactaggcctggccggctgtgtcctggagttggctaagggaggagtcagggccagtggtgagaagtgcaggcccagataccagaactcactcatcccagacatgagctcttagatacacagagagcccatccatgtgtggatttatcttacatctgtaagtagagaacattgactcttacagaacataatttacacacataggtaaatctgaaataaggtgatcagtgtgaagattttatcacagcacagtttcataataagcacaatttctcaaatcccattgttgtcacccatcttcctcaggacactttcatctgccctgggtcctgctctttcttcaggtgtctcaccccagagcttgatatatagtaggagacatgcaaatagggccctcactctgctgaagaaaaccagccctgcagctctgggagaggagccccagccctgggattcccagctgtttctgcttgctgatcaggactgcacacagagaactcaccatggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagacacagtgaggggaagtcaatgtgagcccagacacaaacctcgctgcaggggcatctgagaccacgagggggtgtcctgggccctgtgaactgggctgctctccgtggcagcggctggtggtgctaaaggctgattttctctcagcatctggggctgattcatcaagtttcctcagagacctttcagattacaattctgtacttacgtttaatgtctctgaatgtgacactttccttccctggtgtgtctttgtttttgtgacaagaggacacattctcacctccacagaagcccgagtgtcactttggggacagaaatgaccctgccctggtcaccagaatcagagtcccgaggaagcccaggaggacctgggaagtgtttttcaatcagactcagggcaggcgtctccgtgggaatctctgattggaacaggctttgggattcagattgggaccaagagggaggctcacccagggccagggtccttagaatcctgacagttttcacagtaaccccatcgtcctttaaaactgaacatctaactcagaactgacccatttggtcctttctctgtaatccattttccttttctctaggcttcattcttacacttcccttttcaccttcattctgaaaatggagggtgtgcttcctgtggtctaaaccacaggcctcagatgcattacctggaactcaggtgtggctctggctatggctcctgtggacctggcaggctgagggatctttctcattccctggtgcctgcatgcccctgctgtcttctatgcgtggatgcatttgggaaatgcaagtggacactcatagtcgtttcctcaaatgggatactgttgtagagctgatcttgtgcttctcaccctgtcacagagcccccactctcacttgtggatttttgggagagctgaggatggacactttattgggctgtgagctctgcatgatggcaatcgtgaggtctgggtgggcacagccagagccaatggagctggccaaaaggaaagacaggacggaattcctgggaagtcctacagctgctgtctaccatagagttccattgtcttctcctctgctaggattaaaccagaccgaccaagttcatctaggacaatcttcctgacctagagaagtgatgacaggctttaataacacctgtattatacatcagagcaacacctagattagtgtttgattgaataattgagactatgtcctagtcaaggtgacacacaaaatcaattattaccatgattaatattttatattgattaatattttaatactaatattaatcaatgcaatattgatttaatattaattaatattttatatttgacaatagaatcagactcatgttataaataattttgcaaatatatgtatattattattggtcttctgagcataaatctccattagccgattaagtgtgcatgcattggtcgaaggagggcatccacccttttgagggaaaatgcatggagggtaaatgaggctgggaagctgatggcatatgatggaagcctgtccctgagtgaaggagagagggaggcaggattgggtggaactttcctacatttctgtgctgtgcaaagaaactccagattatcactgagtctcctgcaagtcaaagttgcccctcaggaaacccccatcactcccagcaatgactctgccccagatgagtgcaaggctcactctattcctgagaaaggagcacaggatatgggatttagcatgagccacgtcatggacgtcagagagcaggagctgggtgcatgatccaagtgcactgttctgcctgtaggtggagagaggaaggtgcattcccagagctaacacactgtggatttttatacagaatacacgttttcacctgatttattggaaggcatatgaaaaaatgtgcagtccacaaacaattgtaatttccaaatttaccacaattgcattatttcttcattctgcatgatgtcctggaacagagttacatttttcatgggtattgtttcatggattgaggacatgaatttcacacctgcctttttagccatgtgcagagaccttgagtagagcatgtctggacctcatctacactatctctcatgccccaagggaaagagtggagacttgactgactgcaaactttggggtgggagctgctcctgcacacctcagaggctgcagagaccccaagtgcagttttattgagttggggtgtctttctatgtgggggaattatgggctgccccactcctggtgattgctggtcagctctaaagacgggttcagaatgaggtcacctggtaacctttctcactgcagctccattatgtaaattaccactaccagattccaggtaataatgcaagctctatttttgcgacctcattttcttatcttttttgttcataagtttggttatagaaaatgttaccacctagtttcctgattcaaatcctataatttgctgaactactcaggatgttttttaatgtgcccagtatttgggaaatataaatgaacagccatgtgtggttcactgagtggacatagtcagttaagtacattagtaaaaacatgattgctgaattgtatagtgaaactacaattagatttgcagaaaattgcctccagtaatgtctgaagggagagccacccagccaatcccgtctctgggcttggaggaagctcactacacaggccaggtcacatgggtgatatccaaggtgcagctcccagcgttactgtgctattttccaggaaccagacatacaacttggcatctcagaagaagatctccaagatttcctttacctggacttcatgggcagcacaggaccagcaggagatgctcagaactcacccaaggcagaaccccagggaagagcaggtgcggagaggaaccagccagggaacctctgaccaccatctagggttgccttcttttccttcacagcatggagccctcaggagaccacccactaggttgtctgtaactcacgtccttcttgtcttgaagcaaaacagacataaatatgaatttgcagttagccccttgaacctgggtcaattttttctgagaatgccaacttaatcgtcaacatcatgggcattatatcaaaatcgagatggatatttagtcaccaggaagacagtttgtctcatggtagaagtgcttattcaattaaaaattacattttaatgaaaaggtctctaagtgaggactgttcaaataataaagtattagtgaagtatcagaaaaataaatggcaccattatctttggtatccagttaatctataaagcatatcaatattcatgttgtaagtggcctaggacaagttatggcagtatttaggctataaggagacattgctttccttcttagataggaatgtccttttttatcatactttaagttctggggtacatgtgcagaatgtgcatgtttgttacataggtatacaagtgccatggtggtttgctgcactcatcaacccgtcatcgacattaggtattactcctaatgctatccctcccctagccccccaccccccaacaggccccagtgtgtgatgttcccctccctgtgtccatgtgttctcattcttcaactcccacttatgagtgagaacatgcagtgtttggttttctgttcctgtgttagtttgctgagaatgatggtttccagcttcatcaatgtccctgcaaaggacatgaacaggaaattccttctaaatgaactgtgttttgacaaatattatccagtgactcttcctacatggggtttctacatgatctatttttcttttccaaaaacaaatcactcaagtttctcatctcctgagtgagaaaaattttccccaaacacaatctcagtctaggtagacaccttcatgaatgagccttcattctcagacaggcacacactgtccccatgtggatgcttctctcagacaggcacacatatccccacatggttgggatcatctgctgggactttgccccatggggatctctattatcactggggcaagttagcacctcgatcctcatccacactgtggcctttgatgtctgtgacctgccacaccctgtctgtccctcagtgtctagaatggtgaagtagtccaccccagggatggaggaagatcccagattttcctgtgaaggagacagaatgccaatcacagagatggaaatctctcacatccctcctgggtcatattctgggcagggtctctgaagtcacccctggatcagtgtcctaagatgtctccctctatttccttgggaagaacccatagaaagacaaataagtttttctattagggcatcgtagaacttgtgctttcttgtgtgacaaatataggtattttgactattttaaaaattagatataatattaagtctaccaaatatgtaaatatataagctttataatgtatccctagagggatgtctcctggaattgagaattgaatatatatatatatatatatatatatatattccaagataaatcttggtgaacattataaaattgactctaatatttatgttcaagatgatcatttcaaaaaagttttaacaacagagaccagatgtatctttccaccagaaataactgtaaaacacagtgaaattttttaaacatgattttaaggtattttacatcaaataacaaggacagtgattgctgaggtggggaaaatatacaaaaggagccctctgatgaacgcatgctttgctactttttaaagagagtttccaggccacagtgctgaagaggaaccaggtagatttgaagttgaggaaaaggagctgagatcccagacaggccaagaagtgtggagatcacagactagacagtggatagcagaacactgttcagagagtgagccccgtagatggccggagagagacccagccttggtagagtgtggatcagtgtatgggaaccctggaaagttgtacatgagaacacaccttactctagggggcttgccttaaaatctaaaattaccctaaaatttaaacataatcagttacaaattttaaaacataaagaaagacaccatgttaaagaaagataaaggaaaggtggatttgcaatattaaaatataaaaagtgtatttctgaacaaatgataatactgatgaggggggacaagtttattccatcacggcaaatgatttaagagaagaacattccaaatgcttatggccctaatgaggaagcttcaatcacgaatataaatagagttatctcattaccagtgtgatggtgtgagaaacttcaagatttccaaagagacaacctaatctggtaaaaattatagaaacccaacctgtactatcaaggaaaattattctgagaacacacagtaaatgaaatccattgactgacataaagtcctcctgttgactgaagggttatgaccccccacaaggcatatgttggaatctcaactccatcgtgacggtggcaggaggtggggcctttggtctcctttgaaaggtgttgaggctccgtttcatgaatggaatcagcgtcctgatacaataccccagagagctccctctttccttccaccaggtgtggtcacagtaggaaggtggcaatctataaataaagaattgggacatcaccagataccaatctttaagtcatctttatcttgaacttcccagccttgaaaattgggacaaatgcatttatgttatttctgagccacccagtccatgatatcctcttatagcaactcaaacagtcttagcaagtgcagtgaggatcacaatacttgagagaagagcattacctcttctttacatctcaggatcacagaatcttgaccttgggctgaactgacaagaaggtggtgcttccctctcccccagatctcagttgaggtcatgatgtcagatctttatggtggagactaaattcctgcctgtgatttgatggatgcctttctcctcccagcctcactcacaagacatagtttccatgtcaggtgttatagaccaagaacaggtggaccccgattattctcaccccagctcacttgtagggcagaggttctgggagcttcctgggagaggaacagtgagatcatacaagtccatcacttccatgcagttttctgtacttaaagcaggagtgtccttctgagctgcttgaaccctttcttctgcaccaaggtagagacttccatgtttttctagtgatacagtcagggcataaaaacagtgatagaaagctctctctaaagggcgtgaccagatttgcagtagaatgagaatagtttcaaccctaaagttcatctaaaatgttagtggttttcatggtaagtaattaatagaaacctgacagctccatgacagcaatatagaaatcaatagtccactcattttaacagagagaattagaaaacctataatttaagaataaccagctgggcactgtggctctgcctgcagttgaatttacttgggagtttgaggtgagaggatcacttgagcccaggatttcaagcgcagcctggaaacatagctagacttaatttcttgaaaaaaatcagtgctattttggggtcagaacaatcctcaaaaatggtccccggaaagaagcccaaatttaattgcaccagattgttgaacgatttatgctacaaaacagtgtcacatgtcagaaatgagcacagtgtaacatctacatggtttgttaagagacacaaatggtcaagtagaacaatcaggtaattaggctgtcgaaggcaacactgccaaatgacagcatttccgtggaaactgcgtgtacatccaggactgcacctgtgaacgatgacatcatacccttcacagtgtcgaggaaagagacatcactcaaacagacaagccaagggacttcagaaaatataaggggaaatacagtgtgcaaatatgtaaaaaatgcaataagatgattactcctaaatgaatatcaagacacaatcacataatatgaaattaaattttcctgaatgataggattactaccaatcaccccccaggacaccctcatctactctgtgcacagccttctcgtcaggcgtcccagcccagaccttgctatgtagcagaagacatgcaaataagacccccctttttgctgatgaaaagcagcccagccctgaccctgcagctctgggagaggagctccagccttgggattcccagctgtctccactcggtgatcggcactgaatacaggagactcaccatggagtttgggctgagctgggttttccttgttgctattttaaaaggtgattcatggggaactagagatactgagtgtgagtggacatgagtgagagaaacagtggacgtgtgtggcactttctgaccagggtgtctctgtgtttgcaggtgtccagtgtgaggtgcagctggtggagtccgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagacacacagtgaggggaggtcagtgtgagcccggacacaaacctccctgcaggggcgcgcggggctaccagggggcgctcgggactcactgagggcgggacaggtcccaggaacaggtgcagcggtaggttttctttctcctcagctggagaagtcaggtttgtgttttcagaactctggagtcttacaggttgctacattttcatacagttattagtatgtatttattatcattggtatttaagttttaataattttaaaccttttatgtagtgttattttttaaaactgtttactttcatttgcagttattcttccagagtttcattaacatctattgctatgagcaactacatagctatgagagcataaatttacacctgtagacgtaggtctaaatgccacaaacctgtgcataaatgtatagtgacttatatttaacattataataagataatttttaaaatatatcctaaacgatcaaacttactgatgaactaaatataaattatcagagtaatgcataattgatttcaataattttatattgtttatattaattaatatctatttctttactgaaacataatatattggtcatttcaaaatagctacgatacatttcaaatggccttgatgctaataatgaaaagattttgaggtgattaatatgctaattagttatatttaattattccatattgtattaatatatcaaaacattgctttgtacctcataaatagatacagtcgtaatttgtcaattttcaagacagttatttaatccatcctgggtcataatctttttttcccctgtccatggctgattggattgtcctgataaacccatccatacgcctgcctcccgcaggcttctgagacgggctccatgaaggccagaagcaggcaccctgtgagagtccacacgacctggagcctccctctccttggattaggccatctcctcgggatcgcagggctcttcattatcctcacccccttgttgtaccaaacaagcaacatcacactttaattcatcacgctttgctttaattttccccccaaaaatcaaggtaataattttagcaataaacatcacaacctactatgagtaagtcctttccaacgtactaaggtttcttctgaggactttacatgtattacacagtttaaatttctttatgaaatgagataccaatatctctatattagagattgtttttctaagtcgtttctgacaattaatttgtccaagggcctgtaaggattctgttagaaatccaggataaaatttgaaatttcagcagtaatatctggaaaaacattgagactatatttaacttcgtacacgtgctctcaaatatccttatttccctaaaggttgttctcagttattattttacacatgatatctataattaatactaataacacaggttaaaatattatctcaattatttaacgacttttgataataacaaaatacatattccacatatttttgaccagtttgttttctctatgaaacacgtgtttattaacttattattatcagatgtcacccttatctttctgatttctgggatttaattctagcaggtatcgtttagtatattttattaattcatattatattgttcacattttcagtatgattttattatttgcattgtttattgaatttgaaactttcttacataatttttttaattttttattttcatgagatgtaatttcaatataagtaataatgcatagatctgcaatgtaccattacaggatttctggcaaatgtaaacacacttgtccccaacagccaaggttggatgtagaggaggcccattcccccagcacaagcgaccttctctgtgcttccagtcagctcccacatttaacacaatgttttgatttctgccactgtagattcactgttcatgttttgattttcatataaatggattcaaacattatagaccccttttctggtaaggggctacttttgttatttttgacgtttatttatgctatttcatatgtaaaattgcatctgtatattgtcatttattcacttaacagactgcctctgatataaaatctcaaagttttgatatatatggataaagagagagggagagagagagagggagaaattttatacttgagtcagtgcatttaacaataaacgacagtaagctgatccatttccctgtcaatgcacttttaatttgtttctggtttattaacatcataagcaaagctgctgcacatattttttgtttttctatttaccttttctcatttttattgagaaagtatgtggaaatatccatttcttcatcataaaagtaaacttcatttgttcagctttatggaactataattgacaataaaattgtatatatttgaggtgcaccacttgatattttggtataaattgtgaaattttcactatgaccaaggagttagcatgtctgttaccctacatttgccattttcttcctttaatttatatgtgtgtgtccaatgagagcacctgagatctatccctttaccagagatcaagtttgcaatacagtgttgatagctatagtcccattgctttacattagacctccagaacccattcaacctgcacaactgaaactttatgcccttgacaagaatcattctatttccactcctcccagccctgggaaccacttttctactctctgcttctaagaatttgaatattttagattccccatataaatgagataatgcagcatttgtctttctgtgtctggcttattccacttagcataatgttctccaggcccatccatgttgttggaaatgttataatttccttatttttagaggtcacataatattccattgtatgtgtatacatttgctttatacacccatttacctacggtcactaactttaaaaaaattctaggccattatgaataatcttgtaataaacatattttttacttactgattttatttcctttgaatacgcagccagaagtgggatcaccagatcacgtaatagttttactttcaatttataaaataacttttactaccactccactgaataattctcttttcaccagctaactgtcagcattttgttatcttttgtgtctgtgataatagtcatggtaatgaacgtgaggtgatatctcattgtggttttgatttggattcccctgatgattagcaatgtggaacacctttccatgtcctgctggccatgcatatgtcttcccttgaatacaatgtctatgtacagttttgccctcttaaaaatcaggggttttttttgctattgtatgggttctttacacatttagatagatccctttgtcagatatatagcttcaagtaatttatttgtctatgattttggtgtcgaatccaaataaatcattttccagatcaactctggaaagcttcttttttccctgttttcttctatgagtttacaattttagatatcatattatatccaagccttgagtacatttttaggtgatttttgtatatgagatgagatgaggtctcatttttttctgcatatggatgctcagtttttacaccatttgttaaaaaggcaatcttttccttattgtgtgtccttggaacaaaaaatctgggagatacacaaaagaggaaaatcttagaccaatattcctgatgatagacctaaaaatcttcaagaaaatactaggaaattgaatatagcagcacattaaaaagttattacattatgatccagtagattttactcctttcatgcaaggctagttcaacatacacaaatcaataaaggtgctccaccacataagcagaatcaaaagcaaaaactatataattatctcaatagatccagagaaactttcaataagattcaacattcttcatgataaaaaccttcaacagactagacatcaaagaaacttagcttaaaatgataagagccatgtatgacaaacccacaactgacaccatactgaatcaacaaaagctcaaatcattccccttcataactggaacaaaacaagaatacccactctcaccactcctattcaacatagtagtggatgttctagccagagaaatcaaacaagagaaagaaataaaaggcatccccacaagtaaataagaattcaaactatctatttttgctgttgataggatcctatgcatagaaaattctatagactctgccaaaagaatcctagaattgataaacaactttagtaatgtttcaggatacagaataaatatacgaaaatcagtagcttttttatacaccaacgacatccaggcatagagaaaaataaatgacacgatcccacttacaacatctacaaaaaaagaaatacctaggaatacagctaaccaaggaggtgaaagatctctacaagaagaactacaacctctccagcacatgttgtttcctgactttttaatgatcgccattctagctggtgtgagatggtatctcattgtggttttgatttgcatttctctgatggcgagtgatgatgagcagtttttcatgtgtctgttgactgcataaatgtcttctttcgagaagtgtctgttcatatccttcacccactttttgatggggttgtttgtttttttcttgtaaatttgtttgagttcattgtagattctggacattagccctttgtcagatgagtagattgcaaacattttctcccattcgttaggttgcttgttcactctgttggtagtttcttttgctgtgcagaagctctttagtttaattagatcccatttgtcaattttggcttgtgttgccattgcttttggtgttttagacatgaagtccttgcccatgcttatgtcctgaatggtattgcctagattttcttctagggtttctatggttttaggtctaacatttaagtttttaatccatcttgaattaatttttctataaggtgtaaggaagagatccagtttcagctttctacatatggctagccagttttcccagcaccatttattaaatagggaatcctttccccatttcttgtttttgtcaggtttgtcaaagatcagatagttgtagatatgtggcattatttctgagggctctgttctgttccattggtctatatctctggtttggtaccagtaccatgctgttttggttactgtagccttggaaacaacaggtaacaacaggtgctggagaggatgtggagaaataggaacacttttacactgttggtgggactgtaaactagttcaaacattgtggaagtcagtatggcaatttctcagggatctagaactagaaataccatttgacccagccattccattactgggtatataccaaaaggattataaaccatgctgctataaaaacacatgcacacgtatgtttattgtggcactattcacaatagcaaagacttggaaccaactcagatgtccaacaatgatagactggattaagaaaatgtggcacatatacaccatggaatactatgcagccataaaagatgatgagttcacgtcctttgtagggacatggatgaagctggaaaccatcattctcagcaaactatcgcaaggacaaaaaaccaaacaccgcatattctcactcataggtgagaattgaacaatgagaacacatggacacaggaaggggaacatcacacaccggggcctgttgtggggtggggggaggagggagggatagcattaggagatacacgtaatgctaaatgatgagttaatgggtgcagcagaccaacatggcacatgtatacatatgtaacacacctgcacattgtgcacatgtaccctaaaacttaaagtataattaaaaaaaacaattacaacgaaaatccttaatgtctcttgagtaggtagagaacatatattttatctaagtaacttgctctaaactgcaaaccaaaataagatgattcaataaagtattttgtaaactagtctttagcaaaaaaaaaaaaagaactacaaaacactgcagaaagaaatcagaaagtaaacaaataaattgaaaaaaaaattccatgctcatggaatgggagaaataatattgcaaaaatggcaatattatccttcaatatttatcgattcaatgttattatcaaaataccagttttattcttctagaaatataagaaaagtattctaaaatttgtatgtaaccaaaaaagaacaaaaacagccaaagtaatcctcagccaaaagaacaatgccagggacatcacactatttgacttcaaactctactataaagccacagtaacaaaaactgtttggtacttgtacaagaataaacacatagacaaatgaaatagaatagaaaactcagaaataaagctgcacacccacaaccatctgttttttgacaaccctgaaaaaaataaacaagcaatggggaaaagacaacctagtcaataaatggtgctgggataacttgctagccatatgcagaagatgggaacttgactcgtgactatcaccatatgaaaaagaaaattaactcaaaatggattatagctttaactaagacttcaaactacaaaaagctaggaagacaacctaggaatactcttctctgcattggccttggcaaagaattactggctctgagaccaggcattttttgtaacagtgatttattttctggggtacatatcatggagtgggattgctgattgaacgatagatctattttagttgcttgataagcctttacaatttttttcacataggttgcactaatttacattaccaacaacagtgtataaatgcattttctctgaattctcatccatatctataattttttgacttcataataataggcactttgactgatgtaaagttgtgtctcactgttgttttaatattaatttatctaatgattaatgttgttcagctttttatttccatatgctttttggccatttgaatatcttcttcacaaaatgtctgttgatgtcttttgcccacattttaatggtgtaatttatttatttgttgttgttgctgagttgtctgtgttcttgttagattctagatgttagttatttattgcacagtaatgtaataaatctaaaagtcaggaaacaacaggtgctggagaggatgtggagaaataggaacacttttacactgttggtgggactgtaaactagttcaaccattgtggaagtcagtgtggcgattcctcagggatctagaactagaaatatcatttgacccagcaatcccattactgagtatataccatcccatgactgggtatatacccaaaggattataaatcatgctgctataaagacacatgcacacgtatgtttattgtagcactattcacaatagcaaagacttggaaccaacccaagtgtccaacaatgaatagactggattaagaaaatgtggcacatataccccatggaatactatgcagccataaaagatgatgagttcacgtcctttgtagggacatggatgaagctggaaaccatcactctcagcaaactatcgcaaggacaaaaaaccaaacaccgcatgttctcactcataggtgggaattgaacaatgagagcacatggacacaggaaggggaacatcacacactggggcctgttgtggggttggaggggcggtggggagggatagcattaggagaaatacctaatgttaaatgatgagttaatgggtgcagcacaccaacatggcacacatatacatatgcaacaaacctgcacattgtgcacacataccctaaaacttaaagtgtaataaaaaaaataaaattaaaaaaatcaattgcaaaaaaactgttgaaactatgaaaatatgaggtaattaaacaacacaattttgaccaatcattggctcaccaataaaattaagatattaatttttaaaaatacaaagaaaaatgaagccacaacataccaaaccctctgagatacaggaagagcagtactaagagcttatagttctaaatgtctatgaaaaaatagaaagattataaactagcaatctattgatttacttcaaggaactagatattttagaacaaaccaaccccagagctagaagaagaaaataaataaagatcatggcagatttcaaaaatattgaaacaaaaaagcaaaatataaggaatcaacaaaacaaaatgtggttattttaaaacatgaacacaattgatagagcactaactaggtcaacaaaaaaaaaaaacagaagattcaagttagcacaagcagaaatgataaaggtggtattataactgacccaatggaaatacaaaagattatcaagacatactgaatatgtctatgctcacaaactagaaaatctagaggaaatcaataaatttctggaaatattcaactaccccccaaaattgaaccaggaaggaaaagaaatcctaaatagaccaatgcgagtaataagactgaatcagaaactgaaaatctcccaaccccaaaagcccagaaccagataaattcaaggtctaattttaccagatgtaccattcttactggaactattccaaaggattaaagagaagagattctttcccacctttgtctaaaatactcgtatcacctaataccaaaatcaggcaaggatacaactacaacaacaaaaagctacagggaaatatccctaatgaacatagatacaaaatagtaaacagaatactagctagctgaaaccaacagcacaataaaagataatacatcatgaccaattgggttttattccagggatgcaaagcttgtctaaaataaacacatcaataaatatgattcaacatataacataattaaaaacagaaaacataagaccatctcaatgaatgcaaaaaagtcattcaataaaattcaatatcccttcatgaaaaattcctcaacaagcttagcatcaaagaaacctacctcaaaactataaaagccacatacaacaaccaacaaaaaactcttaaataaatcaatagaacagggatcttcttaaatttagtaaaaacctatgcaactgatttattcaatatgctggacttggtagtgaagaaatgaatgactttaactaaaaaaaaaagccacatataacaaaacaacattatatggaatgagaaaaagttgcaaaaatattcctgaagaacgtgagcaagaatgcacactttcaccactcctactggacgtattactgaaagtcctagccagaataatcagaaaggaaaaaagaaatgtatgtttcaagttccaagctaaggaatccaggagtggcagacccagagacttgttttttaatctatgaataacatccaaacccctgaccgatcccttggaacacaggatttaggggtgattgaggctctttcttttgggttaaatgaaagttgctaggtggagggtgttaagtaaaaattactacataagctgcatagttttgtcaaatggttgtagttttcttgtccagcctgctgttcctgaaccaccctatatgaaagccctcaataaactatatgtcttgttcactggtttggcctgttgtacacgatgccagtcctattggagtccatattggtctggcgtgacatccagtcaacctggtaaaaggtccaaaaaaaatctcatgagcacccagacaatggaatctaaaaagaggagacccctgcagaaaccttgggcaggctgttaatttctacgtggggcccaacagccgttatccatgatggatggggcccagtgcctgagtatggggataccgccacaatgccaaaagttctggaagaacctcttcagtgggtgcatcttacaaagtgaaagtcagatgtccaggctgttgcattatcaggtgagtggttcttcctgactgcactctgagaagccactgagacagtgctcactgtgtatgctacagtgtgtcagtcacaagaagagctactcctagaaaaacatgccagactagctcagactaatcagagacctttctgtcctgcctgtggaagcagaatggcaaattggggaccgccgcttgtggagcagcacatttgaaggatttctggccatcacatgaattagttacggctgtgatgactaaactgtcatgggaccctaagacctagttcccataaagagttctagggggaggaggatgaagtttggggtaagcccatgggggttcctgtcctctctacacatctggtggctaccaccaaggtaaaaatgggccaactgcaccagcagaggcgagccccagagacatttcccacctgaaataaggaaacacaccacaatgtgggattccaccactgcggaattgctgacacttagaaatgggttcagacagaagaagagagagttgatcacagggtggcttcttcatctgtgaggcatgcgggaggagggcattatactccctgaactcaagattagtaaagtgacatccatcacaaaccatctgggcctgaggcagcacctcgatggtgtcagtcatggagattggaccatccctctccttagctgggtggttgcagactgcaagggaccctggtgaaacgagggagatgtccgctttattagtgtgttctcgtattgctagaaagaaatacctgaaactgagtaattcacatagaaaataggtttaatttactcatgattctgctggctgtatgggaagcatgatgaaaaatctgttcagcttctggagaggcctcaggaaatttacaatgatagtgaaaggcaaaaggggagcagccactttatatggtgggagcaggagcaaagaaagagtgagggtggaggtcccacacactgttaaacaaccaaatctcatgagaactcatttactatcatagcaaggggaccgtgctaaccattattcataaatccggacccacgatacaaccaccgggccccgcctcaaacattagggaagcaaaatcacatacagttgttatgacaggtgggacatcctgaaaacctctctaggcatgtcccacacggccctggagctgtctcaggggagcagtctcctccagtgtttagacgcacaggcacggataatagggctaagtctggccagatgtgtgatattcaacacattgcacaactgctctgttctgtatgtaatttatcttctctacaaatgtaacattgatggttgcattaaatatattctgcaaatatgtaaaaataaaataagatgatgattgctaaatgattatcaaggcacaatcacataatccgaagttacaatttcctgagagataggattactgcccatgcttaccaggacactcacatctgctctgggcactgccttctcctcaggcgtcccaccccagagcttgctatatagtaggagacatgcaaatagggtcctccctctgctgatgaaaaccagcccagccctgaccctgcagctctgagagcggagccccagccccagaattcccaggtgttttcatttggtgatcagcactgaacacagaggactcaccatggagtttgggctgagctgggttttccttgttgttattttacaaggtgatttatggagaactagagatgttaagtgtgagtggacgtgagtgagagaaacagtggatttgtgtgacagtttctgaccagggtgtctctgtgtttgcaggtgtccagtgtgaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagagacacagcgaggggaggtcagtgtgagcccggacacaaacctccctgcaggggcgcgtggggccaacagggggcggtcgggaaccactgaggacgggacaggtcccaggagcaggtgcagggggcggtttcctgtcagctgcaggaggcgggtttgtttttgcaggactctggagccttatgaggttgcactattttattatggttatttatcatgtgttttttattgatatttgtgttttagtaatttttacatttatatgtaggggtattttttaaaaatacataacttcaagaaataattcttcctagtaatttacacctattcctctaaagttttattaacacctgttgatatcagcaactacatagctatgcaacattaatttacatctataaacatatgtgtgaatacatgaaactatgcatacatgtgtagtcatttatatttaacattataataaaataataaattaaataaaataattgaattaaattaaaagaattaaacttaatgatgaactaaatataaattagagtaatccataattgatttcaatccttctctatggtttacataaatcgatgtctacttgtaagcttaagtatagtgaattggtcattttaaaatagccaagaacaaatttcaaatgttcttgtcaccaaaaaatgataaggatttgaagatttgaggtgatatatatgtcaattagctcgattcaattattccacattgcattcataaatcataacatagctttgtaccccatcagtatatggggtacaaccaaaatttctcaatttccaatgaaattttaattatatattttttaatttgatgcctctctttggatcaggccacctcctcaggattacagtgctgttcagttttctcaacatgctgttgtaccagataagcacaaaacatttaatttcattatgctttgctttagtttttcaaaacaacataaaggtaataattttcccaacaaacatattacagcctattctatacttttttattttttgtttttatgagataaaatttacatgtaagaaaaaatttatagatctgaaatgtgtcactagagagtttctggcaaatgtgaatacccttgtcccgaacacctaaggtagactaaagagcaagtccatacccacaacatgcgtctttctctgtgcctgcggtcaacccctacatggggaagtgttttgatttctgacaccatagatccatttttttctgttttgagctttatataaatggaatcaaacattatagacctttttttggtaagggactactttgctattttttaggttaattcatgctatttaatgtatgaaattagatcatcatagtgtcatttactcaattaatgggctgactctgatatgaaacctcaaagtattcatgtatatatggagagagagagagggaggaaggacagagattttatatctgagacagtccattgagtaaatatatgacaatatattgatctatttgttgtcgatggattttacatttgtttccagtatatgaatattataagcaaagctgttacaaatacattagtgtaagtttctctatctatgttgtctcaattttgttgagaaaatatgaagtatgtttttctctattgtaagaggaagtttaatttttttcagttttattgaggtaaaaataaaatattttttaaaatgtataggtttaaggtgccccacttgacgttttgatgttgtattagtccattctcacactgctataaaaaatgcctgagactgagtaatttgaaaaaaaatatgtttaattggctcacggttctgcaggctgtacaggaagtgcagtggcttctccttctggggagagtcaggaaagttaggatcatggcagaagacaaaggggagcaggtgtgtcacatggccagagcggaagccacaggaagcggaccggggtgtggggggactgcacactgttagacaactagatcttgtgagaactcaggcactgtagtgagaacagcaccaggagtcggtgctgaaccactcatgaaagacccaccccacgacccagtcatctcccaccaggtcccaccccaggactgaggattataatataacatgagatttggtccaagacacagatccaaaccatatcatatgcacattgtgaaatgctcatcatggttaaggtaatttgtatattcatcttctcactattttttgtttgatttttgagacagagttttgctcttgtcgtccaggctggagttcaatgtcaccatctcagctcactgcaacctccaacccctggattcaagggattctcctgcctcagcttcccgagtagctgggattacaggagctcaacaccacaccccgctatttttttttttttttggtacttttagtaaagacagagtttcgccacgttggccaggctggcctcgaacagctgacctcaagagatctgtccgcctcggcctcccaaagtgctgggattacagatgcaagccacagcgcccagccaattttattattttaaaaatgtactgcatgtgagtgtgtgcgtgtgtgtgtgtttcatgagaacacctaagatctacccttttagcaaaaatcacttttacagtacagtattaaatacaggaaaattggcgtaggttagatctccagaactcattcaccctgcacagctgaaactctgtaccctctgaccaacttcacccaatttccctctcctcccaggtcctgggacccactattctactctctgctttcaagagcttgaatatttcagatcccacacgtaaatgagatcatgcagcatttgtctttctgcatctggcttattccactcagcgtcatgtcctccaggcccatccatgttgcaaaagccagaacttccttcttttcaaagctaaataaaattcagttttatgtgtacacattttctttaaacattctttaatctatggtcattaaactctttcacaaatctacactattataaataatctggcagtgaacatgtgtttaacataataattttatttcatttgaatatataacaagaagtgggatcaccagatcgtacgataactttatttttcaatttattgagtaacaagtcctaccacaccatatcatgatttcctttcccaacattcttgtgaacacttgttatattttgtcttgctgataatagtcattttaagtggtttgaggcaatatttcatcgtgctttagatttgaatttctctgaaaattagtaatgttgggaacctttttaggctctgtttttcatgagtgggttttctgaaaaaaagtctatccaggttttttcccttttatcaggtcgtttgtaatttgcacatagttttttttcctgtgcttttggtattaaattcaaataaatcatttctgaatcaatgacaggaagtttttccatgctgtctatgagtttgtggtttcaggttttgctcatttttagttgatttttgtacatggtgtgagagaaggtctaattttatttcttctgcatatggatgtccagttatacatcatttattgaggagactgtctttcttcactgtgtctttttggcatacaaaatcaggaagagacataattaaaaaagaaaatattagatgaatattcgtgatgaacatagacctaaaagttctcaacaaaatattagcaaatagaatccagaagcactttaaaatgtaacacatcatgatcaagtgggctttaccctgggatgcaaagttcattcaacatccacaaatcaataactgtgattcattgtgtaagcagaataaaagcaaaaaccatatgttatctcaatagatgctgagaaagcttttgatatgctccaacactcactcataataaaaaccctcagcaaactaggcatcaaaaaacatacctcaaaataataagagccatctatgaccaacccacagtcaacattatactgagtgagcaaaagctcaaaccccatgagaattggaacaagacaaggatgccctctcaccactcctattgaacatagcactgaaaatcctagtcagggcaaccaggcaatagcgaaaataaaacgcagccgatatggtttgtactagtgccccattcaaatttcatgtcaaattgtaattcccagggttggggctggggtctggtgaagggtgattctatcatgaggatgggtctccgtctcttgtgctgctctcgtgatggtcctcaggagatctggtttcaaagtatgtgtcatctccctcctctctcttcctcctgctcctgccctgtaagacatgatgcttccctttctgtcatgattgtcatgttcctgaggcccccccaagagccataattcctgtacagcctgcagaaccataagacaattaaaaacctttatacattacacagtctcaggtgtttctttagagtgtgcgaatggactaatacagaatcaaaataggaaaagaagtcaatgtatctgtccaaattgatgatataattctatacctagaaaattctaaagactctgccaaaataattctagaatagataaacaactttggtaaagtgtcgggatacaaaatcaatgtacaaaaatcactagcatttgtgtaccccaactacatccaagctgagagtgaaatcaagaacacaattctatcccacttacaatatccacaaagaaaaggaaatgcctgggaacacagataacaaataagatggaagatccctgcaagatgaatgacaaaacacagcagaaataagtcataaatgacacaaaaaatgggaaaacatttcatgctcatgattggaataatcaatattgtaaaaattgtcatactgccaatagaaatttacagattcaatactattcacataaaactatcaccatcattcttcacagaattagaaaaaaaagtatatatattctaaaagttatatggaaccaaaaagcccctgagtagccaaagcaattctaagcagaaagaaccatgacagaggcaacatgatacctgactttaaactacaccataaagtcacagtaaccaaaacagcttggaagtggtacatgagcagacatgcagaaaaatggaacaagagagaaaacagaaataaacctgcacacataaaccatctgatatttgataaggctgacgaaaacaagcaatggggataaaactctgtattcactaaacggtgctgggataactggattctgtcagatatacagaatgcaaaagtgaaggaggctgggcagcttcgacttagatttcagaggatatgtagagggccctgcaggcccagcaggaggccggctgcagcagtgcagccaccacagacagcctctactaggacagtgccaaagatggggaaagagagggttggagccccaattcacaatccccactagggcattttacactaaagctgtgggaatgtggccacgaccctcaaggtccctgaatggtagaaccaaaggcagcttgcactctcagcctggaaaaacttctggtactcgactctaacctatgaatgcaggccaataagctgtgctcagctataaggatgaaaagcctgaggtttctgggactccttcaacccagggtgccctggttgaagtacatcaagtgaagcgagattattttgtagctttaagatttaatatctgcccagacaggctttaggtgtgtggggggcctattggccattcctttggacatttttttccttttgtattaacaatatttaaccaatatctgtggcagtcttgcatgttggaagtaaataactctttaaaacattttgccagctcatagctggagggaaattgccttaagactcggataagactttggaattttcagttggaggttgaccaagttaagacttttggaaactattaaaaaaaaaagatcattttgcaacctctgaaggacgtaagatcagggggcacaagagtgaaatgatatagtttagatgatggtcccctccaaatctcatggcgaaatgtgctccacaatatttgaggtggggccgactgggagattttgggacatggggaaaaatccttcaggaatggctcaataaccaccctatggcagtaggtgaattattcctgtattaactactgggagatctgattattaaaaggagtctgatgaccctcctcccctctcacgtccacccctcaccatatgacaccgcctgcttcccctttgccttccaccataactgtaagcttcctgaggccctcacaagaagcagatgctggtgccatgctcctcacacagcctgccgaactctaagccaaatgagcctcttttatttgtaaattacttggcctcaggcattaatttagagcaatgcaaaatagactaaaacacactgcccaaagcaatatacagagtcaatgcagtttccatcaaataaccaatataattaagtacattttttttaaatgtcctaaaatttatatggaagcaaaaaagagccttttgattccatataatagcaaaagccgtcctaagacaaggaaacaaagctgaaagccccacattccctgacttcaaattatactacacagttataggaagaaagacagcacggtactggtaccaaaacaaaacaaaaaatattagaccaggtgtgcctgtaatcccagcactttaggtggcagaagctggtggatcacctgaggtcaggagctcaagaccagcctgaccaacatggtgaacccctgtctctagtaaaaatatgaaaaactagccaggcctagtggcccatacctgtaatctcagctacatcggaagctgaggcaggagaatcacttaaacccgggaggtggaggtttcagtgagctgacatcttgccattgcactccagcctgggtgacaagagcaaaactccatctcaaaaaaaaaattaatgtaacagaatagagagcccagaaagaaagccaaatatctacaaccaactattctttgacaaaactgacaaaaatatacactggagaaatagtcctctattcagtaagtcgtgctgggaaaattggatggccactcatagaagaataaaaccaggcttctatctcaccacagacaaaaagtaactgaatatggattcaataattaaatgtataaactgaaactataaaaatacttgaagaaaatgtagggagaattctcgggacattggcctaggcagataaaatatgactaagacttcaaaagcaaatgcaacgaaaacaaaaatagatgaagaggacttagttgaactaaagatcttctgcagagaaaaagaaataatcaacatggtgaacaaacagcctacacaatggtagaaggtatcacttcatctcagttaagaaggctcttatcaaaaaagaaaaaaaacagccaggcgtggtggctcatgcctgtaatcccagcactttgcgaggccgaggcaggtggatcacttgagatcaggagtttaagaccaacttgtccaacatggcaaaaccctatccctcctaaaaatgcaaaaattagctgggcgtgttggccagcacctgtagccccagctactcggtaggttaaggcaagataatcgattgaacctgggagttggaggttgcagtgaggtgagatcacgccactacactccagcctgggtgacaggagtgaaactctgtctcaaaaagaaggaaggaaggaaggaaggaaggaaggaaggaaggaaggaaggaagggagggagggagggagggagggagggagggagggaggaagggaaagaaagaaagaaagaacgaaagaaagaaagaaagaaaaagaaattaagaaagaaaaaaattaagaaagaaaagaaagaaagaaaaagaaggaaagagagaaaaaagaaagagagagagaaaaaaggaaggaaggatggaaggacagaaggagggatagaaggaagaaaaggaaaggagaaagaaagaaaaaagagaaagagataaaaagaaataaagagagaaagaaagaaaactaacaaatgctggtaaggttggggagaaggtaaactagtacagtcagtcactatggagagcagcgtggaggtacctcaaaaaactacaaatacaactaccaaacgacccagcaattccactactgcaaatgtatccaaacgcatgaaaataattatatcagagatttcttactcccagtgtatatggcaggacctttcacaatggccaagatatgaaatcaaaccaggtgtcagagagcagatgaatggttaaagattatgtggtgcatatacatgatgagatgctcatcatccacaggaaaggaaatcctgttgtttttggcaatgaagacggaactggaggacattatgtgaaatgaaataaaccaggaacttcaagtaatacaccttgttttctcactcatacgtggaaggaaaaagaaaatttgtctcatagaaataaatagtagaacagaggatactagagtctgggaagtgcaggggaagagataggcagagatttgtgaaggaatacattagagttaggtgggaggaatagattataatgttctttaccactgtgggataaatgtatttaacaattatatgtagtttcaaacagttagaagaaggatattgagtgttcccaacaaaaagaaaatgtttcagatgacggatatattaattatccttatctgtcccctacacactgtttcacaacatcgctatgtacctcatgaatatttacaatcattatttgtctattaaaattatattagaaaacacaaaattcactggaagtaacataaatggaggctctgggatagacctggagataacggagggtctgggatacattacaggcctagcactcaatggtcagaggagctcccattagctttttgtatttgtagcactgagtaaatctaacatttctcagagcttccctttgcaatagagacgtgaggcctagggagatcttgcagatagtcagatatgcagagggaagaagcaggggtagagggttgctggcccattcaacaatagttggaatgataattacgcatgtagagaagccaacattgatttaaataagttgcatcactgaaagccatgaaaaaaaatccaaaggaaatctttaaataaaattgagagtgaaactgttttgggtgaagaaatgttcactgaagttaggtgtgccagtaaagctgttaagaactacagtaaccactggattggattcagctttgtctgaattcagtgtgattattactggctcaggcaggggtcagaaatcaacatggataggtggtagcaacacgaaaaagccagggaccagaaagattcacagccgaattttttcagatgtataaagaagtgttgctaccaatcctagtgtaactatttcccaaaaatgaggataaaggtctcctccctaaattattccatgaagccagcattattctgataacaaaacctggcagagacttaataaaaaaacatgactttagaccaatatacctgatgcaaaaatgctcaatgaaatactcacaaactgaatacagcagcacatcaaaaagcctaacctaccaacactttgttcctgggatgcaaggttggttcaacatatgcaaatcaggaaatgtgattcatcacataaacagaacgaaaaacaaaaaccatgttaaccggcagggcacggtgactcatgcctgtaatcccagcactttgggaggctgaggcgggcggatcataaggtcaggagctcgagaccatcctggttaacatggtgaaaccccgtctctactaaaaatacaaaaaaattagctgggcatggtggcgggctcctgtagtcccagctactcgggaggttgaggcaggagaatggcgtgagtccaggaggtggagcttgcagtgagccgagatcacgccactgcagttcagcctgggcgacagagtgagactctgtctcaaaaaaaaaaaaaaaaaagaaagaaaacgttaaccatagacacagaaaaggttttgaaaaaactttaagattctttcatgttaaaaaccatcaaccaggcatgaaggaatacaccacaaaatataagggccatctatgacaaactcacagccaacattatactgactgagcaaaacctggaagcattttcccctgaaaactagaacaagaaaaggacacccattctaaccactcctgttcatcattgtcatggaagtcccagccagagcatgcaggcaagagaaagaaataaaaagcatcaagtcaggaagagagggtgtcaaactatccctgcagatgaaatgatcacattcctgagtctgtagtttggtgactgacattaatttaaaatattgtcagtcatcattgtttaaatatttcttcttagcctttctctatttttttctatttcccattatgtgcatgttatacctctgtcactgtctcaccattcttgggtaagctgttcttcttttgtatatatgtgtgtgtgtgtatatatatatatgtatatatgtgtgtatatatgtatatatatatgtatatatatgtgtgtatatatgtatatatgtgtatatatgtatatatgtgtatatatgtgtgtgtgtgtgtctgtgtgtgtgtatatttgtatttgtgtgtttactgttctaacaatatattttttgtttttgcttttcagtttgggaaatttctattgagatagttttaagttcagatttttgtccgcagctatgccccatctacatataagtccattgaaagcattacttaattctgttacaaaattcttgatatctacttttttttccttttttggaatttgcttcattctacttgcatgacacagctgttccagcatgctgtctgcttaattcattacagaccttagcatattaatcacagtggtcttaaattctttgtttggtaattccaaactaaatggcatggctgaatctcattttgatgcttgttttgtcttgtcacactatgatatatttcttttagtatatatcatgatttttatagggagaaattatgtatcaggtgaaagaaactgctgtaaagaggcctttagtgatatcattgtaagttgtggagagaaacctgtttcataatcctctggccatggcttcatctaggaagcttaaggcctttgaaaataaatccttacatgaacgtgaaaagataagccacagatatggagaatttcctcacaaatcaaatatttggaaaaggacttttatcaggaataataaaaattaccttacagttgaatgacaaaaccacatgatctaattttaaaatagacaaagacctgtatagaaagaagcctcatctaaaaataaatataaaaagtgatcagcatgatttttaggtatatgtatgtttaatattcaaaaaatactatatcctgttagaatgggtaaaatacacattttagacagtaccaaatggtgatgaggaagttgaaaaacaggaacattcactccttgtgggtgggaaggggatgctgaagaggcacatacacaactacagttagtactttattgtatatttcagaatagctagaagataagatttagaatgttcttaacacaaagaagtaatcagtgatggaggtgagggatatcccaggtatctggatttgatcattgcagataacatgcttttatcaaaatatcatgcgtatcccttaaaggttttgaactgttatgtatctatataaattaaacatttttaaaaaaacggtgcaggggaatccagctaattgcagattcccattagatgggatgtgctgcataatctgtaaactaatcagagtctgtgggtttgtaaaggactttagaaatcaactggactacttgtttctaaagataagtctcgcaagtttaattattttcttaatgggtggcgattttgaacatagaagaaatgttaacagttgtttctcaagttttcttctagtttcagaagaaaaggtttgcagggaggaatctgggaccatctttcaagtctcaacttcaatgtgattatctcagtaaatattttctgatacccctgagcagggtaagtctcatatatgtcctagaaactgcaagtagaattctaggcaccccaactgactgaagggaacttttctcttcaacgaggagttccatggacactagaaaacctagtttaggccgtgtcagaaatggaggatcagaaatccctcattataccctcctccctttggaattcaagcacaagtaaccagcattttccttaaaacagatttaaggctcagaaaacagattccgtgtagcagtaaaacaccaaattctaacctgactcaagaatagcatcacatgacagagaaaaggccttggaaggaatcaaagtcttttaccctaaaatatatgtctttgacatatttttaaatcaccctgcacagttatctttttgcaagagaaatttacattctgtagagaatcgcttccctttgaaggtgtttgtgttatactgaggtatcgggggaaattcagccagatactgggagaaattcacccccgatatttcacgtagattcttttctattttccttaagtgtcagccggtctgagaaataaagggacaaagtacaaaagagagaaattttaaagctgggtgtctgggggagacatcacagttcggcaggttctgtgatgccccctgagccgcaaaaccagcaggtttttattagtgattttcaaaaggggagggagtgtacgaatagggtgtgggtcacagagatcactgcttcacaaggtaatagaatatcacaaggccaatggaggcagggcgagatcacaggaccacaggaccagggcgagattaaaattgctaatgaagtttcaggcacgcattgtcatcgataacatcttatcaggagacagggtttgagagcagacaaccggtctgaccaaaatttattaggcgggaatttcctcgtcctaataagcctgggagcgctacgggagactggggcttatttcatccccccgctgcgaccgtaaaggacagccgccccccgaagcggccattttagaggcgtttagagcgtttctgtcttctctcccagtttctacgagttcctccagtgcctgtaacacagagctgttagtggctttgcccctgcctgttaattctttggtcatgtaatggtggtgagggagctgggtctggatgcatttcagccacagctgctgttccacttccccacacagaaccatctcgggaggaagaggttggagatttttctgatgtgtcctcaatcctggcagaaaaggtttcaatagcaataggattccctcagttgtatgtcctctgataatttaaacaataacttctttattatactcaactctaaacggtctaaagagtatgtctaagttaaccctgcattcgcttatatgacatttgctctaccccagataaggtcatattctctttctgtttgttcctgcaagtcactgactttcacaagatgtaaggtcccttgtttgtcctataacatcaataatctgatgttttaaagaaaatatgctaatttgcagctcagtaagtttagttgtaaaaataatacatttttttctgagatgcctacatctccatgctgagttggagctttatttgtaacactcagaaactggaaataacaaaaatattaaggagctttataaatagtacgggcacactcattaactggaattctgttcctcattacaaggaacatattctttatacacaaccaaactactgactcacaagtaattgtgctgagcaagaagccacacaaatgaaatggaaactgtaaggtttcactttaaccaaattttgaaaaatagacctgaagtagcaaacatgagagactgactcagtgtagtgagcggaaaatgactgggaggcaggaattagaggaaactagggaaaccttgaagtgtaattcattgttatcttgattgttattttggatgcacaggtaagcacatgtgaaactgcattttttcctttggagacatggtcttgctttgtcatccaggctggagctcagtggtgagatcatagctctctgcagcctcagaccctgcgtctccaccaatcttcctccctctgccatacgtagctgggactactggtatgtaagacaatacttggcttcagtgcttattaaaatgcacactttaattaagcagtatttgttatacagcaatcatgcctcaataagagtattgcaaataagtggatagataatttgttcaataaagattgatggaaagatagatgctaacatgagaaaatgcatgacacccaagaaaataacactgtaggaaacatgcttttctttacaattgttaggtaatcacaacagagcatacacatcacaccatgttctcgttacagagaaaaggttctgcaaacctcactagctgcaacccctgtgtgctgggcttggttcagggagaagtcaggtccagtggtgagaagcacaggcccagataaccaagctcactctgaccgaatgtgagccctgggtacattgtacaacccatctgtgcttctgctggtaatttttcatctgtaacatggaaatgacattgatactatataccatgtttcctctgcatatgtaaaaataaaaggtgattggtgctaactttaaatatacacagtttatgtacattgattgcacctcaatacaacagttttaaatagatattacaacattataagttttataggtttaatattttatcacagaacaaactttcaataagaaaccacaatttccaaatgctatcaatatcacaaatctcccccaggacactctcacatgctctgagccccactctctcctcaggcgtcccatcccagagcttggcatgtagtaggagacatgcaaatagagccctccctcggcttatgaaaaccagcccagccctgaccctgcagcactggaagaggagccccagccctgggattttcaggtgttttcatttggtgatcaggactgaacacagaggactcaccatggagtcatggctgagctgggtttttcttgccgctattttaaaaggtaattcattgagaactattgaaattgagtgtgagtggataagagtgagagaaacagtggatttgtgtggcagtttctgaccagggtttctttttgtttgcaggtgtccagtgtgaggtgcagctggtggagtccgggggaggcttggtccagcctggggggtccctgagactctcctgtgcagcctctggattcaccttcagtgactactacatgagctgggtccgccaggctcccgggaaggggctggagtgggtaggtttcattagaaacaaagctaatggtgggacaacagaatagaccacgtctgtgaaaggcagattcacaatctcaagagatgattccaaaagcatcacctatctgcaaatgaacagcctgagagccgaggacacggccgtgtattactgtgcgagagacacagtgaggggaagtcagtgtgagcccggacacaaacctccctgcaggggtccccaggaccaccagggggcgccagggacactgtgcacggggctgtctccagggcaggtgcaggtgctgctgagggctggcttcctgtgatggtctggggctgcctcattgtcaaatttccccagggaacttctccagatttacaatcctgtactaacatttcatgtctctaaatgcaaaacttttttgttctttctgtatttttgtttttgtaacaggaggacacattctcgcctccacagaagccacagtgtcactttgggggcagatgatccttctgtagtcagcaggatgaaagtgcagaggaatctcagtggaacctgggaagtcttttccagttagactcagggcagagacctccatgggaatctctgattagaacaggctttgagttctgataggagccaagagagaggctcacccagggtcagagtccttaaaacctgacggttttcacagctatctcacctcgtcttgaaaaactgtgcacatctgactcacactaattcagttgaccttatttctgctaatctattttccttctctgcagacttgattctcacagttccctttcttcttctcttgcctgaaaacagaggatgtgttttctgtagtcaaaatcccagagatcaggtctgcaggacctgggtaggctgaggggactttctcactcaccatagcctggtgacactcctgctgtattttgtatgtggaggtgtttggaaaacgaagtgaacattagtcatgaagggaataatactagttttctccaaagggatgtggatgtagagctgatcttgtgcttctcacactgtcacagaatttatgctctcccctgtgactttaggagagctgaggatggacactccattgtgctgtgagctctggtaatagtaattatagggtctcgctaggcagcctaaggtcaatactgctggccttcgggaaaggcaggctgggattcctaggaaaacctgcatctgccatccagcatggagtcccatcgtcttctgttatgctctgattgaatcaggcacacctaggttatctagaacactcttcgtgacttgggaaaaattagtggcaggctctactaagacctgcattatgccatgggagcaacacctaggctagtgtgtgattgagtaggtgagactatggtctagtcaaggtgacagggaaaattgttgccattattatgttttattttatatttggcaatatagtcatgctcatattacaaatatctttctacattttttgtgtcagaagcttttgaacaagagcaacttcatcttgaatagaggctaggaaaaataagactgagacctgctgggctacattcccagtaagttaaggcgttcttagtcacagggatgagataggaggtctgcacacgatccaggttataaagaccttgctggtaaaggttacagtaaagaagctggccacagcccaccaaaaccaggatggcaaaaaagtgatctctgttcgtcctcactgctcattataatgccttaacatgctgaaagccactcccctcagcgccatgacggtttacacatgccatggcaacatcaggaaggttccctagatggtgtacaaaggaggggaggacacctcagcttcgggaattgcccagggactcatgaataatccatccgttgtgagaaatacattcatccctttgagagaaaatgcacagggggtggaatgaggctgggaagccgatggcacatggtggaagcctgtccctgagtgaaagagagggggaagctggattgggtggaaggtccctatatttctgtgctgtgcaagggaggggcaaaatataattgagtctcttgtaagtcagtgtttcctctcaggggatccccatgactcccagaaataatggctctgcttaggaatccctgcagagtcactcctttcattagagtagaccacaggacatgggcctcagcacacgccatgccacagatgtcagaaagcagctgctgggaacctgacccacctgcattttgctgcctgtaggaggaggaggacgtgcattctcagggccaacacactgtttttggatttttatagagaacacctgcttttactttattttttggaaaaatatatgaacaaatgtgcacctgcaaacaattgtaattttcacatttattttaattgcatttgtttataattgtgcatgaggtcctggtgcagagttgagtttttcatgggaattgttccaagaatcaagaacatccattttccactttcactggtttttccccatgtgcagagaccttgagtagagcacatctggcccttatccacactatctcttgtgtccccgggaaagacagggatttgcctgactgcagaatctagggtaggagtctgcacacgtctgagcctgcagagaagcccagtgaagtttaatgaagtcagagagctttcccgtgtggggaacctctagcttcctcactctcagagattgctggtcagctgtaattgaggggttaagaattagagcacctgggagcctgtctcatcacagctccattctgtaacttaccaccatcagactgcaggtaataactcaatcttgatttttctgaccccatcttattcatttgtaagtttggttatacaaaatgttacattttagtttctgattcaaacctcagaattttgtgaattcatcaggaataattaaaaatgtgtccagtttttgggaaatgtaaattaatgtccatactgtttatgtgtgtgtgcgtgtgtgtgtggggggatatgtcagtcacttacaccccagtaaaaatgattccttaattatatagtcgaactgcaattagatttgctgaaaattgtctacagtaatttctgtgaagctgaccttgccaccttgctgtgaggatgctcagtgcacaggtgaggtctcacaggagagatccaaggttcagcccccaggactggctgagctcctggcttggagccagacataggactgggcatctcagaagcagatcccctggccctcctttatctgggctgtatgggcagcagaggacaagcagaagaaactcagaattcagccaagacagagccccatttaggagcaggtgcatcaaggaaccagccagagaacctctgactacaatctagggttgtcttcttttgcttcacagcatccagtcctaaggagaccacccactatatcatctgtaactcagaactttcttgttacaaaggaaaacacaaatgaatataactctgcagttaacttcttgaacctggattgatgagtttccacggccttatgagcacagagaccacatccagtgcaggtgcagagcaaggctgcaacagtcagcctgctgctccctcaggagtcttccaattcccttgtccaaagcatccgctcattcatgggctgagaaaagggaagtcattcatgcagtctactctctccacagaactgacggggcacaaggacaacatcgatttttcatggaacatgcctctaggaatgcagctgtgtgcacacacactgctaagcacacacttctttacataattacttgtaactgtattttcttatttattctctccaatttttttacacaaattcatcacttttccccataatcaaagaggattttgatcagaatgcttgtggggagccccttgccccccagatgcccaccatcattattcttgaagggaggaggaacggcagctctcttgatttctactctaatcctctaggactaaaaccagaaggttgcatgtccggtgcgggagcatcgaagaagatcctgtctgtagaagcaggagcgtcaagacttgactgagagccatggtgctgaaatgagatagattccctgatggagagcacacgtggacccccacacctgagggctcactgctcctcaccacagatgcactcccctactgagtcctgagacctgagtgcaccccatagagtagggctcagatgaggggatgcaaatctccaccagctccaccctcccctgggttcaaaagacgaggacagggcctcgctcagtgaatcctgctctccaccatggacatactttgttccacgctcctgctactgactgtcccgtcctgtgagtgctgtggtcaggtagtacttcagaagcaaaaaatctattctctcctttgtgggcttcatcttcttatgtcttctccacaggggtcttatcccaggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggataccacagagacacagcccagggcgcttcctgtacaagaacccaggtgtttttcagtggtgctccctccccacttctgcagaacaggatagtgtggctgagatgccatttcctgcccagggcgtgcgtttcccatccccatctgactcagagccttgttttcctcctttttctttactaataaatggcatgtcccctgttagtggttcatgcaagcagaagctgtatcctgtttgacaaagattcagcatgaaaggtcctgctacctaaaaaaaaatagacagatgagatttaattaacctaaataatttttttcacaacaacagagtgaatacgcaatttacagaatgacagaaaacttttgcacactttgcctgtgacagggaactaatatgaagaatttgcaaggaactcaaacaactctacaacaacaacagcaacaagaaccaaataactccgttaaaatgagcaaaggacatgagtagacattttcaaaagaacacatagaaatggataataaatatataaacaatactcaacatcactaaccatcagggaaatgcaaattaaaaccacaataagatatcatcttccaccagtcacaatgactattactaaaaactcaaataatatcagatgttgctgaggatgggaaatgaaggcaactcttagacattgttgatgaggatgtagatgagtacaacctctgtggaaaatggtatggagatttcccagaaaactagaaatagaactgtcatttggtccagcaatcccactactgggtaactacccaaaggaaaataaactattatttcaaaaagatacccaccttctatgcttaccataaaactactctcaatagcacatatgtcaaactgagtgtctgccaaccgatgattttataaaagaatatagcatgtatgcacaattcaatactagtcagccacaataaggaatgaaactgtgtcttttgcagcaagatgcatagaagtgggggacaatataattagtgaactaactcacaaacagaatgtcacatgtcacacattattacttgtaagtgggaggtaaacagcgtgtacacaaggatttgtagagagaaattacacacattggagacttacaaggatgggcgggcagaaggtgggagcatgatgagtcattacataacaggcacaatataaaataattaagaattgaccaatgatcttaaaattaaaatgtagaatatgatcaataaatgaacttgatattagttgacctcattaaatttaaaaactttttctactcaaatgactgtaagaaaatgaatgcccggttacagatgagaaactgtttgcgagtcaaataaccaccaatgtaactataataagaaacttcagaactcaactgtgaataaaaaagaaacaactgatggataaattaggcaagggtttctacagacatttcgtcaaagaagatgtgcagatgacactgaagcatataaacaggatctcaacaggattttccgttagagaaattcaaatcaagcccgcaaagagacaccactgtacactttttaaaatggctgaaattaagaagaaatacagataacatcaatgctggtgagcataccaggttgctagaggctaaaacattgctaacaggaatgcaaaatgaaacagatactcaggaaaataatttttagttttctctaaaatcaaacatacccttaacacctgaatatttgcatcagagaaaaacaatcttacattcacgcataacttctattcaaatattcaagatatcgtgtgtatgtgtgttagaaagtaaaaataacataaatgtctcaaaatttgaataggtgaagaactaggaagcatctataaattgaataccaccagcaataaaaaaataacaagtgaccgatacataaactattacaggtgaactccagacattgtgctaagtgagagaagccagtctcaaagatcaaagggacacagctgtaagcaccacggtcatcctcaggtgtcagtggtttgggctggactttctgtgtctctttcctgaccagacccagatattgagctccaccacttgcagatggaaaatcctattttcaaccatgcagtgaggtttgaactgcttcacagactgaacgaaacaaacacgggctcctttgaacagcgtccggcatttgttccaaccacaagagaacgtccctcagctctcccacctcctcggttctctcctgcaagccagcagccctgcagtttagcctgcatctcccgtgcatccacccatctccctccaagcaccttcccccacaccctccactgtttctgagagcacaggcaggctttgaacttttccgcattctgttgttattgaagttaggatgtttaggaccaacttaaggatcatattttatgactgaattccagtgccccttctctcctgggacagagtgcataaccaagtttctgcaggtggagacgaagttgagcttttttcttcctcagcctaggagatgagcgctaattggagggttggcagaagcttcccaccatcccagcactttggttctggtggggcggaatcggtgccatagggcagagctagaaaccgcggactgaatgttcccagtggcactggacccagggcagagcctccatccacgagtggggctctatggaagaagtgagtctctggctctcagtagctctcgtccagcactgaacctcagcatcatgtgctgtgtgcagggtcagagggccaacgtactggcccctgggaaagcgtttcctctggtgggagttggtagaaggtgtcctgtcttcttggctgcatctgtccgcagtggagtttacatcatgctgagctgggatgtggaaggaaggaagagcatcttagatcaaatatgatgactggccttactgagttttctagattttcctgaataaatgtttcttcactcactgtgtgctgttagagtctttccaaacctgtaatttcccaaaataattttcactggtctcatgagggcatggattcattgagcccctcatgctgtcaaagagaaatagaactgtttttttttttcacttcatagcgaacatccatgggttatcaaataatgggctggcttttcttccaacactttacagacaccatcaattttcttcttgcttataaggttttaaccagaagaatgctgtcatggtcttttctgttcttttggaaggaatgccccctctactcacctccacttgtctgcctgtatttctatttgtctttggttttcaacaattttaataagatttacctaaatgtgtgtggggggagcatggggtgttattctgctgttctgtgttctctgagatgcatggattcaccatttactctgtctccatttttgtgaaaacaattagaaaaaaagtcagtatgagcccagaaacaagcctccctgaagtgggcacaggaccacctgggggcgctcaggacccactgaacacaagagccagccccagggcaggtgcagatgcgggttaagttctggtttcctgtcaaccctgtggcttcctctccataaaacagtttcctttgtggcatatctctggattccttatcctgttcttcctgtgaagtctctgaagaagaaacatttgtcgtaacaagagaaaaactttctcacatgcaccaaaggcagagtcacctacagtcacttactcctgtttctcaatgtcaataagttaccaatgcttctgaagttaatcagctaaatctataaaaggtgcggtgtttaactcagcattacagcccagctcaacagaactccaaaggtcagccagcagcagccaggaaaaagtgcatgctgggcattggggcagagggagttaccatccagtgcaagagaagaaagcccccgtggtggtcattgtcaggactccaatcccacagttccaattgtaggtgatgccaggcaaaggaagagagaccccaccaatggttagtgtggatgtcgagtttgatgtttccacactcacactccaggtgaatatgaaaagatttattagctctatttctgaggtgtctgctgagagcagcacagtcctctcaagaaattacagattggaatttcctcagtagagcaggaaaggaggctggctcagggctttataatgatttggtggtggggtcggcgggggggggggggcgtttctactcaggagaaggagcttgtgtgatttaaacctcacactgacatcacatgagggagcttccatgatttcttactagatttcccatgtgtgggggacaaggatgagggagaataaaccttaattcatcagcatcaaggcaccaaaaataggacctgacactttattctccctagcagcttaagaaaatgagtgaaaaagagagataagagtccacccatgtgctgaaaagcatagctcttggtaaagacgagaaaaaggcactcctacgaagaaggggttgggcagaagctttatgctgaagggtttggctaaagagacataatcaacaggttacaggaggggctactgatgttcatggaggtggtcctcacacatgcatactgaacaaacatgtctgtaacgtatgacccctgttcacttaccagtggagacttagcatttaaattcattccagtcaggccctatgtgcaaacagcagaagcagagacacaaaggtactcagggtgcagcctctgtgaacggccagagccaggccatggtcagcggtctcggattaggagaaagttcctgatatcactgtagtgttcaatcaaagctggggttatggtttgtggaacaggggtcagttcatcagggggtgggctgcaattgtcttcatagtgcttgtctcagtgccggtgcttactgagccactagagaaaaaggtttaattgagcttctttaaaatcaacattttgaattatttatcagacgtttcaaatatgtcatgttgtttagattctattgctggagagttaaggtgatatttggggttttgtaactctgttttttcatacttcctgaattgcttatctgtttgcttttcattagctaaactatcgcttcttcttattttttaattcattctgattttgatgaatatttaattccctttagaatgtgaatataatgtacattgtgtgggtattttgattttggttcttggtttacttagtggcaaagactctgtaagagttccttgtctatagatagccattatttagtggctttctgaaatggtggttttagtaccaaagtactggacttgtgagtaggctcactgccccctgcaggtcctagatagtggaggcctcaggaactgtttctcatttggaatgcctttgtttcagcagattttgtgttgggttgttaagttcaccctccacattagtagatgtccttacagattagagctgactctggtagaagcagttgagtgcatgcttgatatctgtgcacagggagaagctctctgttgcctcaggcgatggactggtctatgaaatgcacagtgacctgagttccctgctcagcccctgagaggtggaccaagctggacacacatgagccaccgagcctggcaagcaaaagcgccagccttgatggaaatggcgagctgagcggcatctactcagtgtggtttcttttgttattaagagctttagtgtggtggctgtttcaaattcccgttgtagtagtaatatactgggtatgtgagcaggcccgtggtcttttgcggggttggaatcaccgaagtaatgagaagctaatctcattttcaactgctgtacactggtggtattgagtttgtatgaggtcatgcagtttgaacgtcaggccagtaggtggtgctcgcaggtaagagccggctatggtggcagcagaagggtttatgctttactggtgattaaagtgggaaacttggcgtgttccagatcttagagaaaagatttttagttatttctcattcaacctgatactacctgaaagtctctcgaatgtaacttttattttgtcgagatgggttctttctatacccattttttatgttttttttgtgaaaggatgttgtttcatcaaatgcgttttcagcatcaattgaaaaaagttatatgtggattaaagatcaaaatgtaaaacctaacactataaaacctctggataataacataggaaacagaatttaggaggtaagaactgacaaaggttttataatgaaaatgctagaagtagttgcaacaaaattgaaaattgacaaatgggacctaagtaaattaaagaacttctgtacagcaaaagacactatcgacagagtaaacaggcaacctacagaatgggaaataaaatatttgcagcctatacatctgacaaaggtccgacacttagtatatacatggaaatttaacaaacatacaagaaataaaaagtgaccaaaggacatgaaaagacacttcaaaaaagacctacatgtggccaacaagcataggaaaaaatgctgaatatcactatcattagagaaatacatatcaaaacctcaatgaggtaccgtctcacatcagtcaggatggctaatcttaaaaaaaaaataacagatttttaaggttacagaaaaaaggggaaatttatacacttttggcgggaatataaatgagttcaaccattgtggaaagcagtgtggtgatccctccaataacctaaaacagaagtttcatttgacccaacaatcctacaactggacatatacctaaaggaatataaacatgtaggttcactgcagcactatccacaatagcatagacatggaatttacctaaatccccatcactggcagaatgatagagaaaaatgtggtacatacaaccatggaatactatgcagctaaggaaagaatgaaactatgtcctttgtaggaacatgatggaactggcagtcaatactcttagaaaactaattcaggaacagaaaaccagatattatatattctcccttatttgttggagataaataaaagcaaatattcttccagggcctgagtcttccttattcaacaagtcattctaaattaagtgttcagcaagttgctgatactcatctaaatattctatttcatctgggccacttacatcactcaaaaagcaatgagagctatatttctaaggggggttctaggataataaatacctgaatagtgagaatatgaaggatatggaaactgggccacttatatcactcaaaaaggaatgagagctatatttataaggggggttctaggataataaatacctgaatagtgagaatatgaaggatatggaaactgggccacttatatcactcaaaaagcaatgaaagctatatttacaaggggggttctaggataataaatatctgaatagtgagaatatgaaggatatggatggtttttttttaactcaatgggcacataactgtgggagatactatattcctatgaagaaggtattcagacttcagagataagtaatgtttcctacattgtgcttgtgacttggaagcagtggattgaagagtgtgataagtgcccagaccaagcagaacagaaatcagcatgtaaagatgatgatctatggatatgatctaaaaccatgtaaatacttcaaataattctatttaatgcagtttgaaataaaacacaaacttattcaaaatacaaattacttggtaattattttgggagctatgagttcaccaagaaactcaaattcctatttctatttcaacccctgattcctactgtcaatgggagggaagtctcagaaccaatcacacatcagacggcaaatctgtcaaccaagagtctttccactgaaggacctgggaggtcaggaccctcaggaaagtgctggggaccctgtcttgggagtgcccagcagatctcagaactctccatgggtcctgctggacactcatgtagggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagcgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatggactggacctggaggttcctctttgtggtggcagcagctacaggtaaggggcttcctagtcctaaggctgaggaagggatcctggtttagttaaagaggattttattcacccctgtgtcctctccacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagacacagtgtgaaaacccacatcctgagagtgtcagaaaccctgagggagaaggcagctgtgccgggctgaggagatgacaggggttattaggtttaaggctgtttacaaaatgggttatatatttgagaaaaaaagaacagtagaaacaagtacatactctaattttaagataaatattccattcaagagtcgtaatataagccaaattcacagagtggaaaaggccacactctataacgttgatacaaacattccatgaaggtgctactgtgaacaagttttcaaattggatgaatacatgatttggagcaaggttatttgatcatgtggtgagactaagaatgattcttaaaaagtgccaaaagtttccttcaaatgtttctgtcactccttatcataaagtttattttacagcagttttaggattacaaagaaattgcacaggaggcgtgagaattcccatgactccctgccctacacaggcacagcctcctccactacgaccatcctgcaccgcagtcacaaatcagttacaatggaggaatctccaaggacgcttggttctttctttttctggtgatctcctaatataacaagcctaagtatctcaagattccacggttttttcagtgttttctagaactgatattagtcagagggaaagtgggtaaggctattactatttgaactctttcttccaaaatccacaaaatatatattaatttagagcttatcttacttctggtttacaatgctccttcccagagagtaagatttttaagcttttagaggccagttcatgtctctaaaagaccagaaagttctggggaatcccataatgaacatcctttcattggaattggagaccctggcaatgagagattccatgtataatgccctagagttggattaggtgcactgtgagctcttgggtggtgagtctgagtaaggtggtttgtgcagcaaatgcaaacacattcatgggacccaggtaggaacaaaagcttccctctgcaaagggagtgtgcacctgaagcagccctcacagaggtgggcactgcttgcccttgatgagtgcacattagccagaggcatgatcatgattggtcttgcagataaagagcaccactgaggtcataggttatgaaaatgtttgtcatcctccagctgagcaagtccatctgcttgtttgtgggtgtcaactccatagagggtgcactttgggagatgacaagatgcgcacaaacctgctctcactaattatccactaccacacactcaagaccaacctgtgctccggaaaggaatacgtgcctgtggaaatagacagagcttaagtattttgtaacctggtgaacatactgcccaaagccacacgtttcaggaagattagctcagaaatgttcaccaattgactgaagggcagtggcgggtgaggtgatgggacagcctcagggctgcacatgaggagggctccctcccccatgcaggcttttcctccaggagctgcatcaggacctcaaggaagatcagggagaattctgagaacacccttctgtggagctgcctagagaagaagaataaatatgaaaaaatacaactctgagtaatgcatggatatttgttcatgaaaactctctctgaaagcttgtgaaggtctcgaaatacccctgattagctgaagacaaacatttaaaccctccttccacagggagttcaagcaggctggatgtgtccttctatggatgatcttccccaaccccttcctcttcccagctcatccctggctctctgtgtaaacagttctcatcagtggaatgtggttgatgaagtgacgtcttcaatttcctcatcttcttggtggtcatgttattttttttcatctgagggttaaaaactcacctgcatgcagcacatgacaggctaaaatctcttgtggacaaaacggtaacaaaggcacccaccagggttgagcatccgtgttgctgacaacgaccaccaggggtcaacgtcctcttcacaatcctgtgtcagagcatcactggaatgatttcattagcaacttcccaggagaatcagcttaaaaaatacttgtcccattttccatgcagatataacccatcccttttcctgaagaaacagagctcaatactaaatacactgaatgttgtttttgctggttttgtaagtttgtgactttatcactttctaatttctgagttaggtggaccactctacatatttctctcatgggtgtgaccagccttctggatgtcaaatataactgactttgttcgtgtaaatgtcaacacaagctcttcatggtttcggtgctcactgactactctaaacttacacaatgtgtttctttattagctttgttttctggtatctctacgtggcttattcatcacgcccattttatgttacttgtacaggtaattgattgttatttttaaaatttactattgcttactttaattaaataagtaggcaatagcttagaatagaaaataaggtaaagtaaaagtacatgcagtaatatttaatataagtaagcaggcagctgaaagcaagggaaatctactctgctgtgtaaaagggtatggagacctcacagtatgatgatcttctgcagtttaatccatactgctacataagtgacaacgtccactctgtttatatcagagcttcccctaatatgtacaatgaagactgagcccaggtacctccaaccagaatggccagcaccttttaccaagtgcctgaactgacagcaatagagatcacgtttgtctaaaaaaaaactcacactgtaaggatacccaccagtcatatggagacaattagaaaaaattaaagcccaagcctgataggtagtaatttgtacttagaagaattaacttttatctccacatgttttttttttaatcatagagaaactactagtggtagttccactgtccaagttactgaatatcttattcctagaatgagacactgcacaatgtgttctgacacgacagaacgtagctgtagttaagggacaggagatgtgatcacgggaacacgagtctgcgatccagcagttcttgctccttcacatttgcccagaaacaaagaccccagcagaacaatggaagcattgacttaggtctccacagtctgatgtgagataaacagcaggtagtttatgcctctgttttatagcaggaaatttaatctctaaagaagggccattttggagataatactattctccaggatgcttaaatgcccccaaacagagagcactgcagagccccgtggccaggagctacaacacctgggtctggaaaccactggacagaaatatgagattctcttaccacatgtatgatggaaccttgaagaatgtttccttccttctccataatcacagcctgcgatgaactggaggtcccagtacataaaaaaacatgtgtctgcattggatacagaattagtcccataaaataattgtgctactgtggtgctcacactgttggaccagctgaccaagaaagtgttgtaagagctgcagtgtctactgattcctaccatatttggggctacattcacatctatcaagggaaagagagataagaaaatctctctaggccgggcgcggtggctcacgcctgtaatcccagcactttgcggggccaaggtgggcggatcacgaggtcaggagatccggaccatcctggccaacatggtgaaacctccatctctcctaaaaatacaaaaaattagctgggcgtggtggtgggcgcctgtagtcccagctactcgggaggctgaggcaggagaatggcatgaacctgggaggcgtagcttgcagtgagccgagattgtgccacttcaactccagcctgggggacagagctagactccataaaagaaagaaagaagaaagaaagaaagagagagagagagaaagaaaaataaagaaagaaagaaagaaagaagaaagaaagaaagagaaagaaagaaagaaagaaagaaagaaagaaagaaagaaagaaagaaagaaagaaagaaagaaaggaagaaagaaagagaaagaaagaaagaaaaaggaaatctggcctagtctattgatttattttcatttcttcaaaatgaggtaaaaaaatcgcatcaactaaaatgcggacgatgatgaagtgatcaaagacaacagccactcagtgtttcaggtgtgttttaacccaaagtcacacaacatgtgagaagctgaactccggagccaggggcatctctgaggagaatgtggaaggtgggcgggaggaaacagtgattcatgtctagggaaatttcagggctgcctgctctaggggaaaggaaagcctgagcctctcccacagtgtgctctgttaaaaattttggctgacttattgtattcttcgacacaaatatttttgtttcgtcctcttttaaatttttttatctctattaaaattctcactttgttcttgcatgctgccactagcttattaaacatgtttctaatatttacttcaaattttctgccaggacatccatatgtttttttattcattgatgccagattctggacctctatgttgtctgtctgtttggaccatgttcccgtgtttcttcattttccttgacggtctttcttgctatccgttcatcataacaaacagctctttgctcatcataacaaagagccacactcttccaagcctaggctcatagagacaaccctcaccaatcactccatcagtgattgcggatctctcaaaacttcataatgacttcataatattccaacttctgtctctgttcttagtggcccccaggtttagagaacgttgggttgtgccaggactcccaaataggagaggtagaaactatatactcaagtagcctcttgaacagtgtaccattagacacagttttattattgaggtatgattcatcaaaataatgttagttcaagcgcctctcaaccagtcatctaacattgaaatgttataatttcttatttggatctctctggagtttatcttttgaatatttaggtaaaattacaaactagtataaagctatttggcaaaattatctgctgcagaagtttcatcttctgtgtatctctttttgcacttaaatatgtttttattagtcatgtatccagaatatagaaatatgtcttaaagttccttcatttttttaaggttatggggttttcagaagaggtgagaaccttctttatttataaaatacctgattccacattgctggatagtttcttctttaatttgtctcctcctctcacattatactatattaagcaaggagaaatcaaaacacaccatccacactttgcttagaaatccccaaagctaaattttaagtatcacttactaattctaattttacccattgtaacttaattaacatgaaatctataacaaggttacacatactttcccgttctaataccatgttcgtgacagaaacaaagcaggtgtcttctctgcaatgtggtcatctgaggtctgagatgaggggtcacacaggtattttataatatttaagggtttggacaacagtagtgataacaattctgaacagcactggctcctaattgaaaggttataggtaaatttaatgctgcaattttatattaacaccttgtccgaatgaagctcagaggaagatgccacatcccaggtcacacagtgaggaggaatggagctgtgctctgctctccacaccacctacaaatcccaggccccagcccaggttccactggcgcacggcacctagagcacagttcccaggggatgatctcagggcacctgcttcctcgggcagggcgctgccttctctcagttgtgcacttgctcctcctgcaggttctgtagtgagcacttgcattgccgtgatttatacatgttctctcccctaatatggaaaaaatatttattaaattttcaactcattttctctgatgcacactgcattagcagaaaggaataaatcacattccttatcctcccataaagccaaagattcccgaagacagagctgatgtgatgtactcataggtggatctctgcccctcaagggaggccttggtcttcaagtttcagtaattctaggaagcgaaggacacctacatctcctgctccctgctctgtagctcacctgagaacagctttctcattggaatgtcttgtgtttaaggaataagagtccatgtttcaggttcgggagcccaggtgcacctactggatgcagcccaggattggagacactttccagaagacaacatcacctgagacatgaccagtcccactgtttcactttcacaatttcaacttcctcagaagaaaattaaaattgctgagacttgttcataagtgttgtgccatgtccttactctgttttcttgcctgttcatttatgtcataccaggtgtctgttatatgtaataagatcaaaattctgcctccagtaacacatcaatggagacctttgattgtacttttggtttatgcactgacacatagattatgatgttcatcacattcatttttatgtcaaaggaaatctgcataatctgaatgccaatactttttggaatctgctaagtaactgaaattgaaaaaaaaatacccactcaagaacctggatagacagccatgtccagaatggcagttgacacttgtttaactggaagagaatctacagaagccacaagttgttgagggcacttacatgataagcactatagttgtcttaaagacagatgtggactcagtaaatgtgactgttccagagggtcttatatttctatgttttatggactttctcaccagaaacctccagattctaaaaaatactatccaaatacgtttcttatttgtcagattggagaagattaattactgaaaaatattacgggagactttttcaaaagcttctacatggaaggacttttcgagaaccttgtcctatgtaaaggaagacaaatctcccattccagatttctctcccattcttccattattatagaaatgagcaaagttagccaataggggtaagatgtaagtaaatagtccagggacactgaaaccacaaaagggagtaatggccaaagtagcttttcccctggagattcctggtcaaagtcacagcccagaagaggaagccgatcgcatctctaggtttccattgtcaaaacaggcagtgcttgcctgcactgcacaatccattctaaccagtgtgatagctctggattaaagatggaagtgtggcaatgcacagactctatgtgagaagaacactggaaaactaaaggacaaaggcagagagtaggacaaggacgacaaagcaatctgaagcctctgacatcaccatttttaagatcaaggtcttggaaactccctcattgacctttagatctctaggagaaaaaagtcagatacctgggcctagtgtcagtgtaggaggaattttctataggctaggcattaggaagaagggaaaattcttccttattaggaagttattgttatagtgttatagtgttattggaatagtggatatggagtgggctttcatctcgatcaatctgcacctgctggtattttccaatgctacattcacctgcaagagcccaatgaagaaagaaggcactcccaaatcttttgcaagtttttgtattcactgtgggtccactgcttaagtgcatctggagcttcagagaaggggctctgtcctgtgtcatagaacccttgctttgagtctcacggcagagttcaatctgtttagtaaagttgatcaattatttcaagagatggtgtcaccagcatatggtgtcactgagggagtattctacactagcacacagccatttcacgctgggctagagaagctggggggaaatgctttgtgagccccaacaggaacctccttgcaaggcaagggctgggcgggagggggcactcaggagccactcagcacgggttccagccctgcagctggtgcacaggaggctgctgagaaggtttcctctcaggggctgggtcttcctttgggagaaaaaagctaaaattcaataagttgctggtgtgcccttaaatattctatcacatctgagctgctcccacaattcaagagaaggagaactatctttttaaatattctatcacatctgagctgctcccacaattcaagacaaggagaactatctttttaaagtcggcttctaagattataaataccttaatagtgagaatatgaagaatagggatggtcttactaattcaatgcagagagaattatgggagtcactatatttccatgaataataatttcagatttcaggctggggctggtggctcatgcctgtaatcccagcactttgcgaggctgaggtgagcgggtcacgaggtcaggagctggagaccatcctggctaacaccatgaaaccctgtctctattcaaaatacaaaaaattagccaggtggggtggcatgaacctgtagtcccagctactcgggaggctgaggcaggagatttaatcctacaagacctacagcctcagacaatgctgctgtcagtgatcgtgggaagcagagtttgcagtgagctgagattgtgccactgcactccagcttgggcaacaaagcgagattccatctccaaaaaaaaaattcagatttcagtgttaagtaaagttgcctacattgtgtgagtgacagggcagtggtggatccgagagtgtggatctgagagtgagtcagaaatcagcatgtaaagatgaggatctatgcacatgaactgaaagtatgtaaacagttcatgaaattctaataaatccagtaggaaataaaacccaaacttatccaaaacacaaattcccttgaaattattatgggagcatgagttcataaagaactcctaactcctgtttcaacttctgaatcctagtgtccatgacataagaaaatcatctccaattatgcatcacagggcaaatctgtaaactaagagtttttctgttgacgatcctggggaatcaggacaccaggaaggtgctggagaaactgtctcaggagcgccccagggatctcagaggaacgtgctggcactcacgtgggacatcagcgtcacttgctcagagtcatcagtgagctgtgctggtgtctgacgggtccagcatagggccaaggcacctgctctgtgtcatggaccgagatggtccccagaatgatccaagtggtctctgtgctaatctaatgtagtttcacagtgagaggccgttctgagggggcttcttcttcagtgaaaggacctctgtccacaaatattcctaaatggacaggggcatgcatttcctcaagcaggattagggcttggaccatcagcatctcactcttgcaaggctgatgtgtcatttgtcttccctttcttatcatggatcaggctttgagctatgaaatgccctgtctcatgaatatgtaaatacctgagatccactgaggtaaatatggtctgtgccctgagagcttcacccaacaatcacatcccgcctctagagaatcccccgagagcatggctcctcaccatggacaggacctagagctaatcttcctggtggcagcagctacaggtaaggtgctcccaagtcccagtgatgagaaggggattgagtacagtcaaggaggctttcatccactcctgtgtcccaccctccaatgggtgtctgctcccaggtgcagctggggcagtctgaggctgaggtaaagaagcctggggcctcagtgaaggtctcctgcaaggcttccggatacaccttcacttgctgctccttgcactggttgcaacaggcccctggacaagggcttgaaaggatgagatggatcacactttacaatggtaacaccaactatgcaaagaagttccagggcagagtcaccattaccagggacatgtccctgaggacagcctacatagagctgagcagcctgagatctgaggactcggctgtgtattactgggcaagatacacggtgcgagaacccacatcctgagagagtcaggaaccccagggaggaggcagctgtgctggcatggaggagatgacaaagattattagattgaagactttcttagaaaatagcattaagtcatttacgaaaaggaacaatataaatgtgtatttgagaaattgtaattatttgagagatttcttatgcaacatttattctgtaagcaaattctagggattggagaattaatcaaattaataaagctgacatagaaattcctctgaaggtatctttgtaaacatcaatttctgaatcagtgttgtaaatgttttggaacacagacacaagatcacattttaactctacttttgtctctataaaaaatgccaaaaagagtctcattttgaccatgtacctctttgaattcccaccatcaatgaatgattgttcttgggtttccacattatattaccatttatcattatgagaattgtgtgttttaaccattataataggtgagtaatggtatctaatttttgcttaaatgcacatgcccctaataaaattcatatttaacaattttcatataatttttggtgagatgcctctcctgatatttggttcattttcaatagcactgttttcttttgattagttgtaagtttacttgcatattgattataaaagttaattaacaaattaaaagaattcatttaacaaatatgtgacttggaagttgcaggggtttgtcctgcacaccctgacacaatgacgaatgaataaagtgcactgacacaaagatattctgctttgccagctcgactgagcatctgggccacttagtcacagccaccgcctcgatcagtcagggagacttacatttattcagtaaagattaattgacaaaggccgtgagtaaacaccactagaaggtaattgacattgtggacctcttgagtggaaagcaattaagcacccgcggtagatcaaaggttagtcttaggaacacatgagtaaacaagctagttagataagctcctcacattcctttgtttctactctactttatgtaactaaaggtaaggggactaggctgccttcagccagatttattaccaaagttatgcaaactcacaggccttccaagagggtttgtggctattagaactaaaatgtttcccaccagcctgactggaccaccacatctccccattttttgttttctacatcaggtcttttggattggagcgtgcagatatgcgcagcaacatgtctgtcaggtgtggcagtcattgctcttattctggctttgcatcctagaattagcaaataacataaaacaatcatgagtagaattagcaacattcttttccagtcagagtgacccccaggagcggggtctaaccaggagagatgatctcacacacccttccatatgactgtttgttgggggtgtagatctattgcgtgaagagattctaaaattttagtttgaacttactttacgtctgctgttaaattgtcatgaaaggttccccagaggtattgttttacctcatcctaactatgtattgatagattccgtggtagagaagtgatacagatatgttcatgttcccagtcacagtttaaatgctgtcagaatgccactgcatcttgtcgctccctgacatattctaaagcagcctcaagggctagcagacgtgtgataattttttgatctataccttgctgtaagagaagttcattagacacatttttggccagattatctaaaaaggcagcagtttacactaagtcaataatacatgcaacagcaacactagctgttgccaggatgactatggctgagactataaaggccataagtgtaactatgaatcttttgtgtctgacctgggacagggcacgttctaaggttgcaagggcataggaaccttgccaattgcatgccagattgactggtaagaatgcctcagattttctctttaataccatggcgctagtaatttttaagttagatatactgtgattagtgatacatgaggcaaaccaagcctgtccctgcatccaggtcacaaacgtggagttttgaggtgtaatggaaatgttagttcccataaggaaatcgtatggatgggtagtgcaaattaggcactgatcagtgtgattatgaataaagcttatagcatagttgcaacgggaaatatggtatgtcccatgccaggtgttaagggaggtgctaagatgtcccagacaccataaagtaccttggggcggcatggactttacttggggccttggatatgccatccccccttggcacaaattataggggaaaagacatggctatgaaactgattgatgctctgatggatgaaggtattagtaaggctgccctgcaatcagccgctgggactccagtctaagatgttataattacctaactggaggctatgggctcgtctcccatgacagacctcccagctaaagtcgaaccgattactgtcccagctttgttccttagcacaggaaggaatgtttgggaaagaagcagcggcatggattgcattgctcagtttgaggctatctgcagctaagaatgttaaggcatttcctttgctatgatgtaaccatacttgtttttgggcaggtacacagtaagggttagagtctttataacttacacacactgggaggataatagtggattgacatatagtgttatctgccaccttagtccattgtgtgccattattgagggaccccacgtggggtaaatctatccctcctagccaagcagtcacgttactataggctgggaagggagtgtcttcccatttggcagggtgaaagaaaggtgtgtctaagatagaggcccaatagagtgtagcaggtacaggttgcagacaaagtgagagcataaaaaggattaataccctacatgagttgcaatgtacaacagaaatcatagcaccaaacacattatctggagtaaatggtgtctgcatttggagcaggattcgttcagcctcctgagttgtccttttcagtaccccccaggtaatgtctggggattgtgttgttcacagaagctgcatcgtccggggctgtaggtcttgtagggttaactccttcatttctggtactgggttgggtcctagcaatgtcctggtatggtttgatgcgtcgtgctggaacccaaagacgacccgagggggtgtggatacaagcgtaccctcttccccacattaataattcacttggaccacaccattcattactgtttacatctttccataaaactgcaggtttcatgtattgggaaattttagcaaagtgcttttctacagcggattcaaatttgtcttctaaattaaaaaaataaggtttgtgccaaaagtgttgcagggtctgtacccatactcccctgtttttgttttttgtgcatattttaagggtggagttggcaagttctactattgcttgtccttggggtttatatgggatacctgaggaatgtcggatattccacgtgtgacaaaattgttgaaattgtgagctggcctaacctggacaattatcagtcttaatttttgcgggttgttccataaatgcaaaagataaaagaagatgtttaatgacatatcgagtaaactctccaggcggagtatgtgcactacttaaatgagtgttgatatcaaaggatacatgtacctatctaagttttccaaattcagggatgtgtgtaacatctgtttgccacaacagattaggttctagggttaatacctgttgaaggaggagacgtgcctgtgagctggcgatctcagagactgtttagccagtctctgtataagttgagctggcgatctcagagactgtttagccagtctctatataagttggaattgtttagataagtttttccaattttggtggaaaaattggtgcaattgggtggcttggtcaagcagcgatatcataacttgaaggttggcttgattattgtcataagctaatggaccaggcagtgagctgtgggctggaatgtgtgtaataaaataggatgtgtactttgatttagcaattgctgaaatcagagaaaaagagtccacagggctggctccagagttgacttaattagggctgtaaaagttctctagacccaatattaaggactcaactttagctttctgagtgctagtaaacccagattgagtgaatgaattgtgtggtgtccaccagactgctgcttttccatatttaccagatctatcagtaaacagtgctaaagcattaaatatgggggagtaaactatttttgtaggtaacacattaactaggccttttgggaatgatgaaaatgggtaaattctaagggctgttagtagaatattacccataaaatgaataatcttgtagccaggaaactttttcctactagggagcaaagcttgcttttaattgctccctaactaacttaggggctttttgtaatgtctctccctttagaggttactgttttacttaaactggatttggagtcatgttaggaataagagagagataacagtggccattattagaaagaggtttctcaaattcttaaattttacttaactcttatcatagaattataatctgggatgtcgcttgtatacaaagaacataacaaattttgctttgggccgcctgcggagccagagagcggagcaggtgggtcccagggcggctgcggctttgtgcttgcttagaagccacttttcgccaccgcgtttcctctgtgccagctccctccccacactgctcagcagctgctgccggcagaggggagcttcctgcacttgctcctgcccctaatgccttttcttaacccttcgtgtacctgattgccttgctgatcttgcattactgggcagtctaagagctccccttttaatgctgcttgtcgaagacagggacatatagctgtagtgtatttcctctattttttctatctattgaaagagggggctcaggcaaaacctccgtttcctctttgttattttgggaagagcaggtggtaaggcaagtgacgtttcttcctccttcccctttttgggttcttctttgtataatgggactaaagccatccctactaaagcccatagtgttaaagatgatgctgggacccgttgcgcttgtgtgtaatgctgttcaagatttctccctacttgttcccagagctttacttctagctttccttcttctgggaccacagattatgtgagacaacagttttcattaggtgccttaattgagcctgcgaaactgatgctcccctagccttaagcaactgttacaatactttcatatgctgtttgtgttgagctgataactgctgtcccatgatgaaatctcagcctgaacaatctcccctgaacttggagatcccaagtgggcaccaatgacttactgttttactgacttgaccacgcagtcttcctcaaccttcgttttcatggggtctgtcacgctccctttgcagcgatcctcacatggggcaccggctgtgggggtctgtcctgcaggtcctgacacaacaacgggtgaataaagtacactgacacacagatattctgctttgccagcttgactgaccatccaggtagatcaaaggtgagtcttaggaccacatgagtgaacaagctagttagataaactccccacattcttttgtttctactcaaatttactaaactaaaggtaaggggactaggctgccttcagccagatttattactgaagttatgcaatctctcaagccttccacgaggacttgtggctattataactaaaatttttcccacgagcctgactgaaccccacaggaagtattttctccaagtctgtggttgccttttactcccttatcagtaggtatcgcagaaaaatgtgtgtgtttgtgtgtgtgtgtgtttgtacaaatttagattgaaaacatatataattttattcattcatagatcatgcctttggcattatatctgaagtttcattataaaataaactaatagtcattattttttccatatctctaatctcaggccacaatcaactcatgagtgtttaaacttcacctgcttgattggaggaccatcaatctaatgtatttggaatacttctgtaaggagatgtgttcttcttcctattattttttaattaatcatctattaatatcagtattggttgatggatgtccattttatactttgaaaaagatccatgctacatcattcatttaattgttcaaagcaccacagctttattaggtgctgggagctcattttgtttgaatcctgcatccttacagcacacctcatatttttgtttttgaacacttgcgtatttcctggtattaccataaattctaagcttgttttctttattacctttttttacatagaatcaaccacttttataaagattgcatgtttctgatgttaaagaatagtattaaaataaaacattgtgatactggctctgtgtgttgttaatgtggtatgagtacttctagaacctctcaaacaatggtcccagtaaacgtgcatgtttatgtgaacccaagtttatggactcattgaaactatttatgtatctaatcttctgtaaccatatcacattaaaaatgagaacacactggtctctccacccaactatgttagcacatgaacctttctagtcttctttctttgcgtttccataaccacccactgcaaagtgagaaaccccattccaccatatgtaattttattacttagctgcacagtttcaggacacatgcatagcagtatcaaaagtgtaaagctgtgcccttgatggaaccatgtttatctactagaatagagtgcttatgtgcagattttttacacattaaacttatagaatttcttcgttttctgagttgcttaggtcagcaacttcattttccacattcttcaatgaagtcatttcaattacattgtataatttcatttatttgaaattccataaatgcttagactatagtcaagtaaacagacagaggatgttccaggaatttagagagtgggtataaaataagcaaaaaaaaaaatgtggtgtttaggaaaaataaaactatttttagtgatatgcaatggttgagatatgatataactaatttgtctaagctaataattttgtgatagaaaatataaacttaaacatattcaattaaaaaaattcagcagttcattaaccccaggattaaatgcagactgtataaaattatccaataacttatttggtgagagtggggatgttatgagatacatgcaacaaagaatgaagtaattttccccatttgcatatacaatgtttccattcactgaagaccttttattttaaaaaaatcaattttctaccttacccttgtttttaattcccgacaagcaaataacccaaaggattcttttctttcattggttgagaaagattttcccctaacttcagcttagttcaggcatacgctgacctgaatgggcatttaccctcagatgggtacacacctgtcaatatgtggactcttctgtcagacagacacagcttcactcatgtggattcttccctcaaacacaaatgtccccacatggactatttcctcagactaccacatatgtccttacatttacactttcctcagaaaacagacatttcctcatgtggactcttgtctcagataagcaaacatgtctcagtgtgaatgaagtcttcactcagataagtacacatatttcaacactgactgtttcctgacacaagcacatatgtccaatgttaaactgtcttgcgacaaaatgatctcaagataatgataattataaactccaaccctgaaaatctgtagatctgcattttgtctattgtaacataacttcatctcattgtcagaaacagtcgtttgcagctataaatgcactgattacagtcagatttccattttctctggaaatgtatttcttatgttcttactggactaatttgttgataatgtttgctcacatgaagatacctgaacagtgtccacattagagaataaaaaagagcaatgggcagattaaccctgtgcatccagacgcaggaaccatttgactctgccttccctgaaatggagacacagaggatggatgagcaacgctgagtggtgcacctacgaccacaaagagaaagacctggaaatatgtcccatcccctcctcatgaaaggcagctcatcccctgttccttcaggccctggtgaggagccaccccatatctgtgtctttcttcagtgttcacactatggagtctgcactgatctgggtttcccttctcatcaccctccatattagtgtcccttgtaaatcaggtccagctgtggctgctcatagggttgttctcagtctatttcctctgtgttcatagaagtcctgtatgaagttcagtggtggagtcagaggggtaaacgtagtacagcccagtggttcactgagactttcttgcaaagcgtctggattcaccttttctggcaacagcctgaggttggtccagcaggcttcacaacagggattgtggtggctggcaacagtgagtcaacaagtgggagtgctcaggtttactcttcatgagtacaaataaattaactggtccagcgacaccctttcacgtgcactctaccttacaatgactaacctgaaagccaaggacaaggttgtgtaatactgtgagcttcacaggagagagattatctgcacaagcccagacacaaaaatctgcagggagacaggagggaactgcatggtagatgctgctcagaagcaccagggggcactcaacacaagggggcgctcaggacaccaggcggcactcaggatacagctgggggcgctcaggacaccagggggccactcaggatacagcggggggtgctcggatccaccagggggctcttagggcaccagggggcattcaggaccactagggagccctcagggccaccaggaggacctcaggacacgtcagcgtgctggagccaccgggggggggggggggggggggctctgaggaaaccaagagacgctctgaaccgcgaggggaaggaagctcaggaccacgaggggacgctcaggacaccagggggcgctcagaacaccagtgggcactcagaacaggaagaatctcttagaaagcagctccacatcaggatcctgggaggctgtggagaaatgggaacactcataaactgttgagcatttaaattagtacaaccactatagagaacagcttagagtttcctcaaaacctgacgatagagttatcatataatctggcaatcctgctgctggtatatacccctaaaaaggaaatgtttttgcactaccatgtctgtaatagcactgttaaagcactgttcacaacagtcaaaatttgaaagcaacctatgtgccgattaacacatgaatggataaataaaatatggtacatataggatatgggttaatatcatgccataaaaaaatgagattttgtcatttgcagcaacatggatgaaactggagttcactatattaattgaaatgagcccagaacagaaagacaaaattcacatgttcccatttatttgtgaaagctaaaaattaagacaattgaactcgtggagatagagagggaaaagatggtcaccagaagctgggaaggtctatggtagatggggattattggggatggtcaatgggtagaaaaagtgattagaaataataaataacacataatattttataacacaacagaataaatatagtcctcaataacttaattatagattttaaaataaaagtttatgaatggaatgttggtaactctattaatgcttgagaggatgtataaaatattcttcatgatgtgattattatgcagttcatgctgattcagagtcctgtaccccataaatatatacgtgtactatttacccacaaagattcaaaattaaagtgtttatgtgtctaatattctgtaactatattacattaaacctaagaacacactggcttttacacactactgtcatccacatagacattcatagccttctttctctgcttgtccataatcccacactccaaaatgagaaaacctctcctaagatatgacattaatttacttagtgcacaatttcaaaatacacaaatcgtctttaggattgttaaactgacccttgttgaaaatatgtttatcaactagaatacagtgcttatgtggagcttatttatgttttacaatcgcaacatcaaattctttccaagtctcttgtgtccaatatttatgccccaccttcttcagtgtaattagttcaaaccttctgattgttgttagatatttttatacagctgtctttttttgtgtttcatgacctgctaaatactttcgaagttgtatgcatttaggttaacactttgtctcaaaaaatatgagttttcagaaattctttatgccatgtgtccatcattgaatatcataaaaatagtttcattggccgggcgtgttggctcacgcctgtaatcccagcactttgggaggccgaggtgggcggatcacgaggtcaggagatcgagaccatcctggctaacacaatgaaaccccatctctactaaaaatacaaaaattagccgggagtggtggtgggcacctgtagtcccaactactcgggaggctgaggcaggagaatggcgcgaacccgggaggcagagcttgctatgagcagagatcgcgccactgcactccagcctgggtgacagagcaaggctccctctcaaaaaaaaaaaatagtttcattgggattaaaaagtgtgtgtttcacctaatccacacaccttctccccaccttcctgggaaacctcaaatgtttactggatctatatttttatctttggcaaaacagcctggctgttctcttagcctcttttgcaaagcatcaagattcaccttcactgactacagcataaattgagcccagatggctgggggacagaggctggagtgggtggtaacagtgattgattcaagtggaagttctcagtgatattctgcatcagcataatgaagattcacaattcccagggacaccaattaccagcacagtctcccttaaaataatctacttggaagctgagggggctctcacaggggtaggcagtgtattactgtgagagacacagcgagggacatttctgtgagtccagacagaaacctccctgcagggagacaagagaggactttgtgataaatggtgcttaggacaccagggggcactcaggacagcagagggtgctcagcagagcagggtgcgctcaggacatggtgtgtgttgtagggggcattgctcaggatcaccagggggctctcagaaccaccaagggtcactcaggaaaccagggggcgctctggacaccagggggagctcagaaccaccaggtggtactcaggacaccagggggcgctcatgaccagcagggcatgcttgggactctatgaggcatgcaggagaagcaggccatgctcagggcaacagggggctcacagaaccacctgggggtgctcagaaccactaggggtcactcaggacagcagcaggcattcgggacaccatggggccctcaggacctgcagggggcgctcagaacaccaggggaaggtcagaaagacctgggggcacttggaaccaccagggggcactcaggacagcagggggcgctcaggaccactagggggcgctcaggaccagcagggggtgctcaggacaccatggggcatgcaggagcagcaggctgttctcagggtaacagggggctctcagaacaacctgggggtgctcagaaccaccagggggcacccaggacatcatgcggccctcagaacctgcagggggcgctcaggacaccaggggaagctcagaatgacctgggggcactcagaaccaccaggtggcactcaggacagcagggggcgctcagtaccacggggtgcactcaggacagcagggggcgctcaagacacagtgggatgctcaggaccagcaagaggcactcaggaccaccagggggcactcgggggagcaggaggcactcaggacaccagaagaggatcagaaccactcgggggcactcagaaccaccagggggcgctcaggacactgggggcattcagaaccaccagcaggttctcaggacacccagggacactcaggacagcaggggcgctcaggacaccagggagggtgctcaggacaccagggggctctcagaaacacctgggggtgaccaggactagcaggggcatgcaggacaccagggaaggggctcaggacaccagggggtgctcaggacagcagggggcacagaatcacgagggtgcattccagaccaccaagaaatactcaggaccaggggtaacagaaacacctaaagacacacaagaccaccaggaggcactcacgactgctcagagtgctgaggacaccagagcttcaggacgaacagggggtgctcaggccaccatggggggctcaggaaccaccagggtatcactcagacaacagggttctcttaggaggcagctccacatcaggtgcctggggagggtcagatttgcttttagaccttgctgatccctgatctggtcaagcgaaaaccttcccaggatctctcaccatttcttccttgtaacccatggtttctttcacgtacaaatcattaacttagaacaggaattcaattcaacttttaaccctccatatttttagaataacactagcagtaattgaatgttttgataataagaaattgcgtatgcctaattcaagctctggttccatgcatgggtttttgttttctttgctgtgtcagtcacactatagtcaatgcttttctaccaataactcagcataggcatagtgttaagtttctttttctttcatctgccttttgtggagatgaaacaccactttcagggctcagttcccccaccttggttgggttctggtgttctgctcctcacactatttctccaccttcccttcttctgtccaagccttttgtcttcctcagtctcctagcaaggaagcaacaagtccctttattttgcctcttccatgtctggtgaatctgttcacttctcttcatgatcattgaagtcaaccaagtttaggaggataacagttctccttagaatatgctcatctacctgcagactctctgccttcctcacccctttctagggtcctgcagacgtcacctccaccacatccactcctttacctaagtaccacagaatagtctctgcagctcctgctgctctctgtgtgctcagcactaaggctcactagtgctttgatgatgaagttcaaatccctaatgtgttagcaattctcagacaaccctgccattgtgagtgaattttggggatcctgggctgttttcagttccatattactggatgttctctagcatccaggagtactggcaaattaacatctagaatttgtattgcaaatttatacaaaatcaattgtagaaaaaaatcctaatttctaaataagaacatttatcctgacttttataaaaatacaatgtgaatttaaccaataatataatacaattttaaaagtgatgtttatcttatatgttattaagtttattaataattggctggtattttaaaagtatactgatagcaaaactgacaaataaaccaacacagattctatacataaataccattgtccatttaattaatgtgtttggccagaatggcatttacattcagttctaccgaggcatgtctttaaatgtatttgtttattggcattaggatagagaaacactcacacagaaaagcattatttgggactactacattatactataaaagagagtaaattcaattcaattaaataaataaaataaattgaataaattcatgttttggtttggtatttggataggtgttcatttttctattttcacatgtatatttcagtactgggcatatgtgtgtatgtgtatgcatgattatgaacttctaaaatacattactcccctatatagttatataaattgaagttttgcatgttaagagacagtagaaaccacaaaaggggaaaagttatagaaagcatttggaatacctagggcctacagaggagtcatattaaaatgaaaaataatagctgaatattttggaaatgacacatgcagtaagaaatgtctatatgaaattaacaggatattggcacaaaatttagaaaaaaataacatgattatgcccactattggtctatgatatgcctataataataacacattttatttatcatagaaaataaaataattatacaaatagcactctcattgcttgattttagtattaggtacttcacttgttttttgatagaaatgaacatttatcattttttgatgataatttctatattatcttctaaaattaatgtccataattatgattgacatgttataatttccagaaacctattttatcaattgtgacttgtatttttatgatagtatttttacattcatatacatttgattcagaataattgtagattaacaaaataatcataaagttaatacagtgtgttcccatctctaagtatctcctaagatgaacatcctccacaatcataaaacatggtgttaacacatttacgttgataactttaaatatattcaataccaggccttatttgagatttaccatttttccaataatttcttttgtaaaccaaaaataaaattctaagcttcctcatccaactaaatgaactcaactatcagccaaagatattttaaagtaaactttaaaacttgttccagcaataatgggaaaatgagaggtcagatatgcctcattatgtcctcctgtctttggaattaaggcacctgtggccagcattaacactaaaacaaagacccttagactgacaaaactatattttttctataaacactcttcatagtgaaaagataccaaattacaatatgattctagtatagcatcacatgacaaacagcaacgcctaaaaatctattctctatgaagtctgctacctggaggtttcatctacataataacaattatgagctccacaatctcgtatcttaatccagatactcatttgtaatgagtgtgtaacatttcaaccaattgccaatcagaaaatgtttgaattcacgtatgtcctggaagcacttgagtaagcttattctgcctttctggactgtgtaaatggtatagctcacatgtgttgattcatatctgtctataccttctgtaccactaaaatgtataaaatcaagctgtaaccaaaccattttggatacgtgtactcaggaactcctggagttgtgtcatgggccttggccactcatatatatctcagaataaactgtcttaaataattcacggtttgtctcttttagtggatactgtctttttcttttttctttctttttttttttttgttttttgagacaggatctctccttgctgttgtttttgtatatttgtttgttcccagattccaacaaggtcatcactttgaattgagttgtcatgttcctttattctcttctggcaaatcacagtttgtgtttctgaagttttctagtttaatattttatagataaaaatttatcaagtagattgatagtcctatttaggacacttatatctttacttatttgaacactgcttgtcaatcacaatgagtgttgatttctccgtcttctatacatgttattatattttctgttccaaatccattcattaagtatttttaaatgtattttggaaagttgatcctattactttatgtaatatttctcttgagtcccaaaggtcttctttttatgattattttgtcttaaattaacatagctatttaagttttcttttggtttatattttcatgatatatatttttccatctttattttttctatttctgaatatgtaaagaaattttcttggagataaataatagttgggtgtttttttttagtcaactatgaagaattattttttaatgatgttactcttttgtccaattattcttgttttaatttgtatttttacaataaagtttattgtttctattaacttattatttaatcattctgtgactaacattttatgggttaccataaggataacaataaaaagtttctataattgaattctaattcaaaaatttaattcccattcatatatactacagagatattataactctatatatctaattcctctttcctatcccatgttttttattttctgtttgtatctatacatgctataaaatgtaatattggcatttttataattgctttccacacttacataatgaacaaaatacaaatgcaaaaactgtatttcaacctcatttttccattgttgaccatcttctttgtgcatattttcaggtgttggatctatgtcacatggctattcatctagagactctgctaaaatgtctacggaaagattcatgtgctggaaataggttttctcaggttttattggtccgacagaatatttatttgtctttgatttattgtctccagagacaatgtcaagaatatgctatatctgcaaatgggcgatctgtaaaccaagaacacatcagtatatcactgtgcaagaggagcacatgagggaaagccagtgtgtgcccagacataaacctccagggacacatgggcgaattagctgcagggggagcttgggacccactgatgagagtcgaccccagagggaggtgcacagaaaggttaagaactgctttcctgccagtatatttttaaaaaaacaaaacttccatctgacagtttctccagagaacttccctaattttagcattctgtgcctaccaatgccatcactaaataggtttttaaaaaatattgtaatatgaggacatattctcatatggacaaaacgcaaattgacatttacagaaatgaaaagtcctcaaccgtggtcgctagaattggagacctgtagcagctcagtgggacctgttgagtcttctccaatcacactcagaacagagaccttagtgcatctccctgactagaataccctttaggtatcgagataacagcctaaagatggtaaatcatgagagtctcagatgtccagatgacagacatgggtctatgggaatagtactgtaactgacagtaactaattttccataccgccagatgtcctattcaggagcagctacagcagtcatgcctaggtgtgaagatcacacactgacctcacccatgctgtctctggccacttcatcacaaccaatgcttaatattggacgtggatctgccagtccccggggaatgggttgaatggataaaatgcatggctagtgctgctggaaacccattcctactgtggcaaatggcagcatctcttttaaaaggctaaataatattctattctgtatacataccacattgccattatcctttttgtttgtttgtttgtttgtttgtttgagatggagattcactcactctgtcacccaggctggagtgcagtggcgtgatttcaggtcactgtaatccctgccttctgggctcaaacaattctcctgcttcagcctccggagtatctgggattacaggcatgcaccaccactcccggttaatttttgtattttttgtagagatggtgtttcaacatgtttgtcaggctggtctcaaactgtgacctcaaaaaatcaacccacctcagtctttgaaagtgctcagattgcaggcgtgagccatagcatccagccacttttctgcatttttttttcattgacacttagattatttcaatatcttggctcttgtgaatagcgctgcagtaaacataggagtgcagatatctccacaaggtggtgattttttctatcttgggtatattcccagaagggaggttgctggtcatacagtagttctagttctaattgatttaagatcatcttactgctttccatagtggtggagattcagattagtgtagctattatgaaaaacagtttcaatgttggggagtgactgaaaaacaaataatgcttgattatggttctccatgagagtctctaataaacctaatggaagttcagaaagtttcctcaccttgttaagaaaacatttgttttgctcataattctctttcagcccagagttggcagtatgtagaatggaggctgttggctcgtttaaggattctgaagcaaagaacagtaatgagagatgacaatagagaaaaagagagagaaggagatgctgaatcatcatggactcattccacctgcaagcctttcactgtaaagctctattgctaaggatcctgaggattctatactcccatgaatgcataatcaggatccctcaattcaaattttattggcagagacacactgggacccctctaggatatgtgccctacattactggctcaacatgccgcactgagaatcctgcattaactggctttatttgtagtggtgagtaaagttcacagttttcagtcatccatgaaggggagattttttttttggttcatataatttttgcagaaattcaggtattagtgaaaaccaataagattgaggatttctggatattttagcaacactagggatgatggccattttgtagaaaaatttacatttttcaaactgacctacatgttaggctgacataaaaaaatccaactacacagcaatattatggcaacacttcaatacacttggtggtgaaatacctcagaaatgaaatgttacacagattaattgagttaatgaaactgttctgaactatgataaacatttgtttgcacccagatttgcttgaattcaatttgattattatattcagctgctgccctaacttatttgaatgagtaagttactatttaaatgaccctgtctgtttgatttgttatatatacatctgaattaaagaacttaaaggacaggtttgtgctattattagaaagagatattctggggggcccttgtttgctccaggaacctccacatttttaacttagaattaatataaattccttgtgaattgggtcagtcttcaagagacctaatctctaaaacatatacccagaaccttctccagacagaccatccagaggaagaatctttccagtaatttatctccatggtggaaggtccttcattgtcatgcagaaccctctgcccaggcttcctttattatgcaaataattaatattagccagtagggtaagattaaacaaaaatagaccggagatgctgcagacagggaaggaagtaatagaccaaaatcagctttattaatggagactgcttttaaaggtcacagccaagaaatggaagaggataaaatcaatgagatgaaattgtaagaaactttgtccagttcaatcaagccatcagcaaccagatagcttcagtggacattatgtgtattacaacatggctaaagattctagattacacaaggattttctctatagtttgcatattgacaaagtggaagatggcatgggagatctggtggcaaacacttaaggtgcaataatagctatttttcctatatcatgacaatacatggcatgcctctatattcatttcattccaaagaagtccttcagaaggtcaagtaaatggagaatcttacatgcatggcccaatgcagtctccaagaagtgattttgaatgtacagctacaccagttactgcaatgctgtgtctgaccttcccagcatctaagatattcatgagtatcaagggggagatgggtctaggtgaaagaaaaggaagtagtcattcttgagaagaatggtatggccaagataagcctgaactctctatcttcttccatttgctgcagcttcttattgcccgattgaggtcactcgcccatggtggtaatgacatcagcaatgcagtacattctctggtggctttgtatctggtttataatgtgggagatgtttcctcaagagtggtgacgccagtatggcttctgctctctggtggtgatggcatctgcattagcctaggggtttggagaaagagagttattcagaccatggggaagaatctgacactgtcacaccgcctggtgccttcagtactggactggctcctgccctcattggtgattgcatcaaatattggcctcctcatcagtacaacacattgtaacagtcttctttgtacctggctttaatccaagaaagttgttgacttatggctctttcattatattgtatggcctggtttgtcacaatctttatttctggaattatcagtgctgccatcttggctgacttcaagtatgtcatcctcacagtttgaagctgcttgacattaacatttttgtcaatgtttgggactaccttacatattccggctctctgaaagaatgattagcgttagggaaggctgacactaaatttgggagccagagaaggaaagtgttactgtgctacagttgttttgtgataaatatggttaggttaaaaatgagctatgtaatgtagccaggtttccactgagttccactttgcttcttggctgtctattcctgtgcaagtttctatatattttgtatcaggcttcaattccattatgttttaaatgttgtctctaaagataaacaaagattttttaaaactaccaaacatgcagccatttgacagagtgtgcgctgcatttttagtctcaccatcttctgccctaacaagcaccgaatctaacaacacaaatatcatggaagctttccttggagtagctcctgtaattagaatcattggtattctgccctcctgtcagtggtggaacgttctattggcacatgtggaaacctcttagagggatgagtttctttgaacacaataaaattttaagttaggaataacttatttgaaagcaattcattgaaggttattgctaagaagaagttagaagcagtcttttggcagtcatttcttcaagatgtatggcagcgtaggatggatatatgaagtggaataggaacttgggctagttaaatggaatagcctccaatgttaatctgtttaccttttaactgaatgaaaaagcctatagttgtaagaaaagaaaaaacaatacagtgctcccctgtcccacacattaccttctcatcagtataattaatctacattgaagatgagtgtggtaatgcagagactctatttagggaggagaacatagggaaatgcaaagacaatgggagaaaaaagtcaaggacagtagagcaattcgaagcctctgataccaatagcttgaggaccaaggtcatgactcaacttttggtggacacagagtcacttctcttagggaaactgcagtattctaaggtgacaatatgcaaacatgatgaatgtacacttgctacatctccaattgtgtactgttttccttacttctgtttgaagaagggagtcgtatactcaggtctagtatcagtgtagggggtgctccctatttgcaagatactagaaaaaaatggtaagttagaggcccattgttataaaaatctactagctccgtgtcagtgtgtgtgtgtgtgtctgtgtgaatgtgtgtgttattgaaacagaagacatggagtgggcattgatccacaagcatagatacctacaggcactcttagatgtaatatttaactgcaggagtctaaataaaataaaggattcgccaaaagtcttgaaaattttggtgctgactatgtgtccactgattcaatgcatcctgagttccagggaagggaatccccaattctttcatagagttgactgggtctcctaaggtcaatgttttcaaacgatggtgtcagtaacatatggtgtcactgaaagagcattttaaactagggcatggccacttcctatagccctagagacactgagaggtaaatgccctgtgagcccagatggaaacctccatgcagggcagaggcagtgctgcagggggcgcccaggacccacccagaacaggctccagccccagagctggtgcacaggaggctgcagagggagtctctcacaaggactgagtgttactttactgaaaaccaaaaaattataacatgctaaataagaattttatgaggactatttatgtatatttaaataccctaatgtatttagaaataaataaatcaataattcagtgagaaccacatttaaaaggcaaaattattactgctttgagatattttactagtaaaaagataaagaacaagaatggttttattaaataaaaatgtacacatttcagagacacactggtcccgagagtaagactgcggagagcaaatcctggaaagggcggaggctgtccatgtgccatgggaacttgggctcacgctgagaaccatgtccggtgtgagtcagcttcttagcggagaaattctcccttcacaaatttccgggcatataaagggaaacatgtcattaaataagaatgaagatttgtacctcagcatcccacagttgtgtggtccatgtgagatctattttctctttctcgtgctggatcaggtgtaatgctatgaagtagtagtcctcatgaatacgcaaatcacctgaggtgaacactacagataactctgtgccctgagagcatcacccaataaccacatccctcctctagagaagcccctgagagcacagctcctcaccatggagtggacttggaggatcctaattttggtggtcgtagctgcaggtaggataattctcagtccccaggactaaggtgactgggtccagtcaaagggggttttatccactcccgtgtcctctccacaggtgcccagtcccaggtgcagctggtgcagtctggggatgagatgaagaaggctggggcatcagtgaaagtctcctgcaagacttgtggatacacctaccttcaccagttactctatgcactaggtgcgccaggcccatgcacaagggcttgagtggatgggaaggatgtgccctagtgatggcagcataagctacgcagagaagttccagggcagagtcaccatgaccagggacacatccacgagcacagcctacatggagctgagcagcctgagatctgaagacacggccatgtattactgtgggagagacacagtgtgaaaactcatatcctgagagtgtcagtaaccctgagggaggaagcagctgtcccagttttcaggatatgacaggatttatggggtttaatgttgtttagaaaataggttatataattgaggaaaagaagaaatagaaagatatatgcattctaattatataagaaatattcttttcaaatctcaccctataagcaaaattaacagagtgggaaaagcagcaatcaatcaagctgaaacaaacattcccatggaggatttgtgggggcagacgttttaaaattgaatgggtaaatcatttagggcaagattgcttgatccaatactaagactaaatgtaatttctgagaaactgcctatctttttatattaaatttattataaagcagttttagggttacagcagatgtaaaaagtagaaacagagagctcccatgtacccctgcccaacacatacacagccctccccatgatcagctccctgaaacagagtcctaactgcgttatgtgggatgaacctacatggacacactagttctttcctttcctggtggtcccccggtataacaaacataaattattttgaagcgccacgggttcttcaagtgggttactgggaatgatgccagttagagggaaagtgggtggggccattcctttttgcactctttcttcaggaatccataaaatgcacattgacttagagctcatctgactcctggttttctatgccccttcccagagggtaagttctccaagcgtgggaggaaggtccatttcactaaaaggccagaagcttctggtgaattccataatgcacaaccttttttgagaagtggagactttggacatgagggatttcatgtatgatgccctggagttggattaaatacagcataagctcttgggggtatttctggaggaggccctttctgcaggaaattagaatacatgcaggggatccaggcaggaagaatcacttcctttgacatcgtttggcacctgaaggaagccctctcagcattgggcactggtcccacttgctggctgcactttagccagaggcctgcgcctgatcattcttgcagtgggagagccttactggggtcacaggttataaaaatgcctgtcactctccagctgagcaagtccatatgcgtgcttgtcagtgtcaaccccatgatgagtgcactctggaaggtgacaatctgcacacaaacctcctcccactagttacccactactatacactactttaaaaattaatacttataattatataatataatatatattatataactatataaaaatgattatgatgatgacatttggctgttgcaatttctttaggatctctgcatctctcttcactagacatgtaagtcttcaatttcttgtccctgtgacacgttgcccagtgtttgaagtcaaagttgtgccgagtacctgagtatgtcctcagagaagatctcagcaatggttgattgttctacagggacttttccaggagacctgtcccagggagtcatgaggttcctctggaaatactagcccctgcaggagatgagacggaagcaggagaatcaagagcaggtgacaacattgtcagcaggaggctccagatgctttccagacacagtgaaccttcacccacttgccacactgcacagggagtttgcaggggcacaatgaggtgcctgtctgtgggatgttgggggacaaagtgaaaacctgagctcagcatccaggaaagtatgaactccagccctgataatatgtagcctgtgttttctttccttctacgtgaacactttgccttattgttaagaacagaggattctagctctaagtgcacggattgcagacagctatgactttcctccgggccaggctcctcttaatgtacttcttggacttatttgttgatgaaggttggttccattaaataccaaaacagggttcatattaaagaaaaatgaaagagtgacaggtagattaaacccgagcatccagttccagtgattctttgaccctgccttcccaaaaatctccgatatgaacaatgacaatgccctccatggttagattatattataaactagattgagctagagtgtttggtgtattaagtcactattttttttagcttccctgttagttttttgtttgtgtgttagcatttactttaaaattctattaatcagttctctagtaggtagaaattcatctgagagtttcttctcttgttgtccactttgataagatttccagaagacataagaaccctttttgtttgcaaaaatattccaaagttttacactaactaggaacatatatacttaattctaatttttaatttgtttaaaagctctaataagtgcactgttttctgccttttgagttaatttcacaacacataggagaatatatcctaaatgaaagtttgtgctaataatacaaattattggtaaataacctctgttttaattattgaggtattatccattaatatataatcttaaattgatgttctcaatgggactcttacctaaagaatatacaaaatattttcctgatcttgacataaaatagatgtgaacacattcttagtattcagccatgtctcctgtctatcacattatgaaccacatgctaactttgatttacttgggacttgttctaatttcaaactagttatttttttatcttcatgcagctggattattatgtgtggctattttatcagagaatgataaagacaattttaacaattttcactgcaggcacgtctaggcaagccccctgtgcacaatgaccttcgtgggttggacattctatggggactctcccctgtctgcctaggagagttatctgcctcctccctctatcattttcctctttgaataagcgcatctaactgccgttagaatacagacaaaggccaaccttaactgctcccagctgacaggggatgctgttttgggaagatctcccttgaggtctgtctaagggacctagtaaaagggagccattatcccaggcttcacttggatgaccatttggagttgatgcctgaaggtgagaagagacaaaccaggttattagaagacatgtatcaaaaccaaacaaggtggtaaggacagtttgaaaaaaaattccaaggctgctgacacacccagataactggcggctgtagttatgcctgctaagatttgggtgcatggggcttggctttcgttagctccctaggacttattttcccaaacaaagaaacctccgggttagggggaccctatttattccagtcacctggcatgatttgcaggataattgctcagaattaaaatattcgtccagatgtttatatagcccatgcctgtgtttcttctgagctgcagccagagatcattggttcgttcacagcgataagcagagttagtctaaaatggaggcaaatacttaaaacaattggaagagactgtaatttaaagacaaatgtatgatatgttttgaaacataatttttctctctccagttctgatttttgtcagaaactaatcattataggactgagtgatttgcaaaataaactttagtcttgtggttggtctgatcatttgcataaagtggagcaataataattaataataattctgtaggaaaagcctgcaagcaccaggagcttcacagtctaacagtatgagcacatgcatcctccagcaactcactgaatattttcaagtcagctggttcttagcttaaataacatccagttggtatctgtcccaggaacactaatatatggttctctctgcaggcccctctcgccacagatttaaggtttatttttttcctctgtgatatcaactcagatatgttgaaggtttttcccacatttgtggtttttcaggtttgttgttaataaggtcagaataagatcatagtttactcatttttttacattcccatgctgagtagctacttttctctataaaatccattagctgagagaaaaaataacattttcctaacggtgaacaattaaatagtttgacatatatttgtgtaccagtatataatgcagcttcgaatcaaggtgtgcctcaatcataaaaaacatggctaaattctcaaagaattgtgctgagtgaaagaagctaaggaattaagagtaaattttatataattcattgtagaaatattagaagatgccactaccataaattaaaatgaagaagacttaaatttttctgagaaaatggtgttgggaatgatgcggatgtgatttaagtttcagaggaataaggaaaaagatttagggattaatttaattattcaaaagttgattgaagtgccgagtgaatggctgcaaacatagctctacatttttcaaatcattccctataaatctgaattaattatttatttattatacttgaataaagcaataacgaagaaataaatgaatatttttgctaaaatggagcaataaaaagactgatattgacagaagaaatatgactgacttctgaaaacacacatgaaccatggttctctctgcatatttaggtgaattacagaaagttgtcataacagatggggaatcctgcagacttcactaggcatggtccacgctgccctggagttgtctcaggggagctgcctcctccggtgattagagcacaggcccagataataggattacatttttttagatgtgtaaacttagacgcactgcacagctgctgtattctctatgtaaattatcttctgtaaaatacaacattaaaggctgcattaaatatattgtgtaaatatgtaaaaataaaatcagattatgagagctaaatgttaatcaaggcacaatcacataatataaaattatattttcctgaatgatggaattactaccaatctcccccaggagacttcatctgcactgggcccggcctctcctcagatgtcccatcacagagcttgctatataatgggggacatgcaaatagggccctccctctgctgatgaaaaccagcccagccctgaccctgcagctctgggagaggagcccagcactgggattccgaggtgtttccattcagtgatctgcactgaacacagaggactcgccatggagtttgggctgagctgggttttccttgttgctattttaaaaggtgattcatggagaactagagatattgagtgtgagtgaacacgagtgagagaaacagtggatatgtgtggcagtttctaaccaatgtctctgtgtttgcaggtgtccagtgtgaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagctgtggtagcacatactacgcagactccgtgaagggccgattcaccatctccagagacaattccaagaacacgctgtatcttcaaatgaacagcctgagagctgaggacacggctgtgtattactgtgcgagagacacagtgaggggaggccattgtgcgcccagacacaaacctccctgcaggaacgttgggggaatcagcggcagggggcgctcaggagccactgatcagagtcagccccggaggcaggtgcaggtggaggctgtttcctgtcaggatgtgggacttcatcttcttccaacagtttctctaatgaacctctctaattttagaattctgtggttcctaatgtcatctctacatattttcaaaagatcattttaacatgaggacataacctctcatgcaccaaatgcacattgatgcttacaaagatgaaaagttctcaaccattgtgaccaggatcgcagtcctgaggaagctcacgggtgtctgatgagtctcctccattcaggcccaggacagaaacctcagagggattcctggaccagaacggcagggattctgatcacagccaatagagaggctgggccagggtcagtgtcctgtagaagctcacagggttcaagtctgacccttctcctgaccctaaagccaatgagcatcagcactgatctggtgctgcttttgctcccaatacatgttctttctttggagtgtttgttctcccttttttatttgcttttctttcttcctgaaaaagaaacacatggtctctgtgatctacactccagggctcaaggcattttcttagaactcaggcaaggctcaggcttggctactccagccacgtgggagaggctgacgggatttccttctctctccatattctcagggccctcctctgtgttgtgtgtagactcatctgggaatgcaattggctgttagtagtgaaggggatgaactcatttgatcaaaatgggatgtggatgtggaattaaccctgttctatgcacactgtcagagtcatcttcttcagaagtagtgttagaaagagcttgtgaaatttatcggcaacaaaatggatccccttgtgttaaaaccctaatgaatgaagctggggaaggccatgaaggagggttctaacacatattcctgataagatgaactgtcataaatagactctgcacagccacaaccttttacacggagaccaccacagccttaaaagctttatttctccaagtaaatctgccctgcaactgcctgttcaaccttacaccgctgtctcaaaatagctgctgtcaccctcctcatttttccttaattctcttttttattattttttaaattatactttaagttctagggtacatgtgctcaaagtgcaggtttgttacatatgtatacatgtgccatgttggtttgctgcccccattaactcatcatttacattaggtatttctcctaatgctatccctcccccttcccgccaccccatgacaggccccggtgtgtgatgtttcccaccctgtgtccacatgttctccttaattttttatcttccttttcctacctgaatgtacccatacatattttaattgaaatgcacatcctggaaaaaatattattatactttagagtctctttctgtctgttattcaggttgacaagctgcagacggacatgccacatttctgtgagacatagaggatgacagtttttggagatgtctaggaatctccaatgtccataagatcagccatcaataaatgcagactggaggtcccagagagaagtgaagctgctgaatcgccgtggaatttcattttctccagttctgctctgatggaatcaggcccaccaattttatcaatgacaatctacctaacttagagtcaattgataacagtattaatatcatctattaagtaaattcataactatatatatagtaagagagggagagagagagacagagtctcgctctgttacccagggtggagtgcaatgtcatgatcttggctcactgcaacctctgtctccggagttcaagcagttctcctgtctcagcctcctcccagtatctggaaatacaggcacacaccaccatatccggctaatttttgtatttttagtagagacagtgtttcaccacattggtcaggctggtctcaaactactgacctcaggtgatccacccgcctcagcctcccaaagtgctgggattacaggtgcaggccaccaagtccggctaatttttgtatttttagtagagatggggtttcacaatattggtctggctggtctcaaactcatgatctcagcctcccaaagtgctgggattacagatgtgagctactgtgcccggccaacatcacctatattaatgttggattgaataactacaaagtctaccctaaccaagtgtaccatcaaacttaccattacccagagggaagaatctttaacatgagatcgatttcttaagtgttttaaggtgcaaaactgaccactgtacaatcacccaattatgattttgctaatgagttgtagaagttgcatgtacattttggatattaacactttttcagatgcacagcttataattatattctctctttctgtgggttggaaattttttccatgctttgcagcatctttttgctgtgatgtagttctgcttgttcaattctgcttttattgtctgtgcctttaatttgaaatataggaaatcattcttaatattattttctgggtgggggaaattttacaattgcaagccatacgtttaagtatttcgcccatttagagtaaattttggtatttaatctaaactaaaattcttaattctttgcatgtgtagattcagtttttctaacaccctcttagggagagactttgattcatccattgtatattgttagttctcatgctgaaaatcagtttgctgtagatatattagtttatttctgagctctctctatatattaatgccaataccatcctgttattatttatggatcaaaatgtgcatatttgtgaatttcaaaatgttgttcagaacttgtgcatgccaacaactgtgtttctcctgcactcttgggctagtgaagctctcacagaccctctctctcacctgtgctgtctctgcattctccatcacaaccagtgcttcctgctggagctgcatccatcaccccccgcaaggaagggactggagcgaatcaggtgcacaggtcatgagggagtgcacattccaacccactcctcaagagtccagtcactatctccagatccacatccaaaaaacagtgtttcctgtagctgagctacctgagcaacaagtacacaaccatgaattttaatacaaaagacacaacgaggggaagtcattgtgagcccagatacaaacctccctgcaggggagctcagaaagagcaggaggcactcaggacaccagggaacactctggacacatcaaggcaggtgcaatggggggacaaggggctggagatggggtttggcatcaccatcgtatttcaccactggacacccaccaccgtgtttattctcatgtacctgattctgtgtattattagaaaatggcatttatataaatatatagccatatgtaggtgcatcaagttgtcctctccatcgtatgtggacctttccactaagactctgagtccctgtatttatatgagcacctcataaattatggtcaattttgtgggatctctatcttttctttctcccttcttccctccttctctctcactcacaaactcacacacacgcaaaattctacaacttcagttacgtgatgtgttgaagacagtttataatgtgcagcttttccagtttagctgatgttcatattgctgtgagaataggaacattgtgtttctcagctgtgtatttctctaaggtagcagcaacttttttttttaatttgacattttttaaaaattttattattattatactttaagttttagggtacatgtgcacaaagtgcaggtttgttacatatatatacatgtgccatgttggtgtgctgcacccattaactcgtcatttagcattaggtatatctcctaatgcatttttttaaatacccagatactgaaagcaatccaaatatcaatcagcagcttaatcataaacaaattgtggtaaattcattccctggaaaatcacccactgttacattctggtaccgctggatacgttcaacagcaaaggtaaagtcacaagtacatatgatgagtcaaataagacaaataaatacaagtacacacatacaattcctcttttataaattctataaaattaataccaatctaaagttacataaagaaaaccagtagttgactctgggtgtggtgtgagaaggaaaggtgtaggaacaagaaattacaggaaaacaagaggaaattttgagggcacttgagttttttctgtgttgagaaaggtaatggttatgtcactatttgtcaaattgtgcacattatgggatggctattatttgctaatttcacctcattaaaatatgccaaatttaaacacatataatttggtagaaaataagttagacagagatgaataaaatatatgagaaataaaaaaagaaaatcacagaacgttggcataaggacttcactcatcaaactgaagaaattttaaatttctcaacacagaattaaagatttaattaaagatatatgagaaatcaaagactcctggatatatacatgaataaaccctaagcctacctatatttttagggaaacactagaatacaacaaaataatgtcatgaattcattacataatgggcgttaatcaaaccccaccaggcatgtccagctgtgtcctggggttgattcagggaacaggtgtgtcctgtggttaggagaagtggcaacaagctcacagcatctgttctagttgacaccataaaaaggccaagagatcacaaccaaaatgtagtgtggatgtcacatctgtgagtgccgcacactcccccatgtgaatacgaaaaggttgattacctcttgaggtgtctgctgagagtagagctggtctctcaggaatgtccaaaatggctcgatagagcaagaaaggagactggctcaggttgctataccagtttggtggtggaggcagagtgagggttctcactcacagaaatgggtttgtggggtttgaacctccaaatggcatcaaatgagggagttcttgtgattcctaactagatccacctcgtgtcggaaagaaaggagatgatggaggaatgagccttaagttatcagcagtcagacatcaaaacaagtctgatgacttattctatgtagcaactataaataaatgaataaaaatagatcataatgaattattcataataaatgaataaaaatatgaataaaaatagatcagtgtccaatatgggtggacaaaaaccagtctacacaaaacaagaagactgcttataagaataagcaaatcataatgaaaaggaggaaaatgaggtgaaaggcacggggcaggggattacacaagggtcctgttccaatgtcttgtgtggagacttttcattacctaaaatcatccgcttgttctgaatgtcttaggtgagctgtctgctccaaaacatcagaagcgccagaggattcatgaggatattcagtttaatatcttctatttaaggtgccttacaatagtgtaaaattcttaattattattattataataagagatagggtctcactgtgtcacacaggttggagtgcagtggcacaaccatagctcactgtaaccttgaacctctggctcacacaatccttctttataagcctcctgagtagtgaggacatgagaccaagctgatttttttttaattctttcatagaagtggggtctctctatgttatccaggctggtcttgactactagtcttatgtgattcccctggttttgcttctcaaagtggtagaattatagacatgagccacgatattcggtctgaactgattctttagttgcaaaatatcaacccaagaattgactcccttaatttttttctgcagtgttttggttaatattgcctgataaaatttattctaatttgtttcaacagcagaattcttcctgacgttttttccaatatatgtattttcatggttgaagatcacaaaatactgttaaaacatgacataaaaaggcaaccttaaacttttggtgattggagttagtataatacaaaaattacgtccaatattgtgtaacacacacatgcaaaaagacgaaggtgagcaatgagggtaaactttctaaatgtatgaatcttccagctcccaagtcataggatagtaactgatgtggcctgagggaggtaccagagtgacacagtgctagtggcataactcttggccaagagactttcaatattttattaaagtttttatatttttttaatttaatgatgccatttttttcaacatattcagaatattgtgagtgggattgattctggatgatatgaacaatgggaatgccttctatgtttagataatattaaggaccaggttgagattgaaagtttgtagaaatgatgcaccataacattattctagtttccatactaggttttttctctttgcttgcatttcatttcaaatttatatgtcagatatctagaatgtaataatttatttaaaaatttcttttcttgtagtccattttgatggggcttccagaagatttgaaaacctcttctgcctgcaggacattttaaaattctgcactacagtaaaacatctgtatttaatcatggtcttcggtttattgacaagctctagtaagtgcaataacttctgccttctcggctgattttacatcagatagaggaatttatcttaaatgaaagtttgtactagcaatataaattatgagttaataaccaccacctttattatttaggtgttattcatcaagaaataatgttaaatcaaggctctcaacgggaatattatctaagggatgcacaaaatgttttgctaatctagataaaatagatgtgaacacactcttagtatccagtcatgtctcctgtctgtcacattattaaccacacggtaactttgacttcacttcggactcgttctaattttcaaattcgttacatattaagcttaatgtctctagactattctagcagagtgaagaaagacaagctagactgacaaacaacctaggatgacaaacttcttgtcactctctataagaacttgcttctccctgaacttcagacttgtcgcgtgttttgaaacattaattatcaaaagtattaaagaaaatttgcaaagctacatcttctatgtttatgttattgttgatgcaattgtaaaaaataaggaaaatgtattccttttctatgtgcgtttccaaacttaatagcagatttttagtaagacccagaataataaaaaattcaaatattgttcagctgcttaacagaaaaacaaattatggtaaatttgtttgctagaatgctatccagtatttagaataaataaatgtctgctatacccaactataaggtaaaatatcgaattacatatgttaagtaaaataagccaaacaaataagaatatatactgcattgatttgtataaattttaataaactaaatgactcagccgcaatgtatagctcaggagttgccagagaaatggtagaagaatgaaagggaaaaggaggaagaatgtagaagaacaaaaggaaatgttgagaattctcttgtccaccttgataacaatgatggttacattatatttatcaattgtacaatttaaatatgtgaaagtttattatctgtaaactaaaactctcaaaatttattacaagcaaacaaatggaaacttagacaaagaaggagtgatagaaagatagaaaaaatgtatattaaatttcagaaatacctaagaacttatctgcctgaaccctagttctcaccatatttttaggtgaatgctagaatgcagcaaaatcacacgtgttctcactacagaaagtgggttccacaaccacactaggcacgcccagctctgtcctggagttggctcagggagtaattggggccagtgatgaggagcacaggctcaggtactggggcttactcatccagacgtgagctcttagacacatacttagccctttctccatgcgtggttgacttccacatctgtacatggagaaacaattgactccgacaaaacataatttgcacaaatatgtaaaaataaaataggatgatgaattcaaaaagtttatcacagcataattttataataagacagcatattttctgagtaccatcgttgtcaccaaattcttgcagggcacagtcattttatctgggtactgccttctcgtcaggcttcccaccccagagcttgctatatagtaggagacatgcaaatagggccatctctctgctgaagaaaaccagcccagccctgactctgcagctctgggagaggggccccagccctgggatgcccaggtgtttccacttggtgatcagcactgaacaccgactaccaaccatatgaagtctgggctgagccggtttttccttgttgctattttaaaagataattaatggtgaactagagatacttagtgtgaggggacatgagtgagagaaacagtggatagtgtggcagtttctgatcacgatgtctctggaggtgtccagtgtgaggtgcagctggtggagtctgggggaggcttggtacagcctggggggtccctgagactctcctgtgcagcctctggattcaccttccgttaactactgcatgcagtggatctgccgggctccaggaaaggggctggagtgggtaggtttcactaaaaacaaaactaatcgtggaacaacagaatacgccgcgtctgtgaaaggcagattcaccatctcaagcgatgattccaaaagcatcgcctatctgcaaatgaacagcctgaaaaccgaggacacggccgtgtattactataccagagacacagtgaggggaggtcagtgtgagcccagacacaaacctccctgcaggggcgcgcggggccaccgggggcggtcaaaacccactgaggacaggacaggtcccaggagcagatgctggggtagttttcctttctcatcagctggagaagtcaggtttgtatttgcaggactctgcagcattctaggctgtgacattttatcacttgtatttattatgactttattatcgttagtatttaaattttagtaattattaaaattatatataagtacattttaaatatgtactttgaagatataaatattcctaattatttgcactgattcttccagagttttattaacatttgttgacatcagcaactacatagctatagggacataaatttataaacatagaaatatgtatcggcaagacatggtggtctgtgcctgtaaccccagcttgggaagctgaggcagaagtactgcttgaacccagaaggcggaggttgcaatgagccgtgatcgcaccactgcactccagccgggcaactgagactccatctcaaaaaactaaataaataaaaagaaagaaagaaaaaaagatgtacaaatacacagacctatgcatatatgtgtaggcattcatattaaacattaagataaaataattctaaaaacatgtcctgaagaatcaaaattaatgatgaactaaatataaattattagagtaattcataattgattttggttatttttaattgtttacatgttgtgattttacacaaaatattagtcatttatcctgataaaacaggtcaaaatgttatatcaagagtttatttactttgtattactaataatataaaatgttccacatatttttagccatgttttacttatctgtgaaatgtgtttattaatttttcattttaatatgtcacctttatctttctaatttcctggatttaattccactacagctgaatgcattttaataattattgtaatcacatttacattttttatttgaattttcatatatattttattaggattttaatatcaatataaatattttcctaccattgtctattttctatttttatgatataaaattaacataaaaatgcttagattttcaatgtacagcttgagggcttctgacaaatgtgcgcacaaataagaattatttgcttactttttagtgtatactgaggagtgggattgctgggtaaaatgatatctctgttttaagttctttaagaaatctccagtctgctttccaaaggggcaagattaacttatattcttctcatcagggtataagtgttctcttttctccagagtcccaccagcattcattgttttttgactttttcgtgatagccattctgagtggtgtgttgctgcacacctacaatcatctcatatttgataaggctgatgaaaacaagcaataaggaagggactccctgttcaataaatggtgctgggacaactggctagccatgtgatgaagattgaagctggacgtctactttcaacatacataaaattaactcaaaattgatgaaagacttaaatgtaagacctcaaaccataaaaatccttgaagacaacctaggaaatactcttctcgaccccaactttgacgaataatttttggctgagtatccaaaagcaaattcaaccaaaacacaaatagatgagtgggacctaattaactaaggagtttctgcacagcaaacccgccaggagatagatgctcaggacaccagagagaactcagttaaaccagggggcgctcagaacagcagagtgtgctcaggacaccagggggcgctcagaaccaccagcaggagctcagaacagcagagtgtgctcaggacaccagggggcgctcagaaccaccagcaggagctcagaacagcagagtgtgctcaggacaccagggggcgctcagaaccaccagcgggcgctcagaacagcagagtgtgctcaggacaccagggggcgctcagaaccaccagtgggcgctcagaacagcagagtgtgctcaggacaccagggggcgctcagaaccaccagcgggcgctcagaacagcagagtgtgctcaggacaccagggggcgctcagaaccaccagcgggcgctcagaaccaccagggggcgctcaggacggcaggggtcgctcagacaccacggttcccttaggaggcagctccacattaggtccctgggcagggtggggtttcctttttgaacttgctattttttgaccttgtaaagcaaaggtcttccccaggatctcttagtatttcttccttgtaactcatgtcttgtcttgttttgtttttgtttttgttttttgtttttggtcatctacaaaaacttaaacttagaacagttgattcagaaaggcaggaaagcaggcattcctctaagtctcccctgagacaaatacatatatgatttcttcctccccactataatttatgtaaaaatgaagattcactgagtcagactaaattgtgtattcagtggaaggataataaatgacacaaaagaatgcaacctattgtctcttatcttcttctaacctgcaagcccccattttcatttgtcctggcttacaggaaaaaaaaatgtacatttcacatgtattgactgatgtctcatgtctctgtaaaatgtataaaagcaagctgtacttcaatcaccttgtgcacatgtctcaggacttcctgaggctggttatggttgggttcttaactttggcaaaacaaatgtattagtctgttctcctgctgctgataaaaacataatcaagactgggtaacgtataaaggaaagagttttaattgactcacatttccacgtggctggggacacttcgcaatcatgtcagaaaaggaagggacatcttacatggtggaagactagagagagcttgtgcagatgaattcccctttagaaaaccatcagatctaatgaaacttattcactatcatgagttcatcatgggaaagacctgcccctatgattcaattacttctcactgggttcttccaatgacacatgggaaatgtgggactacaattcaaaatgagttttggatggggacacagacaaaccatattaataaacttcctaaattgactgagacctgtctcagatatttgttgtttatcctactcatgtacagccttcagagttccaaagcctatatcagttttccaggattgtttcccctttttgttggttatttcctcctttattttctatgtttagttccctttttctcccactattttttcatcattgggatgtgagacttcacaacatttgaaaggtaggtaacaatgagctatcttaacaacatgggacctgtttatctaagagtaatccattctatcaatgaaagataaaacaaaacaggagaccagaaactcatttggttgtaaaatgcttcctctgaatgattttgaaaaggaacaggtggggaaaatataaaaggaaaataaaaacttgtgctgtcaattcattatgtcatgaggggggaaaatcctaaaggatgatccatgcaagaaactgattttcctttcattcctaagagaatagctacaaataaaaagttaaatatcttcacagatagctactctttgttcattttactttatataaagtgctaacttagttcaggagaactacataatttttctgtttccctatgtgcttctctctcattacaacatgtaaattttcatactgtccctctttcccctctatccagctttccccccttttttgtattgaaagccctgaaaatcatctttggggaacggcactgaccacaaatttttctgtgattacttgtattttcattccaggcatgccctaactttggcaaaattaattttaatttgattgagatctgtctcggaaacctttggtttacactaggaaagatcccaaattacgagtcaattactataaagctcagccttcccactgtgtatgtgtgtgtgcatgtgtgtgtgtgtgtgtgtacacgtgtgttttaatttttgtgggctttgagccatgtagttctctctgtggacatactatttggcgtgatctttgaatagagaattctgaaagaaataagaggctcctatgagttctctgaaagtttctggactcaccatggatcttgactgtgtcattgcatctgacagtcccagggaacagactctctggtggtttcaaagaatctgtgcttgggctccccctgcagtttactgggtacagtaatgttaaatcactgtttcgagagacaatttcgaaagcattagatgctgctgagagaggattgtgaaccaggggacagccccttcattctgggggagcgacattgggagaatatgctctgtgagcccaaacagcatcctcccctgccgggtgagggcagagctgcaggacaggcccagaaaccactcaacacagatgtcagccctggagcagctgcagaggagtctgaggagaaaattttaccagcatctgaattacacttatttcaaaacaaaaatgcaattaaaaagttaaaataagtaattaatgtccaggcacagtggcttacacctataatcccagaaatttgggaggctgaggtgggaagatttccaggagtttgagatcagtctgtgaaacatagtgaggcttcatctttacttttgaaaataaaataaaataaaacataaataatttagcaaaaactaccgatgtgtcttccatatcccatcgtacttagagcattgatgtaacccaatgagtcaatgagaaccaaatttgaaaggagaaaattttagagttttcagatatcttaagagttggaagatgtagaacaaaactaattttatcaattcaatgtgtgcaaacatggagagacacactcatgccagtagttcaacttgcaaagggcaaaaaccaaaaaagtttgaagttgctagtgatccatttgtaggtgagatcattttgaggatcatgtcctgtgagaggctgtttctctattagaggagttctgtgctcatgaagtgctggacatgctaggggacaaatatcagtaaacaaacatcagaacttgaatgtcagcttccccctgctgcattctccgtgtgtcatctctctgttatttctcatgctagatcaggtctttagctatgaaatattccacctaatttacatgtcaatagcttgaagtctactgagtttaattcatattttttctatatgttacagttacagtttgtccacaaggacaaggtttgtccacaagtttgtccacaaggttacagtttgtccacaaggactcaatcatagaagtgcagagtccttttcgggccacatttaattcactttatcagtgcccttcagtatgtggttcctgagaatttcacatgacaacacatttaccacactggaatttaagcaatccaacacatgtttgtagctttatcttgtaataggctgtatttcatatggcagcctctgcttcagtttagctaacactatggctttgtttctctctacaagaacttgtttctcccaagatttccatgtttgtgaaaggaaaagaaatctctgggacccccaaatcactaagccaaagggaaaagtcaagctgaaaactgcttggggcaaacccacctccattctttccctaaaatgatagctactaactgtcaggcctctgagcccaagataagccatcatatcccctgtgacctgcacatacacatccagatggccggttcctgccttaatggatgacattccaccacaaaagaagtgaaaatggcctattcctgccttaactgatgatactatcttgtgaaattccttctcctggctcatcctggctcaaaagctcccctactgagcaccttgtgaccccctctcctgcccgccagagaacaacccccctttttcctttacctacccaaatcctataaaacggccccacccctatctcccttcgctgactctcttttcagactcagcccgcctgcccccaggtgaaataaacagctttattgctcacacaaagcctgtttggtggtctcttcacatggacacgagtgaaactaaggttttaaaaaactacatacctccttcaaaatttgaccacaaggaaaaaccttgtggaccaacaacagacagagtcatttctctgctcacataagtcaaatgcctatctgattgccaccatttctctattgttttgctaagccagactaaggcctacgtgactattcctgtaaattgtgcattcagttaaaggctaatcagaaactcaagagaatgcaagcatttgtctcaaacctacctgtgatctggacgctctctccccacttcagaaagtcctgcctttctgaaccaaaccaatgtacatcttacatatattgattactgtcccatgtctccctaaattgcataaaatcaagctgtgtcccaccaccttgggcatatatcgtcaggactccctgaggcggtgtcactgccatgtccttaatcttggaatacgaacttcctaaatctattgagattagtctcaggtactctttggattacaggtttgttttttatttcataacttcaattatctgaaatactaaacaaaatttgccaaagtgtacattctcttgttgctgttgttgcaaaaaatatatttatgtaacttatatataatttatcatctatgtgtgttaccaagctgagtagcagatttattagtaagacttagagagagtaatgaaaaattcaaatgccagttagtaacttaatagaaaaacaaattatgctacatttgtttgctgaaatgctacccattatttgtaagaaataaacacatgcaacataaaagatttaattctcagtatttctattgagagaaataatccacatacataagagcatatactatattttttcattcttataaattctagaatataaaagctagtcaaaagacatgtgaaaacatcagtagatttataaagaaatggtagaagaagggaaggagaaaaaccaaaaaaatataaaagagcaagaggaatgctgaggggagttgacttgtcaccttcttaaaaatagagattttatttttcaaagtttactattgtacagaataaatatgtgaattttcttatctgtcaattaaacctcataaaatttattacaaaaaaactgaaattttagacaaaaagagggtgataggaaggaacaaataaatatgttaaatgtcaaatatacctaaaaatttatttgtctgacccctagttttctccgtatttttaggtaaatgcagcaaaatcacacaagttgtcgtggcaggaagtggattctgcaaaccacactaggcccgtttatctctgtcctagagttggttaaaagagcaactgaggccagctgtgaggagcataggcccgggtactaggactcactcatgccagatataagcccttagacacatacatagcccctccatgtgtgggttcacttttacatctgtacatgaagaaaccactgattcctaaataacataatttatacacataggtaaaaataattaaaaatgtgatagttattaagtgtttatcacacaacaatttcacaataaaacagcattttcccaaatgtaatcattgtcatcgaaatccccaaggacactctcatctgccctgggccctgccctctcctcaggcatctcaccccagagcttgctatatagtaggagacatgcaaataggtccctccctctcctgatgaaaaccagcccagccctgactccgcagctctgggagaggagcccccgccctgggattcccaggtgttttcatttggtgatcagcactgaacacagaagagtcatgatggagtttgggctgagctgggttttccttgttgctatttttaaaggtgattcatgaggaaatagagatattgagtgtgagtggacatgagtgagagaaacagtggatttgtgtggcagtttctgaccttggtgtctctgtgtttgcaggtgtccagtgtgaggtgcagctggtggagtctggggaaggcttggtccagcctggggggtccctgagactctcctgtgcagcctctggattcaccttcagtagctatgctatgcactgggtccgccaggctccagggaagggactggaatatgtttcagctattagtagtaatgggggtagcacatattatgcagactctgtgaagggcagattcaccatctccagagacaattccaagaacacgctgtatcttcaaatgggcagcctgagagctgaggacatggctgtgtattactgtgcgagagacacagtgaggagaagttaatgtgggaccatgcagaaacctccctgcgggaacgctggggaaagtcatctgcagggggcgctcaggagccactgatcagagtcagccccagcggcaggtgcagatgaaggctgatttcctgtcacgatgtgggacttcatcttcttaaagtttctctactgaacctaagttcggaattctgtgattactagggtcatttctacatatttttaaaatgattgttttaatatgaaaacctattctcctatgcagaaaatgcagattgatcctcacagaggagatgaaaagttctcaaccatggtcaccactgtcagagtcctgaggaagctcagggcttcctggtgagtcttctccagtcagactgaggacaggaactccagaaagaatcctgactagaactgaggtattgctcctcacaagagaactcaagctgcggggggtctgttcctgcagacccctgacccggtggcggatgaataaaacgtacactgacacacagatattctgttttgccagttcagctgagggtccgaggcaacttacagactccaagaagaatcctgtaaagaacggcagcagtggccctgagcagctcgcactccaggcgtgtattgagtatagaattaacaacagaagctctgacaacacacttgtggataattaacatggttaagagagtagttctacgaatgattaaagctcagggactgcggtctaaagtaaataccattagggggcaataccctggttagttaatggaggtagggtaaccagacttaactggggagccctctattgtccctagtatttaccctatgcctaacgctctaaggtaagagctgcccaccttcagcctgttcaattattacaagctatgtaacctttcggccttctagagaggtatatgactattccctataactttccttaatattcccctttaatatttccgccaccatcctgagtgaatcccagcaaggacagtctttggggattctgattacagaaaaaagagagcctgggccagggtcagtgtcatgtagaacctcacaggtttctcgtaggacccttctcctgacactgaagaatgcaaatcagaatcaatactgatctggagcttcttttcctaattcatttactgtcttttttagatgttgttctcatttttccatttgcttttcctgctttccgaaaaaggaagatgtttttgctgtggtcaaaattccagacctcaagcccttttcttggcgctcaggtgggtctcaggctgtggctgctgcagtcacgcaggagagtctggtgggactttcttcactcttcgtcactcaggaccctccactgtgttgcatggagactcatctggaaatgcaagttgccagtgggaactgaagggacaagcttgtctggttaatgtgggatgtggatgtgtttctaatcctgttctgaaaaaccgtcacacagtaacgttcttcactagtggagaaggaagtgggtgtgaacgttgtcagaataaaaatggagccacgtgtgttataatctttacaggcgaagctggaagaggtcatgaatagagggttctcatgcacacatccctgataacaagaattaccctaaaaatactctgcacaaccacaatcttgaacaaaggctaccacaacaataagagaattaatactgaggggatatctgccctgcaactccctgttcagccttaaactgattctacccttgttattgattcttctaccccaggataattgtctcaaaatagctcaggtaatcttctcatttatccttcagacacttgtctttctttaccaacctaaatgtgaccatggataatcccattgcaatgctcattttcaaataaatacgatttgattttggagaatctctttctctctgatgtttaggtttgacaagctgcagagggacacaccactttcctgtgagatgtaggggatgacagttttgggggatggctggaaacgtgcagtatccttagggtcagccatcagtaaatgcaggctggaaatcttagaaagatctcaagctgcttaatcaccacggagttttaccttctccagatctgttctgatggaatcagggccaagtttgttattgatgataatctacctaatattgagtcaactgatcacagttttaataacatctatttaaaaattcacacctggattagtgtttgatcaaataactacaaagtattgcccaaccaagcataccatcaggcagactgttacccacagagaaaaacattgaacatgagtttgaggttctcacattgttaaaggtgtaaaaccgattatgtttaaattatgctatttttattattgttattgagttgtagatgtttcatttacattttggatattaacgctttttcagattcatggcatattatccaattctgtgagttggaattattttgttactttgcggaatctttttttaaatctagtcccacttgtccaattctgtgtttttttatttgtttgaatgtaaaatccagaaaaagattgctaattttttgagggttgagagttttacaattgcacgtgtttcatttaagtatttaatgcatttagaattaatttttttgtttattctaacctaaaattcttaattctttgcatgtaaatatccaattttcataacacgctctttggaagacactataatttagcaattatatattgatggttctcacgctgaaagtcagctggccatcaatatgtgggtttatatctaagctctctgtatgaatttatgcgaatgccattctgatttattactgtatgtttctaataaatgttgaggcctggaagtgtaatacctcaagctttattcttgccttgttacagatattagaccaaaatattctaaaattttactattgagtataataatagctgtggcttttgttaattggtttcattatgtacaagtagttttcttgtcttcctactttgttcagagtttttataatgaaatcctgaatttttttcaagtgttttctgtgtctgatgaaatgttactgagatattttttctttagttttttaattaatcaactgaattgattgatttgagaatgtcaaatcatctgtgcatctcagaataaatttgagttgatcatggtgtatggtcttctataaaatctttagaatttattttactattgttggggggttaatttatgtctactaatgatattggtctatgattttcttttattgtggtgcctttgtctattactggtagtactctaacggtagcctcatagaaagagtttggaaggtgtgttgcagactacctttaaaatagattttatcagtggagaaacggtgatagtttttttcttcagttttctgttgggaagagttttgtttgtttgtttgttttttgtttttttgttttgttttgtttgttttttgagacagaatctcgctctgttgcccaggctggagtgcagtggcagaatctcggctcactgcaagctccgcctcctgggttcatgccattctcctgcctcagcccccaagtagctgggactacaggcgcctgccaccacgcctggctaactttttgtatttttagtagagatggggtttcaccgtgttagccagaatggtctcgatctcctgacctcgtgatccgcccgcctcggcctcccaagggaagagttttatgttgtttaaaagatattctgaataacttaacctgcttaatgggcttacataatattcctttcttccattctttttaaagaactgcttacctttcctaattatttttagggtgatttttcacataaagattgtgcaatacattttgaggtgagacttagtggattttctctaatgaattagaaataataaatcacttaattgactattgtattaaggttgatttgttgaatatttgctaaaggccagttctttaagctgtgccatgtactaaatccctaaccgactattttattcaggttggtttgttgaatatttgttagagaccagttctttacactatggcatgtaataaattccaaatggcagtacgtcattgtttacttagcttttgtgcttatatttttcagaggaaaaaaaatactgtaaattgtaaatacccaatacctaacagtattgtatgcaaatctgtgactgttggcagtgtcatctctgagaaacagataaagtttatttactatatataaaaaaagagtttggaaggtggactcctcaccaatttttgaaagagttagagaagcattagcattaattctttaaatgttaagactaattttatgatgtaacatataacctatcatggagaatgttcaatgggtgcttgagaaggatgtgtattacatggctcttggttggaaggttctgtaaatgtctttcaggtaaatttgttcaatactgttgttcaagttcagaggcttattaagaattttctttctggatttgcaatacattattgctaagtgaggtattaaggttttcccttatttttatatagttttctatttctctctatatatatatcttaaagtttgcttaatgcatttatatttgtatttgtactgtgtaaaaagtaaaataaaataataattgcttagtgagtttcatggcacaatcacgttatgaataatcatattttcccaaatgctgtcattgccactaactcctccagatacatggtatattatccaattctgtgagttggaattatttcgttgctttgcagaatctttttttgttaatctagttccacttgttcaattatgctcctccgggagtctcacatctgctctgggcactgccttcttctcaggcatcccacactggagcttgctatagaggaggaggcatgaaaacagggccctccctctactggtgaaaacaagcccagacctggccctgcagctctgggagaagagccacagccctgggattcccaggggtttccatgtggtaatcagggctgaacacagagctcactatggggtttgagctaaccagaatttttcttgttgctattttaaaaggtgactcatagagaaatagagtgagtgagagtgagtggatataagtgagaaaaacagtagatgtgtttggcagtttctgaccaggacgtttgtgtattttcaggtgttcagtgtgaggtggagctgatagagtccatagagggcctgagacaacttgggaagttcctgagactctcctgtgtagcctctggattcaccttcagtagctactgaatgagctgggtcaatgagactctagggaaggggctggagggagtaatagatgtaaaatatgatggaagtcagatataccatgcagactctgtgaagggcagattcaccatctccaaagacaatgctaagaactcaccgtatctccaaacgaacagtctgagagctgaggacatgaccatgcatggctgtacataaggttccaagtgaggaaacatcggtgtgagtccagacacaaaatttcctgcagaaagaagaaaggattctgggccgaaggggacactcagcactcacaaaacaggtggagccccagggcaggtacagaaaagcagtcaagggctgctgtccttcaggatctgtgctttcctctgcatgtagcagttcccctcgaatcctctgcacttttatgtttctgtgcccaccatgaggtccctggattacaaaactttaatttaaaagaggaaacattcttatatgtcccaaaaacaaaagtaagtattagaggcacaaaagtgcacaggcggccaggtgaggctgtagacactgccaccccgcaatgccagtctcacaactagcactggagagtagtgggcattcagtggagcttcctacctatcctgtggtccgagctaagtccagcaaggccattggtgccttccagagcacagtagtccatcaggaatctcccatgtgtcccagcagcagccttgcctcagtatctccactgtgcacagccattgtctgggagggagctcccaggatgagtgtctttggcacacaccaggtggcgggtgttagagtgcagtacagcagctggctgcctggtctattgggctccctgatgttggagggatttgaggtgcattctcaggcccagcaccctctttgtgaatttttatataaaaaccctgattttacttcattttctcagatgacatagataactaaaaacagaatctgcaaagaaattgtaattttcaactttaccccaaattcattgtttcttaattctgtgcaagatccaaacatatttttggcttccacatgagaaattgttctgtttaaactgaaatcatttcttctcatattctttcttttgggccaagtacagagatcttgttgaaaataagttgggttctttccacacactaaccctcacctcccctagagaaagagcagagattgtcctcactctgagtctaggggaggagctgttcctgtacaattcagagcctgcagagaccccccccccccgccaggtgcagcttcagtgagtcagacatttctccatgtgggcgacctccagtgcctgtgactgcttctcaggcctaattgtgggttaagaattaggacactctttaagttatcacatctgaatcctattctgaaaatcaccataaagagagatagttcaatggctattctcctgacataagtttctctttattacttggttccaagtatggagaaacatgtgacccaatatttgtctgaatccaacatcagaatcccctgcatttctctgggaaactcccggatcgaacacaggtgagctctttattctcacagaaatgtatgtagttgggaatttcaacaaattgtccagaacctgtgcctgccaacaactgtgtttctcggtgcactcttggcctggtgaagcccccaaagaccctctccctcatgtgtgtcatctctgcattctccatcacaaccagtgcttcctcctggatctgcatccatcaccccctctctgggagggaatggagtggattgggtccatagatgatgaagggagcacacattactcccctttcctcaagagtccagtcaccatccccagatccatgtccaaaaacagttcttcctacagctgagctacatgagcaacaatcacatagccatatatttttcagcaatagacacagtgagggaatacacagtgtgaactcacaccaaaacctccctgtggggatgcataggacagaaggggttgctcaggtccccagggggctctcaggacaccaagggtcactcaagaccattgtagaggcacgcaggtagccgggggctctcaggaaccatgggggaaaatcaggacaccaaagggtgcttggtacagcagggggctcaggacaattgtggggattcagaaagaacagcttcaaggctcagcttcagggcaggtgcagctggtgtgaaaaggggctggatgaggcgttttgtgtcaccatcatgaaacaccaccagacaccctccactacatctgttctaatgcatgtgtttgtatgattaggaaatgatattgatataaatatataaccatagctaggtgtgtctttttctgccttccgtcctcccttcttctctctctctcacacagaaatttacatacacccaccccacaacacacataaatctataacttttattacctgatgtattcaataaacctgattaatgtgttttgcagcttcattgtttatggtgttgtagcaataaaaaccatctgtttcccagctgtgtacttctctaagctgagtagcatctttgttcataatacccagaatgaaaaacaacccaaatgtcaatcatcagcttaactggtaaacacattgtggaaaagtcattcattgacatactacccactactaccatcagctaatgttggctatgctccaaagcatggttaaattcacaagtacttcagatgagtaaaatgagccaaagtgataaaagtgcatacataggatacaactttcataggttctatagaaccaaaagtaatctaaagttgccaaaaaaaaatcagtagttcactgtgaatattgtaggagaagggatggtctaggaaggaggaattatggaacaagataaaatgttgagggaattgacttgttatctatgttgatagtgataatgtctatgacaatatttgcccaattgtacacttcatgtggagattattgtttttagtttagccccattaaagatagtattaattagaacaggtataaattggtatcacaggaattaaagataaataaaatacatgtaaagtcagagaatcctgaatatacacatgaatgagcactggacatctctgtatttttagagaagcactagaatacagcaaaataatggcacaattttacatcactaagaaagtttatcaaacccaccaggcatgttgtattagttcattttcacactggtataaaaaactacttgagactgggtagtttacaaggaaaagaggtatagttggctcacagttctgcatggctggggaggccacaggaaacttacaatcatggtggaaggtgaagaggaagcaaggcacgtctcacagcccagcaggagagagatagaggaggggaagtgacacatacttttaaatcatcagcggttgttagaactcactcactatcatgagaacaacatggggaaactgcccccatgatccaatcacctctcacctggtccctcccttgacatgtggggattagaatttgagatgatatttaggtggggcagaaaaccaatctatatcacatgtccagttctgtcctggagttgtttcagggatccagtgtgtcctgttgatagaaacagtgacaccaagttcatattatcagttgtagttgacaccatgcaaagccaagagatctcaagtgagatttagtgtgtatgttgtgtctaatgaagtcacacactcagagcaagtgaatagggaaaagttcattatctacactgtagaggtgtctgctgagtgcagggcaggtctcccaggaaaatctaaaatggcttgaaagaaaaccaaaggagactggctcagggtttttataatggtttagtggtgggggcaaagtgaagcttcccactcacagatgggggtttatagggtttgaaactcccactggcatcaaatgaagaagctcctgtgagtctgaactagattcatcttgtgtggcaaaaaaggagacgatggaggagtgagccttaagtaatcagcagtcatgcaccaaaagatagagtgacaacttattccatgcagcaggaataaaaataatgaataagaaagaagacaagggttcagtatgggtggacaacacccagatctgcagaaatgagatgactttagaaatgtaagcaaagaataatgagaaaaagaagaagtggatggaaaattaaacaggggcctggtctaatgtcttgggtagaagcttttcacaatcaaggactatcagcttattctgcaggtcttaggtcagccatctgcttaaaaacatcagaaacgccagagggtctatgaggatgctcagtttaacatctcctatttgagtagctttacagttgtgtggaattcttaattgattcttttttgtttgtttttagggacaggctctcacactgttccacagcttaaagtacagttgtgtgatcatagctcaccttaaatttgagctcctgactcacacaacctttccatcttagctttctgagtatctacaactagatgggcacaccacccctaccctccttattttttaaacattttttcatagaaatagcatctctttatgttgctcaggttggcttcgattgcctggtctcataggatttccctcactttgcttctggaagtggtgtgattatagggaagatccactgcatctgacctgaatttattctttagttgtaaaatatgaagccaataattaactgcctgaatgttttctgcagtgagttagttaaaagcatctgataaggttccttcctatgtgattcacgagcagtattttccgctgatgttccttccagtttccttgctgaagatcacagggaattgtggaaaatatgtagcaaaaaggcagccttaaactcttcattgttagagtggatatcacacaggaatcactttcagtattttgtaacacatgcatgcaatagagcaaacatgatctctgggggtaaagtctataaacgtatgggccttttagctaccaagtcatagggtaataactgatgtaccctgaggactggaccatgattccatagtgctagtggaagaacccttgaccaagggagttttacattttattaaaatatgtagagatgccactttttaaacattcccagaaggttgtgagtgggattgactctgtctcgtatgaacaaagacaatgccctccatgattagataatattataaactagattgagctacagtgtttgctgtattgaatcactgtatattttattttttagctccatgttagttttgtgtctgcgtgttagcgttggctttaaaatgatattaatcagctctgtagtaagtagaaattcatctgagagtttctttccttgttttccagtttgataggatttccagaatacataagaaccctctctgtttgtaaaaatattccaggccaggccagtggctcatgcctgtaaacccagcactttgggaggccgaggtgggggggatcatttcaggtcaggagtttgagaccagcctggccaacatggtgaaacctcctttctactaaaaatacaaaaattagccgggcatggtggtgcttgaggattgcttgaaccctggaggctgatgttgcagtgagccgaaattgtgccactgcactccagcctgagagacacagcgagagtccatctcaaaataaaagaaaacaaaacattccaaagttatgcacaatctagaaacatatgtacttaattctcatttttaatttattaaaaagctctaataagttcaatgttttctgccttctcagttgatttcacaacacatataataatatatcctaaataaaagtttgttctcttaatacaaattactagttaataacccttactttattattgaggtattatccatcagtgttcatcaatgctctcaatgggactcttacatagagaatatacaaaatattttcctgatcatgacataaaacagatgtgaacacattcttagtattcagccatgtctcctatcatgttataaaccacatgctaactttgactttattggaacttgttctaatttcaaactagttattttttatcttcatgcagctggattattatgtgtggttatttattccgagagtgataaagacaacattaacaattttcactgcaggcatgtctaggcaaccccctgtgcacaatgaccttggtgcattggagattctatggggactcttccctacctgcctaggagagttctctgccttctacctctatcattttcctctttgaagaaatacatctaactgtcgttagaatagagacaaagacaaatcttaactgcttccagctgacgggggatgctgtttggggaagatctctcttggaggcctgtctaagggaccccaggaaaagggagccattatcccaggcttcagttgcatgagcatttggagtttgatggtctgaaaacgagaaaaggcaaatcagattattagaagacatgtatccaaacctaacaaggtggtcaaggtggtaaggacagtttgaaagaaaattccaaggctgctgacatgcccagataactgcggctgtagttatgcctgctaaggtttgggcgcatgaggcttggcttttgtcagctccctgggatttattttcccaaacaaagaaacctccaggttaggggcaccctattcattcccatcacctggcatgatttaaaggataattgcttagaattaaaatattgatacagatttttttatattccccatcgctttttgtttcttctgggctgtagccagagatcattgattggcgctcaggaataagcagtcagtctaaaatgcaggcaaatacttaaacaactgaagagattagaatttaaagatgatcgtatgatatgttttgaaatacaatttttctctttccagttctggtttttgtcagaatcaagtaattgtaagactgagttgtttgcaaaataaactttagtcttaaacttggcctgattatttgcataaagtgcagcaagaatattaataataattctgtaggaaaagcctgcaagcaccaggagtttcacagtctaacactatgagcacgtgcatcctcacgcaactcgctgaatatttccaagccagcctgttcctatcttaaatgccatccagttgtatctgccccaggtacactaatatatgggtcctgcttctctgcagcctcctctctcctcagatttcaggttttgtttattgtttgttttctctctgatataaactcaaatatgttgaaggtttttttgtgtgtggttgttcaggtttgttgttaatgagatcagaataagataatactttactctttttatttatttatttatttattttttgagtcggagtctcgctctgtcacccagactggagtgcagtggccctatctcagctcactgcaagctccgcctcccgggttcacgccattctcctgcctcagcctccagagtagctgggactacaggcgcccgccaccatgcccggctaattttttgtagttttagtagagacggggtttcaccgtgttagccaggatggtctcgatctcctgaccttgtgatccgcccgcctcggcctcccaaagtgctgggattacaggcgtgagcctccgcgcccggcccatactttgctccgttttatattcccatgctttagtagctgcttttctctatcaaatccattaactgagagaacaaatcacatttagttacaggtgaacaattaaatagtttggcatatatttatgtactggaacataacgcagcttgaaatcaaggcatgcctcactcatataaacaacatggctaaattctcaagtaattctgtttagtgaaagaaactaaggagttaagagtaaattttatatgatacatttgtaggaattttagaagatgccattattgtaaattaacacggagaagatttgagtttgtctgacaatacgctgttgggagtaatgtggatgtgagttgaatttcagagaaataaaagaaagatttagggattaatttaattattcaaaacttgattgaagtgctgagcaaatggctgcaaacataggtcaacatttttcaaatccttcactataaatttgaattaattacttattaattacacttgaataaagcaataacgaagaaaccaataaaataacatttgactaaaatggagcaataaacagatcgatgttaacacaaggaatatgactgacttatgaaaacatgcacatgaaccatggttcactctacgtattttggtaaattacagaaagttgtcataacagatggggaatcctgcagacttcactaggcatggtccacgctgccctggagttgtctcaggggagctgcctcctccagtggttagagcacaggcccaggtaataggactaatttttttagatgtgtaattttagacacactgcacaactgctgtgttctctgtgtaaattatctcctgtaaaatgtaacattgaaacctgcattaaacatattgtataaatatgtaagaataaaataagattatgagagctaaatattaatcaaggcacaagcacataatataaaattatattttcctgaatgatacaattattccaatctgtcccaggacacttcatctgccctgagcccagcctctcctcagatgtcccaccacagagcttgctatatagtgggggacatgcgaatagggccctccctctgctgatgaaaaccagcccagctgaccctgcagctctgggagaggagcccagcactggaagtcggcggtgtttccattccgtgatcagcactgaacacagaggactcaccatggagtttgggctgagctgggttttccttgttgctattttaagaggtgattcatagataaatagagatgttgagtgggagtggacatgagtgagagaaacagtggatgtgtgtggcagtttctgaccttggtgtctttgtgtttgcaggtgtccagtgtgaggtgcagctggtggagtctggggaaggcttggtccagcctggggggtccctgagactctcctgtgcagcctctggattcaccttcagtagctctgctatgcactgggtccgccaggctccaagaaagggtttgtagtgggtctcagttattagtacaagtggtgataccgtactctacacagactctgtgaagggccgattcaccatctccagagacaatgcccagaattcactgtctctgcaaatgaacagcctgagagccgagggcacagttgtgtactactgtgtgaaagacgcagtgagaagtcagtgtgagcccagacacaaacctcctgcagggtacctgggacaatcagggaaagcctgggacactgtatactgggctgtccccaggggcaagtccaggtgatataagcctgggtttcctgtcatgatctagggttgcattgttagcaaattaccccagggaccatctctagatttccaattctgtaataacatttgatgtcgtctctgactgcacaatgtcccctcaactttgtatcttttttttttttgtaacaggaggacacatcctcaccctgcagaagcctgagtgtcactttgggggcagaaatgacctgccttgatcacattgatcactgtcctgaggaaaataccccacaggggaccccgatgactccagcaaaggctctgcctcaaaaccattgaagagtccttcctttcattagaattgaccacagcacctgggcttcagcacaagccataccacagacgtcacaaagcagcagcttgacacctgatccaggtgcattttctcacctttagaagctgagagaggggtgtattctcaaggtcaacacactctttgtgggtttttacacagaaaacctgcttttactttatttcatttgaagataaatgaacaaatgtgcatctataaatgattgtaattttcacatttattccaaatccaatatttctgaattctgcatgatgtcctggcacagggttgtgttttctacaggtatttttcatcaaaccaagaaaatgcattttccactttccgtggtttttctccatgtgcagaggccttgagtagagcacgtctgactgtgtttgttttcacgctgctgataaagagaccttgagtagatcacgtctgactgtgtttgtctgttttcacgtggctgataaagacataccagagactgggaaacttacaacagaaagagttttattggacttacagttccacgtggctggaaaggcctcacaatcatggtggaaggtgaaaggcacatctgacatggcggcagacaagagaagagagcttgtgctgggtctcccctttttaaaaccatcagatctcatgagactcattcactattaagagaacagtgcaggagagacacaacccccataactcagtcacctcccactggtccctcccacaacattgagaattatgggagctacacatcaaaatgagatttgagtgaggacacatccgacccatatcattccaccccaggttccctcccaaatctcatgtcctcacatttcaaaaccaatcatgccttcccaacagctccccaaaggctaaactcatttcatcattaactcagaagtccacagtacaacatttcatctgaggcaaggcaagtcccttccccttatgagcctgtaaattcagaagcaagttacaagcattgggtaaacacacccattacaaatgacagaaattgcccataacaaaaaggccccatgcaagtccaaaatccagcagggcagtcaaatcatagagctccacaatgatcacctgtaactccccttctcacatccgggacatgttgatgcaataggtgagttccgatggtcttgggcagctccacccctgtggctttgcagggtacagcctccctcctggctgctttcacaggctggtgttcagtgtctgtggattttccaggcacaaagtgcaaactgtcagtggatctaccattccagggtctggaggatagcagccctcttctcacagctccactaggcagtgccccagtaaggactctgtgtgggagctctggccctatatttcctttcctccttgccctagcagaggttctccatgaatgatccaccactgcagccaacttatgactggacatccaggtgttttcatacatctcctgaaatctaggcagaggttctcaaacaccaattcttagcactctgtcgataatggttcattttggtggcctgttcattactggtattttccaaaggaatctcacttgaatctttactcttttgcattttgtctccatgacaatgttgggaagttttacctccaccatcataacatgatctagtgatctcacacatttgtggcaaacaatacctacaaattcagaagctctttgcttttctttccatgaaatataattctttctgttctgtgtataagcatatcttagcaactctgtgcacacccacatagatgtccacaagcctatgaattattctctgtaaataaaaatttatatcaatttccctcaatgttcataattctcctgagggtgaggaagctccttctcgatctgttcaaacaaaatgcccagaaaccatctggtaggtaaggagttcacctggctctggtgtggggtctgtctctttccctctgttgtcacacaggtcagcccagttgttcaggtcctaagaagaaagcccaggtttgtcctgattttaaaacacatcaaacttctgatgactctcctgttacccacatccatggaggatagattatttattatataattcaccaaactaatgtcaaatgcccaagttgcaataccacacatcctagggtatgttcatgcaattcactggaggagaaagtctttcagagacagatggatctgaaatgataaatatgtgggtaaggactctgggcttgagtatcattgtccagccatgtttcacaagtgtgtcctgtcaggggaaggacaagagttccttgtgttctcagagggaaggggtcacagagttcctctctggttcccaggaaagataatcgcactaatcttcatgatcttcatgagactatcctccagtgctgacctgttatagagtttttgtctgaagttctcactgcaatccccaatctacatattttcaatcagaagtgtttagagggcaggacatatcttcacggacacacattgagaaggatgtagatatgtcccactaccttctcctgagatctcagacagaatcccagatttcaaaaggacacagaaggacagctctcaggtgcttttaaaaaatgacccacttccagggacagggagcttccctataaccatggtggatgttctgaactacaataaacattggatggatccaggattgtttgaagtcactgtcattattacattcagctgctgtttcaatgtgtctgaagtagtaaatgacaatttagatgacaatttatatgaatcttcaagggtagaacaatattgaccatattccaaaatctgtccttgatccatgatcacactcatctcccagaccaggtccttcagcacgtctctttacctgaaagaagaggactctgggcttggagaggggagaccccaagaagacaactgagttctcaaagggcacaggcagcatcctactcccagggcgagcccaaaagactgggggctccctcctcctttttcacctctccgtacaaaggcaccacccacatgcaaatccttacttaagcacccacaggaaaccaccacacatttccttaaattcaggttccagctcacatgggaaatactttctgagagtcctggacctcctgtgcaagaacatgaaacacctgtggttcttcctcctcctggtggcagctcccagatgtgagtgtctcagggatccagacatgggggtatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagacacagtgaggggaggtgagtgtgagcccagacacaaacctccctgcatggacgcggaggggaccggcgcaggtgctgctcaggaccagcaggtggcgcgcggggcccccagagcatgaggccgggtcaggagcaggtgcagggagggcggggcttcctcatctgctcagtggtctccgtcctcgccagcacctcgctgtcaccagggctcctctttctttattatctgtggttctgcttcctcacattcttgtgccaggaaagaaacgaggaagacaaattttcgtctatagttgaagcttcaccaattactaggaacttgcctacaagttcctgcatgacccattataacttatcgattaaaaaatatatattctaatgcttctcaccatctcttgatttgtatcatcaactgaattgtaccctctttgaaattcatatgatgaaaccttaaattcaatggatctatattggaattttaatgaaataattaaggttaaatgtggtcataattgtaagaccctaatgcaatagacgtgttgtctttataagaagaggaagagacaccagagacctctcacttttcacgtgcaggcagagaagaggccatgtggagacatagtgcactagaaggtggcccagtgcaagccaggaagaagccgcgccaagaaccagccctgccagcacactaatcttcaacattcagactgcagaattttaagaaaatcagtatttgttgtttaagccacccactcctgttgtcttcttatgaagatccagacagactaataccacataactctgttagtgctgtcccctggatggagaattagcctcctgaggctgggcacatctctcagatttccacataaaacaggtaaaaaatagtagttctgatataaaaacttgtcatgtccctgttggccaatttctgggcaaggtcttttaaataagccaagtttgcggggaaatggagaccatatgtttgtgggactctaaccgtggaatctactgcattgctctagcagaatcaaaaattgaacaccagtgagctctttattctcattaaaatgtgtgtctttggaaatgttaacattctgtccagaatctgtgctcacaaacaactgtgtttcttagtgcactcttggcctggtggagccctcgcagaccctctccctcacctgtgctgtctctggattttccatcacaaccagtgtttcctgctggagctggatccacgagtccacatgggaaggactggagtggaccaggcgcacacgtcatgaagggagcaaaaattcccacccactccttatgaatccagtcaccatctccaaattcgggtccaaaaaacacttgtttttacagtggagctatgtgagcaacaagctcacagccatgttttaaagaagagacagagtgaggggaccacggtgcgagctcacacccaaaccttcctggaggggtgcacaggacagcaggagtcccgatgatggaagggggtggtctggattccaggtcactctcaagatcattgtagaggcactcagctcacccgggggctctcaggagccatcgaggtaaatcaggacaccagaagagactcggtacagcagggggctctggaccattgtggggattcagaaacagcaggttctaggctcagccttagggcaggttgagatggggtgaaaaggggctggatgagggtgtttgtgtcaccataatatttcaccactagacacccgccactacatgtgttctactgcatatgagtgtatgattagaaaatcatacttctataaatatataaccatacgaaggtgtgtcaagttttcctcttcaacttctatcggccttgttcgtaaggactaattccctgtaattacttgaggacctcataaattgtggtcaggtttgtaggtttcctctctttccttgtctccctttccccttattctctctcacacaaatggacataatccccactccacacgcaaagaaatctataactttcattacctgatgtattgaataaaattgattaatgtgcagctttcccatctttgttgttgttcatgctgctgtaagtataagaaccatgagattctcagctgtgtacttctccaagctgagtggcagctttgcttatcatactgagatggccaacaatccaaatgtcaatcatcagcttaactggtaaacaaattgtggaaaagtcattcattggcatactactgactgtgaccacaatatactaatgttggatacgctcaacagtatggttaaattctcatatggtgagtaaaatgagccaaacaaataaaagtacacacatatgataccacttttatacattctcccatccaagtactaaccaggcccgaccctgcttagcttccgagatcagacgagatcgggcgcgttcagggtggtatggccgtagacacacttttatacattctatagaataaaaactaaagttacataaagaatcagtagttcactgtgaatattatagaagggaaagtgtagacaggaggaattacagaaaagagaaaattttaagcataattgacttgttatctatattgataataatgatgtctatgataatatttgtacaattgaacacttcatgtgaagatctttttttttagtttaacatcattaaagatagtactaactgtaacgtgtgtaatttggtagaaacggagtcagacagagataaaatacataaaaagtcagagacacctgaatattcacatgagtgagcctgcacttctctatgtttttatgaaaacactacaatacagcaaaataatgacatcattttatgacgtgaagggggtttattaaaccactccaggcacgttgtattagtttgttttcccactgctacaaagaactacctgagtttgggtaacttataaagaaaataggttattgactcatagttctacatggctggaaaggcctcaggaaacttgcaatcaaggcagagtgtgaaggggaagcaagagacatctcacatggcagcaggagaggtggggagaaccaccacacactttaaattatcagatctcctgagacctcaatctgagacctcaatcatgaaaacagcatggaggaaccaccccatgagcccatcccctctcaccaggtccctccctggacgtgtggcaattccaattcgagatcagatttgggtggggacacagaaccaaaccatatcacatgtccagttctgtcccagagttggttcaggaatcagatgtgtcctgttaacaggagctgtgacaccaagctcccagcaccacttgtagttgacgtcatgcaaaggccaagatatctcaactaagatttagtgtggatgtgatgttggatggtgacacaccctcagaccaagtggatatggaagggataattatctctattctagacgtgtctgctgagagcagggcagttctctcaggaaagtgcaaaatggcttgatagaacagggaaagagagggcctcagggtttggtggtgggggcagattgagtctttccactcacagaaaggggtttatagggtgggaaactcccactggcttcaaataaggaagctcctgtgattctggaccgatattcaccttgtgtgtaaaaaaggaggtaatggaggaatgagccttaagctatcagcagtcacacaccaaaatacagtgtgacaagttattctatgtagcaactataaaaataatgaataaaaaacgagataagggttcaatatgatgggcaacactcaggtctacagaaaatgagatgagtgtttataagtcaaagcaaataataatgagaagcaggaggagggtgtgcagaattagacaggggtccaggtccagtgacttgggtggaaaccttttgctattaaggattatcagcttagtctgaaggtcttaggtcagctatctgtttaaagacatcagaaacaccagagaatctttgaggatgctcagattaacatctcctattttagtagccttgcagttgtgtgaaaaccttttttatcttatttcattttattatttatttatttatctgttgagatggaatttcgctcttgttgcccaggctggagcgaaatggcacgatcttgactcaccgcctcttccacctcccagattcaagcgattctcctgcctcagcctcccagtagctgatattacaggcatgcaccaccaggcccagctaattttgtatttttagtagagatggggtttctccacattggtcaggctggtctcaaactctcaacctccggtgacccgcccaccttggcctcccgaagtgctgggattacaggtgtgagccactgctccgggccgaaaatcttattcattcttttttgtttgtttttagagacagagtctctctctgttgcacgttgaaagtggagtggtgtgataacagctcactgtaaatttgaactcttggctcacagagtatgttcctatgtatcgtgcaggtgtgtctgtgtgtgtttacccggcttggggtcctctaatttttggatctgtagttaggtgtttcacattcttaagatatttttcttaagaaatatatcttaagatataagtatatcttaagatatcattaattcttaagatattttctctgtttctaatttctatttttgaaatttgaaatatatctacactatactttttgatgttagttcttaaatttcttcactctttttccctttgaaattcactttgtgtaatttccagtaacggagttgaagtccctggtttcatttaagagctgagcgaattctactgctgagcgtgcccaaggtatattcaactttatactgcatcacttttgataattttatgaatttccatttgattctttcttagtatttctctctgtcagtcacctatctagtcttgcactgtgtccacattttctttcagatacttttaacaaattcaattactttaaatatcctacttaataagatagttctagtattgtgtcatttacaaatatcaattctgaggatttttccattatgactttggtgttttttataacttatatgactttgatgaatgttataatttttgttgataaccagccatatttggacccttgatattgacgtattattttattttgtgcatttctgcctgtatttgaccacaccctatctgtgctgggccttgattgtggagatgtctgtgcatctcttcagagctacatttgacatttacttttgcaacggctgtagcagttgaagtgtgttcttctatgtccactggagacttcagatactctagtgataccttgttttcatgcctgcttggctttgtctattcacctcattccattctacagagaatctccttcacattcctagggtggattaaagtgttatatttaactgccaattgtgaaattggcggaaggcattagactaaaggaagaaactgacctctcgttgggccataattctagaaagccgttgtgatcctgagtctgagtgtgaccttccaagttttcccgaccctcctgcaagtgagatactggtctgtg